(19) United States
(12) Patent Application Publication
(10) Pub. No.: US 2021/0315907 A1
(43) Pub. Date: Oct. 14, 2021

(54) COMPOSITIONS AND METHODS FOR TREATING BRAIN-GUT DISORDERS

(71) Applicant: Enterin, Inc., Philadelphia, PA (US)

(72) Inventors: Michael Zasloff, Philadelphia, PA (US); Denise Barbut, Philadelphia, PA (US)

(73) Assignee: Enterin, Inc,, Philadelphia, PA (US)

(21) Appl. No.: 17/264,782

(22) PCT Filed: Aug. 2, 2019
(86) PCT No.: PCT/US2019/044917

§ 371 Date: Jan. 29, 2021

Related U.S. Application Data
(60) Provisional application No. 62/714,470, filed on Aug. 3, 2018.
(60) Provisional application No. 62/714,463, filed on Aug. 3, 2018.
(60) Provisional application No. 62/720,453, filed on Aug. 21, 2018.
(60) Provisional application No. 62/732,753, filed on Sep. 18, 2018.
(60) Provisional application No. 62/739,496, filed on Oct. 1, 2018.
(60) Provisional application No. 62/789,492, filed on Jan. 7, 2019.
(60) Provisional application No. 62/789,468, filed on Jan. 7, 2019.
(60) Provisional application No. 62/789,481, filed on Jan. 7, 2019.
(60) Provisional application No. 62/789,438, filed on Jan. 7, 2019.
(60) Provisional application No. 62/789,502, filed on Jan. 7, 2019.
(60) Provisional application No. 62/789,441, filed on Jan. 7, 2019.
(60) Provisional application No. 62/789,439, filed on Jan. 7, 2019.
(60) Provisional application No. 62/789,470, filed on Jan. 7, 2019.
(60) Provisional application No. 62/789,478, filed on Jan. 7, 2019.

(51) Int. Cl. A61K 31/575; A61P 9/12; A61P 1/10; A61P 25/16

(52) U.S. Cl. CPC A61K 31/575; A61P 25/16; A61P 1/10; A61P 9/12

(57) ABSTRACT

The present application relates generally to compositions and methods for treating and/or preventing a variety of symptoms and brain-gut disorders related thereto with aminosterols or pharmaceutically acceptable salts or derivatives thereof.

### CROSS REFERENCE TO RELATED APPLICATIONS

This application claims the benefit of U.S. Provisional Application No. 62/714,468, filed Aug. 3, 2018; U.S. Provisional Application No. 62/714,470, filed Aug. 3, 2018; U.S. Provisional Application No. 62/720,453, filed Aug. 21, 2018; U.S. Provisional Application No. 62/732,753, filed Sep. 18, 2018; U.S. Provisional Application No. 62/789,502, filed filed Jan. 7, 2019; U.S. Provisional Application No. 62/789,438, filed Jan. 7, 2019; U.S. Provisional Application No. 62/789,470, filed Jan. 7, 2019; U.S. Provisional Application No. 62/789,492, filed Jan. 7, 2019; U.S. Provisional Application No. 62/789,496, filed Jan. 7, 2019; U.S. Provisional Application No. 62/789,468, filed Jan. 7, 2019; U.S. Provisional Application No. 62/789,439, filed Jan. 7, 2019; U.S. Provisional Application No. 62/789,478, filed Jan. 7, 2019; U.S. Provisional Application No. 62/789,481, filed Jan. 7, 2019; and U.S. Provisional Application No. 62/789,441, filed Jan. 7, 2019, the contents of which are incorporated herein by reference in their entirety.

### FIELD

The present application relates generally to compositions and methods for treating and/or preventing a variety of symptoms and disorders related thereto with aminosterols or pharmaceutically acceptable salts or derivatives thereof.

### BACKGROUND

Aminosterols are amino derivatives of a sterol. Examples of aminosterols include squalamine and Aminosterol 1436 (also known as trodusquemine and MSI-1436).

Squalamine is a unique compound with a structure of a bile acid coupled to a polyamine (spermidine):

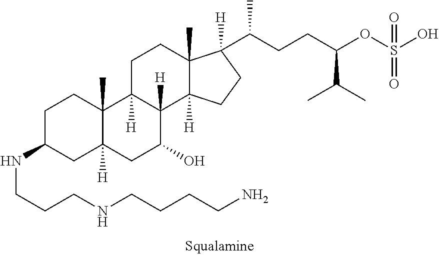

The discovery of squalamine, the structure of which is shown above, was reported by Michael Zasloff in 1993 (U.S. Pat. No. 5,192,756). Squalamine was discovered in various tissues of the dogfish shark (Squalus acanthias) in a search for antibacterial agents. The most abundant source of squalamine is in the livers of Squalus acanthias, although it is found in other sources, such as lampreys (Yun et al., 2007).

Several clinical trials have been conducted relating to the use of squalamine, including the following:

(1) ClinicalTrials.gov Identifier NCT01769183 for “Squalamine for the Treatment in Proliferative Diabetic Retinopathy,” by Elman Retina Group (6 participants; study completed August 2014);

(2) ClinicalTrials.gov Identifier NCT02727881 for “Efficacy and Safety Study of Squalamine Ophthalmic Solution in Subjects With Neovascular AMD (MAKO),” by Ohr Pharmaceutical Inc. (230 participants; study completed December 2017);

(3) ClinicalTrials.gov Identifier NCT02614937 for “Study of Squalamine Lactate for the Treatment of Macular Edema Related to Retinal Vein Occlusion,” by Ohr Pharmaceutical Inc. (20 participants; study completed December 2014);

(4) ClinicalTrials.gov Identifier NCT01678963 for “Efficacy and Safety of Squalamine Lactate Eye Drops in Subjects With Neovascular (Wet) Age-related Macular Degeneration (AMD),” by Ohr Pharmaceutical Inc. (142 participants; study completed March 2015);

(5) ClinicalTrials.gov Identifier NCT00333476 for “A Study of MSI-1256F (Squalamine Lactate) To Treat “Wet” Age-Related Macular Degeneration,” by Genaera Corporation (140 participants; study terminated);

(6) ClinicalTrials.gov Identifier NCT00094120 for “MSI-1256F (Squalamine Lactate) in Combination With Verteporfin in Patients With “Wet” Age-Related Macular Degeneration (AMD),” by Genaera Corporation (60 participants; study completed February 2007); and

(7) ClinicalTrials.gov Identifier NCT00089830 for “A Safety and Efficacy Study of MSI-1256F (Squalamine Lactate) To Treat “Wet” Age-Related Macular Degeneration,” by Genaera Corporation (120 participants; study completed May 2007).

Aminosterol 1436 is an aminosterol isolated from the dogfish shark, which is structurally related to squalamine (U.S. Pat. No. 5,840,936). It is also known as MSI-1436, trodusquemine and produlestan.

Several clinical trials have been conducted relating to the use of Aminosterol 1436:

(1) ClinicalTrials.gov Identifier NCT00509132 for “A Phase I, Double-Blind, Randomized, Placebo-Controlled Ascending IV Single-Dose Tolerance and Pharmacokinetic Study of Trodusquemine in Healthy Volunteers,” by Genaera Corp.;

(2) ClinicalTrials.gov Identifier NCT00606112 for “A Single Dose, Tolerance and Pharmacokinetic Study in Obese or Overweight Type 2 Diabetic Volunteer,” by Genaera Corp.;

(3) ClinicalTrials.gov Identifier NCT00806338 for “An Ascending Multi-Dose, Tolerance and Pharmacokinetic Study in Obese or Overweight Type 2 Diabetic Volunteers,” by Genaera Corp.; and

(4) ClinicalTrials.gov Identifier: NCT02524951 for “Safety and Tolerability of MSI-1436C in Metastatic Breast Cancer,” by DepyMed Inc.

Even in view of these trials, the full potential of aminosterols for use in treatment has yet to be determined.

### SUMMARY

In a first embodiment, the disclosure relates to a method of treating, preventing, and/or slowing the onset or progression in a subject in need of a condition selected from the group consisting of Parkinson's disease (PD) and/or a related symptom, autism spectrum disorder (ASD) and/or a related symptom, Alzheimer's disease (AD) and/or a related symptom, depression and/or a related symptom, or constipation and/or a related symptom, wherein the method comprises administering to the subject a therapeutically effective amount of at least one aminosterol, or a salt or derivative thereof, provided that the method of administering does not comprise oral administration.

In a second embodiment, the disclosure encompasses a method of treating, preventing, and/or slowing the onset or progression in a subject in need of a condition selected from the group consisting of schizophrenia and/or a related symptom, erectile dysfunction and/or a related symptom, high blood pressure (HBP) and/or a related condition, low blood pressure (LBP) and/or a related condition, multiple system atrophy and/or a related symptom, Cardiac Conduction Defects and/or a related symptom, wherein the method comprises administering to the subject a therapeutically effective amount of at least one aminosterol, or a salt or derivative thereof. The method of administration can comprise, for example, oral nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof.

In a third embodiment, the disclosure encompasses a method of treating a subject in need, wherein the subject has a condition amenable to treatment and/or prevention and/or amelioration with an aminosterol, comprising determining a dose of an aminosterol or a salt or derivative thereof for the subject, wherein the aminosterol dose is determined based on the effectiveness of the aminosterol dose in improving or resolving a symptom being evaluated, wherein the symptom is related to the condition, followed by administering the aminosterol dose to the subject for a period of time, wherein the method comprises: (a) identifying a symptom to be evaluated; (b) identifying a starting aminosterol dose for the subject; (c) administering an escalating dose of the aminosterol to the subject over a period of time until an effective dose for the symptom being evaluated is identified, wherein the effective dose is the dose where improvement or resolution of the symptom is observed, and fixing the aminosterol dose at that level for that particular symptom in that particular subject; and (d) optionally wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

In one aspect, the therapeutically effective amount of at least one aminosterol, or a salt or derivative thereof can comprise: about 0.1 to about 20 mg/kg body weight of the subject; and/or about 0.1 to about 15 mg/kg body weight of the subject; and/or about 0.1 to about 10 mg/kg body weight of the subject; and/or about 0.1 to about 5 mg/kg body weight of the subject; and/or about 0.1 to about 2.5 mg/kg body weight of the subject; and/or about 0.001 to about 500 mg/day; and/or about 0.001 to about 250 mg/day; and/or about 0.001 to about 125 mg/day; and/or about 0.001 to about 50 mg/day; and/or about 0.001 to about 25 mg/day; and/or about 0.001 to about 10 mg/day; and/or about 0.001 to about 6 mg/day administered intranasal; and/or about 0.001 to about 4 mg/day administered intranasal; and/or about 0.001 to about 2 mg/day administered intranasal; and/or about 0.001 to about 1 mg/day administered intranasal; and/or about 1 to about 300 mg/day administered orally; and/or about 25 to about 300 mg/day administered orally.

The present application also relates to compositions and methods for treating and/or preventing a variety of symptoms and disorders related thereto with aminosterols or pharmaceutically acceptable salts or derivatives thereof. Certain embodiments describe the determination and administration of a “fixed dose” that is not age, size, or weight dependent but rather is individually calibrated.

In one embodiment, the invention encompasses methods of treating a subject in need comprising determining a dose of an aminosterol or a salt or derivative thereof for the subject, wherein the aminosterol dose is determined based on the effectiveness of the aminosterol dose in improving or resolving a symptom being evaluated, followed by administering the aminosterol dose to the subject for a period of time. The method comprises the steps of (a) identifying a symptom to be evaluated; (b) identifying a starting aminosterol dose for the subject; (c) administering an escalating dose of the aminosterol to the subject over a period of time until an effective dose for the symptom being evaluated is identified, wherein the effective dose is the dose where improvement or resolution of the symptom is observed, and fixing the aminosterol dose at that level for that particular symptom in that particular subject.

In the methods of the invention, the aminosterol or a salt or derivative thereof can be administered via any pharmaceutically acceptable means. For example, the aminosterol or a salt or derivative thereof can be administered orally, intranasally, by injection (IV, IP, or IM) or any combination thereof. The aminosterol or a salt or derivative thereof can be formulated with one or more pharmaceutically acceptable carriers or excipients.

In one embodiment, starting dosages of the aminosterol or a salt or derivative thereof for oral administration can range, for example, from about 10 mg up to about 150 mg. In another embodiment, the composition is administered orally and the dosage of the aminosterol or a salt or derivative thereof is escalated in about 25 mg increments. In yet another embodiment, the composition is administered orally and the dose of the aminosterol or a salt or derivative thereof for the subject following dose escalation is fixed at a range of from about 25 mg up to about 500 mg.

In another embodiment, the composition is administered intranasally and the starting aminosterol or a salt or derivative thereof dosage ranges from about 0.001 mg to about 3 mg, or any amount in-between these two values. For example, the starting aminosterol dosage for IN administration, prior to dose escalation, can be, for example, about 0.001, about 0.005, about 0.01, about 0.02, about 0.03, about 0.05, about 0.06, about 0.07, about 0.08, about 0.09, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 1.0, about 1.1, about 1.25, about 1.3, about 1.4, about 1.5, about 1.6, about 1.7, about 1.75, about 1.8, about 1.9, about 2.0, about 2.1, about 2.25, about 2.3, about 2.4, about 2.5, about 2.6, about 2.7, about 2.75, about 2.8, about 2.9, or about 3 mg.

In another embodiment, the composition is administered intranasally and the dosage of the aminosterol or a salt or derivative thereof is escalated in increments of about 0.01, about 0.05, about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg.

Finally, in yet another embodiment, the composition is administered intranasally and the dose of the aminosterol or a salt or derivative thereof for the subject following escalation is fixed at a range of from about 0.001 mg up to about 6 mg. In yet a further embodiment, the aminosterol composition is administered intranasally and the dose of the aminosterol or a salt or derivative thereof for the subject following dose escalation is a dose which is subtherapeutic when given orally or by injection.

In one embodiment, the dosage of the aminosterol or a salt or derivative thereof is escalated every about 3 to about 5 days.

In one aspect of the methods of the invention, the improvement or resolution of the symptom is measured using a clinically recognized scale or tool. Further, in the methods of the invention the improvement in the symptom can be, for example, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%.

In another embodiment, the starting aminosterol or a salt or derivative thereof dose is higher if the symptom being evaluated is severe.

In another aspect of the methods described herein, progression or onset of the condition is slowed, halted, or reversed over a defined period of time following administration of the fixed escalated dose of the aminosterol or a salt or derivative thereof, as measured by a medically-recognized technique; and/or the progression or onset of the condition, and/or a related symptom, is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; and/or the condition is positively impacted by the fixed escalated dose of the aminosterol or a salt or derivative thereof, as measured by a medically-recognized technique; and/or the fixed escalated dose of the aminosterol or a salt or derivative thereof reverses dysfunction caused by the condition and treats, prevents, improves, and/or resolves the condition-related symptom being evaluated; and/or the improvement or resolution of the condition-related symptom is measured using a clinically recognized scale or tool; and/or the improvement in the condition-related symptom is at least about 3%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale or tool.

In the methods of the disclosure, the condition can be any indication amenable to treatment with an aminosterol. In some embodiments, the condition is selected from the group consisting of a neurodegenerative disease, Parkinson's disease, Alzheimer's disease, schizophrenia, autism spectrum disorder, depression, erectile dysfunction, cardiac conduction defects, high blood pressure, low blood pressure, cognitive impairment, multiple system atrophy, and constipation.

In one embodiment, the subject is at risk for developing, or is suffering from, neurodegeneration, and the method results in treating, preventing, and/or delaying the progression and/or onset of neurodegeneration in the subject. The neurodegeneration can be, for example, age-related; correlated with age-related dementia; correlated with a neurodisease; and/or correlated with one or more conditions or diseases selected from the group consisting of Alzheimer's disease, Parkinson's disease, Lewy Body dementia, fronto temperal dementia, supranuclear palsy, multi-system atrophy, Parkinsonism, amyotrophic lateral sclerosis (ALS), Huntington's Disease, schizophrenia, Friedreich's ataxia, Multiple sclerosis (MS), spinal muscular atrophy, progressive nuclear palsy, degenerative processes associated with aging, dementia of aging, Guadeloupian Parkinsonism, spinocerebellar ataxia, and vascular dementia. In an exemplary embodiment (a) progression or onset of the neurodegeneration is slowed, halted, or reversed over a defined time period following administration of the fixed escalated dose of the aminosterol or a salt or derivative thereof, as measured by a medically-recognized technique; and/or (b) the neurodegeneration is positively impacted by the fixed escalated dose of the aminosterol or a salt or derivative thereof, as measured by a medically-recognized technique. In yet another embodiment, (a) the positive impact and/or progression of neurodegeneration is measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis; and/or (b) the progression or onset of neurodegeneration is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

The methods of the invention also encompass methods where the subject suffers from, is or at risk of developing, depression. In an exemplary embodiment, the method results in improvement in a subject's depression, as measured by one or more clinically-recognized depression rating scale. For example, the improvement can be in one or more depression characteristics selected from the group consisting of mood, behavior, bodily functions such as eating, sleeping, energy, and sexual activity, and/or episodes of sadness or apathy. In another embodiment, the improvement a subject experiences following treatment can be about 5, about 10, about 15, about 20, about 25, about 30, about 35, about 40, about 45, about 50, about 55, about 60, about 65, about 70, about 75, about 80, about 85, about 90, about 95 or about 100%.

In another embodiment, the methods of the invention encompass methods where the subject suffers from, is or at risk of developing, autism or autism spectrum disorder. For example, the method can result in improvement: (a) in one or more of the subject's autism characteristics or behaviors, as measured by a clinically-recognized rating scale; and/or (b) in one or more autism characteristics or behaviors selected from the group consisting of social skills, repetitive behaviors, speech, nonverbal communication, sensory sensitivity, behavior, social interaction, and communication skills, as measured using a clinically-recognized scale. In another embodiment, the improvement a subject experiences following treatment in one or more autism characteristics or behaviors is about 5, about 10, about 15, about 20, about 25, about 30, about 35, about 40, about 45, about 50, about 55, about 60, about 65, about 70, about 75, about 80, about 85, about 90, about 95 or about 100%.

The methods of the invention also encompass methods where the subject suffers from, is or at risk of developing, schizophrenia. In one embodiment, the method results in improvement in one or more schizophrenia characteristics or behaviors, as measured using a clinically recognized rating scale. In another embodiment, the schizophrenia characteristics or behaviors can be selected from the group consisting of unclear or confusing thinking, reduced social engagement, reduced emotional expression, abnormal social behavior, failure to understand reality, lack of motivation, and hearing voices that others do not hear, as measured using a clinically-recognized scale. In yet a further embodiment, the improvement a subject experiences in one or more schizophrenia characteristics or behaviors following treatment is about 5, about 10, about 15, about 20, about 25, about 30, about 35, about 40, about 45, about 50, about 55, about 60, about 65, about 70, about 75, about 80, about 85, about 90, about 95 or about 100%.

The methods of the invention also encompass methods where the subject suffers from, is or at risk of developing, an inflammatory disease or condition caused by excessive expression or concentration of alpha synuclein in the subject. In one embodiment, the method results in a decrease in intensity of inflammation, blood levels of inflammatory markers, inflammatory markers in tissue, number of inflammatory cells in tissue, or any combination thereof, as compared to a control or as compared to the qualitative or quantitative amount from the same patient or subject prior to treatment. In yet another embodiment, the method results in a decrease in concentration of alpha synuclein in the subject. The decrease in alpha-synuclein concentration can be measured, for example, qualitatively, quantitatively, or semi-quantitatively by one or more methods. Such methods include for example (a) first determining the concentration of alpha-synuclein in a tissue sample from the subject prior to treatment, followed by: (i) after treatment determining the alpha-synuclein concentration in the same tissue type from the same subject; or (ii) after treatment comparing the alpha-synuclein concentration in the same tissue type to a control; (b) measuring the intensity of inflammation over time; (c) measuring the amount of inflammatory markers over time; (d) measuring the amount of inflammatory markers in blood, plasma, or tissue over time, either qualitatively or quantitatively; (e) measuring the amount of one or more inflammatory marker cytokines in blood, plasma, or tissue over time, either qualitatively or quantitatively; (f) measuring the amount of one or more plasma markers of inflammation such as TNF, IL-8, or CRP in blood, plasma, or tissue over time, either qualitatively or quantitatively; or (g) measuring the amount of inflammatory cells in blood, plasma, or tissue over time, either qualitatively or quantitatively. The decrease can be, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In one embodiment, the method is applied to a patient population susceptible to excessive expression of alpha-synuclein, resulting in an excessive or high concentration of alpha-synuclein.

Various symptoms that can be evaluated in the conditions described herein are detailed below. In yet another embodiment where the condition is Parkinson's disease, the symptom to be evaluated can be selected from the group consisting of: (a) at least one non-motor aspect of experiences of daily living as defined by Part I of the Unified Parkinson's Disease Rating Scale selected from the group consisting of cognitive impairment, hallucinations and psychosis, depressed mood, anxious mood, apathy, features of dopamine dysregulation syndrome, sleep problems, daytime sleepiness, pain, urinary problems, constipation problems, lightheadedness on standing, and fatigue; (b) at least one motor aspect of experiences of daily living as defined by Part II of the Unified Parkinson's Disease Rating Scale selected from the group consisting of speech, saliva and drooling, chewing and swallowing, eating tasks, dressing, hygiene, handwriting, turning in bed, tremors, getting out of a bed, a car, or a deep chair, walking and balance, and freezing; (c) at least one motor symptom identified in Part III of the Unified Parkinson's Disease Rating Scale selected from the group consisting of speech, facial expression, rigidity, finger tapping, hand movements, pronation-supination movements of hands, toe tapping, leg agility, arising from chair, gait, freezing of gait, postural stability, posture, body bradykinesia, postural tremor of the hands, kinetic tremor of the hands, rest tremor amplitude, and constancy of rest tremor; (d) at least one motor complication identified in Part IV of the Unified Parkinson's Disease Rating Scale selected from the group consisting of time spent with dyskinesias, functional impact of dyskinesias, time spent in the off state, functional impact of fluctuations, complexity of motor fluctuations, and painful off-state dystonia; (e) constipation; (f) depression; (g) cognitive impairment; (h) sleep problems or sleep disturbances; (i) circadian rhythm dysfunction; (j) hallucinations; (k) fatigue; (l) REM disturbed sleep; (m) REM behavior disorder; (n) erectile dysfunction; (o) apnea; (p) postural hypotension; (q) correction of blood pressure or orthostatic hypotension; (r) nocturnal hypertension; (s) regulation of temperature; (t) improvement in breathing or apnea; (u) correction of cardiac conduction defect; (v) amelioration of pain; (w) restoration of bladder sensation and urination; (x) urinary incontinence; and/or (y) control of nocturia.

Other symptoms for various conditions are described herein.

In one embodiment, the symptom to be evaluated is constipation, and the fixed escalated aminosterol dose for constipation is defined as the aminosterol dose that results in a complete spontaneous bowel movement (CSBM) within 24 hours of dosing on at least 2 of 3 days at a given dose. In another embodiment, the symptom to be evaluated is constipation, and if the average complete spontaneous bowel movement (CSBM) or average spontaneous bowel movement (SBM) is greater than or equal to 1 per week, then the starting aminosterol dosage prior to escalation is 75 mg; or if the average CSBM or SBM is less than 1 per week, then the starting aminosterol dosage prior to escalation is 150 mg.

In another embodiment, the aminosterol or a salt or derivative thereof is administered in combination with at least one additional active agent to achieve either an additive or synergistic effect. For example, the additional active agent is administered via a method selected from the group consisting of (a) concomitantly; (b) as an admixture; (c) separately and simultaneously or concurrently; or (d) separately and sequentially. In another embodiment, the additional active agent is a different aminosterol from that administered in primary method. In yet a further embodiment, the method of the invention comprises administering a first aminosterol which is aminosterol 1436 or a salt or derivative thereof intranasally and a administering a second aminosterol which is squalamine or a salt or derivative thereof orally.

For all of the methods of the invention, in one embodiment each aminosterol dose is taken on an empty stomach, optionally within about two hours of the subject waking. In another embodiment for all of the methods of the invention, no food is taken or consumed after about 60 to about 90 minutes of taking the aminosterol dose. Further, in yet another embodiment applicable to all of the methods of the invention, the aminosterol or a salt or derivative thereof can be a pharmaceutically acceptable grade of at least one aminosterol or a pharmaceutically acceptable salt or derivative thereof. For all of the methods of the invention the subject can be a human.

The aminosterol or a salt or derivative thereof utilized in the methods of the invention can be, for example: (a) isolated from the liver of Squalus acanthias; (b) a synthetic aminosterol; (c) a squalamine isomer; (d) squalamine or a pharmaceutically acceptable salt thereof; (e) a phosphate salt of squalamine; (f) an aminosterol comprising a sterol nucleus and a polyamine attached at any position on the sterol, such that the molecule exhibits a net charge of at least +1; (f) an aminosterol comprising a bile acid nucleus and a polyamine, attached at any position on the bile acid, such that the molecule exhibits a net charge of at least +1; (g) an aminosterol comprising a derivative modified to include one or more of the following: (i) substitutions of the sulfate by a sulfonate, phosphate, carboxylate, or other anionic moiety chosen to circumvent metabolic removal of the sulfate moiety and oxidation of the cholesterol side chain; (ii) replacement of a hydroxyl group by a non-metabolizable polar substituent, such as a fluorine atom, to prevent its metabolic oxidation or conjugation; and (iii) substitution of one or more ring hydrogen atoms to prevent oxidative or reductive metabolism of the steroid ring system; (h) a derivative of squalamine modified through medical chemistry to improve bio-distribution, ease of administration, metabolic stability, or any combination thereof; (i) aminosterol 1436 or a pharmaceutically acceptable salt thereof; and/or (j) the phosphate salt of aminosterol 1436. In one embodiment, the aminosterol is selected from the group consisting aminosterol 1436, squalamine, or a combination thereof. Other exemplary aminosterols are described herein.

Further, the aminosterol composition can comprise, for example, one or more of the following: an aqueous carrier, a buffer, a sugar, and/or a polyol compound.

Both the foregoing summary and the following description of the drawings and detailed description are exemplary and explanatory. They are intended to provide further details of the invention, but are not to be construed as limiting. Other objects, advantages, and novel features will be readily apparent to those skilled in the art from the following detailed description of the invention.

### BRIEF DESCRIPTION OF THE DRAWINGS

FIG. 1 (panels A and B) shows prokinetic activity of squalamine (ENT-01, a synthetic squalamine salt comprising squalamine as the active ion). As shown in panel A, in Stage 1 (single dose), cumulative prokinetic response rate was defined as the proportion of patients who had a complete spontaneous bowel movements (CSBM) within 24 hours of dosing. In Stage 2 (daily dosing), a prokinetic response was defined as the fraction of patients who had a CSBM within 24 hours of dosing on at least 2 out of 3 days at any given dose. As shown in panel B, the prokinetic dose of squalamine was significantly related to baseline constipation severity (p=0.00055). Patients with baseline CSBM<1 required a higher dose (mean, 192 mg) of squalamine than patients with CSBM≥1 (mean, 120 mg).

FIG. 2 is a schematic (flowchart) showing patient disposition in Stage 2. (1) Patients first enrolled (n=40); (2) 6 patients failed to meet dosing criteria and were excluded; (3) 34 patients were dosed; (4) 5 patients were discontinued; 3 patients withdrew consent (with 1 patient lost to follow up and 2 patients withdrew because of diarrhea); and 2 patients discontinued because of an adverse event (recurrent dizziness after medication); (5) 31 patients had an assessable prokinetic response; and (6) 29 patients completed dosing.

FIG. 3 is a chart of total sleep time in relation to squalamine dose. Total sleep time was obtained from the sleep diary by subtracting awake time during the night from total time spent in bed. Total sleep time per night was logged for each patient at baseline, each dosing period and at washout, and the means were determined. The light grey bar represents the baseline value for each cohort at a given dose level and the dark grey bar represents the value for the same cohort at the stated dose of squalamine (ENT-01; Kenterin™). The number of patients represented at each value are: Baseline, 33; 75 mg, 21; 100 mg, 28; 125 mg, 18; 150 mg, 15; 175 mg, 12; 200 mg, 7; 225 mg, 3; 250 mg, 2; washout, 33. P values were as follows: 75 mg, p=0.4; 100 mg, p=0.1; 125 mg, p=0.3; 150 mg, p=0.07; 175 mg, p=0.03; 200 mg, p=0.3; 225 mg, p=0.5; 250 mg, p=0.3; wash-out, p=0.04 (paired t test).

FIG. 4 shows the effect of squalamine (ENT-01) on circadian rhythm. The figure depicts the mean waveform of temperature under three conditions per patient: baseline (Line #1), treatment with highest drug dose (Line #2), and washout (Line #3). Each mean waveform is double plotted for better visualization. Low temperatures indicate higher activation, while higher values are associated with drowsiness and sleepiness. The top black bar indicates a standard rest period from 23:00 to 07:00 h.

FIGS. 5A-F show the effect of squalamine (ENT-01) on circadian rhythm. The figures depict the results of circadian non-parametric analysis of wrist skin temperature rhythm throughout each condition (baseline, treatment with highest dose of squalamine (ENT-01) and washout). The following parameters were measured: Inter-daily variability (FIG. 5A), inter-daily stability (IS) (FIG. 5B), relative amplitude (RA) (FIG. 5C), circadian function index (FIG. 5D), M5V (FIG. 5E), which refers to the five consecutive hours with the highest temperature or high somnolence, and L10V (FIG. 5F), which indicates the mean of the ten consecutive hours with lowest temperature or high activation. The circadian function index (CFI) is an integrated score that ranges from 0 (absence of circadian rhythm) to 1 (robust circadian rhythm). Student's paired t-test, *p<0.05, **p<01, ***p<0.001. Values expressed as mean±SEM (n=12 in each condition).

FIG. 6 shows the accumulation of Aminosterol 1436 within the centers of the brain that control growth, maturation, and senescence following intravenous administration to a rat via a peripheral vein via intravenous (IV) administration (FIGS. 6B and 6C), or injected via intracerebroventricular (ICV) administration directly into the 3^{rd} ventricle of the brain (FIG. 6A).

FIG. 7A shows the in vivo distribution of the aminosterol 1436 administered intraperitoneal (IP) or ICV as compared to vehicle (administered IP) in the Arc (arcuate nucleus of the hypothalamus), PVN (paraventricular nucleus of the hypothalamus), LH (lateral hypothalamus), VMN (ventromedial nucleus of the hypothalamus), CcA (central amygdala), and NTS (Nucleus Tractus Solitarius, a longitudinal structure in the medulla). FIG. 7B shows the effect on food intake over a 10 day period for animals administered vehicle ICV, vehicle IP, Aminosterol 1436 at 10 and 40 μg ICV, and Aminosterol 1436 at 5 mg/kg intraperitoneal injection (IP). Finally, FIG. 7C shows the percent change in body weight for the experiment detailed in FIG. 7B, with a decrease in body weight correlating with a decrease in food intake shown in FIG. 7B.

FIG. 8A shows the plasma concentration (ng/mL) vs time for squalamine lactate after 0.5 mg/kg administered intranasally (IN) in Sprague Dawley® (SD) rats, and FIG. 8C shows the CSF concentration (ng/mL) vs time profile for squalamine lactate following 0.5 mg/kg administered IN to SD rats. Similarly, FIG. 8B shows the plasma concentration (ng/mL) vs time for Aminosterol-1436 (“MSI-1436”) after 0.5 mg/kg administered IN in SD rats, and FIG. 3D shows the CSF concentration (ng/mL) vs time profile for Aminosterol 1436 following 0.5 mg/kg administered IN to SD rats. No squalamine lactate or Aminosterol 1436 was found in CSF following intranasal administration.

FIG. 9 shows the hypothalamus in relation to the intercavernous sinus, with the figure clearly showing the intercavernous sinus flowing next to the hypothalamus.

FIG. 10 depicts the hypothalamus in relation to the cavernous sinus.

FIG. 11 shows a side-on picture through the nasal cavity showing the turbinates which are highly vascularized.

FIG. 12 shows the vessels in the nasal cavity, with the cavernous sinus portion of the internal carotid artery (ICA) and the medial basal hypothalamus (MBH), ophthalmic artery (OA), internal carotid artery (ICA), and anterior ethmoidal artery (AEA) identified on the figure.

FIG. 13 shows the weight change (mean %) following administration to mice of (i) intraperitoneal (IP) administration of 1 mg/kg or 10 mg/kg of Aminosterol 1436, (ii) intranasal (IN) administration of 0.4 mg/kg Aminosterol 1436, or (iii) IN administration of saline control.

FIG. 14 shows pharmacokinetic parameters in a rat of intranasal administration of 0.5 mg/kg as compared to an intravenous bolus of 2/mg/kg (190 μg*hr/ml). Intranasal bioavailability of Aminosterol 1436 (MSI-1436) was found to be about 20%.

FIG. 15 shows characteristics of autism, including for example, the core autism symptoms of social deficits, language impairment, and repetitive behaviors; associated neurological issues of sleep disorders, mood, anxiety, hyperactivity, seizures, and attention; associated systemic issues of immune dysfunction and GI disorders; and related disorders, such as sleep disorders, mood disorders, anxiety disorders, OCD, and ADHD.

FIG. 16 shows total sleep time vs the dose of squalamine (ENT-01), with total sleep time increasing progressively from baseline to 250 mg.

FIG. 17 shows total sleep time vs the dose of squalamine (ENT-01), with total sleep time increasing progressively from baseline to 250 mg.

FIG. 18 shows REM-behavior disorder in relation to squalamine (ENT-01) dose, with arm and leg thrashing episodes (mean values) calculated using sleep diaries. The frequency of arm or leg thrashing reported in the sleep diary diminished progressively from 2.2 episodes/week at baseline to 0 at maximal dose.

FIG. 19A-F show intraluminal squalamine increased colonic PCC velocity and frequency in 3 commonly used mouse strains: Swiss Webster, C57BL/6, and CD-1. FIGS. 19A-C show spatiotemporal heat maps for Swiss Webster (19A), C57BL/6 (19B), and CD-1 (19C), showing propagating contractile clusters (PCCs) traveling from the oral to anal ends (top to bottom) where red on the left of each graph represents contraction and green on the right of each graph represents relaxation over time (left to right). Luminal application of squalamine (right) increased the velocity and frequency of PCCs as compared to the Krebs control in all strains. FIG. 19D shows intraluminal (10-30 μM) squalamine increased colonic PCC velocity in the three strains, ex vivo. FIG. 19E shows intraluminal squalamine had minimal effect on PCC amplitude in the three strains, ex vivo. FIG. 19F shows intraluminal squalamine increased PCC frequency in the three strains, ex vivo. From left to right: Swiss Webster, C57BL/6, and CD-1. Data represent mean±SEM, (N=8, 5, ad 3 respectively), (t-test paired, 2-tailed).

FIGS. 20A-D shows A53T PD mice had reduced colonic motor activity compared to WT control mice, but was improved by intraluminal squalamine. In FIG. 20A, A53T PD mice (black) had reduced PCC velocity compared to WT (gray) at baseline and threshold. Intraluminal squalamine (30 μM) significantly increased colonic PCC velocity in WT (gray patterned) and A53T (black patterned) at baseline (N=6-12 mice/group, 1-way ANOVA). FIG. 20B shows the results of feeding of squalamine for 5 days increased fecal pellet output in non-Tg (WT) and A53T mice at several doses. (N=10 mice/group/dose, 2-way ANOVA). FIG. 20C shows the results of feeding of squalamine increased the percent change in fecal water content from day 0 to day 5 in WT and A53T mice at increasing doses. (N=10 mice/group/dose, 1-way ANOVA). All data represented as mean±S.E.M, *P<0.05.

FIG. 21A-H shows FVB PD mice had decreased intrinsic excitability of myenteric intrinsic primary afferent neurons compared to FVB control mice. FIG. 21A shows representative action potential firing response to injected depolarizing square wave current stimulus (FIG. 21B) of 2× threshold intensity (FVB PD). FIG. 21C shows representative action potential firing response to current stimulus (FIG. 21D) of 2× threshold intensity (FVB control). FIGS. 21E-H show probabilities under the null hypothesis of no difference given the above dot plots, sample mean values given by open bars, error bars represent SEM. In FIG. 21E, the sample threshold intracellular current (AP threshold) required to evoke a single action potential (AP) was larger for FVB PD (N=20) than for FVB control mice (N=16) (t=2.2, t-test unpaired 2-tailed). In FIG. 21F, the sample number of action potentials evoked at 2 times threshold current intensity (No. APs 2× threshold) was greater for FVB control (N=19) and for FVB control mice (N=16) (t=1.9, t-test unpaired 2-tailed). In FIG. 21G, the sample post action potential slow after hyperpolarisation area under the curve (sAHP AUC) showed little difference between FVB PD (N=19) and FVB control mice (N=14) (t=1.4, t-test unpaired 2-tailed). In FIG. 21H, the sample resting membrane potential (RMP) was more hyperpolarised for FVB PD (N=20) then for FVB control mice (N=16) (t=2.2, t-test unpaired 2-tailed).

FIG. 22A-F shows the application of squalamine onto the intestinal epithelium or directly onto the exposed myenteric plexus increased excitability of intrinsic primary afferent neurons (IPANs) in FVB PD mice. FIG. 22A shows representative action potential firing increase to injected square wave current stimulus after acute application of 30 μM squalamine onto the intestinal epithelium using the divided hemidissection preparation. FIG. 22B shows Texas Red fluorescence image of neuron recorded from in FIG. 22A after tissue fixation revealing flattened oval soma and circumferentially directed neurites (Dogiel type II morphology) characteristic of chemosensitive myenteric intrinsic primary afferent neurons.

FIG. 22C shows addition of squalamine to the epithelial layer decreased sample action potential firing threshold (AP Threshold) (t=2.3, t-test paired 2-tailed), increased the number of action potentials discharged (No. AP 2× threshold) (N=15, t=4, t-test paired 2-tailed), decreased the sample area under the curve for the post action potential slow afterhyperpolarisation (sAHP AUC) (N=14, t=3.6, t-test paired 2-tailed) and depolarised the neuron sample resting membrane potential (RMP) (N=15, t=5.9, t-test paired 2-tailed). FIG. 22D shows representative action potential firing increase to injected square wave current stimulus after application of 30 μM squalamine onto the myenteric plexus. FIG. 22E shows Texas Red fluorescence image of neuron recorded from in FIG. 22D reveals Dogiel type II morphology. FIG. 22F shows addition of squalamine to the myenteric plexus decreased sample AP threshold (N=5, t=2.6, t-test paired 2-tailed), increased sample No. AP 2× threshold (N=5, t=2.2, t-test paired 2-tailed) decreased the sample post action potential sAHP AUC (N=5,t=2.1, t-test paired 2-tailed), and depolarised RMP (N=5, t=5.2, t-test paired 2-tailed). In FIGS. 22C and 22F, probabilities under the null hypothesis of no difference given above individual value barbell plots, sample mean values given by open bars, error bars represent SEM.

FIG. 23 shows a picture of a Bristol Stool Chart, which is a diagnostic medical tool designed to classify the form of human faeces into seven categories.

FIG. 24 shows a Constipation Assessment Scale (CAS), developed by McMillan and Williams (1989). The CAS was based on earlier research and clinical literature and includes eight commonly identified characteristics of constipation.

FIG. 25 shows an example of a Mini-Mental State Examination (MMSE) questionnaire that may be used to measure cognitive impairment.

FIG. 26 shows instructions for a typical trail making test that may be used to measure cognitive impairment.

FIGS. 27A and 27B show trail making tests that may be used to measure cognitive impairment according to the instructions shown in FIG. 26.

FIG. 28 shows a graph of the % of patients in 8 different systolic blood pressure (BP) intervals (90-99, 100-109, 110-119, 120-0129, 130-139, 140-149, 150-159, and 160-169) during Stage 2 of the clinical study described in the examples. Baseline; n=34; fixed aminosterol dose; n=34; and wash-out; n=28.

FIGS. 29A and 29B show the results of the effects of ENT-01 on blood pressure during Stage 2 of the clinical study described in the examples. FIG. 29A shows that average blood pressure (BP) was reduced from 138 mm at baseline to 129.9 mm at the aminosterol fixed dose (p=0.005). FIG. 29B shows a graph of systolic blood pressure (BP) at baseline vs at the aminosterol fixed dose, with the data clearly showing that subjects with high blood pressure demonstrating decreased blood pressure, and subjects with low blood pressure demonstrating increased blood pressure, when the subjects were administered a fixed aminosterol dose.

FIGS. 30A-C show the result of ENT-01 on patients' BP during Stage 2 of the clinical study described in the examples. FIG. 30A shows the change in systolic BP between pre-medication as compared to post-medication, with a final BP demonstrating normalization (e.g., low blood pressure patients exhibiting raised blood pressure and high blood pressure patients exhibiting lowered blood pressure. FIG. 30B shows a graph of initial vs final blood pressure (BP), and FIG. 30C shows a graph of initial BP, pre-dose BP, and BP 2 hours post dose.

FIG. 31 graphically shows normalization of systolic blood pressure (SBP) in subjects during Stage 1 of the clinical study described in the examples below. Normalization refers to lowering high blood pressure and raising low blood pressure into normal ranges. The numbers shown in the graph represent baseline BP then highest treatment BP, both in the lying position, pre-medication. The result is significant despite the small sample size (p=0.01).

### DETAILED DESCRIPTION

### I. Overview

The present application relates generally to compositions and methods for treating and/or preventing a variety of brain-gut disorders and symptoms related thereto. The methods comprise administering one or more aminosterols or pharmaceutically acceptable salts or derivatives thereof to a subject in need.

In a first aspect, described is a method of treating, preventing, and/or slowing the onset or progression of various indications described herein and/or a symptom related to the condition in a subject in need comprising administering to the subject a therapeutically effective amount of at least one aminosterol, or a salt or derivative thereof.

In a second aspect, for certain indications described herein the method of administration does not comprise oral administration. For example, the method can comprise administration selected from nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof. In one aspect, administering comprises nasal administration.

In a third aspect for all indications described herein, the method comprises determining an “optimized aminosterol dose” or “fixed aminosterol dose”, as described in detail herein. For example, the method can comprise the following steps: (i) identifying a starting aminosterol dose for a subject; (ii) administering an escalating dose of the aminosterol over a period of time until an effective aminosterol dose is identified, wherein the effective aminosterol dose is the dose where improvement or resolution of the indication and/or indication-related symptom is observed, and (iii) fixing the aminosterol dose at that level in that particular subject.

As described in Example 1, a study was conducted in patients with Parkinson's disease (PD). PD is a progressive neurodegenerative disorder caused by accumulation of the protein α-synuclein (αS) within the enteric nervous system (ENS), autonomic nerves and brain.

While the study described herein assessed patients with PD, many symptoms assessed and contemplated to be resolved by aminosterol treatment are not restored by the replacement of dopamine and are, thus, not unique to PD but rather common across a variety of disorders which involve impaired function of neural pathways, referred to herein as “brain-gut” disorders. Examples of such symptoms include, but are not limited to, constipation, disturbances in sleep architecture, cognitive impairment or dysfunction, hallucinations, REM behavior disorder (RBD), and depression. Other relevant symptoms are described herein. All of all of these symptoms result from impaired function of neural pathways not restored by replacement of dopamine.

In 2003, Braak proposed that PD begins with the formation of toxic αS aggregates within the ENS and manifests clinically as constipation in a majority of people years before the onset of motor symptoms. It was recently reported that αS is induced in the ENS in response to viral, bacterial and fungal infections and that excessive intraneuronal accumulation of αS promotes formation of toxic aggregates. As a result of the normal trafficking of αS aggregates from the ENS to the central nervous system (CNS) via afferent nerves such as the vagus, neurotoxic aggregates accumulate progressively within the brainstem and more rostral structures. Thus, inhibiting αS aggregation in the ENS may reduce the continuing PD disease process in both the ENS and CNS.

A strategy that targets neurotoxic aggregates of αS in the gastrointestinal tract represents a novel approach to the treatment of PD and other neurodiseases and conditions described herein that may restore the function of enteric nerve cells and prevent retrograde trafficking to the brain. Such actions may potentially slow progression of the disease in addition to restoring gastrointestinal function.

Accordingly but not to be bound by theory, the methods described herein are expected to apply to the treatment of any of the described symptoms as well as treatment and/or prevention of brain-gut disorders other than PD sharing such symptoms. Examples of such brain-gut disorders include but are not limited to (i) age-related neurodegeneration, (ii) age-related neurodegeneration correlated with age-related dementia, (iii) neurodiseases such as Alzheimer's disease (AD), Huntington's Disease, Multiple Sclerosis, Amyotorphic Lateral Sclerosis (ALS), multiple system atrophy (MSA), schizophrenia, Friedreich's ataxia, vascular dementia, Lewy Body dementia or disease, spinal muscular atrophy, supranuclear palsy, fronto temperal dementia, progressive nuclear palsy, Guadeloupian Parkinsonism, spinocerebellar ataxia, and autism.

Not to be bound by theory, it is believed that aminosterols target neurotoxic aggregates of αS in the gastrointestinal tract, and restore function of the enteric nerve cells. The now-functional enteric nerve cells prevent retrograde trafficking of proteins, such as alpha-synuclein, to the brain. In addition to restoring gastrointestinal function, this effect is believed to slow and possibly reverse disease progression.

Constipation serves as an early indicator of many neurodiseases such as PD to the extent that it is suspected to correlate with the formation of toxic αS aggregates within the enteric nervous system (ENS) (Braak et al. 2003). As a result of the normal trafficking of αS aggregates from the ENS to the central nervous system (CNS) via afferent nerves such as the vagus (Holmqvist et al. 2014; Svensson et al. 2015), neurotoxic aggregates accumulate progressively within the brainstem and more rostral structures. Inhibiting αS aggregation in the ENS may, thus, reduce the continuing neuro disease process in both the ENS and CNS (Phillips et al. 2008). This relationship between the ENS and CNS is sometimes described herein as “brain-gut” in relation to a class of disorders or the axis of aminosterol activity.

Not to be bound by theory, based on the data described herein, it is believed that aminosterols improve bowel function by acting locally on the gastrointestinal tract (as supported by the oral bioavailability <0.3%). An orally administered aminosterol such as squalamine, the active ion of ENT-01, stimulates gastro-intestinal motility in mice with constipation due to overexpression of human αS (West et al, manuscript in preparation). Perfusion of an aminosterol such as squalamine through the lumen of an isolated segment of bowel from the PD mouse model results in excitation of IPANs (intrinsic primary afferent neuron), the major sensory neurons of the ENS that communicate with the myenteric plexus, increasing the frequency of propulsive peristaltic contractions and augmenting neural signals projecting to the afferent arm of the vagus.

Systemic absorption of the aminosterol following oral administration was negligible both in this study and in prior studies involving mice, rats and dogs. Prior studies demonstrated that intravenous administration of squalamine was not associated with increased gastrointestinal motility, despite reaching systemic blood levels one thousand-fold greater than that achieved by orally administered squalamine. These data suggest that the effect is mediated by local action in the GI tract. The topical action would also explain why adverse events were largely confined to the gastrointestinal tract.

Several exploratory endpoints were incorporated into the trial described in Example 1 to evaluate the impact of an aminosterol on neurologic symptoms associated with a neurodisease such as PD. Following aminosterol treatment, the Unified Parkinson's Disease Rating Scale (UPDRS) score, a global assessment of motor and non-motor symptoms, showed significant improvement. Improvement was also seen in the motor component. The improvement in the motor component is unlikely to be due to improved gastric motility and increased absorption of dopaminergic medications, since improvement persisted during the 2-week wash-out period, i.e., in the absence of study drug.

Improvements were also seen in cognitive function (MMSE scores), hallucinations, REM-behavior disorder (RBD) and sleep. Six of the patients enrolled had daily hallucinations or delusions and these improved or disappeared during treatment in five. In one patient the hallucinations disappeared at 100 mg, despite not having reached the colonic prokinetic dose (e.g., fixed escalated aminosterol dose) of 175 mg for this particular patient. The patient remained free of hallucinations for 1 month following cessation of dosing. RBD and total sleep time also improved progressively in a dose-dependent manner.

Interestingly, most indices related to bowel function returned to baseline value by the end of the 2-week wash-out period while improvement in the CNS symptoms persisted. The rapid improvement in certain CNS symptoms is consistent with a mechanism whereby nerve impulses initiated from the ENS following aminosterol administration augment afferent neural signaling to the CNS. This may stimulate the clearance of αS aggregates within the afferent neurons themselves as well as the secondary and tertiary neurons projecting rostrally within the CNS, since it is known that neural stimulation is accompanied by increased neuronal autophagic activity (Shehata et al. 2012). It is believed that after cessation of aminosterol administration, the neurons of the CNS gradually re-accumulate an αS burden either locally or via trafficking from αS re-aggregation within the gut.

Disturbance of the circadian rhythm has been described in neurodiseases such as PD both clinically and in animal models and might play a role in the abnormal sleep architecture, dementia, mood and autonomic dysfunction associated with neurodiseases such as PD (Breen et al. 2014; Videnovic et al. 2017; Antonio-Rubio et al. 2015; Madrid-Navarro et al. 2018). Circadian rhythm was monitored through the use of a temperature sensor that continuously captured wrist skin temperature (Sarabia et al. 2008), an objective measure of the autonomic regulation of vascular perfusion (Videnovic et al. 2017). Circadian cycles of wrist skin temperature have been shown to correlate with sleep wake cycles, reflecting the impact of nocturnal heat dissipation from the skin on the decrease in core temperature and the onset of sleep (Sarabia et al. 2008; Ortiz-Tuleda et al. 2014). Oral administration of ENT-01 had a significant positive impact on the circadian rhythm of skin temperature in the 12 patients with evaluable data. Not to be bound by theory, it is believed that aminosterols could he affecting neuronal circuits involving the master clock (the suprachiasmatic nucleus) and its autonomic projections and opens the possibility of therapeutic correction of circadian dysfunction.

As described in Example 1, aminosterol dosing is patient specific, as the dose is likely related to the extent of neuronal damage, with greater neuronal damage correlating with the need for a higher aminosterol dose to obtain a desired therapeutic result. As described in greater detail herein, aminosterol dosing can range from about 0.01 to about 500 mg/day, with dosage determination described in more detail below.

### II. Overview of Aspects of the Methods Described Herein

In one aspect of the methods described herein, the aminosterol or a salt or derivative thereof is taken on an empty stomach, optionally within two hours of the subject waking; and/or no food is taken after about 60 to about 90 minutes of taking the aminosterol or a salt or derivative thereof.

In another aspect, the aminosterol or a salt or derivative thereof is a pharmaceutically acceptable grade of at least one aminosterol or a pharmaceutically acceptable salt or derivative thereof.

In yet another aspect, the aminosterol or a salt or derivative thereof is comprised in a composition further comprising one or more of the following: an aqueous carrier; a buffer; a sugar; and/or a polyol compound.

In other embodiments, where the aminosterol or a salt or derivative thereof is formulated for oral administration, the composition is a liquid, capsule, or tablet designed to disintegrate in either the stomach, upper small intestine, or more distal portions of the intestine.

In addition, where the aminosterol or a salt or derivative thereof is formulated for nasal administration, the aminosterol or a salt or derivative thereof can be formulated into a dry powder or nasal spray or liquid nasal spray.

Further, in all of the methods described herein, the subject can be a human; and/or the subject can be a member of a patient population at risk for developing the indication.

In one aspect of the methods described herein, the aminosterol is administered in combination with at least one additional active agent to achieve either an additive or synergistic effect; and/or the additional active agent is administered via a method selected from the group consisting of concomitantly, as an admixture, separately and simultaneously or concurrently, and separately and sequentially; and/or the additional active agent is a different aminosterol from that administered in the primary method; and/or the method comprises a first aminosterol which is aminosterol 1436 or a salt or derivative thereof administered intranasally and a second aminosterol which is squalamine or a salt or derivative thereof administered orally; and/or the additional active agent is an active agent used to treat the indication or a symptom thereof.

In the methods described herein, the therapeutically effective amount of the at least one aminosterol or a salt or derivative thereof: (a) comprises about 0.1 to about 20 mg/kg body weight of the subject; and/or (b) comprises about 0.1 to about 15 mg/kg body weight of the subject; and/or (c) comprises about 0.1 to about 10 mg/kg body weight of the subject; and/or (d) comprises about 0.1 to about 5 mg/kg body weight of the subject; and/or (e) comprises about 0.1 to about 2.5 mg/kg body weight of the subject; and/or (f) comprises about 0.001 to about 500 mg/day; and/or (g) comprises about 0.001 to about 250 mg/day; and/or (h) comprises about 0.001 to about 125 mg/day; and/or (i) comprises about 0.001 to about 50 mg/day; and/or (j) comprises about 0.001 to about 25 mg/day; and/or (k) comprises about 0.001 to about 10 mg/day; and/or (l) comprises nasal administration and wherein the therapeutically effective amount of the at least one aminosterol, or a salt or derivative thereof comprises about 0.001 to about 6 mg/day; and/or (m) comprises nasal administration and wherein the therapeutically effective amount of the at least one aminosterol, or a salt or derivative thereof comprises about 0.001 to about 4 mg/day; and/or (n) comprises nasal administration and wherein the therapeutically effective amount of the at least one aminosterol, or a salt or derivative thereof comprises about 0.001 to about 2 mg/day; and/or (o) comprises nasal administration and wherein the therapeutically effective amount of the at least one aminosterol, or a salt or derivative thereof comprises about 0.001 to about 1 mg/day. Other exemplary dosages are described herein.

In addition, unless oral administration is excluded for a certain indication, the aminosterol or a salt or derivative thereof is administered orally, intranasally, or a combination thereof.

In all of the methods described herein, the aminosterol dose can be (i) given once per day, every other day, once per week, twice per week, three times per week, four times per week, five times per week, six times per week, every other week, or every few days; and/or (ii) given for a few weeks, followed by skipping a few weeks, followed by restarting aminosterol treatment; and/or (iii) incrementally reduced after the fixed dose of aminosterol or a salt or derivative thereof has been administered to the subject for a period of time; and/or (iv) varied plus or minus a defined amount to enable a modest reduction or increase in the fixed dose; and/or (v) increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

Where the aminosterol or a salt or derivative thereof is administered orally and the method comprises determining a “fixed” aminosterol dose, (a) the starting aminosterol dose ranges from about 1 mg up to about 175 mg; and/or (b) the starting oral aminosterol dose is about 25 mg/day; and/or (c) the dose of the aminosterol or a salt or derivative thereof for the subject following escalation is fixed at a range of from about 1 mg up to about 500 mg; and/or (d) the dose of the aminosterol or a salt or derivative thereof for the subject following escalation is fixed at a dose of about 1, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, or about 500 mg/day.

In other aspects of the methods described herein, where (a) the subject experiences moderate level of the indication or a related symptom (e.g., for constipation this is defined as a baseline rate of CSBM or SBM in the subject of one or more CSBM or SBM per week), then the starting oral aminosterol dose can be from about 10 to about 75 mg/day; and/or (b) the subject experiences a moderate level of the indication or a related symptom (e.g., for constipation this is defined as a baseline rate of CSBM or SBM in the subject of one or more CSBM or SBM per week), then the starting oral aminosterol dose can be about 10, about 15, about 20, about 25, about 30, about 35, about 40, about 45, about 60, about 65, about 70, or about 75 mg/day; and/or (c) the subject experiences a severe level of the indication or a related symptom (e.g., for constipation this is defined as a baseline rate of CSBM or SBM in the subject of less than one CSBM or SBM per week), then the starting oral aminosterol dose can be at least about 75 mg/day; and/or (d) the subject experiences a severe level of the indication or a related symptom (e.g., for constipation this is defined as a baseline rate of CSBM or SBM in the subject of less than one CSBM or SBM per week), then the starting oral aminosterol dose is from about 75 to about 175 mg/day; and/or (e) the subject experiences severe constipation or a related symptom, which is defined as a baseline rate of CSBM or SBM in the subject of less than one CSBM or SBM per week, and wherein the starting oral aminosterol dose is about 75, about 80, about 85, about 90, about 95, about 100, about 105, about 110, about 115, about 120, about 125, about 130, about 135, about 140, about 145, about 150 about 155, about 160, about 165, about 170, or about 175 mg/day; and/or (e) the subject experiences severe constipation or a related symptom, which is defined as a baseline rate of CSBM or SBM in the subject of less than one CSBM or SBM per week, and wherein the starting oral aminosterol dose is at least about 175 mg/day.

In other aspects of the methods described herein where the method comprises determining a “fixed” dose, the dose of the aminosterol or a salt or derivative thereof is escalated in increments of about 5, about 10, about 15, about 20, about 25, about 30, about 35, about 40, about 45, or about 50 mg; and/or the dose of the aminosterol or a salt or derivative thereof is escalated in about 25 mg increments.

In the methods of the invention comprising determining a “fixed” aminosterol dose, and where the composition is administered intranasally, then (i) the starting dose of the aminosterol or a salt or derivative thereof ranges from about 0.001 mg to about 3 mg; and/or (ii) the starting dose of the aminosterol or a salt or derivative thereof is about 0.001, about 0.005, about 0.01, about 0.02, about 0.03, about 0.05, about 0.06, about 0.07, about 0.08, about 0.09, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 1.0, about 1.1, about 1.25, about 1.3, about 1.4, about 1.5, about 1.6, about 1.7, about 1.75, about 1.8, about 1.9, about 2.0, about 2.1, about 2.25, about 2.3, about 2.4, about 2.5, about 2.6, about 2.7, about 2.75, about 2.8, about 2.9, or about 3 mg; and/or (iii) the dose of the aminosterol or a salt or derivative thereof for the subject following escalation is fixed at a range of from about 0.001 mg up to about 6 mg; and/or (iv) the dose of the aminosterol or a salt or derivative thereof for the subject following escalation is fixed at about 0.001, about 0.005, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, or about 6 mg; and/or (v) the dose of the aminosterol or a salt or derivative thereof for the subject following escalation is a dose which is sub-therapeutic when given orally or by injection; and/or (vi) the dose of the aminosterol or a salt or derivative thereof is escalated in increments of about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg.

In the methods of the invention comprising determining a “fixed” aminosterol dose, the dose of the aminosterol or a salt or derivative thereof can be escalated every about 1 to about 14 days; and/or every about 3 to 5 days; and/or every about 1, about 2, about 3, about 4, about 5, about 6, about 7, about 8, about 9, about 10, about 11, about 12, about 13, or about 14 days; and/or about 1×/week, about 2×/week, about every other week, or about 1×/month.

### III. Methods of Determining and Compositions

Comprising a “Fixed Dose” of Aminosterol

In one embodiment, the present application relates to the surprising discovery of a method to determine a “fixed dose” of an aminosterol composition that is not age, size, or weight dependent but rather is individually calibrated. The “fixed dose” obtained through this method yields highly effective results in treating the symptom(s) based on which the “fixed dose” was determined, related symptoms along the “brain-gut” axis, and the underlying disorder. Further, contemplated herein are methods of leveraging this same “fixed dose” method for methods of prevention of the underlying disorder. Not all of the methods described herein require determining a patient-specific optimized or fixed dose of an aminosterol.

A. “Fixed Aminosterol Dose”

A “fixed aminosterol dose”, also referred to herein as a “fixed escalated aminosterol dose,” which will be therapeutically effective is determined for each patient by establishing a starting dose of an aminosterol composition and a threshold for improvement of a particular symptom. Following determining a starting aminosterol dosage for a particular patient, the aminosterol dose is then progressively escalated by a consistent amount over consistent time intervals until the desired improvement is achieved; this aminosterol dosage is the “fixed escalated aminosterol dosage” for that particular patient for that particular symptom. In exemplary embodiments, an orally administered aminosterol dose is escalated every about 3 to about 5 days by about 25 mg until the desired improvement is reached. Symptoms evaluated, along with tools for measuring symptom improvement, may be specifically described below, including but not limited to constipation, hallucinations, sleep disturbances (e.g. REM disturbed sleep or circadian rhythm dysfunction), cognitive impairment, depression, or alpha-synuclein aggregation.

This therapeutically effective “fixed dose” is then maintained throughout treatment and/or prevention. Thus, even if the patient goes “off drug” and ceases taking the aminosterol composition, the same “fixed dose” is taken with no ramp up period following re-initiation of aminosterol treatment.

Not to be bound by theory, it is believed that the aminosterol dose is dependent on the severity of nerve damage relating to the symptom establishing the “fixed dose” threshold—e.g. for constipation, the dose may be related to the extent of nervous system damage in the patient's gut.

The aminosterol can be administered via any pharmaceutically acceptable means, such as by injection (e.g., IM, IV, or IP), oral, pulmonary, intranasal, etc. Preferably, the aminosterol is administered orally, intranasally, or a combination thereof.

Oral dosage of an aminosterol can range from about 1 to about 500 mg/day, or any amount in-between these two values. Other exemplary dosages of orally administered aminosterols include, but are not limited to, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, or about 500 mg/day.

Intranasal dosages of an aminosterol are much lower than oral dosages of an aminosterol. Examples of such intranasal aminosterol low dosages include, but are not limited to, about 0.001 to about 6 mg, or any amount in-between these two values. For example, the low dosage of an intranasal administered aminosterol can be about 0.001, about 0.005, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, or about 6 mg/day.

For intranasal (IN) administration, it is contemplated that the aminosterol dosage may be selected such that it would not provide any pharmacological effect if administered by any other route—e.g., a “subtherapeutic” dosage, and, in addition, does not result in negative effects. For example, Aminosterol 1436 is known to have the pharmacological effects of a reduction in food intake and weight loss. Therefore, in the IN methods of the invention, if the aminosterol is Aminosterol 1436 or a salt or derivative thereof, then if the IN Aminosterol 1436 dosage is administered via another route, such as oral, IP, or IV, then the Aminosterol 1436 dosage will not result in a noticeable reduction in food intake or noticeable weight loss. Similarly, squalamine is known to produce the pharmacological effects of nausea, vomiting and/or reduced blood pressure. Thus, in the IN methods of the invention, if the aminosterol is squalamine or a salt or derivative thereof, then if the IN squalamine dosage is administered via another route, such as oral, IP, or IV, then the squalamine dosage will not result in noticeable nausea, vomiting, and/or a reduction in blood pressure. Suitable exemplary aminosterol dosages are described above.

Dose Escalation:

When determining a “fixed aminosterol dosage” for a particular patient, a patient is started at a lower dose and then the dose is escalated until a positive result is observed for the symptom being evaluated. For example, constipation is exemplified in Example 1. Aminosterol doses can also be de-escalated (reduced) if any given aminosterol dose induces a persistent undesirable side effect, such as diarrhea, vomiting, or nausea.

The starting aminosterol dose is dependent on the severity of the symptom—e.g. for a patient experiencing severe constipation, defined as less than one spontaneous bowel movement (SBM) a week, the starting oral aminosterol dose can be about 150 mg or greater. In contrast, for a patient having moderate constipation, e.g., defined as having more than one SBM a week, the starting aminosterol dose can be about 75 mg. Thus, as an example, a patient experiencing moderate constipation can be started at an aminosterol dosage of about 75 mg/day, whereas a patient experiencing severe constipation can be started at an aminosterol dosage of about 150 mg/day.

In other embodiments, a patient experiencing moderate symptoms (for the symptom being used to calculate a fixed escalated aminosterol dose) can be started at an oral aminosterol dosage of from about 10 mg/day to about 75 mg/day, or any amount in-between these values. For example, the starting oral aminosterol dosage for a moderate symptom can be about 10, about 15, about 20, about 25, about 30, about 35, about 40, about 45, about 60, about 65, about 70, or about 75 mg.

In yet further embodiments, when the patient is experiencing severe symptoms (for the symptom being used to calculate the fixed escalated aminosterol dose), the patient can be started at an oral aminosterol dosage ranging from about 75 to about 175 mg/day, or any amount in-between these two values. For example, the starting oral aminosterol dosage for a severe symptom can be about 75, about 80, about 85, about 90, about 95, about 100, about 105, about 110, about 115, about 120, about 125, about 130, about 135, about 140, about 145, about 150 about 155, about 160, about 165, about 170, or about 175 mg.

In some embodiments, the starting oral aminosterol dose may be about 125 mg or about 175 mg; again dependent on the severity of the symptom, such as constipation.

Starting IN aminosterol dosages prior to dose escalation can be, for example, about 0.001 mg to about 3 mg, or any amount in-between these two values. For example, the starting aminosterol dosage for IN administration, prior to dose escalation, can be, for example, about 0.001, about 0.005, about 0.01, about 0.02, about 0.03, about 0.05, about 0.06, about 0.07, about 0.08, about 0.09, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 1.0, about 1.1, about 1.25, about 1.3, about 1.4, about 1.5, about 1.6, about 1.7, about 1.75, about 1.8, about 1.9, about 2.0, about 2.1, about 2.25, about 2.3, about 2.4, about 2.5, about 2.6, about 2.7, about 2.75, about 2.8, about 2.9, or about 3 mg.

In exemplary embodiments, the aminosterol dose is given periodically as needed. For example, the aminosterol dose can be given once per day. The aminosterol dose can also be given every other day, 2, 3, 4, or 5× per week, once/week, or 2×/week. In another embodiment, the aminosterol dose can be given every other week, or it can be given for a few weeks, followed by skipping a few weeks (as the effects persist following treatment), followed by restarting aminosterol treatment.

When calculating a fixed escalated aminosterol dose, the dose can be escalated following any suitable time period. In one embodiment, the aminosterol dose is escalated every about 3 to about 7 days by about a defined amount until a desired improvement is reached. For example, when the symptom being treated/measured is constipation, threshold improvement can be an increase of one SBM per week or at least a total of three bowel movements per week. In other embodiments, the aminosterol dose can be escalated every about 1, about 2, about 3, about 4, about 5, about 6, about 7, about 8, about 9, about 10, about 11, about 12, about 13, or about 14 days. In other embodiments, the aminosterol dose can be escalated about 1×/week, about 2×/week, about every other week, or about 1×/month.

During dose escalation, the aminosterol dosage can be increased by a defined amount. For example, when the aminosterol is administered orally, the dose can be escalated in increments of about 5, about 10, about 15, about 20, about 25, about 30, about 35, about 40, about 45, or by about 50 mg. When the aminosterol is administered intranasally, then the dosage can be increased in increments of about, for example, about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg.

Other symptoms that can be used as an endpoint to determine aminosterol dosage for a patient's fixed escalated aminosterol dosage are described herein and include, but are not limited to, (a) at least one non-motor aspect of experiences of daily living as defined by Part I of the Unified Parkinson's Disease Rating Scale, such as for example cognitive impairment, hallucinations and psychosis, depressed mood, anxious mood, apathy, features of dopamine dysregulation syndrome, sleep problems, daytime sleepiness, pain, urinary problems, constipation problems, lightheadedness on standing, and fatigue; (b) at least one motor aspect of experiences of daily living as defined by Part II of the Unified Parkinson's Disease Rating Scale, such as for example, speech, saliva and drooling, chewing and swallowing, eating tasks, dressing, hygiene, handwriting, turning in bed, tremors, getting out of a bed, a car, or a deep chair, walking and balance, and freezing; (c) at least one motor symptom identified in Part III of the Unified Parkinson's Disease Rating Scale, such as for example, speech, facial expression, rigidity, finger tapping, hand movements, pronation-supination movements of hands, toe tapping, leg agility, arising from chair, gait, freezing of gait, postural stability, posture, body bradykinesia, postural tremor of the hands, kinetic tremor of the hands, rest tremor amplitude, and constancy of rest tremor; (d) at least one motor complication identified in Part IV of the Unified Parkinson's Disease Rating Scale, such as for example, dyskinesias, functional impact of dyskinesias, time spent in the off state, functional impact of fluctuations, complexity of motor fluctuations, and painful off-state dystonia; (e) constipation; (f) depression; (g) cognitive impairment; (h) sleep problems or sleep disturbances; (i) circadian rhythm dysfunction; (j) hallucinations; (k) fatigue; (l) REM disturbed sleep; (m) REM behavior disorder; (n) erectile dysfunction; (o) apnea; (p) postural hypotension; (q) correction of blood pressure or orthostatic hypotension; (r) nocturnal hypertension; (s) regulation of temperature; (t) improvement in breathing or apnea; (u) correction of cardiac conduction defect; (v) amelioration of pain; (w) restoration of bladder sensation and urination; (x) urinary incontinence; and/or (y) control of nocturia.

B. Aminosterols

U.S. Pat. No. 6,962,909, entitled “Treatment of neovascularization disorders with squalamine,” discloses various aminosterols, and this disclosure is specifically incorporated by reference with respect to its teaching of aminosterol compounds. Any aminosterol known in the art, including those described in U.S. Pat. No. 6,962,909, can be used in the disclosed compositions. In some embodiments, the aminosterol present in the compositions of the invention is Aminosterol 1436 or a salt or derivative thereof, squalamine or a salt or derivative thereof, or a combination thereof.

An aminosterol such as squalamine (ENT-01 in the examples) inhibits the formation of αS aggregates in vitro and in vivo, reverses motor dysfunction in the C. elegans αS model, and restores gastrointestinal motility in mouse models of PD.

For instance, useful aminosterol compounds comprise a bile acid nucleus and a polyamine, attached at any position on the bile acid, such that the molecule exhibits a net positive charge contributed by the polyamine.

Thus, in some embodiments, the disclosed methods comprise administering a therapeutically effective amount of one or more aminosterols having the chemical structure of Formula I:

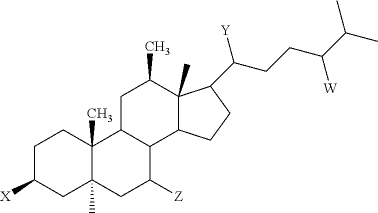

wherein,
W is 24S—OSO₃ or 24R—OSO₃;
X is 3β-H₂N—(CH₂)₄—NH—(CH₂)₃—NH— or 3α-H₂N—(CH₂)₄—NH—(CH₂)₃—NH—;
Y is 20R—CH₃; and
Z is 7α or 7β-OH.

In another embodiment of the invention, the aminosterol is selected from the following group:

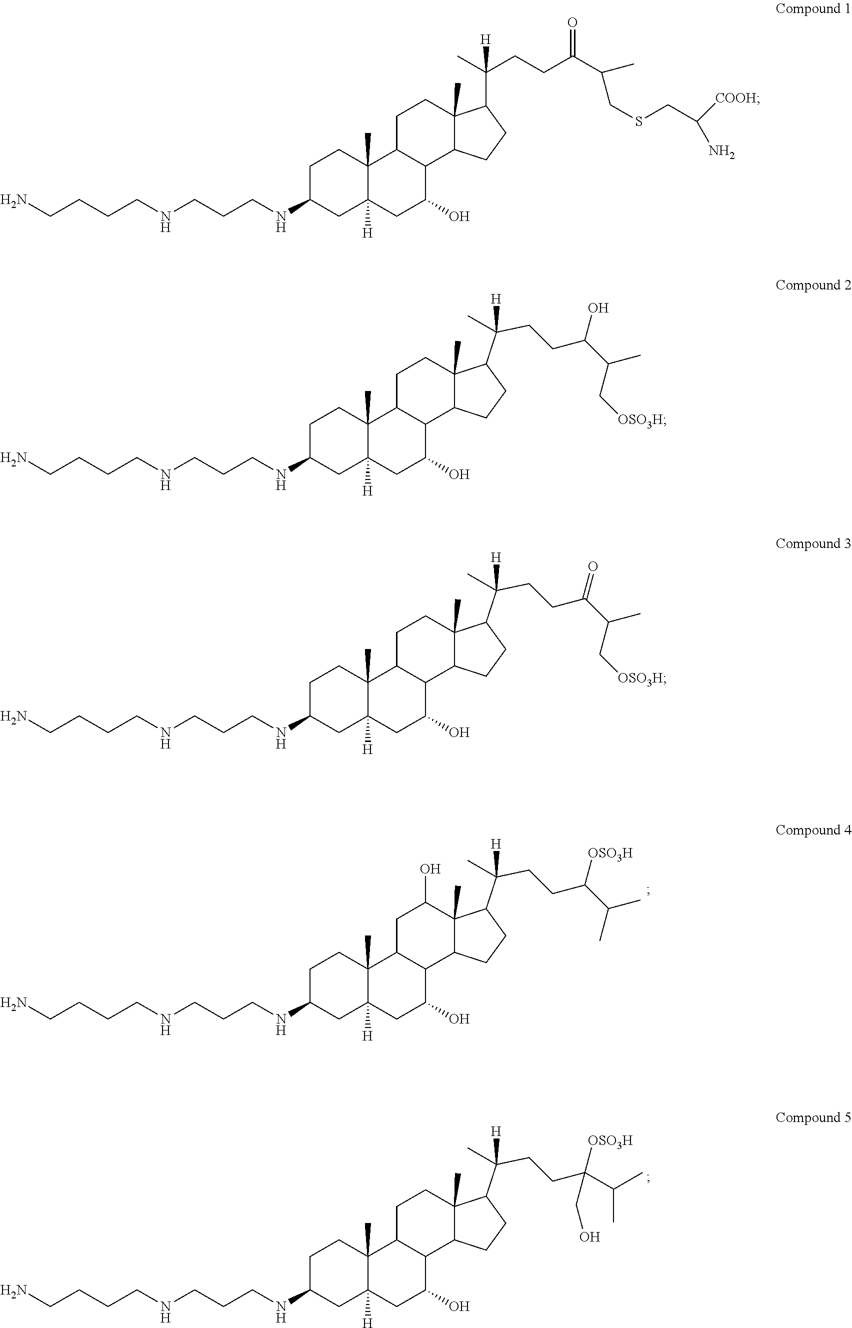

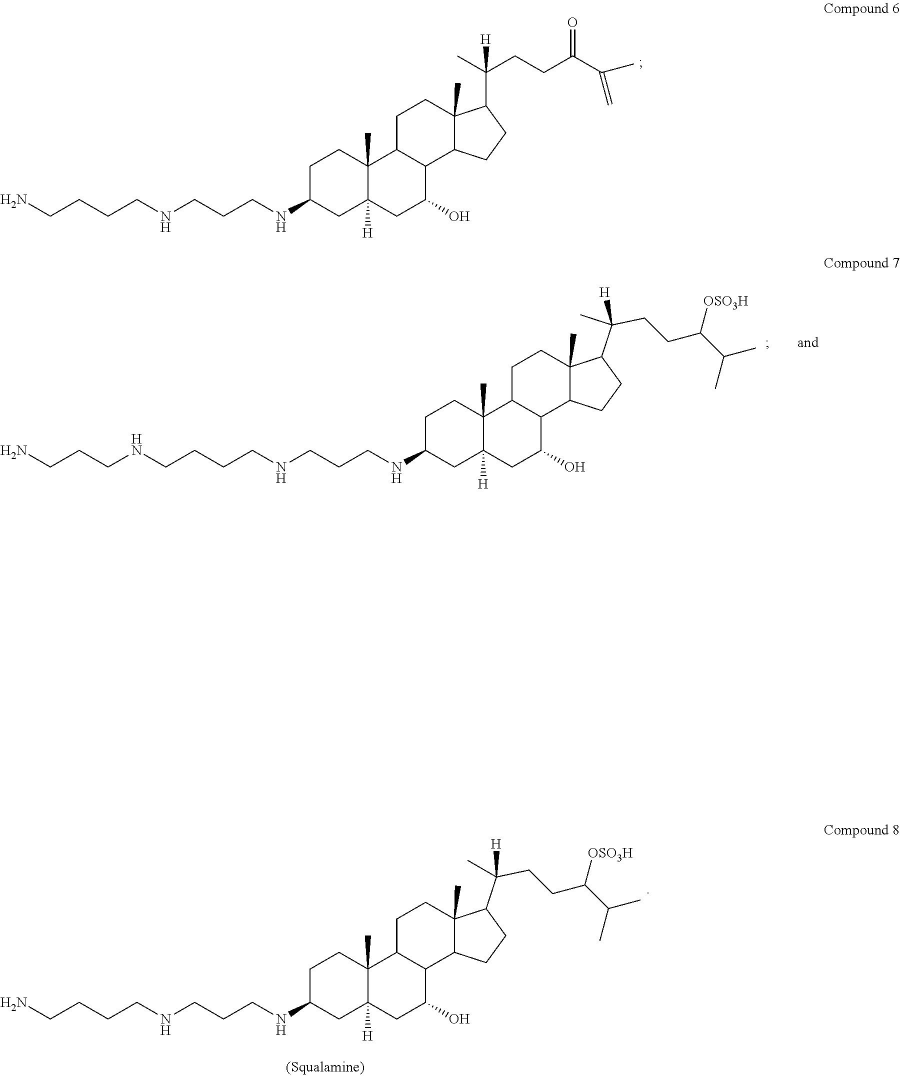

Variants or derivatives of known aminosterols, such as squalamine, Aminosterol 1436, or an aminosterol isolated from Squalus acanthias, may be used in the disclosed compositions and methods.

In one embodiment, the aminosterol is Aminosterol 1436 or a squalamine isomer. In yet another embodiment of the invention, the aminosterol is a derivative of squalamine or another naturally occurring aminosterol modified through medical chemistry to improve biodistribution, ease of administration, metabolic stability, or any combination thereof. In another embodiment, the squalamine or aminosterol is modified to include one or more of the following: (1) substitutions of the sulfate by a sulfonate, phosphate, carboxylate, or other anionic moiety chosen to circumvent metabolic removal of the sulfate moiety and oxidation of the cholesterol side chain; (2) replacement of a hydroxyl group by a non-metabolizable polar substituent, such as a fluorine atom, to prevent its metabolic oxidation or conjugation; and (3) substitution of various ring hydrogen atoms to prevent oxidative or reductive metabolism of the steroid ring system.

In yet another embodiment, the aminosterol comprises a sterol nucleus and a polyamine, attached at any position on the sterol, such that the molecule exhibits a net charge of at least +1, the charge being contributed by the polyamine.

In yet another embodiment, the aminosterol comprises a bile acid nucleus and a polyamine, attached at any position on the bile acid, such that the molecule exhibits a net positive charge being contributed by the polyamine.

In some embodiments, the compositions used in the methods of the invention comprise: (a) at least one pharmaceutical grade aminosterol; and optionally (b) at least one phosphate selected from the group consisting of an inorganic phosphate, an inorganic pyrophosphate, and an organic phosphate. In some embodiments, the aminosterol is formulated as a weakly water soluble salt of the phosphate. In some embodiments, the phosphate is an inorganic polyphosphate, and the number of phosphates can range from about 3 (tripolyphosphate) to about 400, or any number in-between these two values. In other embodiments, the phosphate is an organic phosphate which comprises glycerol 2 phosphates.

In some embodiments, the aminosterol is selected from the group consisting of: (a) squalamine or a pharmaceutically acceptable salt or derivative thereof; (b) a squalamine isomer; (c) Aminosterol 1436; (d) an aminosterol comprising a sterol or bile acid nucleus and a polyamine, attached at any position on the sterol or bile acid, such that the molecule exhibits a net charge of at least +1, the charge being contributed by the polyamine; (e) an aminosterol which is a derivative of squalamine modified through medical chemistry to improve biodistribution, ease of administration, metabolic stability, or any combination thereof; (f) an aminosterol modified to include one or more of the following: (i) substitutions of the sulfate by a sulfonate, phosphate, carboxylate, or other anionic moiety chosen to circumvent metabolic removal of the sulfate moiety and oxidation of the cholesterol side chain; (ii) replacement of a hydroxyl group by a non-metabolizable polar substituent, such as a fluorine atom, to prevent its metabolic oxidation or conjugation; and (iii) substitution of various ring hydrogen atoms to prevent oxidative or reductive metabolism of the steroid ring system; (g) an aminosterol that can inhibit the formation of actin stress fibers in endothelial cells stimulated by a ligand known to induce stress fiber formation, having the chemical structure of Formula I (above); or (h) any combination thereof.

In some embodiments, the methods of the invention can employ a formulation of Aminosterol 1436 as an insoluble salt of phosphate, polyphosphate, or an organic phosphate ester. In some embodiments, the methods of the invention can employ a formulation of Aminosterol 1436 (Zasloff, Williams et al. 2001) as an insoluble salt of phosphate, polyphosphate, or an organic phosphate ester.

Any pharmaceutically acceptable salt of an aminosterol can be used in the compositions and methods of the invention. For example, a phosphate salt or buffer, free base, succinate, phosphate, mesylate or other salt form associated with low mucosal irritation can be utilized in the methods and compositions of the invention.

C. Routes of Administration

It is appreciated that the “fixed dose” disclosed herein can be administered via any suitable route of administration, including but not limited to oral or intranasal delivery, injection (IP, IV, or IM) or a combination thereof.

Further, co-administration of the “fixed dose” with injectable (e.g., 1P, IV, IM) aminosterol formulations is also contemplated herein. For injectable dosage forms, the dosage form can comprise an aminosterol at a dosage of, for example, about 0.1 to about 20 mg/kg body weight. In other embodiments, the effective daily dosing amount is about 0.1, about 0.5, about 1, about 2, about 3, about 4, about 5, about 6, about 7, about 8, about 9, about 10, about 11, about 12, about 13, about 14, about 15, about 16, about 17, about 18, about 19, or about 20 mg/kg body weight.

The invention also encompasses methods of treatment using a combination of an aminosterol composition administered via one route, e.g., oral, with a second aminosterol composition, comprising the same or a different aminosterol, administered via a different route, e.g., intranasal. For example, in a method of the invention, squalamine can be administered orally and aminosterol 1436 can be administered IN.

Also encompassed are methods of treatment comprising administering low dosage aminosterol intranasal compositions of the disclosure to a subject in need. The subject to be treated can be a human, such as an infant, toddler, school-aged child, teenager, young adult, adult, or elderly subject.

The methods of the invention encompass combination treatment, where the intransally administered aminosterol is administered in combination with at least one additional active agent to achieve either an additive or synergistic effect. In one embodiment, the additional active agent is an aminosterol which is delivered orally. For example, the aminosterol administered intranasally can be aminosterol 1436 or a salt or derivative thereof, and the aminosterol administered orally can be squalamine or a salt or derivative thereof. The additional active agent the additional active agent can be administered via a method such as concomitantly, as an admixture, separately and simultaneously or concurrently, or separately and sequentially.

D. Dosing Period

The pharmaceutical composition comprising an aminosterol or a derivative or salt thereof can be administered for any suitable period of time, including as a maintenance dose for a prolonged period of time. Dosing can be done on an as needed basis using any pharmaceutically acceptable dosing regimen. Aminosterol dosing can be no more than 1× per day, once every other day, once every three days, once every four days, once every five days, once every six days, once a week, or divided over multiple time periods during a given day (e.g., twice daily).

In other embodiments, the composition can be administered: (1) as a single dose, or as multiple doses over a period of time; (2) at a maintenance dose for an indefinite period of time; (3) once, twice or multiple times; (4) daily, every other day, every 3 days, weekly, or monthly; (5) for a period of time such as about 1, about 2, about 3, or about 4 weeks, about 1, about 2, about 3, about 4, about 5, about 6, about 7, about 8, about 9, about 10, about 11, or about 12 months, about 1 year, about 1.5 years, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, or about 25 years, or (6) any combination of these parameters, such as daily administration for 6 months, weekly administration for 1 or more years, etc.

Yet another exemplary dosing regimen includes periodic dosing, where an effective dose can be delivered once every about 1, about 2, about 3, about 4, about 5, about 6 days, or once weekly.

In a preferred embodiment, the aminosterol dose is taken in the morning, i.e. on an empty stomach preferably within about two hours of waking up and may be followed by a period without food, such as for example about 60 to about 90 minutes. In other embodiments, the aminosterol dose is taken within about 15 min, about 30 min, about 45 min, about 1 hr, about 1.25 hrs, about 1.5 hrs, about 1.75 hrs, about 2 hrs, about 2.25 hrs, about 2.5 hrs, about 2.75 hrs, about 3 hrs, about 3.25 hrs, about 3.5 hrs, about 3.75 hrs, or about 4 hrs within waking up. In yet further embodiments, the aminosterol dose is followed by about period without food, wherein the period is at least about 30 min, about 45 mins, about 60 mins, about 1.25 hrs, about 1.5 hrs, about 1.75 hrs, or about 2 hrs.

Not to be bound by theory, it is believed that since aminosterols have an impact on circadian rhythms, likely due to ENS signaling thereof, taking the aminosterol dose in the morning enables the synchronization of all the autonomic physiological functions occurring during the day. In other embodiments of the invention, the aminosterol dosage is taken within about 15 mins, about 30 mins, about 45 mins, about 1 hour, about 1.25 hrs, about 1.5 hrs, about 1.75 hrs, about 2 hrs, about 2.25 hrs, about 2.5 hrs, about 2.75 hrs, about 3 hrs, about 3.25 hrs, about 3.5 hrs, about 3.75 hrs, or about 4 hrs of waking up. In addition, in other embodiments of the invention, following the aminosterol dosage the subject has a period of about 15 mins, about 30 mins, about 45 mins, about 1 hours, about 1.25 hrs, about 1.5 hrs, about 1.75 hrs, about 2 hrs, about 2.25 hrs, about 2.5 hrs, about 2.75 hrs, or about 3 hours without food.

E. Composition Components

In some embodiments, a pharmaceutical composition disclosed herein comprises one or more pharmaceutically acceptable carriers, such as an aqueous carrier, buffer, and/or diluent.

In some embodiments, a pharmaceutical composition disclosed herein further comprises a simple polyol compound, such as glycerin. Other examples of polyol compounds include sugar alcohols. In some embodiments, a pharmaceutical composition disclosed herein comprises an aqueous carrier and glycerin at about a 2:1 ratio.

The formulations may conveniently be presented in unit dosage form and may be prepared by any of the methods well known in the art of pharmacy. An exemplary oral dosage form is a tablet or capsule. An exemplary intranasal dosage form is a liquid or powder nasal spray. A nasal spray is designed to deliver drug to the upper nasal cavity, and can be a liquid or powder formulation, and in a dosage form such as an aerosol, liquid spray, or powder.

The present invention is also directed to low dose, intranasal dosage forms of aminosterols. In one embodiment, encompassed is a pharmaceutical composition formulated for intranasal administration, comprising a low dosage of at least one aminosterol or a pharmaceutically acceptable salt or derivative thereof, wherein the dosage of the aminosterol does not result in a pharmacological effect when given orally or by injection.

The low dosage of the aminosterol can be, for example, between about 0.001 to about 6 mg. In another embodiment, the low dosage of the aminosterol can be, for example, about 0.001 to 4 mg/kg. In another embodiment, the low dosage of an aminosterol is a dosage which is subtherapeutic when given orally or by injection.

The aminosterol may be combined or coordinately administered with a suitable carrier or vehicle depending on the route of administration. As used herein, the term “carrier” means a pharmaceutically acceptable solid or liquid filler, diluent or encapsulating material. A water-containing liquid carrier can comprise pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories can be found in the U.S. Pharmacopeia National Formulary, 1857-1859, and (1990). Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art. Examples of filling agents include lactose monohydrate, lactose anhydrous, and various starches; examples of binding agents include various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil® 200, talc, stearic acid, magnesium stearate, calcium stearate, and silica gel. Examples of sweeteners may include any natural or artificial sweetener, such as sucrose, xylitol, sodium saccharin, cyclamate, aspartame, and acesulfame. Examples of flavoring agents are Magnasweet® (trademark of MAFCO), bubble gum flavor, and fruit flavors, and the like. Examples of preservatives include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Any pharmaceutical used for therapeutic administration can be sterile. Sterility is readily accomplished by for example filtration through sterile filtration membranes (e.g., 0.2 micron membranes). Any pharmaceutically acceptable sterility method can be used in the compositions of the invention.

The pharmaceutical composition comprising an aminosterol derivatives or salts thereof will be formulated and dosed in a fashion consistent with good medical practice, taking into account the clinical condition of the individual patient, the method of administration, the scheduling of administration, and other factors known to practitioners.

F. Kits

Aminosterol formulations or compositions of the invention may be packaged together with, or included in a kit along with instructions or a package insert. Such instructions or package inserts may address recommended storage conditions, such as time, temperature and light, taking into account the shelf-life of the aminosterol or derivatives or salts thereof. Such instructions or package inserts may also address the particular advantages of the aminosterol or derivatives or salts thereof, such as the ease of storage for formulations that may require use in the field, outside of controlled hospital, clinic or office conditions.

The invention also provides a pharmaceutical pack or kit comprising one or more containers filled with one or more aminosterol pharmaceutical compositions disclosed herein. The kits may include, for instance, containers filled with an appropriate amount of an aminosterol pharmaceutical composition, either as a powder, a tablet, to be dissolved, or as a sterile solution. Associated with such container(s) can be a notice in the form prescribed by a governmental agency regulating the manufacture, use or sale of pharmaceuticals or biological products, which notice reflects approval by the agency of manufacture, use or sale for human administration. In addition, the aminosterol or a derivative or salt thereof may be employed in conjunction with other therapeutic compounds.

In other aspects, a kit comprising a nasal spray device as described herein is disclosed. In one aspect, the kit may comprise one or more devices as disclosed herein, comprising a disclosed low dose aminosterol composition, wherein the device is sealed within a container sufficient to protect the device from atmospheric influences. The container may be, for example, a foil, or plastic pouch, particularly a foil pouch, or heat sealed foil pouch. Suitable containers sufficient to adequately protect the device will be readily appreciated by one of skill in the art.

In one aspect, the kit may comprise one or more devices as disclosed herein, wherein the device may be sealed within a first protective packaging, or a second protective packaging, or a third protective packaging, that protects the physical integrity of the product. One or more of the first, second, or third protective packaging may comprise a foil pouch. The kit may further comprise instructions for use of the device. In one aspect, the kit contains two or more devices.

In one aspect, the kit may comprise a device as disclosed herein, and may further comprise instructions for use. In one aspect, the instructions may comprise visual aid/pictorial and/or written directions to an administrator of the device.

G. Patient Populations

The disclosed compositions can be used to treat a range of subjects, including human and non-human animals, including mammals, as well as immature and mature animals, including human children and adults. The human subject to be treated can be an infant, toddler, school-aged child, teenager, young adult, adult, or elderly patient.

In embodiments disclosed herein relating to prevention, particular patient populations may be selected based on being “at risk for” the development of one or more disorders. For example, genetic markers of Alzheimer's disease (e.g. APOE4) or family history may be used as signs to identify subjects likely to develop Alzheimer's disease. Thus, in some embodiments relating to disorders for which certain genetic or hereditary signs are known, prevention may involve first identifying a patient population based on one of the signs. Alternatively, certain symptoms are considered early signs of particular disorders. For example, constipation is considered an early sign of Parkinson's disease. Thus, in some embodiments relating to Parkinson's disease, a patient population may be selected for being “at risk” for developing Parkinson's disease based on age and experiencing constipation. An exemplary population is young adults between the ages of about 20 and about 40 experiencing constipation characterized by less than 3 bowel movements per week. These patients can be targeted and monitored for prevention of Parkinson's disease onset. Further genetic or hereditary signs may be used to refine the patient population.

### IV. Methods of Prevention and/or Treatment with a “Fixed Dose” of Aminosterol

Aspects of this disclosure relate to methods of (1) treatment of certain symptoms and/or (2) treatment and/or prevention of disorders associated with one or more of these symptoms by administration of an aminosterol composition, and optionally with (i) administration via non-oral means; and/or (ii) a “fixed dose” of aminosterol as disclosed herein. As noted herein, one or more of the symptoms disclosed herein can be used to determine the fixed dose during the dose escalation process.

Example 1 provides a detailed protocol for determining a “fixed dose” based on improvement of one symptom associated with Parkinson's disease (PD), e.g., constipation. This example further details how this “fixed dose” successfully treated not only constipation, but also other non-dopamine related symptoms of PD.

As dopaminergic activity distinguishes PD from other neurodegenerative disorders and these data relate to symptoms that do not relate to this distinguishing feature, this dosing regime is believed to be extrapolatable both to other symptoms and other disorders.

Not to be bound by theory, it is believed that establishing a patient-specific “fixed dose” based on hitting a threshold improvement in any of the symptoms listed below and administering this therapeutically effective fixed dose will successfully treat the initial symptom and one or more of the other symptoms. Further, to the extent that these symptoms are tied to an underlying disorder, administration of the therapeutically effective fixed dose is also believed to offer a means of treating, preventing, and/or delaying onset of the underlying disorder.

A. Symptoms

1. Constipation

In one aspect of the disclosure, encompassed is a method of treating, preventing, and/or slowing the onset or progression of constipation and/or a constipation-related symptom in a subject in need comprising administering to the subject a therapeutically effective amount of at least one aminosterol, or a salt or derivative thereof, provided that the administering does not comprise oral administration. For example, the method of administering can comprise nasal, sublingual, buccal, rectal, vaginal, intravenous, intra-arterial, intradermal, intraperitoneal, intrathecal, intramuscular, epidural, intracerebral, intracerebroventricular, transdermal administration, or any combination thereof. Nasal administration is preferred.

In another aspect, encompassed is a method of treating, preventing, and/or slowing the onset or progression of constipation and/or a constipation-related symptom in a subject in need comprising administering to the subject a therapeutically effective amount of at least one aminosterol, or a salt or derivative thereof, wherein the method further comprises determining a “fixed” aminosterol dose, as described herein.

Constipation is a common problem worldwide, affecting 2% to 27% of the population, with most estimates varying from 12% to 20%. The prevalence of constipation increases to 30%-40% among people aged >65 years and women are disproportionately affected. In North America, 63M people meet the Rome IV criteria for constipation and in the US alone, constipation is responsible for over 2M physician visits annually. Laxatives are prescribed to 2-3M patients every year and furthermore, in most patients, the condition is chronic requiring lifelong treatment.

Constipation is much more common among patients with PD than in the general population. There are 1M people suffering from Parkinson's Disease in the US, of which roughly 60%, or 600,000 suffer from chronic constipation and in most, the condition is chronic, severe and unresponsive to standard therapy. This represents an economic burden to the individual with PD and to the healthcare system. According to the most recent Federal Supply Schedule (FSS; April 2016), the average 30-day reimbursed price for a basket of orally administered drugs for constipation is approximately $260 or $3120 per year. This represents about $1.8B of prescription laxatives just for patients with PD.

Constipation not only constitutes a major economic burden, but it also significantly affects the quality of life of the individual, contributing to social isolation and depression. Furthermore, the severity of the symptoms correlates negatively with patient reported quality of life.

An effective pro-kinetic medication for individuals with constipation would be a useful addition to the currently available treatments for this condition.

Constipation is defined as a lower than normal frequency of bowel movements in a fixed duration of time (e.g. less than 3 bowel movements per week). While often dismissed as strictly a gastrointestinal symptom, constipation is believed to be an early indicator of neurodegenerative disease to the extent that ENS degeneration can be indicative of later CNS degeneration. Indeed, not to be bound by theory, but constipation is believed to be one of the earliest indicators of PD pathology. Accordingly, method embodiments disclosed herein relate to the treatment of constipation or the treatment and/or prevention of an underlying disorder associated with constipation.

Constipation is common in PD and often becomes symptomatic years before the onset of the motor dysfunction and the subsequent diagnosis of PD. There is substantial evidence that the neurodegenerative process associated with PD, namely the accumulation of toxic aggregates of alpha-synuclein, occurs within the enteric nervous system years before they appear within the brain. It is believed that the enteric nervous system (ENS), with its vast surface area, is subject to continuous insults from infectious agents and toxic substances. Although the function of alpha-synuclein is not known, inflammation within the nervous system leads to an increase in its intracellular levels. In individuals with PD the increase in alpha-synuclein leads to the formation of neurotoxic aggregates, perhaps because of a failure by the neuron (due to genetic factors) to effectively dispose of them. The aggregates of alpha-synuclein then traffic along the vagal nerve to the dorsal motor nucleus within the brainstem, and from there to more rostral structures.

The individual with PD suffers from a form of constipation that is believed to be caused principally by delayed transit through the colon. In addition, defecation is often impaired by dysfunction of the PD subject's anorectal reflex. For many individuals, bowel issues represent a significant detriment to quality of life. Failure to effectively manage this problem can also lead to bowel obstruction, especially as the terminal phase of PD approaches. A limited number of therapies have been subjected to clinical trials and they include agents that increase the fluid content of the stool, either by blocking fluid resorption or increasing the osmolar load within the intestine.

Constipation is a major clinical component of PD and is reported to occur in greater than 60% of affected individuals. The pathophysiological basis of constipation in PD is generally believed to be due to delayed transit through the colon. Several studies have demonstrated that transit of stool through the colon of an individual with PD is about 50% that measured in age matched controls. As a consequence, both stool frequency and stool consistency are abnormal in PD. For many patients, as well as those caring for these individuals, constipation remains a significant morbidity associated with the condition.

Few placebo-controlled clinical trials have been conducted in the PD population to assess the efficacy of therapeutics that could be of value. Addition of fiber to the diet, although increasing stool volume, is reported to have no effect on colon transit time. An osmotic laxative, polyethylene glycol (Magrogol) has been studied in a small placebo controlled clinical trial of individuals with mild constipation, and shown to provide benefit with respect to stool frequency and consistency. A short term placebo controlled trial of Lubiprostone, a chloride channel activator which increases intestinal fluid secretion, was only effective in about 50% of those treated, and resulted in passage of loose stools/diarrhea in place of constipation. Furthermore, Lubiprostone delays gastric emptying, a function already compromised in PD.

The pathophysiology of the gastrointestinal (GI) dysfunction in PD involves deposition of alpha-synuclein within both the ENS as well as within the brainstem. For reasons that remain unknown alpha-synuclein, which is a protein normally produced in neurons, forms neurotoxic intracellular aggregates in PD. Numerous studies suggest that the alpha-synuclein aggregate formation begins in the ENS of the PD individual many years before the onset of the motor symptoms. As a consequence of the normal retrograde neuronal trafficking that occurs within the vagus nerve, toxic aggregates are transported from the neurons of the ENS to the dorsal motor nucleus of the vagus, and then, gradually to sites within the brain that are involved in physical movement and balance. Because the constipation is fundamentally of an acquired neurodegenerative nature, it differs from other forms of this condition.

Example 1 describes several tools used to measure and evaluate the effect of aminosterol treatment on constipation, including for example:

(1) Rome-IV Criteria for Constipation (7 criteria, with constipation diagnosis requiring two or more of the following: (i) straining during at least 25% of defecations, (ii) lumpy or hard stools in at least 25% of defecations, (iii) sensation of incomplete evacuation for at least 25% of defecations, (iv) sensation of anorectal obstruction/blockage for at least 25% of defecations; (v) manual maneuvers to facilitate at least 25% of defecations; (vi) fewer than 3 defecations per week; and (vii) loose stools are rarely present without the use of laxatives;

(2) Constipation—Ease of Evacuation Scale (from 1-7, with 7=incontinent, 4=normal, and 1=manual disimpaction);

(3) Bristol Stool Chart (see FIG. 23), which is a patient-friendly means of categorizing stool characteristics (assessment of stool consistency is a validated surrogate of intestinal motility) and stool diary;

(4) Unified Parkinson's Disease Scale (UPSRS), section 1.11 (Constipation Problems);

(5) Patient Assessment of Constipation Symptoms (PAC-SYM); and

(5) Patient Assessment of Constipation Quality of Life (PAC-QOL).

Yet another diagnostic tool used to assess constipation and related symptoms is the Constipation Assessment Scale (CAS), developed by McMillan and Williams (1989). The CAS (FIG. 24) was based on earlier research and clinical literature and includes eight commonly identified characteristics of constipation, including: (1) abdominal distension or bloating; (2) change in amount of gas passed rectally; (3) less frequent bowel movements; (4) oozing liquid stool; (5) rectal fullness or pressure; (6) rectal pain with bowel movement; (7) small stool size; and (8) urge but inability to pass stool (FIG. 24).

Examples of characteristics of constipation that can be positively affected by the method of the invention include, but are not limited to, frequency of constipation, duration of constipation symptoms, bowel movement frequency, stool consistency, abdominal pain, abdominal bloating, incomplete evacuation, unsuccessful attempts at evacuation, pain with evacuation, and straining with evacuation. Potentially all of these characteristics can be positively impacted by the methods of the invention. Further, assessments of these characteristics are known in the art, e.g. spontaneous bowel movements (SBMs)/week, stool consistency (Bristol Stool Form Scale) (Lewis and Heaton 1997; Heaton et al. 1992), ease of passage (Ease of Evacuation Scale) (Andresen et al. 2007), rescue medication use and symptoms and quality of life related to bowel function (PAC-SYM (Frank et al. 1999) and PAC-QOL (Marquis et al. 2005)).

The methods of using a therapeutically effective fixed dose of an aminosterol composition according to the invention to treat and/or prevent constipation preferably results in an increase in the number of spontaneous bowel movements per week and/or an improvement in other stool conditions. The increase can be, for example, an increase of between 1 to 3 spontaneous bowel movements in a week, or, optionally, full restoration of regular bowel function.

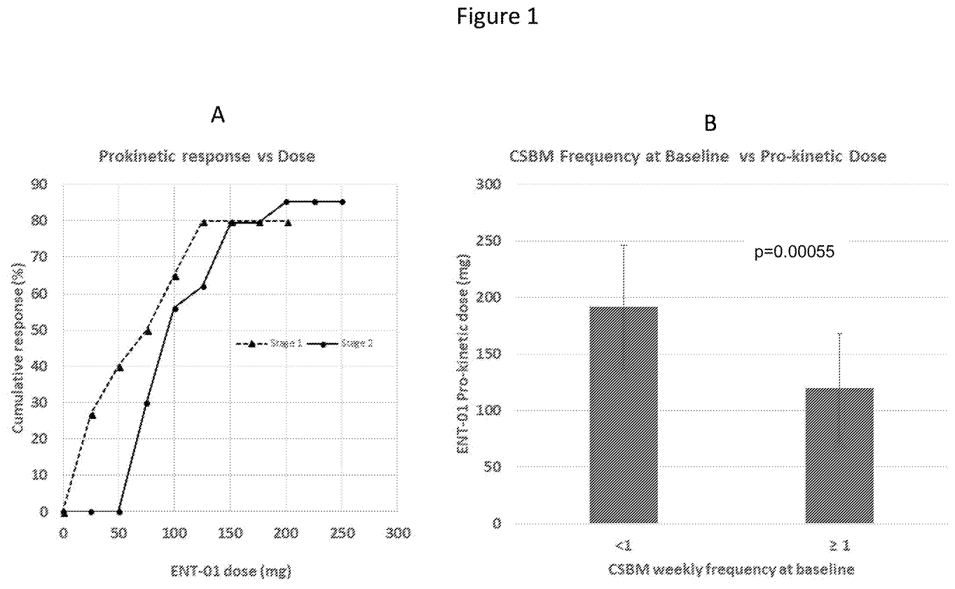

Data detailed in Example 1 shows that 80% of subjects responded to aminosterol treatment with improved bowel function (see FIG. 1A), with the cumulative response rate increasing in a dose-dependent fashion from 25% at 25 mg to a maximum of 80% at 200 mg (Stage 1, FIG. 1A). In Stage 2 of the study, the response rate increased in a dose-dependent fashion from 26% at 75 mg to 85.3% at 250 mg (FIG. 1A). The dose required for a bowel response was patient-specific and varied from 75 mg to 250 mg. The median efficacious dose was 100 mg.

The average CSBM/week increased from 1.2 at baseline to 3.8 at fixed dose (216% improvement) and SBM increased from 2.6 at baseline to 4.5 at fixed dose (73% improvement). Use of rescue medication decreased from 1.8/week at baseline to 0.3 at fixed dose (83% decrease). Consistency based on the Bristol stool scale also improved, increasing from mean 2.7 to 4.1 (52% improvement) and ease of passage increased from 3.2 to 3.7 (16% improvement). Subjective indices of wellbeing (PAC-QOL) and constipation symptoms (PAC-SYM) also improved during treatment.

The dose that proved efficacious in inducing a bowel response was strongly related to constipation severity at baseline (FIG. 1B); patients with baseline constipation of <1 CSBM/week required higher doses for a response (mean 192 mg) than patients with ≥1 CSBM/week (mean 120 mg).

In one embodiment of the invention, treatment of a subject having constipation with an aminosterol in a method described herein results in an improvement of one or more characteristics of constipation. The improvement can be, for example, about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, about 300, about 325, about 350, about 375 or about 400%. Examples of constipation characteristics that can be improved by the methods of the invention include, but are not limited to, frequency of constipation, duration of constipation symptoms, bowel movement frequency, stool consistency, abdominal pain, abdominal bloating, incomplete evacuation, unsuccessful attempts at evacuation, pain with evacuation, and straining with evacuation. Measurement of a constipation characteristic can be done using any clinically recognized scale or tool.

One surprisingly discovery that resulted from the experiments described herein related to aminosterol dosing. It was surprisingly discovered that the dose of aminosterol required to obtain a positive impact on a symptom being evaluated, referred to herein as a “fixed escalated aminosterol dose,” is patient specific. Moreover, it was discovered that the fixed escalated aminosterol dose is not dependent upon age, size, or weight but rather is individually calibrated. Further, it was discovered that the severity of constipation correlates with a higher required “fixed escalated aminosterol dose.” It is theorized that the aminosterol dose required to obtain a positive effect in a subject for the symptom being evaluated correlates with the extent of neuronal damage. Thus, it is theorized that greater neuronal damage correlates with a higher required aminosterol dose to obtain a positive effect in a subject for the symptom being evaluated. The observation that the aminosterol dose required to achieve a desired response increases with constipation severity supports the hypothesis that the greater the burden of αS impeding neuronal function, the higher the dose of aminosterol required to restore normal bowel function. Moreover, the data described in Example 1 confirms the hypothesis that gastrointestinal dysmotility in PD results from the progressive accumulation of αS in the ENS, and that aminosterol treatment can restore neuronal function by displacing αS and stimulating enteric neurons. These results demonstrate that the ENS in PD is not irreversibly damaged and can be restored to normal function.

In calibrating the fixed aminosterol dose for a specific patient, the starting dose is varied based upon the severity of the constipation. Thus, for subjects with severe constipation, e.g., subjects with 1 or less CSBM or SMB per week, oral aminosterol dosing is started at about 100 to about 150 mg or more (or any amount in-between these values as described herein). For subjects with less severe constipation, e.g., more than 1 CSBM or SBM per week, oral aminosterol dosing is started at about 25 to about 75 mg (or any amount in-between these values as described herein). Dosing for both patients is then escalated by defined amounts over a defined period of time until the fixed escalated dose for the patient is identified. Aminosterol doses can also be de-escalated (reduced) if any given aminosterol dose induces a persistent undesirable side effect, such as diarrhea, vomiting, or nausea.

For example, for patients with severe constipation, a starting oral aminosterol dosage can be from 75 mg up to about 300 mg, or any amount in-between these two values. In other embodiments, the starting oral aminosterol dosage for severely constipated patients can be, for example, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, or about 300 mg. A “fixed escalated” oral aminosterol dose for a severely constipated patient is likely to range from about 75 mg up to about 500 mg. As described in Example 1, a positive effect was defined as a dose that resulted in a CSBM within 24 hours of dosing on at least 2 of 3 days at a given dose.

For patients with less severe constipation, oral aminosterol dosing is started at about 10 to about 75 mg, or any amount in-between these two values as described herein. For example, starting oral aminosterol dosage for patients with moderate to mild constipation can be about 1, about 5, about 10, about 15, about 20, about 25, about 30, about 35, about 40, about 45, about 50, about 55, about 60, about 65, about 70, up to less than or equal to about 75 mg. A fixed escalated oral aminosterol dose for a mild or moderately constipated patient is likely to range from about 5 mg up to about 350 mg, or any amount in-between these two values as described herein.

In one embodiment, a method of treating, preventing, and/or slowing the onset or progression of constipation and/or a related symptom in a subject in need comprising administering to the subject a therapeutically effective amount of at least one aminosterol, or a salt or derivative thereof, is provided via non-oral administration

In another embodiment, the present disclosure is directed to methods of treating constipation and/or a constipation-related symptom in a subject in need, comprising (a) determining a dose of an aminosterol or a salt or derivative thereof for the subject, wherein the aminosterol dose is determined based on the effectiveness of the aminosterol dose in improving or resolving constipation and/or the constipation-related symptom in the subject; (b) followed by administering the dose of the aminosterol or a salt or derivative thereof to the subject for a period of time. The method of determining the aminosterol dose comprises (i) identifying a constipation-related symptom to be evaluated; (ii) identifying a starting aminosterol dose for the subject; and (iii) administering an escalating dose of the aminosterol to the subject over a period of time until an effective aminosterol dose is identified, wherein the effective aminosterol dose is the dose where improvement or resolution of the constipation-related symptom is observed, and fixing the aminosterol dose at that level in that particular subject. Optionally, each defined period of time is independently selected from the group consisting of about 1 day to about 10 days, about 10 days to about 30 days, about 30 days to about 3 months, about 3 months to about 6 months, about 6 months to about 12 months, and about greater than 12 months.

The constipation-related symptom can be any known symptom of constipation. For example, the symptom can be selected from the group consisting of frequency of constipation, duration of constipation symptoms, frequency of bowel movements, fecal incontinence/encopresis, abdominal pain, abdominal distension or bloating, abdominal discomfort, stomach cramps, stool consistency, painful defecation/rectal pain with bowel movement, rectal burning during or after bowel movement, rectal bleeding or tearing during or after a bowel movement, ease of defecation/passing stool, straining during defecation and/or straining or squeezing to try to pass bowel movements, incomplete evacuation or bowel movement, unsuccessful attempts at evacuation, sensation of incomplete bowel evacuation, sensation of anorectal obstruction/blockage, bowel movements that were too hard, bowel movements that were too small, change in amount of gas passed rectally, less frequent bowel movements, oozing liquid stool, rectal fullness or pressure, small stool size, urge but inability to pass stool, or personal judgement of constipation.

In another embodiment, the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%. The improvement can be measured, for example, using a clinically recognized scale or tool.

In one embodiment, the constipation-related symptom is frequency of bowel movements, and the improvement or resolution comprises a desired rate of complete spontaneous bowel movement (CSBM) or spontaneous bowel movement (SBM). In another embodiment, the constipation-related symptom is frequency of bowel movements, and the improvement or resolution comprises a rate of CSBM or SBM in the subject of one or more CSBM or SBM per week, 2 or more CSBM or SMB per week, or 3 or more CSBM or SBM per week. In yet a further embodiment, the improvement or resolution comprises an increase in bowel activity, an induction of nausea, an induction of secretory diarrhea, or any combination thereof.

In one embodiment, the starting dose of an aminosterol or a salt or derivative thereof is based on a baseline rate of complete spontaneous bowel movement (CSBM) or spontaneous bowel movement (SBM) in the subject. In another embodiment, the starting dose of the aminosterol or a salt or derivative thereof is higher if the constipation is severe, where “severe” is defined as less than one CSBM or SBM per week.

In another embodiment, a subject experiencing moderate constipation or a related symptom, which is defined as a baseline rate of CSBM or SBM in the subject of one or more CSBM or SBM per week, is administered a starting oral dose of aminosterol or a salt or derivative thereof of from about 10 to about 75 mg/day. For example, in this embodiment the starting oral aminosterol dose can be about 10, about 15, about 20, about 25, about 30, about 35, about 40, about 45, about 60, about 65, about 70, or about 75 mg/day.

In yet another embodiment, a subject experiencing severe constipation or a related symptom, which is defined as a baseline rate of CSBM or SBM in the subject of less than one CSBM or SBM per week, is administered a starting oral aminosterol dose of at least about 75 mg/day. For example, in this embodiment the starting oral aminosterol dose may be from about 75 to about 175 mg/day, or higher. For example, the starting oral aminosterol dose may be about 75, about 80, about 85, about 90, about 95, about 100, about 105, about 110, about 115, about 120, about 125, about 130, about 135, about 140, about 145, about 150 about 155, about 160, about 165, about 170, or about 175 mg/day. In one aspect of this embodiment, the starting oral aminosterol dose is at least about 175 mg/day.

In one aspect of the invention, the subject is suffering from a disorder of gastrointestinal motility. In another aspect of the invention, the subject is suffering from a condition or disorder selected from the group consisting of chronic idiopathic constipation, Irritable bowel syndrome, Opioid-induced constipation, and Inflammatory Bowel Disease. In yet a further aspect of the invention, the subject is suffering from a neurodegenerative disease. For example, the neurodegenerative disease can be Parkinson's Disease, Alzheimer's disease (AD), Huntington's chorea and/or Huntington's disease, Multiple Sclerosis, Amyotorphic Lateral Sclerosis (ALS), multiple system atrophy (MSA), schizophrenia, Friedreich's ataxia, vascular dementia, Lewy Body dementia or disease, spinal muscular atrophy, supranuclear palsy, fronto temperal dementia, progressive nuclear palsy, Guadeloupian Parkinsonism, spinocerebellar ataxia, autism, dementia of aging, neuropathy of diabetes, peripheral sensory neuropathy, cerebral palsy, epilepsy, diabetic neuropathy, traumatic head and/or spine injury, stroke, or depression.

2. Hallucinations

A hallucination is a sensory impression or perception of an object or event, in any of the 5 senses (sight, touch, sound, smell, or taste) that has no basis in external stimulation. Hallucinations can have debilitating impact on the subject's health and life by causing harm to self or others, by making it difficult for the subject to function normally in everyday situations, and by causing sleep disruption. Examples of hallucinations include “seeing” someone not there (visual hallucination), “hearing” a voice not heard by others (auditory hallucination), “feeling” something crawling up your leg (tactile hallucination), “smelling” (olfactory), and “tasting” (gustatory). Other examples of hallucination types include hypnagogic hallucination (a vivid, dreamlike hallucination occurring at sleep onset), hypnopompic hallucination (a vivid, dreamlike hallucination occurring on awakening), kinesthetic hallucination (a hallucination involving the sense of bodily movement), and somatic hallucination a hallucination involving the perception of a physical experience occurring within the body.

Hallucinations can be a result of psychiatric conditions or correlated with diseases, such as a neurodisease. Hallucinations, especially auditory hallucinations, are characteristic of certain psychiatric conditions such as schizophrenia, occurring in up to 70-80% of subjects. They also occur in 30-50% of individuals with borderline personality disorder. Auditory hallucinations can take control of actions or behavior and elicit violent defensive behavior or alternatively lead to self-harming behavior. They can also occur in post-partum psychosis. Auditory hallucinations can less commonly occur in severely depressed patients or even in mania. Substance abuse can also be associated with visual hallucinations. Alcohol intoxication or withdrawal, post-traumatic stress disorder (PTSD) and bereavement can also be associated with visual hallucinations.

Hallucinations can be a result of neurological disorders. In one embodiment the neurological disorder is a brain tumor. In some embodiments, the “focal brain lesions.” Formed and unformed visual hallucinations can occur in the presence of temporal and occipital lobe lesions. Occipital lobe lesions typically produce simple geometric patterns or “strings of circles like a bunch of grapes” or stars which can follow the gaze (palinopsia), whereas temporal lobe lesions are associated with complex, formed hallucinations. Temporal lobe lesions and especially lesions of the uncinate gyms are typically associated with olfactory and gustatory hallucinations. Lesions of the cerebral peduncles and substantia nigra are associated with “peduncular hallucinosis” or colorful vivid images. In some embodiments, the hallucinations are a result of diffuse involvement of the cerebral cortex. In some embodiments, of diffuse involvement. Acute metabolic encephalopathies and encephalitis caused by viral infections or diseases associated with a cerebral vasculitis such as Systemic Lupus Erythematosus (SLE) can cause visual hallucinations.

In some cases, hallucination is the result of a psychiatric or neurological disorder. The aminosterol composition can, for example, reverse the dysfunction of the psychiatric or neurological disorder and treat the hallucination. The psychiatric disorder can be, for example, selected from the group consisting of Bipolar disorder, Borderline personality disorder, Depression (mixed), Dissociative identity disorder, Generalized anxiety disorder, Major depression, Obsessive compulsive disorder, Post-traumatic stress disorder, Psychosis (NOS), Schizoaffective disorder, and Schizophrenia.

In other cases, hallucinations can be the result of a neurological disorder. The neurological disorder can be, for example, the result of (a) a brain tumor, (b) a sleep disorder such as narcolepsy, or (c) a focal brain lesion, such as occipital lobe lesions or temporal lobe lesions. In an exemplary embodiment, the temporal lobe lesion can be lesions of the uncinate gyms, cerebral peduncles, or substantia nigra. The neurological disorder can be, for example, the result of (d) a diffuse involvement of the cerebral cortex, such as that caused by a viral infectious disease.

The diffuse involvement of the cerebral cortex can be a result of a cerebral vasculitis condition, and the viral infectious disease can be, for example, acute metabolic encephalopathies, encephalitis, or meningitis. The cerebral vasculitis condition can be caused by an autoimmune disorder, a bacterial or viral infection, or a systemic vasculitis. The autoimmune disorder can be, for example, Systemic Lupus Erythematosus (SLE).

Alternatively, hallucinations can be the result of a neurodegenerative disorder. For example, the neurodegenerative disorder can be, for example, such as Parkinson's disease (PD), supranuclear palsy, multi-system atrophy, Parkinsonism, Alzheimer's disease, Fronto-temporal dementia, amyotrophic lateral sclerosis (ALS), Huntington's Disease, schizophrenia, Friedreich's ataxia, Multiple sclerosis (MS), Lewy Body dementia or disease, spinal muscular atrophy, fronto temperal dementia, progressive nuclear palsy, Guadeloupian Parkinsonism, spinocerebellar ataxia, or vascular dementia. In a preferred embodiment, the aminosterol compositions of the invention reverse the dysfunction of the neurodegenerative disorder and treat the hallucination.

Further still, hallucinations may be caused by a sensory loss. The sensory loss can be, for example, visual, auditory, gustatory, tactile, or olfactory. In a preferred embodiment, the fixed dose aminosterol compositions of the invention reverse the dysfunction of the sensory loss and treat the hallucination. In a preferred embodiment, the aminosterol compositions of the invention reverse the dysfunction of the enteric nervous system and treats the hallucination.

The methods of using a therapeutically effective fixed dose of an aminosterol composition according to the invention to treat and/or prevent hallucinations preferably result in a decrease in hallucinations. The decrease can be, for example, a reduction in occurrences of hallucinations by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The methods of the invention may also result in the subject being hallucination-free. The hallucination can comprise, for example, a visual, auditory, tactile, gustatory or olfactory hallucination. The improvement can be measured using any clinically recognized assessment or tool.

Example 1 describes several tools used to measure and evaluate the effect of aminosterol treatment on hallucinations, including for example:

(1) The University of Miami Parkinson's Disease Hallucinations Questionnaire (UM-PDHQ);

(2) Unified Parkinson's Disease Scale (UPSRS), section 1.2 (Hallucinations and Psychosis); and

(3) direct questioning.

As described in Example 1, the PDHQ score improved from 1.3 at baseline to 0.9 during wash-out. Hallucinations were reported by 5 patients at baseline and delusions in 1 patient. Both hallucinations and delusions improved or disappeared in 5 of 6 patients during treatment and did not return for 4 weeks following discontinuation of aminosterol treatment in 1 patient and 2 weeks in another. In one patient the hallucinations disappeared at 100 mg, despite not having reached the colonic prokinetic dose at 175 mg. Further, unlike stool-related indices, the improvement in many CNS symptoms persisted during wash-out.

3. Erectile Dysfunction

In one aspect, encompassed is a method of treating, preventing, and/or slowing the onset or progression of erectile dysfunction (ED) and/or a related symptom in a subject in need comprising administering to the subject a therapeutically effective amount of at least one aminosterol, or a salt or derivative thereof. The method can comprise, for example, administration selected from oral, nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof and/or nasal administration. In addition, administration can comprise non-oral administration.

In another aspect, encompassed is a method of treating, preventing, and/or slowing the onset or progression of erectile dysfunction (ED) and/or a related symptom in a subject in need comprising: (a) determining a dose of an aminosterol or a salt or derivative thereof for the subject, wherein the aminosterol dose is determined based on the effectiveness of the aminosterol dose in improving or resolving an ED symptom being evaluated, (b) followed by administering the dose of the aminosterol or a salt or derivative thereof to the subject for a defined period of time, wherein the method comprises: (i) identifying an ED symptom to be evaluated; (ii) identifying a starting dose of an aminosterol or a salt or derivative thereof for the subject; and (iii) administering an escalating dose of the aminosterol or a salt or derivative thereof to the subject over a defined period of time until an effective dose for the ED symptom being evaluated is identified, wherein the effective dose is the aminosterol dose where improvement or resolution of the ED symptom is observed, and fixing the aminosterol dose at that level for that particular ED symptom in that particular subject; and (c) optionally wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

Erectile dysfunction can be a sign of a physical or psychological condition. It can cause stress, relationship strain, and low self-confidence. The main symptom is a man's inability to get or keep an erection firm enough for sexual intercourse.

ED can occur manifest through different mechanisms. Based on its mechanism, ED can be classified as psychogenic, neurogenic (failure to initiate erection), artereogenic (failure of the penis to fill with blood), cavernosal (failure of vascular system to retain blood in penis once filled) (Dean et al. 2005).

Psychogenic ED can arise because sexual behavior and penile erection are controlled by the hypothalamus, the limbic system, and the cerebral cortex. Therefore, stimulatory or inhibitory messages can be relayed to the spinal erection centers to facilitate or inhibit erection. Two possible mechanisms have been proposed to explain the inhibition of erection in psychogenic dysfunction: direct inhibition of the spinal erection center by the brain as an exaggeration of the normal suprasacral inhibition and excessive sympathetic outflow or elevated peripheral catecholamine levels, which may increase penile smooth muscle tone to prevent the relaxation necessary for erection.

Neurogenic ED may arise as a result of pathology in the brain. The medial preoptic area, the paraventricular nucleus, and the hippocampus have been regarded as important integration centers for sexual drive and penile erection. Pathologic processes in these regions, in conditions such as Parkinson's disease, stroke, encephalitis, or temporal lobe epilepsy, are often associated with ED. Other lesions in the brain noted to be associated with ED are tumors, dementias, Alzheimer's disease, Shy-Drager (multiple system atrophy), syndrome, and trauma.

Many neurodiseases causing ED such as PD are suspected to correlate with the formation of toxic αS aggregates within the enteric nervous system (ENS) (Braak et al. 2003). ED has been reported to affect in the range of 60-79% of men having PD, while the prevalence of ED in non-Parkinson men is only about 37.5% (Papatsoris, 2006). As a result of the normal trafficking of αS aggregates from the ENS to the central nervous system (CNS) via afferent nerves such as the vagus (Holmqvist et al. 2014; Svensson et al. 2015), neurotoxic aggregates accumulate progressively within the brainstem and more rostral structures. Inhibiting αS aggregation in the ENS may, thus, reduce the continuing neuro disease process in both the ENS and CNS (Phillips et al. 2008), and thereby positively impact ED associated with abnormal αS pathology.

Typically, ED manifests several years after the PD has been established in the patient. Neurodegenerative conditions such as PD may cause damage to brain centers responsible for autonomic processing. It is believed that aminosterols capable of treating or preventing neurodegeneration in PD, may prevent or treat the degeneration of neuronal structure that governs erection either directly or indirectly via the regulation of hormones.

αS is a member of the synuclein family of soluble proteins (αS, β-synuclein and γ-synuclein) that are commonly present in CNS of vertebrates. αS is expressed in the neocortex, hippocampus, substantia niagra, thalamus and cerebellum, with the main location within the presynaptic terminals of neurons in both membrane-bound and cytosolic free forms. Presynaptic terminals release chemical messengers, called neurotransmitters, from compartments known as synaptic vesicles. The release of neurotransmitters relays signals between neurons and is critical for normal brain function. αS can be seen in neuroglial cells and melanocytic cells, and is highly expressed in the neuronal mitochondria of the olfactory bulb, hippocampus, striatum and thalamus.

αS aggregates to form insoluble fibrils in pathological conditions characterized by Lewy bodies, such as PD, dementia with Lewy bodies (DLB) and multiple system atrophy (MSA). These disorders are known as synucleinopathies. αS is the primary structural component of Lewy body fibrils. Occasionally, Lewy bodies contain tau protein; however, αS and tau constitute two distinctive subsets of filaments in the same inclusion bodies. αS pathology is also found in both sporadic and familial cases with AD. Thus, one indicator of αS pathology is the formation of αS aggregates.

At the molecular level, protein misfolding, accumulation, aggregation and subsequently the formation of amyloid deposits are common features in many neurological disorders including Alzheimer's disease (AD) and Parkinson's disease (PD). Thus neurodegenerative diseases are sometimes referred to as proteinopathies. The existence of a common mechanism suggests that neurodegenerative disorders likely share a common trigger and that the nature of the pathology is determined by the type of the aggregated protein and the localization of the cell affected.

Starting two decades ago with the discoveries of genetic links between αS and PD risk and the identification of aggregated αS as the main protein constituent of Lewy pathology, αS has emerged as the major therapeutic target in PD and related synucleinopathies (Brundin et al., 2017). The α-synuclein abnormalities typically found in PD are believed to be responsible for apparent catecholamine-deficits (dopamine is a catecholamine sharing metabolic pathways with other catecholamines) (Frisina et al., 2009). It is known that central dopamine is a key neurotransmitter in the control of sexual function including erection (Giuliano et al 2001). It is thought that dopamine deficiency may be responsible for erectile dysfunction often observed in PD patients (Palma et al 2014). In patients with PD, α-synuclein-related pathology develops in serotonergic and cholinergic neurons in parallel with that seen in the nigral dopamine neurons. Thus, regulation of α-synuclein may play a role in ED in PD via dopaminergic dysfunction.

In one embodiment, the invention encompasses a method of treating, preventing and/or slowing the onset or progression of ED and/or a related symptom in a subject in need comprising administering to the subject a therapeutically effective amount of at least one aminosterol or a salt or derivative thereof.

In another embodiment, the invention encompasses a method of treating, preventing and/or slowing the onset or progression of ED and/or a related symptom in a subject in need comprising (a) determining a dose of an aminosterol or a salt or derivative thereof for the subject, wherein the aminosterol dose is determined based on the effectiveness of the aminosterol dose in improving or resolving a ED symptom being evaluated, (b) followed by administering the aminosterol dose to the subject for a period of time, wherein the method comprises (i) identifying a ED symptom to be evaluated; (ii) identifying a starting aminosterol dose for the subject; and (iii) administering an escalating dose of the aminosterol to the subject over a period of time until an effective dose for the ED symptom being evaluated is identified, wherein the effective dose is the aminosterol dose where improvement or resolution of the ED symptom is observed, and fixing the aminosterol dose at that level for that particular ED symptom in that particular subject.

In one embodiment, the method results in a decrease in the number of instances in which the subject cannot attain erection, and the decrease in number of instances in which the subject cannot attain erection comprises a reduction in number of instances in which the subject cannot attain erection over a defined period of time. In another aspect, the method results in a decreased severity of ED over a defined period of time, wherein the decreased severity of ED is measured by a medically recognized technique selected from the group consisting of bone-pressed erect length (BPEL) measurement, girth measurement, Erection Hardness Scale (EHS), and International Index of Erectile Function (IIEF).

In another embodiment, the starting aminosterol or a salt or derivative thereof dose is higher if the ED symptom being evaluated is severe.

In one embodiment, progression or onset of ED is slowed, halted, or reversed over a defined period of time following administration of the aminosterol or a salt or derivative thereof, as measured by a medically-recognized technique. In another embodiment, the ED is positively impacted by the aminosterol or a salt or derivative thereof, as measured by a medically-recognized technique. Exemplary “defined period of time” can be independently selected from the group consisting of about 1 day to about 10 days, about 10 days to about 30 days, about 30 days to about 3 months, about 3 months to about 6 months, about 6 months to about 12 months, and about greater than 12 months.

In addition, the positive impact and/or progression of ED can be measured quantitatively or qualitatively by one or more techniques selected from the group consisting of bone-pressed erect length (BPEL) measurement, girth measurement, Erection Hardness Scale (EHS), and International Index of Erectile Function (IIEF). Further, the progression or onset of ED can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by the one or more techniques.

In one embodiment, the aminosterol or a salt or derivative thereof reverses dysfunction caused by the ED and treats, prevents, improves, and/or resolves the symptom being evaluated. In another aspect, the improvement or resolution of the ED symptom is measured using a clinically recognized scale or tool; and/or the improvement in the ED symptom is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale.

In one embodiment, the ED symptom to be evaluated is selected from the group consisting of: (a) a symptom from the International Index of Erectile Function (IIEF) selected from the group consisting of likelihood of getting an erection during sexual activity, likelihood that erections are hard enough for penetration, ability to maintain erection after penetration, ability to maintain erection to completion of intercourse, satisfaction with intercourse attempts, likelihood of ejaculation during sexual intercourse or stimulation, likelihood of orgasm during sexual intercourse or stimulation, prevalence of sexual desires, intensity of sexual desires, satisfaction with sexual relationship with partner, and confidence level in ability to get and maintain erection; (b) constipation; (c) sleep disorder or sleep disturbance; (d) neurodegeneration; (e) cognitive impairment; (f) bone-pressed erect length (BPEL) measurement; (g) hardness as specified in the Erection Hardness Scale (EHS); (h) erect penile girth; (i) high blood pressure; (j) diabetes; (k) atherosclerosis; (l) heart disease; (m) high cholesterol; (n) multiple sclerosis; (o) obesity; (p) depression; and (q) anxiety.

In one embodiment, the ED symptom to be evaluated is a sleep disorder or sleep disturbance, and wherein: (a) the sleep disorder or sleep disturbance comprises a delay in sleep onset, sleep fragmentation, REM-behavior disorder, sleep-disordered breathing including snoring and apnea, day-time sleepiness, micro-sleep episodes, narcolepsy, or any combination thereof; (b) the sleep disorder or sleep disturbance comprises REM-behavior disorder, which comprises vivid dreams, nightmares, and acting out the dreams by speaking or screaming, or fidgeting or thrashing of arms or legs during sleep; (c) the method results in a positive change in the sleeping pattern of the subject; (d) the method results in a positive change in the sleeping pattern of the subject, wherein the positive change is defined as: (i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (e) as a result of the method the subject obtains the total number of hours of sleep recommended by a medical authority for the age group of the subject.

In one embodiment, the ED symptom to be evaluated is bone-pressed erect length (BPEL) measurement and wherein: (a) the method results in an increase in BPEL measurement in the subject; and/or (b) the method results in an increase in BPEL measurement in the subject and the increase in BPEL measurement is defined as an increase in BPEL measurement selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%.

In one embodiment, the ED symptom to be evaluated is erect penile girth and wherein: (a) the method results in an increase in erect penile girth in the subject; and/or (b) the method results in an increase in erect penile girth in the subject and the increase in erect penile girth is defined as an increase in erect penile girth selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%.

In one embodiment, the ED symptom to be evaluated is hardness as specified in the Erection Hardness Scale (EHS) and wherein as a result of the method: (a) the subject's maximum attainable hardness increases 1 grade; (b) the subject's maximum attainable hardness increases 2 grades; (c) the subject's maximum attainable hardness increases 3 grades; and/or (d) the subject that was previously not able to attain a hardness of grade 1, 2, 3, or 4, can attain a hardness of grade 1, 2, 3, or 4; wherein grade 1 is defined as an enlarged but not hard penis, grade 2 is defined as a penis that is hard but not hard enough for penetration, grade 3 is defined as a penis that is hard enough for penetration but not completely hard, and grade 4 is defined as a penis that is completely hard and fully rigid.

In one embodiment, the ED symptom to be evaluated is cognitive impairment, and wherein: (a) progression or onset of the cognitive impairment is slowed, halted, or reversed over a defined time period following administration of the fixed escalated dose of the aminosterol or a salt or derivative thereof, as measured by a medically-recognized technique; and/or (b) the cognitive impairment is positively impacted by the fixed escalated dose of the aminosterol or a salt or derivative thereof, as measured by a medically-recognized technique; (c) the cognitive impairment is positively impacted by the fixed escalated dose of the aminosterol or a salt or derivative thereof, as measured by a medically-recognized technique and the positive impact on and/or progression of cognitive impairment is measured quantitatively or qualitatively by one or more techniques selected from the group consisting of ADASCog, Mini-Mental State Exam (MMSE), Mini-cog test, Woodcock-Johnson Tests of Cognitive Abilities, Leiter International Performance Scale, Miller Analogies Test, Raven's Progressive Matrices, Wonderlic Personnel Test, IQ tests, or a computerized test selected from Cantab Mobile, Cognigram, Cognivue, Cognision, and Automated Neuropsychological Assessment Metrics Cognitive Performance Test (CPT); and/or (d) the progression or onset of cognitive impairment is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In some embodiments, the ED symptom to be evaluated is constipation, and (a) treating the constipation prevents and/or delays the onset and/or progression of the ED; (b) the fixed escalated aminosterol dose causes the subject to have a bowel movement; (c) the method results in an increase in the frequency of bowel movement in the subject; (d) the method results in an increase in the frequency of bowel movement in the subject and the increase in the frequency of bowel movement is defined as: (i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; (e) as a result of the method the subject has the frequency of bowel movement recommended by a medical authority for the age group of the subject; and/or (f) the starting aminosterol dose is determined by the severity of the constipation, wherein: (i) if the average complete spontaneous bowel movement (CSBM) or spontaneous bowel movement (SBM) is one or less per week, then the starting aminosterol dose is at least about 150 mg; and (ii) if the average CSBM or SBM is greater than one per week, then the starting aminosterol dose is about 75 mg or less.

In one embodiment, the ED symptom to be evaluated is neurodegeneration, and (a) treating the neurodegeneration prevents and/or delays the onset and/or progression of the ED; and/or (b) the method results in treating, preventing, and/or delaying the progression and/or onset of neurodegeneration in the subject. In an exemplary embodiment (a) progression or onset of the neurodegeneration is slowed, halted, or reversed over a defined period of time following administration of the fixed escalated dose of the aminosterol or a salt or derivative thereof, as measured by a medically-recognized technique; and/or (b) the neurodegeneration is positively impacted by the fixed escalated dose of the aminosterol or a salt or derivative thereof, as measured by a medically-recognized technique. The positive impact and/or progression of neurodegeneration can be measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis. In addition, the progression or onset of neurodegeneration can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In one embodiment, the ED symptom to be evaluated is depression and wherein: (a) treating the depression prevents and/or delays the onset and/or progression of ED; (b) the method results in improvement in a subject's depression, as measured by one or more clinically-recognized depression rating scale; (c) the method results in improvement in a subject's depression, as measured by one or more clinically-recognized depression rating scale and the improvement is in one or more depression characteristics selected from the group consisting of mood, behavior, bodily functions such as eating, sleeping, energy, and sexual activity, and/or episodes of sadness or apathy; and/or (d) the method results in improvement in a subject's depression, as measured by one or more clinically-recognized depression rating scale, and the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%.

In another embodiment, the aminosterol or a salt or derivative thereof is administered in combination with at least one additional active agent to achieve either an additive or synergistic effect. For example, the additional active agent can be administered via a method selected from the group consisting of (a) concomitantly; (b) as an admixture; (c) separately and simultaneously or concurrently; or (d) separately and sequentially. In another embodiment, the additional active agent is a different aminosterol from that administered in primary method. In yet a further embodiment, the method of the invention comprises administering a first aminosterol which is aminosterol 1436 or a salt or derivative thereof intranasally and administering a second aminosterol which is squalamine or a salt or derivative thereof orally.

In another embodiment, the at least one additional active agent is an active agent used to treat ED or a symptom thereof. In some embodiments, the active agent is selected from the group consisting of PDES inhibitors such as sildenafil (Viagra®), vardenafil (Levitra®), and tadalafil (Cialis®); prostaglandins such as alprostadil (Prostin®); antispasmodics such as papaverine; and alpha-adrenergic antagonists such as phentolamine (Regitine®).

In another embodiment, the subject to be treated according to the methods of the invention can be a member of a patient population at risk for being diagnosed with ED.

4. Cardiac Conduction Defects

In one aspect, encompassed is a method of treating, preventing, and/or slowing the onset or progression of a cardiac conduction defect (CCD) and/or a related symptom in a subject in need comprising administering to the subject a therapeutically effective amount of at least one aminosterol or a salt or derivative thereof. In addition, the method of administration can, for example, (i) comprise administration selected from oral, nasal, sublingual, buccal, rectal, vaginal, intravenous, intra-arterial, intradermal, intraperitoneal, intrathecal, intramuscular, epidural, intracerebral, intracerebroventricular, transdermal, non-oral administration, or any combination thereof; and/or (ii) non oral administration; and/or (iii) nasal administration.

In another aspect, encompassed is a method of treating, preventing, and/or slowing the onset or progression of a cardiac conduction defect (CCD) and/or a related symptom in a subject in need comprising: (a) determining a dose of an aminosterol or a salt or derivative thereof for the subject, wherein the aminosterol dose is determined based on the effectiveness of the aminosterol dose in improving or resolving a CCD symptom being evaluated, (b) followed by administering the dose of the aminosterol or a salt or derivative thereof to the subject for a defined period of time, wherein the method comprises: (i) identifying a CCD symptom to be evaluated; (ii) identifying a starting dose of an aminosterol or a salt or derivative thereof for the subject; (iii) administering an escalating dose of the aminosterol or a salt or derivative thereof to the subject over a defined period of time until an effective dose for the CCD symptom being evaluated is identified, wherein the effective dose is the aminosterol dose where improvement or resolution of the CCD symptom is observed, and fixing the aminosterol dose at that level for that particular CCD symptom in that particular subject; and (c) optionally wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

CCDs relate generally to defects concerning transmission of electrical impulses to and through the heart. These electrical impulses are responsible for the dynamics and rhythm of heartbeat. Disorders or CCDs, can result in irregular heartbeat, also known as arrhythmia. Cardiac conduction defect (CCD) is a serious and potentially life-threatening disorder. CCD refers to a number of aberrations concerning the conduction of around the heart. Different types of CCD result from problems in different circuits or nodes in the hearts electrical circuitry. These problems may include an alteration of cardiac conduction through the atrioventricular (AV) node, the His-Purkinje system with right or left bundle branch block, and widening of QRS complexes as observed via electrocardiogram (ECG or EKG). CCD can lead to complete AV block and cause syncope and sudden death. Originally CCD was considered a structural disease of the heart with anatomic changes in the conduction system underlying abnormal impulse propagation. In a substantial number of cases, however, conduction disturbances are found to occur in the absence of anatomical abnormalities. In these cases, functional rather than structural alterations appear to underlie conduction disturbances.

CCDs may occur along with a variety of neurological and psychiatric disorders, as discussed below. For example, the neurodegenerative condition Parkinson's disease will often also involve CCDs (Scorza et al. 2018). PD patients experience dysregulation in the electrical activity of the heart that can put them at risk to develop cardiac dysrhythmias. Prolongation in the corrected QT (QTc) interval, which describes ventricular depolarization and repolarization corrected for heart rate, predicts cardiovascular mortality and has been reported in PD (Joers et al. 2014).

The autonomic nervous system plays an important role in the modulation of cardiac electrophysiology and arrhythmogenesis (Shen et al. 2014). Parkinson's disease (PD) patients often exhibit impaired regulation of heart rate by the autonomic nervous system (ANS) that may precede motor symptoms in many cases. In PD, accumulation of α-synuclein, precedes damage to dopaminergic neurons. Mice expressing a mutant form of α-synuclein that causes familial PD, exhibit aberrant autonomic control of the heart (Griffioen et al. 2013). The autonomic nervous system is a major element of the cardiac conduction system (Igarashi et al 1989), thus αS pathology may result in CCDs, while also being associated with neurological and psychological conditions discussed herein.

Overexpression of α-synuclein causes loss of norepinephrine-producing cells in the sympathetic system of the heart (Singleton et al 2004). Catecholamines such as norepinephrine have a governing effect over cardiac processes. They can increase inotropic effect, causing contractility of the cardiac muscle thus increasing the cardiac output by increasing the stroke volume. Catecholamines increase of the bathmotropic effect increases the excitability of the cardiac muscle which also increases the cardiac output through stroke volume alteration. Increase of the dromotropic effect by cateholamines increases the AV nodal conduction velocity which increases the cardiac output by increasing the heart rate (Hall et al. 2010). Thus, regulation or dysregulation of α-synuclein can result in downstream effects on cardiac conductivity associated processes. This makes α-synuclein regulation a potential therapeutic strategy to address CCD and related pathology.

αS is a member of the synuclein family of soluble proteins (αS, β-synuclein and γ-synuclein) that are commonly present in CNS of vertebrates. αS is expressed in the neocortex, hippocampus, substantia niagra, thalamus and cerebellum, with the main location within the presynaptic terminals of neurons in both membrane-bound and cytosolic free forms. Presynaptic terminals release neurotransmitters, from synaptic vesicles. The release of neurotransmitters relays signals between neurons and is critical for normal brain function. αS can be seen in neuroglial cells and melanocytic cells, and is highly expressed in the neuronal mitochondria of the olfactory bulb, hippocampus, striatum and thalamus. As such, there exists an association of αS with CCDs and neurological and psychological conditions.

αS aggregates to form insoluble fibrils in pathological conditions characterized by Lewy bodies, such as PD, dementia with Lewy bodies (DLB) and multiple system atrophy (MSA). These disorders are known as synucleinopathies. αS is the primary structural component of Lewy body fibrils. Occasionally, Lewy bodies contain tau protein; however, αS and tau constitute two distinctive subsets of filaments in the same inclusion bodies. αS pathology is also found in both sporadic and familial cases with Alzheimer's disease (AD). Thus, one indicator of abnormal αS pathology is the formation of αS aggregates.

At the molecular level, protein misfolding, accumulation, aggregation and subsequently the formation of amyloid deposits are common features in many neurological disorders including Alzheimer's disease (AD) and Parkinson's disease (PD). Thus neurodegenerative diseases are sometimes referred to as proteinopathies. The existence of a common mechanism suggests that neurodegenerative disorders likely share a common trigger and that the nature of the pathology is determined by the type of the aggregated protein and the localization of the cell affected.

Starting two decades ago with the discoveries of genetic links between αS and PD risk and the identification of aggregated αS as the main protein constituent of Lewy pathology, αS has emerged as the major therapeutic target in PD and related synucleinopathies. Brundin et al., 2017. The α-synuclein abnormalities typically found in PD are believed to be responsible for apparent catecholamine-deficits in PD (Frisina et al., 2009). In patients with PD, α-synuclein-related pathology develops in serotonergic and cholinergic neurons in parallel with that seen in the nigral dopamine neurons, and there are data to suggest that the development of cognitive impairments and depression correlate with the extent of damage seen in these systems.

In one embodiment, a method of treating, preventing, and/or slowing the onset or progression of a CCD and/or a related symptom in a subject in need is provided, the method comprising administering to the subject a therapeutically effective amount of at least one aminosterol, or a salt or derivative thereof.

In one embodiment, the invention encompasses a method of treating, preventing and/or slowing the onset or progression of a CCD and/or a related symptom in a subject in need comprising administering to the subject a therapeutically effective amount of at least one aminosterol or a salt or derivative thereof. In one aspect, the at least one aminosterol or a salt or derivative thereof is administered via oral, nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof. In another aspect, the at least one aminosterol or a salt or derivative thereof is administered nasally. In another aspect, administration of the at least one aminosterol or a salt or derivative thereof comprises non-oral administration.

In another embodiment, the invention encompasses a method of treating, preventing and/or slowing the onset or progression of a cardiac conduction defect (CCD) and/or a related symptom in a subject in need comprising (a) determining a dose of an aminosterol or a salt or derivative thereof for the subject, wherein the aminosterol dose is determined based on the effectiveness of the aminosterol dose in improving or resolving a CCD symptom being evaluated, (b) followed by administering the aminosterol dose to the subject for a period of time, wherein the method comprises (i) identifying a CCD symptom to be evaluated; (ii) identifying a starting aminosterol dose for the subject; and (iii) administering an escalating dose of the aminosterol to the subject over a period of time until an effective dose for the CCD symptom being evaluated is identified, wherein the effective dose is the aminosterol dose where improvement or resolution of the CCD symptom is observed, and fixing the aminosterol dose at that level for that particular CCD symptom in that particular subject.

In another embodiment, the fixed dose of the aminosterol or a salt or derivative thereof is given once per day, every other day, once per week, twice per week, three times per week, four times per week, five times per week, six times per week, every other week, or every few days. In addition, the fixed dose of the aminosterol or a salt or derivative thereof can be administered for a first defined period of time of administration, followed by a cessation of administration for a second defined period of time, followed by resuming administration upon recurrence of CCD or a symptom of CCD. For example, the fixed aminosterol dose can be incrementally reduced after the fixed dose of aminosterol or a salt or derivative thereof has been administered to the subject for a period of time. Alternatively, the fixed aminosterol dose is varied plus or minus a defined amount to enable a modest reduction or increase in the fixed dose. For example, the fixed aminosterol dose can be increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

In another embodiment, the starting aminosterol or a salt or derivative thereof dose is higher if the CCD symptom being evaluated is severe.

In one embodiment, progression or onset of CCD is slowed, halted, or reversed over a defined period of time following administration of the fixed escalated dose of the aminosterol or a salt or derivative thereof, as measured by a medically-recognized technique. In addition, the CCD can be positively impacted by the fixed escalated dose of the aminosterol or a salt or derivative thereof, as measured by a medically-recognized technique. The positive impact and/or progression of CCD can be measured quantitatively or qualitatively by one or more techniques selected from the group consisting of echocardiography, electrocardiography (ECG or EKG), magnetic resonance imaging (MRI), positron-emission tomography (PET); coronary catheterization, intravascular ultrasound, Holter monitoring, stress test, computed tomography angiography (CTA), and coronary CT calcium scan. In addition, the progression or onset of CCD can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In another embodiment, the aminosterol or a salt or derivative thereof reverses dysfunction caused by the CCD and treats, prevents, improves, and/or resolves the symptom being evaluated. The improvement or resolution of the CCD symptom is measured using a clinically recognized scale or tool. In addition, the improvement in the CCD symptom can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale.

In yet another embodiment, the CCD symptom to be evaluated can be selected from the group consisting of (a) QT interval (QTc)≥440 ms; (b) syncope; (c) presence of delta wave in electrocardiogram (EKG); (d) pseudo-right bundle branch block in EKG; (e) ST elevations in V1-V3 in EKG; (f) a QRS complex>100 ms in EKG; (g) PR interval<120 ms in EKG; (h) heart rate above 100 beats per minute (BPM); (i) heart rate below 60 BPM; (j) PR interval>200 ms in EKG; (k) QRS not following a P wave in EKG; (l) no repeating relation between P wave and QRS complex in EKG; (m) differing atrial and ventricular rates; (n) QS or rS complex in lead V1 in EKG; (o) notched (‘M’-shaped) R wave in lead V6; (p) T wave discordance in EKG; (q) left axis deviation between −45° and −60° in EKG; (r) qR pattern (small q, tall R) in the lateral limb leads I and aVL in EKG; (s) rS pattern (small r, deep S) in the inferior leads II, III, and aVF in EKG; (t) delayed intrinsicoid deflection in lead aVL (>0.045 s) in EKG; (u) frontal plane axis between 90° and 180° in EKG; (v) rS pattern in leads I and aVL in EKG; (w) qR pattern in leads III and aVF in EKG; (x) chest pain; (y) palpitations; (z) difficulty breathing; (aa) rapid breathing; (bb) nausea; (cc) fatigue; (dd) sleep problem, sleep disorder, or sleep disturbance; (ee) constipation; and (ff) cognitive impairment.

In one embodiment, the CCD symptom to be evaluated is a sleep problem, sleep disorder, or sleep disturbance comprising a delay in sleep onset, sleep fragmentation, REM-behavior disorder, sleep-disordered breathing including snoring and apnea, day-time sleepiness, micro-sleep episodes, narcolepsy, hallucinations, or any combination thereof. In addition, the REM-behavior disorder can comprise vivid dreams, nightmares, and acting out the dreams by speaking or screaming, or fidgeting or thrashing of arms or legs during sleep.

In one embodiment, the CCD symptom to be evaluated is a sleep problem, sleep disorder, or sleep disturbance, and (a) the method results in a positive change in the sleeping pattern of the subject; (b) the method results in a positive change in the sleeping pattern of the subject, wherein the positive change is defined as: (i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (c) as a result of the method the subject obtains the total number of hours of sleep recommended by a medical authority for the age group of the subject.

In one embodiment, the CCD symptom to be evaluated is a QT interval (QTc)≥about 440 ms in EKG and wherein: (a) the method results in a decreased QTc in the subject; (b) the method results in a decreased QTc in the subject and the decreased QTc is defined as a reduction in QTc measured via EKG selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (c) the method results in the subject having a QTc<about 440 ms.

In one embodiment, the CCD symptom to be evaluated is a QRS complex>100 ms in EKG and wherein: (a) the method results in a decreased QRS complex in the subject; (b) the method results in a decreased QRS complex in the subject and the decreased QRS complex is defined as a reduction in QRS complex measured via EKG selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (c) the method results in the subject having a QRS complex≤about 100 ms.

In one embodiment, the CCD symptom to be evaluated is a heart rate above about 100 beats per minute (BPM) and wherein: (a) the method results in a decreased heart rate in the subject; (b) the method results in a decreased heart rate in the subject and the decreased heart rate is defined as a reduction in heart rate measured selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (c) the method results in the subject having a heart rate≤about 100 BPM.

In one embodiment, the CCD symptom to be evaluated is a heart rate below about 60 BPM and wherein: (a) the method results in an increased heart rate in the subject; (b) the method results in an increased heart rate in the subject and the increased heart rate is defined as an increase in heart rate measured selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (c) the method results in the subject having a heart rate≥about 60 BPM.

In one embodiment, the CCD symptom to be evaluated is cognitive impairment, and wherein: (a) progression or onset of the cognitive impairment is slowed, halted, or reversed over a defined period of time following administration of the fixed escalated dose of the aminosterol or a salt or derivative thereof, as measured by a medically-recognized technique; and/or (b) the cognitive impairment is positively impacted by the fixed escalated dose of the aminosterol or a salt or derivative thereof, as measured by a medically-recognized technique; (c) the cognitive impairment is positively impacted by the fixed escalated dose of the aminosterol or a salt or derivative thereof, as measured by a medically-recognized technique and the positive impact on and/or progression of cognitive impairment is measured quantitatively or qualitatively by one or more techniques selected from the group consisting of Mini-Mental State Exam (MMSE), Mini-cog test, and a computerized test selected from Cantab Mobile, Cognigram, Cognivue, Cognision, or Automated Neuropsychological Assessment Metrics; and/or (d) the progression or onset of cognitive impairment is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In some embodiments, the CCD symptom to be evaluated is constipation, and (a) treating the constipation prevents and/or delays the onset and/or progression of the CCD; (b) the fixed escalated aminosterol dose causes the subject to have a bowel movement; (c) the method results in an increase in the frequency of bowel movement in the subject; (d) the method results in an increase in the frequency of bowel movement in the subject and the increase in the frequency of bowel movement is defined as: (i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; (e) as a result of the method the subject has the frequency of bowel movement recommended by a medical authority for the age group of the subject; and/or (f) the starting aminosterol dose is determined by the severity of the constipation, wherein: (i) if the average complete spontaneous bowel movement (CSBM) or spontaneous bowel movement (SBM) is one or less per week, then the starting aminosterol dose is at least about 150 mg; and (ii) if the average CSBM or SBM is greater than one per week, then the starting aminosterol dose is about 75 mg or less.

In another embodiment, the aminosterol or a salt or derivative thereof is administered in combination with at least one additional active agent to achieve either an additive or synergistic effect. For example, the additional active agent can be administered via a method selected from the group consisting of (a) concomitantly; (b) as an admixture; (c) separately and simultaneously or concurrently; or (d) separately and sequentially. In another embodiment, the additional active agent is a different aminosterol from that administered in primary method. In yet a further embodiment, the method of the invention comprises administering a first aminosterol which is aminosterol 1436 or a salt or derivative thereof intranasally and administering a second aminosterol which is squalamine or a salt or derivative thereof orally.

In another embodiment, the at least one additional active agent is an active agent used to treat CCD or a symptom thereof. In some embodiments, the active agent is selected from the group consisting of beta blockers, propanolol (Inderal®), acebutolol (Sectral®), and sotalol (Betapace®); antiarrhythmics such as amiodarone (Cordarone®), adenosine (Adenocard®), propafenone (Rhythmol®), and dronedarone (Multaq®); calcium channel blockers such as diltiazem (Cardizem®) and verapamil (Verelan®); and digitalis derived drugs such as digoxin (Lanoxin®).

In another embodiment, the subject to be treated according to the methods of the invention can be a member of a patient population at risk for being diagnosed with a CCD.

5. Blood Pressure

In one aspect of the disclosure, encompassed is a method of treating, preventing, and/or slowing the onset or progression of high blood pressure (HBP) and/or a related symptom in a subject in need, or a method of treating, preventing, and/or slowing the onset or progression of low blood pressure (LBP) and/or a related symptom in a subject in need, comprising administering to the subject a therapeutically effective amount of at least one aminosterol, or a salt or derivative thereof. In another aspect, the method of administration comprises oral, nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof. In yet another aspect, the method of administration comprises non-oral administration or nasal administration.

Another method of the disclosure is directed to a method of treating, preventing, and/or slowing the onset or progression of high blood pressure (HBP) and/or a related symptom in a subject in need, or a method of treating, preventing, and/or slowing the onset or progression of low blood pressure (LBP) and/or a related symptom in a subject in need, comprising: (a) determining a dose of an aminosterol or a salt or derivative thereof for the subject, wherein the aminosterol dose is determined based on the effectiveness of the aminosterol dose in improving or resolving an HBP symptom being evaluated, (b) followed by administering the aminosterol dose to the subject for a defined period of time, wherein the method comprises: (i) identifying an HBP symptom to be evaluated; (ii) identifying a starting aminosterol dose for the subject; and (iii) administering an escalating dose of the aminosterol or a salt or derivative thereof to the subject over a defined period of time until an effective dose for the HBP symptom being evaluated is identified, wherein the effective dose is the aminosterol dose where improvement or resolution of the HBP symptom is observed, and fixing the aminosterol dose at that level for that particular HBP symptom in that particular subject; and (c) optionally wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

High blood pressure (HBP), also referred to as hypertension, is a long-term medical condition in which the blood pressure in the arteries is persistently elevated. Long-term high blood pressure, is a major risk factor for coronary artery disease, stroke, heart failure, atrial fibrillation, peripheral vascular disease, vision loss, chronic kidney disease, and dementia.

Around 85 million people in the United States have high blood pressure. Worldwide it affects one billion people and is the most common risk factor for death around the world. World health statistics in 2012 estimated the prevalence of hypertension to be 29.2% in males and 24.8% in females. Approximately 90% of men and women who are non-hypertensive at 55 or 65 years will develop hypertension by the age of 80-85 (Kumar 2013).

Low blood pressure (LBP), also referred to as hypotension, is generally classified as a systolic blood pressure of less than 90 millimeters of mercury (mm Hg) or diastolic of less than 60 mm Hg. Primary symptoms include lightheadedness, vertigo and fainting. Severely low blood pressure can deprive the brain and other vital organs of oxygen and nutrients, leading to a life-threatening condition called shock. For some people who exercise and are in top physical condition, low blood pressure is a sign of good health and fitness. For many people, excessively low blood pressure can cause dizziness and fainting or indicate serious heart, endocrine or neurological disorders.

Many neurodiseases causing HBP or LBP, such as PD, are suspected to correlate with the formation of toxic αS aggregates within the enteric nervous system (ENS) (Braak et al. 2003). In a study of 11.55 million PD patient doctor visits in the US, the most commonly recorded comorbidity was hypertension, in 37.8% of visits (Lingala et al. 2017). Orthostatic hypotension (OH) is one of the commonly occurring nonmotor symptoms in patients with idiopathic Parkinson's disease (IPD) (Fereshtehnejad et al. 2014).

Studies suggest that a persistent hypertension can cause abnormal accumulation of phosphorylated αS in rats (Sato et al. 2014; Fukui et al. 2014). Also, mice genetically engineered to overexpress human alpha-synuclein showed differing cardiac responses to chemically induced hypotension compared to wildtype mice (Fleming et al. 2013).

One in five patients with PD are affected by orthostatic hypotension, which may manifest as a drop in blood pressure upon standing up. Elevated systolic blood pressure predicts worsening motor function among patients with Parkinson's disease (Lineback 2016). Neurodegenerative conditions such as PD may cause damage to brain centers responsible for autonomic processing, essential for regulation of blood pressure. It is believed that aminosterols capable of treating or preventing neurodegeneration in PD, may prevent or treat the degeneration of neuronal structure that governs regulation of blood pressure either directly or indirectly via the regulation of hormones.

αS is a member of the synuclein family of soluble proteins (αS, β-synuclein and γ-synuclein) that are commonly present in CNS of vertebrates. αS is expressed in the neocortex, hippocampus, substantia niagra, thalamus and cerebellum, with the main location within the presynaptic terminals of neurons in both membrane-bound and cytosolic free forms. Presynaptic terminals release chemical messengers, called neurotransmitters, from compartments known as synaptic vesicles. The release of neurotransmitters relays signals between neurons and is critical for normal brain function. αS can be seen in neuroglial cells and melanocytic cells, and is highly expressed in the neuronal mitochondria of the olfactory bulb, hippocampus, striatum and thalamus.

αS aggregates to form insoluble fibrils in pathological conditions characterized by Lewy bodies, such as PD, dementia with Lewy bodies (DLB) and multiple system atrophy (MSA). These disorders are known as synucleinopathies. αS is the primary structural component of Lewy body fibrils. Occasionally, Lewy bodies contain tau protein; however, αS and tau constitute two distinctive subsets of filaments in the same inclusion bodies. αS pathology is also found in both sporadic and familial cases with AD. Thus, one indicator of αS pathology is the formation of αS aggregates.

At the molecular level, protein misfolding, accumulation, aggregation and subsequently the formation of amyloid deposits are common features in many neurological disorders including Alzheimer's disease (AD) and Parkinson's disease (PD). Thus neurodegenerative diseases are sometimes referred to as proteinopathies. The existence of a common mechanism suggests that neurodegenerative disorders likely share a common trigger and that the nature of the pathology is determined by the type of the aggregated protein and the localization of the cell affected.

Starting two decades ago with the discoveries of genetic links between αS and PD risk and the identification of aggregated αS as the main protein constituent of Lewy pathology, αS has emerged as the major therapeutic target in PD and related synucleinopathies (Brundin et al., 2017). The α-synuclein abnormalities typically found in PD are believed to be responsible for apparent catecholamine-deficits (dopamine is a catecholamine sharing metabolic pathways with other catecholamines) (Frisina et al., 2009). It is known that dopamine is a key neurotransmitter regulating blood pressure (Jose et al. 2003). Dopamine's actions on renal hemodynamics, epithelial transport and humoral agents such as aldosterone, catecholamines, endothelin, prolactin, pro-opiomelanocortin, renin and vasopressin place it in central homeostatic position for regulation of blood pressure. Dopamine also modulates fluid and sodium intake via actions in the central nervous system and gastrointestinal tract, and by regulation of cardiovascular centers that control the functions of the heart, arteries and veins. Abnormalities in dopamine production and receptor function accompany a high percentage of human essential hypertension and several forms of rodent genetic hypertension. Id. In patients with PD, α-synuclein-related pathology develops in serotonergic and cholinergic neurons in parallel with that seen in the nigral dopamine neurons. Thus, regulation of α-synuclein may play a role in blood pressure dysregulation in PD via dopaminergic dysfunction.

In some embodiments, a method for treating, preventing, and/or slowing the onset of high blood pressure (HBP) and/or a related symptom is provided. The methods comprise administering to a subject in need a composition comprising a therapeutically effective amount of at least one aminosterol, or a salt or derivative thereof. Certain embodiments describe the determination and administration of a “fixed aminosterol dose” that is not age, size, or weight dependent but rather is individually calibrated. In some embodiments, the dose is a fixed dose or varies according to any method described herein.

In addition, the present application relates generally to methods for treating, preventing, and/or slowing the onset of low blood pressure (LBP) and/or a related symptom. The methods comprise administering to a subject in need a composition comprising a therapeutically effective amount of at least one aminosterol, or a salt or derivative thereof. Certain embodiments describe the determination and administration of a “fixed aminosterol dose” that is not age, size, or weight dependent but rather is individually calibrated.

In another embodiment, the invention encompasses a method of treating, preventing and/or slowing the onset of HBP and/or a related symptom, or LBP and/or a related symptom in a subject in need comprising (a) determining a dose of an aminosterol or a salt or derivative thereof for the subject, wherein the aminosterol dose is determined based on the effectiveness of the aminosterol dose in improving or resolving an HBP or LBP symptom being evaluated, (b) followed by administering the aminosterol dose to the subject for a defined time period, wherein the method comprises (i) identifying an HBP or LBP symptom to be evaluated; (ii) identifying a starting aminosterol dose for the subject; and (iii) administering an escalating dose of the aminosterol or a salt or derivative thereof to the subject over a period of time until an effective dose for the HBP or LBP symptom being evaluated is identified, wherein the effective dose is the aminosterol dose where improvement or resolution of the HBP or LBP symptom is observed, and fixing the aminosterol dose at that level for that particular HBP or LBP symptom in that particular subject.

In one embodiment, the method is directed to treating or preventing HBP and/or a related symptom, wherein the symptom to be evaluated relates to HBP. In another embodiment, the method is directed to treating or preventing LBP and/or a related symptom, wherein the symptom to be evaluated relates to LBP.

In another embodiment, the dose or fixed dose of the aminosterol or a salt or derivative thereof is administered once per day, every other day, once per week, twice per week, three times per week, four times per week, five times per week, six times per week, every other week, or every few days. In addition, the fixed dose of the aminosterol or a salt or derivative thereof can be administered for a first defined period of time of administration, followed by a cessation of administration for a second defined period of time, followed by resuming administration upon recurrence of HBP and/or a related symptom, or LBP and/or a related symptom. For example, the fixed aminosterol dose can be incrementally reduced after the fixed dose of aminosterol or a salt or derivative thereof has been administered to the subject for a period of time. Alternatively, the fixed aminosterol dose is varied plus or minus a defined amount to enable a modest reduction or increase in the fixed dose. For example, the fixed aminosterol dose can be increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

In another embodiment, the starting aminosterol or a salt or derivative thereof dose is higher if the HBP or LBP symptom being evaluated is severe.

In one embodiment, the method of the invention results in slowing, halting, or reversing progression or onset of HBP and/or a related symptom, or LBP and/or a related symptom, over a defined time period following administration of the aminosterol or a salt or derivative thereof, or the fixed escalated dose of the aminosterol or a salt or derivative thereof, as measured by a medically-recognized technique. For example, the progression or onset of HBP and/or a related symptom, or LBP and/or a related symptom, may be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique. In addition, the method of the invention can result in positively impacting the HBP and/or a related symptom, or LBP and/or a related symptom, as measured by a medically-recognized technique. The positive impact and/or progression on HBP and/or a related symptom, or LBP and/or a related symptom, may be measured quantitatively or qualitatively by one or more techniques selected from the group consisting of, for example, sphygmomanometry, arterial penetration, palpitation, asuculatoration, oscillometry, continuous noninvasive arterial pressure (CNAP), pulse wave velocity, and ambulatory monitoring.

In one embodiment, the fixed escalated aminosterol dose reverses dysfunction caused by the HBP or LBP and treats, prevents, improves, and/or resolves the symptom being evaluated. The improvement or resolution of the HBP or LBP symptom can be measured using a clinically recognized scale or tool. For example, the improvement in the HBP or LBP symptom can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale.

In yet another embodiment, the symptom to be evaluated can be selected from the group consisting of (a) a systolic blood pressure (BP)≥120 and a diastolic BP<80; (b) a systolic blood pressure (BP)≥130 or a diastolic BP≥80; (c) headache; (d) lightheadedness; (e) vertigo; (f) tinnitus; (g) altered vision; (h) fainting; (i) hypertensive retinopathy; (j) palpitations; (k) excess sweating; (l) a systolic blood pressure≤80; (m) a diastolic blood pressure≤50; (n) fatigue; (o) stiff neck and/or upper back; (p) dyspepsia; (q) dysuria; (r) seizure; (s) shortness of breath; (t) constipation; (u) hallucinations; (v) depression; (w) sleep disorder, sleep problem, and/or sleep disturbance; (x) cardiovascular disease; and/or (y) cognitive impairment.

In one embodiment, the symptom to be evaluated is selected from the group consisting of (i) a systolic blood pressure (BP)≥120 and a diastolic BP<80; (ii) a systolic blood pressure (BP)≥130 or a diastolic BP≥80; (iii) a systolic blood pressure≤80; and (iv) a diastolic blood pressure≤50, and wherein: (a) the method results in a positive change in the systolic BP or diastolic BP of the subject; (b) the method results in a positive change in the systolic BP and/or diastolic BP of the subject, wherein the positive change is defined as: (i) an increase in the systolic BP and/or diastolic BP, if the symptom is (iii) or (iv), of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; or (ii) a decrease in the systolic BP and/or diastolic BP in the subject, if the symptom is (i) or (ii) of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (c) as a result of the method the subject has the systolic BP and/or diastolic BP recommended by a medical authority for the age group of the subject.

In one embodiment, the symptom to be evaluated is lightheadedness and wherein (a) the method results in a decreased number or severity of occurrences of lightheadedness of the subject; (b) the method results in a decreased number or severity of occurrences of lightheadedness which is defined as a reduction in occurrences or severity of lightheadedness selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (c) the method results in the subject being lightheadedness-free.

In one embodiment, the symptom to be evaluated is headache and wherein: (a) the method results in improvement in a subject's headache, as measured by one or more clinically-recognized headache rating scales; (b) the method results in improvement in a subject's headache, as measured by one or more clinically-recognized headache rating scales and the improvement is in one or more headache types selected from the group consisting of tension, cluster, migraine, hypertension headache and hypotension headache; and/or (c) the method results in improvement in a subject's headache, as measured by one or more clinically-recognized headache rating scales, and the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%.

In yet another embodiment, the symptom to be evaluated is tinnitus, and wherein: (a) progression or onset of the tinnitus is slowed, halted, or reversed over a defined time period following administration of the fixed escalated dose of the aminosterol or a salt or derivative thereof, as measured by a medically-recognized technique; and/or (b) the tinnitus is positively impacted by the fixed escalated dose of the aminosterol or a salt or derivative thereof, as measured by a medically-recognized technique; (c) the tinnitus is positively impacted by the fixed escalated dose of the aminosterol or a salt or derivative thereof, as measured by a medically-recognized technique and the positive impact on and/or progression of tinnitus is measured quantitatively or qualitatively by one or more techniques selected from the group consisting of speech recognition, pure tone audiogram, tympanogram, acoustic reflex test, and optoacoustic emission test; and/or (d) the progression or onset of tinnitus is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In one embodiment, the HBP or LBP symptom to be evaluated is hallucinations and wherein: (a) the hallucinations comprise a visual, auditory, tactile, gustatory or olfactory hallucinations; (b) the method results in a decreased severity and/or number of hallucinations over a defined period of time in the subject, optionally as measured by one or more medically-recognized techniques; (c) the method results in a decreased severity and/or number of hallucinations over a defined period of time in the subject selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (d) the method results in the subject being hallucination-free. The one or more medically recognized techniques can be, for example, selected from the group consisting of Chicago Hallucination Assessment Tool (CHAT), The Psychotic Symptom Rating Scales (PSYRATS), Auditory Hallucinations Rating Scale (AHRS), Hamilton Program for Schizophrenia Voices Questionnaire (HPSVQ), Characteristics of Auditory Hallucinations Questionnaire (CAHQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA).

In another embodiment, the HBP or LBP symptom to be evaluated is depression and wherein: (a) the method results in improvement in a subject's depression, as measured by one or more clinically-recognized depression rating scales; (b) the method results in improvement in a subject's depression, as measured by one or more clinically-recognized depression rating scales and the improvement is in one or more depression characteristics selected from the group consisting of mood, behavior, bodily functions such as eating, sleeping, energy, and sexual activity, and/or episodes of sadness or apathy; and/or (c) the method results in improvement in a subject's depression, as measured by one or more clinically-recognized depression rating scales, and the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%.

In one embodiment, the HBP or LBP symptom to be evaluated is cognitive impairment, and wherein: (a) progression or onset of the cognitive impairment is slowed, halted, or reversed over a defined period of time following administration of the fixed escalated dose of the aminosterol or a salt or derivative thereof, as measured by a medically-recognized technique; and/or (b) the cognitive impairment is positively impacted by the fixed escalated dose of the aminosterol or a salt or derivative thereof, as measured by a medically-recognized technique; (c) the cognitive impairment is positively impacted by the fixed escalated dose of the aminosterol or a salt or derivative thereof, as measured by a medically-recognized technique and the positive impact on and/or progression of cognitive decline is measured quantitatively or qualitatively by one or more techniques selected from the group consisting of ADASCog, Mini-Mental State Exam (MMSE), Mini-cog test, Woodcock-Johnson Tests of Cognitive Abilities, Leiter International Performance Scale, Miller Analogies Test, Raven's Progressive Matrices, Wonderlic Personnel Test, IQ tests, or a computerized tested selected from Cantab Mobile, Cognigram, Cognivue, Cognision, and Automated Neuropsychological Assessment Metrics Cognitive Performance Test (CPT); and/or (d) the progression or onset of cognitive impairment is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In another embodiment, the HBP or LBP symptom to be evaluated is a sleep problem, sleep disorder, or sleep disturbance and: (a) the sleep problem, sleep disorder, or sleep disturbance comprises a delay in sleep onset, sleep fragmentation, REM-behavior disorder, sleep-disordered breathing including snoring and apnea, day-time sleepiness, micro-sleep episodes, narcolepsy, circadian rhythm dysfunction, REM disturbed sleep, or any combination thereof; (b) the sleep problem, sleep disorder, or sleep disturbance comprises REM-behavior disorder, which comprises vivid dreams, nightmares, and acting out the dreams by speaking or screaming, or fidgeting or thrashing of arms or legs during sleep; (c) treating the sleep problem, sleep disorder, or sleep disturbance prevents or delays the onset and/or progression of the schizophrenia; (d) the method results in a positive change in the sleeping pattern of the subject; wherein the positive change is defined as: (i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (f) as a result of the method the subject obtains the total number of hours of sleep recommended by a medical authority for the age group of the subject.

In another embodiment, the symptom to be evaluated is constipation, and wherein: (a) the fixed escalated aminosterol dose causes the subject to have a bowel movement; (b) the method results in an increase in the frequency of bowel movement in the subject; (c) the method results in an increase in the frequency of bowel movement in the subject and the increase in the frequency of bowel movement is defined as: (i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; (d) as a result of the method the subject has the frequency of bowel movement recommended by a medical authority for the age group of the subject; and/or (e) the starting aminosterol dose is determined by the severity of the constipation, wherein: (i) if the average complete spontaneous bowel movement (CSBM) or spontaneous bowel movement (SBM) is one or less per week, then the starting aminosterol dose is at least about 150 mg; and (ii) if the average CSBM or SBM is greater than one per week, then the starting aminosterol dose is about 75 mg or less.

In another embodiment, the aminosterol or a salt or derivative thereof is administered in combination with at least one additional active agent to achieve either an additive or synergistic effect. For example, the additional active agent can be administered via a method selected from the group consisting of (a) concomitantly; (b) as an admixture; (c) separately and simultaneously or concurrently; or (d) separately and sequentially. In another embodiment, the additional active agent is a different aminosterol from that administered in primary method. In yet a further embodiment, the method of the invention comprises administering a first aminosterol which is aminosterol 1436 or a salt or derivative thereof intranasally and administering a second aminosterol which is squalamine or a salt or derivative thereof orally.

In another embodiment, the at least one additional active agent is an active agent used to treat HBP or LBP or a symptom thereof. In some embodiments, the active agent is selected from the group consisting of thiazide-diuretics such as chlorthalidone and hydrochlorthalidone; calcium channel blockers such as amlodipine (Norvasc®), felodipine (Plendil®), isradipine (DynaCirc®), nicardipine (Cardene®), nifedipine (Adalat®), diltiazem (Diltia XL®), nisoldipine (Sular®), and verapamil (Covera-HS®); angiotensin converting enzyme (ACE) inhibitors such as captopril (Capoten®), enalapril (Vasotec®), ramipril (Altace®), quinapril (Accupril®), perindopril (Coversyl®), lisinopril (Listril®), and benazepril (Lotensin®); angiotensin receptor blockers such as losartan (Cozaar®), irbesartan (Avapro®), olmesartan (Benicar®), candesartan (Atacand®), valsartan (Diovan®), fimasartan (Kanarb®), and azilsartan (Edarbi®); beta-blockers such as acebutolol (Sectral®), atenolol (Tenormin®), betaxolol (Kerlone®), bisoprolol (Zebeta®, Ziac®), carteolol (Cartrol®), carteolol (Cartrol®), metoprolol (Lopressor®, Toprol-XL®) and propanolol (Inderal®); corticosteroids such as fludrocortisone (Astonin®); and vasopressors such as midodrine (Orvaten®).

6. Sleep Disturbance/Sleep Problems

(e.g., REM Disturbed Sleep or Circadian Rhythm Dysfunction)

Normal sleep is critically important for the proper functioning of many organ systems, the most important of which is the brain. Disturbances in normal sleep patterns are closely associated with the normal aging process, with the development of cognitive impairment, with impaired memory deposition and consolidation and with the occurrence of neurodevelopmental, neuroaffective and neurodegenerative disorders. The alternating pattern of sleep and wakefulness occurring every 24 hours is known as the circadian rhythm. The rhythm is set by the “zeitgeber” (time setter), an entity known as the suprachiasmatic nucleus (SCN) and located in the hypothalamus. The SCN is normally “entrained” or synchronized by the external light-dark cycle. This relationship between external light and dark and the sleep wake cycle synchronized to it by the SCN can be over ridden during periods of hunger by neural signals emanating in the gut and relayed to the hypothalamus. The circadian sleep-wake cycle can also shift in response to changes in external light-dark cycles, such as the desynchronization that occurs during travel from one time zone to another (jet-lag). Under such circumstances, a progressive adjustment occurs until the SCN is resynchronized with the external light-dark cycle. A similar “phase-shift” and adjustment occurs in night-shift workers.

Under normal circumstances, the properly functioning SCN, synchronized to the external light-dark cycle and to neural signals emanating from the enteric nervous system, will regulate the sleep-wake cycle by sending neural and chemical signals to the surrounding structures and to portions of the brain stem involved in sleep and wakefulness. An individual with a properly functioning hypothalamus and brain stem will go to bed and fall asleep within minutes, remain asleep throughout the night, wake up in the morning and remain awake and alert throughout the day. During the night, the asleep individual will experience several cycles of sleep, beginning with light sleep, progressing through rapid eye movement sleep (REM-sleep) to deep sleep and back. Each complete sleep period lasts about 90 minutes. Periods of REM-sleep are closely associated with dreaming. During REM-sleep, neural signals emanating from certain parts of the brain stem ensure that skeletal muscles become “atonic” or are paralyzed, such that the individual can't “act out” their dreams.

Certain diseases and conditions may impair the normal functioning of the “zeitgebber” or circadian clock. These conditions may be reversible, such as desynchronization resulting from jet-lag, night-shift work or hunger, conditions easily remedied by adaptation or food intake. In contrast, damage to the nerves carrying light-dark related information from the retina to the SCN (conditions which may lead to blindness), or damage to the enteric nerves and neural structures which relay messages from the intestine to the SCN (conditions which may lead to neurodegenerative disorders) can cause permanent dysfunction of the circadian rhythm and abnormal sleep behavior.

Dysfunction of the circadian rhythm manifests first and foremost by abnormal sleep patterns. Such abnormalities typically are mild at onset and worsen progressively over time. A common symptom of sleep disorder is a delay in the onset of sleep. This delay can be as long as several hours, and the individual may not be able to fall asleep until the early hours of the morning. Another common symptom is sleep fragmentation, meaning that the individual awakens several times during the course of the night. Once awakened, the individual may not be able to get back to sleep, and each awake fragment may last an hour or more, further reducing “total sleep time,” which is calculated by subtracting total time of the awake fragments from total time spent in bed. Total sleep time also diminishes with age, from about 14 to about 16 hours a day in newborns, to about 12 hours by one year of age, to about 7 to about 8 hours in young adults, progressively declining to about 5 to about 6 hours in elderly individuals. Total sleep time can be used to calculate an individual's “sleep age” and to compare it to their chronologic age. Significant discrepancies between sleep age and chronologic age are a reflection of the severity of the sleep disorder. “Sleep efficiency,” defined as the percentage of the time spent in bed asleep is another index that can be used to determine the severity of the sleep disorder. Sleep efficiency is said to be abnormal when the percentage is below about 70%.

Sleep disorders and/or sleep disturbances include but are not limited to REM-behavior disorders, disturbances in the Circadian rhythm, delayed sleep onset, sleep fragmentation, and hallucinations. Other sleep disorders or disturbances that can be treated and/or prevented according to the disclosed methods include but are not limited to hypersomnia (i.e., daytime sleepiness), parasomnias (such as nightmares, night terrors, sleepwalking, and confusional arousals), periodic limb movement disorders (such as Restless Leg Syndrome), jet lag, narcolepsy, advanced sleep phase disorder, non-24 hour sleep-wake syndrome.

Individuals with severe sleep disorders also typically suffer from day-time sleepiness. This can manifest as day-time “napping” for an hour or two, to “dosing off” for a few minutes during a film or to “micro-sleep” episodes lasting seconds to minutes, and of which the individual may or may not be aware. Narcolepsy is a rare and extreme form of day-time sleepiness, with the sudden onset of sleep causing the individual to fall down. Another form of sleep disturbance involves periods of loud snoring alternating with periods of “sleep apnea” (arrested breathing), a condition known as “sleep-disordered breathing.” “REM-behavior disorder” (RBD) or “REM-disturbed sleep”, is yet another sleep disturbance which occurs as a result of dysfunctional neural communication between the enteric nervous system, structures responsible for sleep in the brain stem and the SCN. In individuals with RBD, neural signaling which causes the paralysis (atonia) of muscles under voluntary control is impaired or altogether absent. As a consequence, “acting-out” of dreams occurs. This can range at one end of the spectrum from an increase in muscle tone detectable by electromyography (EMG) and accompanied by small movements of the hands and feet during REM sleep, to violent thrashing of arms and legs, kicking or punching a bed partner, speaking out loud or screaming, at the other end of the spectrum. Episodes of RBD can occur several times a night or very infrequently, once every few months. They can also be clustered, several occurring within a week, followed by periods of normal sleep. Unless the condition can be treated with a medication that restores normal functioning of the circadian rhythm and improves sleep patterns, individuals with RBD progress to neurodegenerative disorders.

Sleep disturbances include but are not limited to RBD, circadian rhythm dysfunction, delayed sleep onset, Restless leg syndrome, daytime sleepiness, and sleep fragmentation.

Sleep is increasingly recognized as important to public health, with sleep insufficiency linked to motor vehicle crashes, industrial disasters, and medical and other occupational errors. Unintentionally falling asleep, nodding off while driving, and having difficulty performing daily tasks because of sleepiness all may contribute to these hazardous outcomes. Persons experiencing sleep insufficiency are also more likely to suffer from chronic diseases such as hypertension, diabetes, depression, and obesity, as well as from cancer, increased mortality, and reduced quality of life and productivity. Sleep insufficiency may be caused by broad scale societal factors such as round-the-clock access to technology and work schedules, but sleep disorders such as insomnia or obstructive sleep apnea also play an important role. An estimated 50-70 million US adults have a sleep or wakefulness disorder.

A “normal” or “restful” sleep period is defined as a sleep period uninterrupted by wakefulness. Alternatively, a said period can be defined by the recommended or appropriate amount of sleep for the subject's age category, e.g., (i) infants 0-3 months=about 11 to about 19 hours; (ii) infants about 4 to about 11 months=about 12 to about 18 hours; (iii) toddlers about 1 to about 2 years=about 9 to about 16 hours; (iv) preschoolers about 3 to about 5 years=about 10 to about 14 hours; (v) school-aged children about 6 to about 13 years=about 7 to about 12 hours; (v) teenagers about 14 to about 17 years=about 7 to about 11 hours; (vi) young adults about 18 to about 25 years=about 6 to about 11 hours; (vii) adults about 26 to about 64 years=about 6 to about 10 hours; and (viii) older adults≥65 years=about 5 to about 9 hours. Thus, for treating sleep disturbance in a subject, the treatment can result in a restful sleep period of at least about 4, about 5, about 6, about 7, about 8, about 9, about 10, about 11, or about 12 hours.

How much sleep is needed by a subject varies between individuals but generally changes with age. The National Institutes of Health suggests that school-age children need at least 10 hours of sleep daily, teens need 9-10 hours, and adults need 7-8 hours. According to data from the National Health Interview Survey, nearly 30% of adults reported an average of ≤6 hours of sleep per day in 2005-2007. Further, in 2009, only 31% of high school students reported getting at least 8 hours of sleep on an average school night. Similar recommendations are provided by the National Sleep Foundation (https://sleepfoundation.org/press-release/national-sleep-foundation-recommends-new-sleep-times/page/0/1):

TABLE 1

| | | May be | Not |
|---|---|---|---|
| Age | Recommended | appropriate | recommended |
| Newborns | 14 to 17 hours | 11 to 13 hours | Less than 11 hours |
| 0-3 months | | 18 to 19 hours | More than 19 hours |
| Infants | 12 to 15 hours | 10 to 11 hours | Less than 10 hours |
| 4-11 months | | 16 to 18 hours | More than 18 hours |
| Toddlers | 11 to 14 hours | 9 to 10 hours | Less than 9 hours |
| 1-2 years | | 15 to 16 hours | More than 16 hours |
| Preschoolers | 10 to 13 hours | 8 to 9 hours | Less than 8 hours |
| 3-5 years | | 14 hours | More than 14 hours |
| School-aged | 9 to 11 hours | 7 to 8 hours | Less than 7 hours |
| Children | | 12 hours | More than 12 hours |
| 6-13 years | | | |
| Teenagers | 8 to 10 hours | 7 hours | Less than 7 hours |
| 14-17 years | | 11 hours | More than 11 hours |
| Young Adults | 7 to 9 hours | 6 hours | Less than 6 hours |
| 18-25 years | | 10 to 11 hours | More than 11 hours |
| Adults | 7 to 9 hours | 6 hours | Less than 6 hours |
| 26-64 years | | 10 hours | More than 10 hours |
| Older | 7 to 8 hours | 5 to 6 hours | Less than 5 hours |
| Adults ≥65 years | | 9 hours | More than 9 hours |

There are several different scientifically acceptable ways to measure a sleep period uninterrupted by wakefulness. First, electrodes attached to the head of a subject can measure electrical activity in the brain by electroencephalography (EEG). This measure is used because the EEG signals associated with being awake are different from those found during sleep. Second, muscle activity can be measured using electromyography (EMG), because muscle tone also differs between wakefulness and sleep. Third, eye movements during sleep can be measured using electro-oculography (EOG). This is a very specific measurement that helps to identify Rapid Eye Movement or REM sleep. Any of these methods, or a combination thereof, can be used to determine if a subject obtains a restful sleep period following administration of at least one aminosterol or a salt or derivative thereof to the subject.

Further, circadian rhythm regulation can be monitored in a variety of ways, including but not limited to monitoring wrist skin temperature as described by Sarabia et al. 2008. Similarly symptoms of RBD can be monitored using a daily diary and RBD questionnaire (Stiasny-Kolster et al. 2007).

In some embodiments, administration of a therapeutically effective fixed dose of an aminosterol composition to a patient with disturbed results in improvement in frequency of normal or restful sleep as determined by a clinically recognized assessment scale for one or more types of sleep dysregulation, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The improvement can be measured using any clinically recognized tool or assessment.

Example 1 describes several tools used to measure and evaluate the effect of aminosterol treatment on sleep, including for example:

(1) Sleep Diary (participants completed a sleep diary on a daily basis throughout the study. The diaries included time into bed and estimated time to sleep as well as wake time and duration during the night.);

(2) I-Button Temperature Assessment. The I-Button is a small, rugged self-sufficient system that measures temperature and records the results in a protected memory section. The Thermochron I-Button DS1921H (Maxim Integrated, Dallas, Tex.) was used for skin temperature measurement. I-Buttons were programmed to sample every 10 mins., and attached to a double-sided cotton sport wrist band using Velcro, with the sensor face of the I-Button placed over the inside of the wrist, on the radial artery of the dominant hand. Subjects removed and replaced the data logger when necessary (i.e., to have a bath or shower). The value of skin temperature assessment in sleep research is that the endogenous skin warming resulting from increased skin blood flow is functionally linked to sleep propensity. From the collected data, the mesor, amplitude, acrophase (time of peak temperature), Rayleight test (an index of interdaily stability), mean waveforms are calculated);

(3) Unified Parkinson's Disease Rating Scale (UPDRS), sections 1.7 (sleep problems), 1.8 (daytime sleepiness) and 1.13 (fatigue);

(4) Parkinson's Disease Fatigue Scale (PFS-16);

(5) REM Sleep Behavior Disorder Screening Questionnaire; and

(6) Parkinson's Disease Sleep Scale.

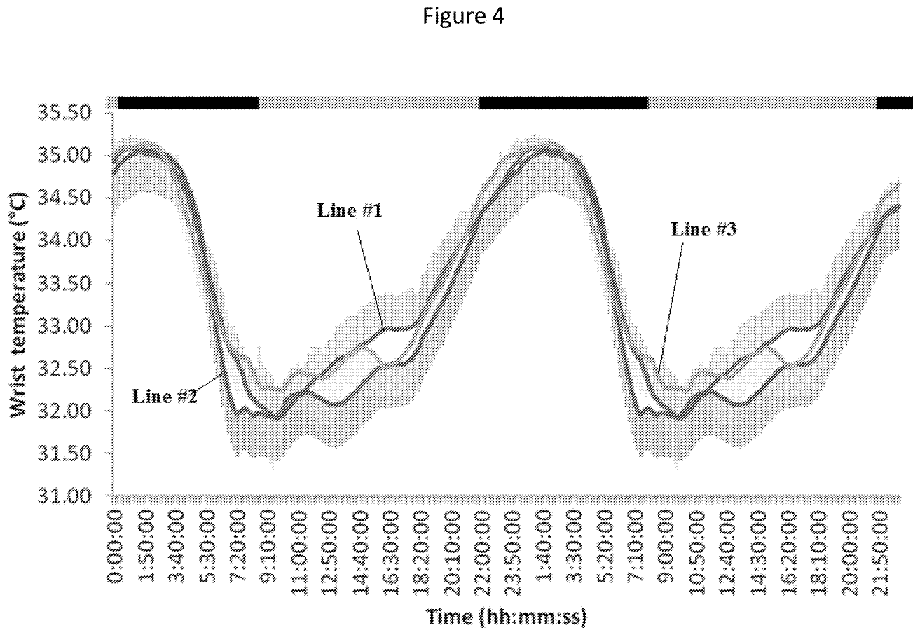

The data detailed in Example 1 described how circadian system status was evaluated by continuously monitoring wrist skin temperature (Thermochron iButton DS1921H; Maxim, Dallas) following published procedures (Sarabia et al. 2008). Further, an analysis was done with respect to the sleep data, the body temperature data, and fatigue data. The frequency of arm or leg thrashing reported in the sleep diary diminished progressively from 2.2 episodes/week at baseline to 0 at maximal dose (100% improvement). Total sleep time increased progressively from 7.1 hours at baseline to 8.4 hours at 250 mg (an 18% increase) and was consistently higher than baseline beyond 125 mg (FIG. 4). Unlike stool-related indices, the improvement in many CNS symptoms persisted during wash-out.

Circadian rhythm of skin temperature was evaluable in 12 patients (i.e., those who had recordings that extended from baseline through washout). Circadian system functionality was evaluated by continuously monitoring wrist skin temperature using a temperature sensor (Thermochron iButton DS1921H; Maxim, Dallas, Tex.) (Sarabia et al. 2008). Briefly, this analysis includes the following parameters: (i) the inter-daily stability (the constancy of 24-hour rhythmic pattern over days, IS); (ii) intra-daily variability (rhythm fragmentation, IV); (iii) average of 10-minute intervals for the 10 hours with the minimum temperature (L10); (iv) average of 10-minute intervals for the 5 hours with the maximum temperature (M5) and the relative amplitude (RA), which was determined by the difference between M5 and L10, divided by the sum of both. Finally, the Circadian Function Index (CFI) was calculated by integrating IS, IV, and RA. Consequently, CFI is a global measure that oscillates between 0 for the absence of circadian rhythmicity and 1 for a robust circadian rhythm.

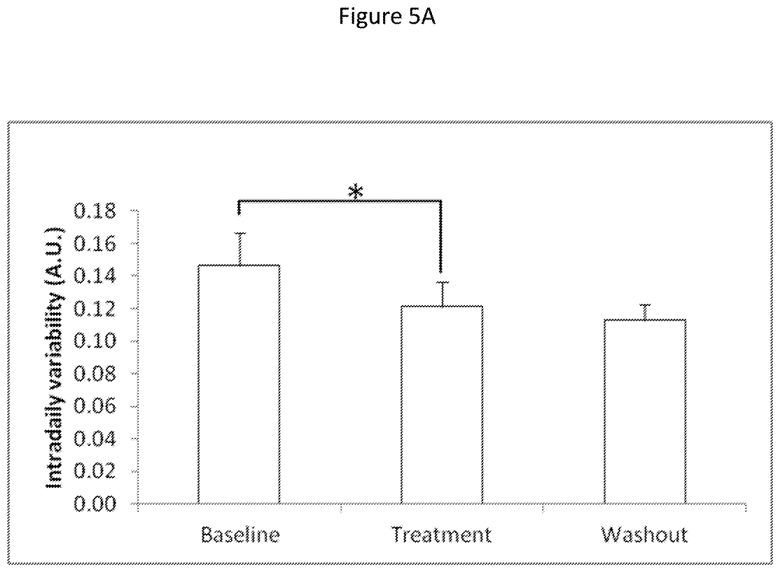

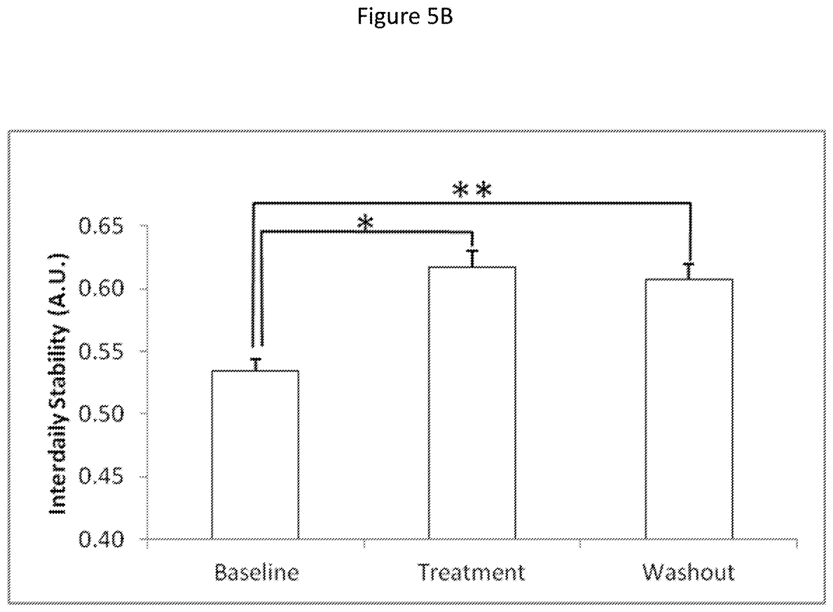

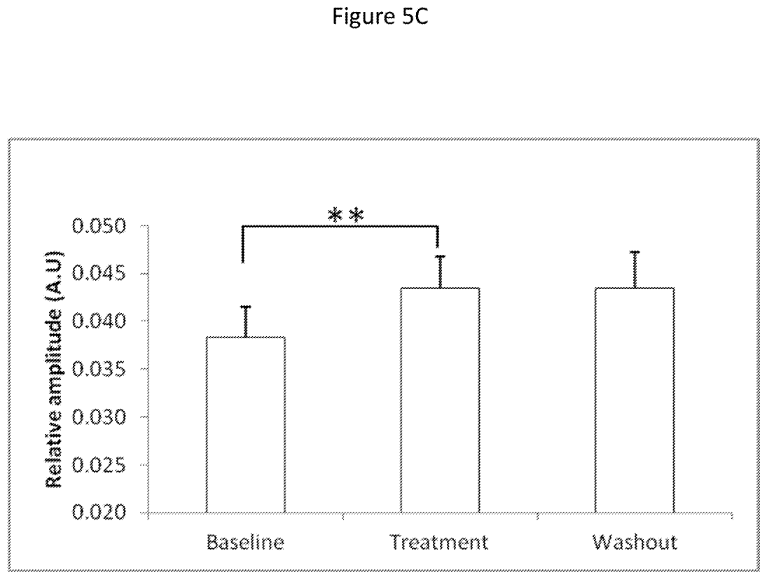

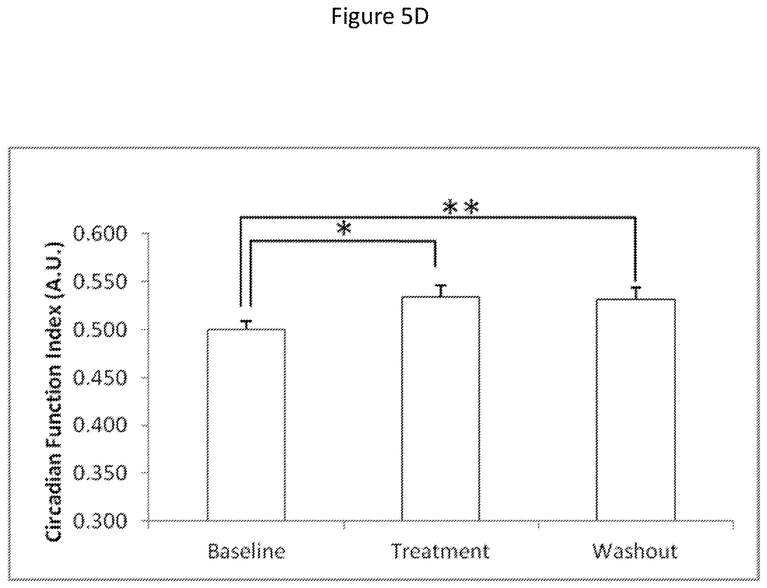

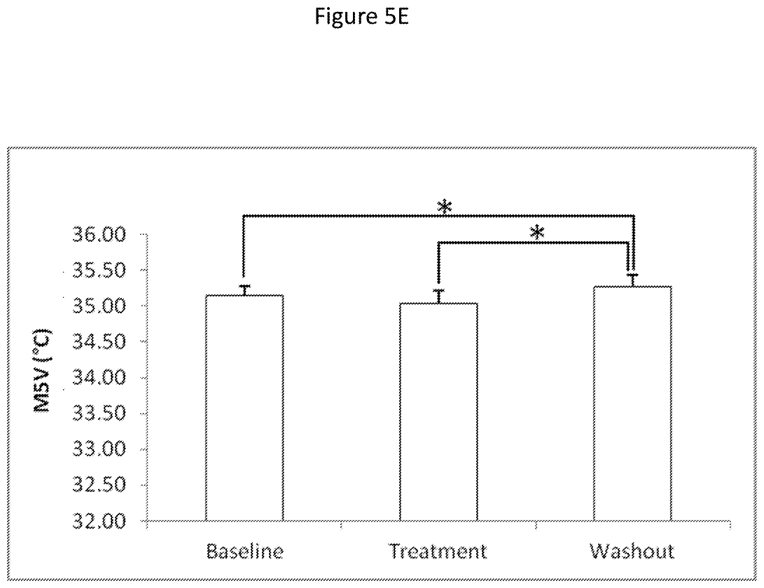

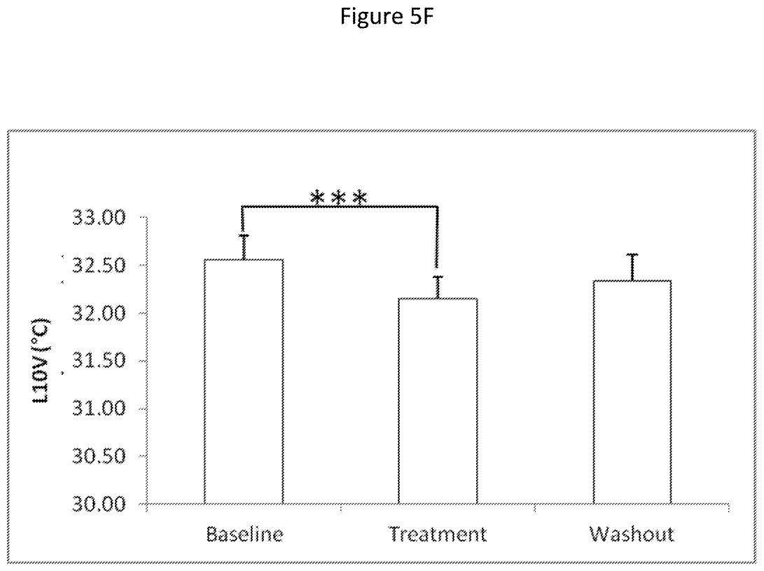

A comparison was performed of circadian rhythm parameters during the baseline, fixed dose and washout periods. Aminosterol administration improved all markers of healthy circadian function, including increasing rhythm stability, relative amplitude, and circadian function index, while reducing rhythm fragmentation. The improvement persisted for several of these circadian parameters during the wash-out period. (FIG. 5). Improvements were also seen in REM-behavior disorder (RBD) and sleep. RBD and total sleep time also improved progressively in a dose-dependent manner.

7. Cognitive Impairment

In one aspect of the disclosure, encompassed is a method of treating, preventing, and/or slowing the onset or progression of cognitive impairment (CI) and/or a related symptom in a subject in need, wherein the CI is correlated with abnormal α-synuclein (αS) pathology and/or dopaminergic dysfunction, the method comprising administering to the subject a therapeutically effective amount of at least one aminosterol, or a salt or derivative thereof. The method of administration can comprise, for example, oral, nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof. In another aspect, the method of administration can comprise non-oral administration or nasal administration.

In another aspect of the disclosure, encompassed is a method of treating, preventing, and/or slowing the onset or progression of cognitive impairment (CI) and/or a related symptom in a subject in need, wherein the CI is correlated with abnormal α-synuclein (αS) pathology and/or dopaminergic dysfunction, comprising: (a) determining a dose of an aminosterol or a salt or derivative thereof for the subject, wherein the aminosterol dose is determined based on the effectiveness of the aminosterol dose in improving or resolving a CI-related symptom being evaluated; (b) followed by administering the dose of the aminosterol or a salt or derivative thereof to the subject for a defined period of time, wherein the method comprises: (i) identifying a CI-related symptom to be evaluated; (ii) identifying a starting dose of the aminosterol or a salt or derivative thereof for the subject; and (iii) administering an escalating dose of the aminosterol or a salt or derivative thereof to the subject over a defined period of time until an effective dose is identified, wherein the effective dose is the dose where improvement of the CI-related symptom is observed, and fixing the aminosterol dose at that level in that particular subject; and (c) optionally wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

Cognitive impairment, including mild cognitive impairment (MCI), is characterized by increased memory or thinking problems exhibited by a subject as compared to a normal subject of the same age. Approximately 15 to 20 percent of people age 65 or older have MCI, and MCI is especially linked to neurodegenerative conditions such as Alzheimer's disease (AD) or synucleopathies like Parkinson's disease (PD). In 2002, an estimated 5.4 million people (22.%) in the United States over age 70 had cognitive impairment without dementia. Plassman et al. 2009.

Cognitive impairment may entail memory problems including a slight but noticeable and measurable decline in cognitive abilities, including memory and thinking skills. When MCI primarily affects memory, it is known as “amnestic MCI.” A person with amnestic MCI may forget information that would previously have been easily recalled, such as appointments, conversations, or recent events, for example. When MCI primarily affects thinking skills other than memory, it is known as “nonamnestic MCI.” A person with nonamnestic MCI may have a reduced ability to make sound decisions, judge the time or sequence of steps needed to complete a complex task, or with visual perception, for example.

Related disorders and conditions include, but are not limited to, dementia, Alzheimer's, delirium, Parkinson's, diabetes, high blood pressure, high cholesterol, depression, psychological and behavioral conditions, amnesia, Lewy body diseases, or Huntington's disease, among others.

Mild cognitive impairment is a clinical diagnosis. A combination of cognitive testing and information from a person in frequent contact with the subject is used to fully assess cognitive impairment. A medical workup includes one or more of an assessment by a physician of a subject's medical history (including current symptoms, previous illnesses, and family history), assessment of independent function and daily activities, assessment of mental status using brief tests to evaluate memory, planning, judgment, ability to understand visual information, and other key thinking skills, neurological examination to assess nerve and reflex function, movement, coordination, balance, and senses, evaluation of mood, brain imaging, or neuropsychological testing. Diagnostic guidelines for MCI have been developed by various groups, including the Alzheimer's Association partnered with the National Institute on Aging (NIA), an agency of the U.S. National Institutes of Health (NIH). Jack et al. 2011; McKhann et al. 2011; Albert et al. 2011. Recommendations for screening for cognitive impairment have been issued by the U.S. Preventive Services Task Force. Screening for Cognitive Impairment in Older Adults, U.S. Preventive Services Task Force (March 2014), https://www.uspreventiveservicestaskforce.org/Home/GetFileByID/1882. For example, the Mini Mental State Examination (MMSE) may be used (see FIG. 25). Palsetia et al. (2018); Kirkevold, O. & Selbaek, G. (2015). With the MMSE, a score of 24 or greater (out of 30) may indicate normal cognition, with lower scores indicating severe (less than or equal to 9 points), moderate (10-18 points), or mild (19-23 points) cognitive impairment. Other screening tools include the Informant Questionnaire on Cognitive Decline in the Elderly (IQCODE), in which an average score of 3 indicates no cognitive decline and a score greater than 3 indicates some decline. Jorm, A. F. 2004. Alternatively, the 7-Minute Screener, Abbreviated Mental Test Score (AMTS), Cambridge Cognitive Examination (CAMCOG), Clock Drawing Test (CDT), General Practitioner Assessment of Cognition (GPCOG), Mini-Cog, Memory Impairment Screen (MIS), Montreal Cognitive Assessment (MoCA), Rowland Universal Dementia Assessment (RUDA), Self-Administered Gerocognitive Examination (SAGE), Short and Sweet Screening Instrument (SAS-SI), Short Blessed Test (SBT), St. Louis Mental Status (SLUMS), Short Portable Mental Status Questionnaire (SPMSQ), Short Test of Mental Status (STMS), or Time and Change Test (T&C), among others, are frequently employed in clinical and research settings. Cordell et al. 2013. Numerous examinations may be used, as no single tool is recognized as the “gold standard,” and improvements in score on any standardized examination indicate successful treatment of cognitive impairment, whereas obtaining a score comparable to the non-impaired population indicates total recovery.

In some embodiments, administration of a therapeutically effective fixed dose of an aminosterol composition to a patient in need results in improvement of cognitive impairment as determined by a clinically recognized assessment scale, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The improvement can be measured using any clinically recognized tool or assessment.

As detailed in Example 1, cognitive impairment and the improvement following aminosterol treatment were assessed using several tools:

(1) Mini Mental State Examination (MMSE) (see FIG. 25);

(2) Trail Making Test (TMT) Parts A and B (see FIGS. 26 and 27); and

(3) Unified Parkinson's Disease Rating Scale (UPDRS), sections 1.1 (cognitive impairement).

Another clinically recognized tool that may be used for measuring cognitive impairment is the trail making test that assesses visual attention and task switching. (Arnett et al. 1995.) The trail making test consists of two parts in which the subject is instructed to connect a set of 25 dots as quickly as possible while still maintaining accuracy. Instructions for a typical trail making test is shown in FIG. 26, and FIG. 27 shows an example of a trail making test. The test can provide information about visual search speed, scanning, speed of processing, mental flexibility, as well as executive functioning. It is sensitive to detecting cognitive impairment associated with dementia, for example, AD.

Assessments were made at baseline and at the end of the fixed dose and washout periods for Example 1, and an analysis was done with respect to the cognition symptoms. The results showed that the total UPDRS score was 64.4 at baseline, 60.6 at the end of the fixed dose period and 55.7 at the end of the wash-out period (a 13.5% improvement). Part 1 of the UPDRS (which includes section 1.1, cognitive impairment) had a mean baseline score of 11.6, a fixed aminosterol dose mean score of 10.6, and a wash-out mean score of 9.5, demonstrating an almost 20% improvement (UPDRS cognitive impairment is rated from 1=slight improvement to 4=severe impairment, so lower scores correlate with better cognitive function). In addition, MMSE improved from 28.4 at baseline to 28.7 during treatment and to 29.3 during wash-out (the MMSE has a total possible score of 30, with higher scores correlating with better cognitive function). Unlike stool-related indices, the improvement in many CNS symptoms persisted during wash-out.

In one embodiment, the invention encompasses a method of treating, preventing and/or slowing the onset or progression of cognitive impairment (CI) and/or a related symptom in a subject in need comprising administering to the subject a therapeutically effective amount of at least one aminosterol or a salt or derivative thereof.

In another embodiment, the invention encompasses a method of treating, preventing and/or slowing the onset or progression of CI and/or a related symptom in a subject in need, where optionally the CI is correlated with abnormal α-synuclein (αS) pathology and/or dopaminergic dysfunction, comprising (a) determining a dose of an aminosterol or a salt or derivative thereof for the subject, wherein the aminosterol dose is determined based on the effectiveness of the aminosterol dose in improving or resolving a CI symptom being evaluated, (b) followed by administering the aminosterol dose to the subject for a period of time, wherein the method comprises (i) identifying a CI symptom to be evaluated; (ii) identifying a starting aminosterol dose for the subject; and (iii) administering an escalating dose of the aminosterol to the subject over a period of time until an effective dose for the CI symptom being evaluated is identified, wherein the effective dose is the aminosterol dose where improvement or resolution of the CI symptom is observed, and fixing the aminosterol dose at that level for that particular CI symptom in that particular subject.

In another embodiment, the starting aminosterol or a salt or derivative thereof dose is higher if the CI symptom being evaluated is severe. For example, the starting aminosterol dose can be based on a baseline score of a cognitive test or tool, wherein if the baseline score correlates with an assessment of mild cognitive impairment, then the starting aminosterol dose is lower than if the baseline score correlates with an assessment of severe cognitive impairment. In another aspect, a subject experiencing moderate or mild cognitive impairment as determined by a clinical scale or test is administered a starting oral aminosterol dose of from about 10 to about 75 mg/day; or a subject experiencing severe cognitive impairment as determined by a clinical scale or test is administered a starting oral aminosterol dose greater than about 75 mg/day.

In one embodiment, the method results in slowing, halting, or reversing progression or onset of CI over a defined period of time following administration of the fixed escalated dose of the aminosterol or a salt or derivative thereof, as measured by a medically-recognized technique. In addition, the method of the invention can result in positively impacting the CI, as measured by a medically-recognized technique.

The positive impact and/or progression of CI, and/or improvement or resolution of the CI symptom being evaluated, may be measured quantitatively or qualitatively by one or more clinically recognized scales, tools, or techniques). Examples of such techniques include computed tomography (CT), magnetic resonance imaging (MRI), magnetic resonance spectroscopy, functional MRI (fMRI), diffusion tensor imaging, single photon emission computed tomography (SPECT), and positron emission tomography (PET). In addition, the progression or onset of CI may be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In one embodiment, the fixed escalated aminosterol dose reverses dysfunction caused by the CI and treats, prevents, improves, and/or resolves the CI symptom being evaluated. Again, the improvement or resolution of the CI-related symptom can be measured using a clinically recognized scale or tool. Examples of such scales or tools include, for example, the Uniformed Parkinson's Disease Scale (UPDRS), Mini Mental State Examination (MMSE), Mini Mental Parkinson (MMP), Informant Questionnaire on Cognitive Decline in the Elderly (IQCODE), The 7-Minute Screen, Abbreviated Mental Test Score (AMTS), Cambridge Cognitive Examination (CAMCOG), Clock Drawing Test (CDT), General Practitioner Assessment of Cognition (GPCOG), Mini-Cog, Memory Impairment Screen (MIS), Montreal Cognitive Assessment (MoCA), Rowland Universal Dementia Assessment (RUDA), Self-Administered Gerocognitive Examination (SAGE), Short and Sweet Screening Instrument (SAS-SI), Short Blessed Test (SBT), St. Louis Mental Status (SLUMS), Short Portable Mental Status Questionnaire (SPMSQ), Short Test of Mental Status (STMS), Time and Change Test (T&C), Test Your Memory (TYM) test, and Addenbrooke's Cognitive Examination-Revised (ACER). Further, the improvement in the CI-related symptom is at least about 3%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale or tool.

In one aspect, the CI correlated with abnormal αS pathology and/or dopaminergic dysfunction is related to or correlated with a neurodegenerative disease or neurological disease associated with neural cell death. In another aspect, the neurodegenerative disease or neurological disease or related symptom associated with neural cell death is: (a) selected from the group consisting of septic shock, intracerebral bleeding, subarachnoidal hemorrhage, multiinfarct dementia, inflammatory diseases, neurotrauma, peripheral neuropathies, polyneuropathies, metabolic encephalopathies, and infections of the central nervous system; or (b) selected from the group consisting of synucleopathies, Alzheimer's disease, Parkinson's disease, dementia with Lewy bodies, multiple system atrophy, Huntington's disease, multiple sclerosis, parkinsonism, amyotrophic lateral sclerosis (ALS), schizophrenia, Friedreich's ataxia, vascular dementia, spinal muscular atrophy, frontotemporal dementia, supranuclear palsy, progressive supranuclear palsy, progressive nuclear palsy, degenerative processes associated with aging, dementia of aging, Guadeloupian parkinsonism, spinocerebellar ataxia, hallucinations, stroke, traumatic brain injury, down syndrome, Gaucher's disease, Krabbe's disease (KD), lysosomal conditions affecting glycosphingolipid metabolism, cerebral palsy, and epilepsy.

In another aspect, the CI correlated with abnormal αS pathology and/or dopaminergic dysfunction is related to or correlated with a psychological or behavioral disorder. For example, the psychological or behavioral disorder can be selected from the group consisting of aberrant motor and obsessive-compulsive behaviors, sleep disorders, REM sleep behavior disorder (RBD), depression, major depressive disorder, agitation, anxiety, delirium, irritability, ADHD, apathy, bipolar disorder, disinhibition, addiction, illusion and delusions, amnesia, and autism.

In one embodiment, the CI correlated with abnormal αS pathology and/or dopaminergic dysfunction is related to or correlated with a cerebral ischemic disorder or a general ischemic disorder. For example, the cerebral ischemic disorder can be selected from the group consisting of cerebral microangiopathy, intrapartal cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, and diabetic retinopathy; or the general ischemic disorder can be selected from the group consisting of high blood pressure, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, and pulmonary edema.

In another embodiment, the cognitive impairment-related symptom is selected from the group consisting of: cognitive impairment as determined by an IQ score; cognitive impairment as determined by a memory or cognitive function test; decline in thinking and reasoning skills; confusion; poor motor coordination; loss of short term memory; loss of long term memory; identity confusion; impaired judgement; forgetfulness; depression; anxiety; irritability; obsessive-compulsive behavior; apathy and/or lack of motivation; emotional imbalance; problem solving ability; impaired language; impaired reasoning; impaired decision-making ability; impaired ability to concentrate; impaired communication; impaired ability to conduct routine tasks such as cooking; self-care, including feeding and dressing; constipation; eurodegeneration; sleep problem, sleep disorder, and/or sleep disturbance; hypertension; hypotension; sexual dysfunction; cardiovascular disease; cardiovascular dysfunction; difficulty with working memory; gastrointestinal (GI) disorders; attention deficit and hyperactivity disorder; seizures; urinary dysfunction; difficulty with mastication; vision problems; and muscle weakness.

In one aspect, the CI-related symptom to be evaluated is cognitive impairment as determined by an IQ score or as determined by a memory or cognitive function test and wherein: (a) progression or onset of the CI is slowed, halted, or reversed over a defined period of time following administration of the fixed escalated dose of the aminosterol or a salt or derivative thereof, as measured by a medically-recognized technique; (b) the CI is positively impacted by the fixed escalated dose of the aminosterol or a salt or derivative thereof, as measured by a medically-recognized technique; (c) the CI is positively impacted by the fixed escalated dose of the aminosterol or a salt or derivative thereof, as measured by a medically-recognized technique and the positive impact on and/or progression of cognitive decline is measured quantitatively or qualitatively by one or more medically-recognized techniques selected from the group consisting of ADASCog, Mini-Mental State Exam (MMSE), Mini-cog test, Woodcock-Johnson Tests of Cognitive Abilities, Leiter International Performance Scale, Miller Analogies Test, Raven's Progressive Matrices, Wonderlic Personnel Test, IQ tests, or a computerized tested selected from Cantab Mobile, Cognigram, Cognivue, Cognision, and Automated Neuropsychological Assessment Metrics Cognitive Performance Test (CPT); and/or (d) the progression or onset of CI is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In one embodiment, the CI-related symptom to be evaluated is depression and (a) the method results in improvement in a subject's depression, as measured by one or more clinically-recognized depression rating scale; (b) the method results in improvement in a subject's depression, as measured by one or more clinically-recognized depression rating scale and the improvement is in one or more depression characteristics selected from the group consisting of mood, behavior, bodily functions such as eating, sleeping, energy, and sexual activity, and/or episodes of sadness or apathy; and/or (c) the method results in improvement in a subject's depression, as measured by one or more clinically-recognized depression rating scale, and the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%. For example, the one or more clinically-recognized depression rating scale can be selected from the group consisting of the Patient Health Questionnaire-9 (PHQ-9); the Beck Depression Inventory (BDI); Zung Self-Rating Depression Scale; Center for Epidemiologic Studies-Depression Scale (CES-D); and the Hamilton Rating Scale for Depression (HRSD).

In one embodiment, the CI-related symptom to be evaluated is constipation, and (a) treating the constipation prevents and/or delays the onset and/or progression of the CI; (b) the fixed escalated aminosterol dose causes the subject to have a bowel movement; (c) the method results in an increase in the frequency of bowel movement in the subject; (d) the method results in an increase in the frequency of bowel movement in the subject and the increase in the frequency of bowel movement is defined as: (i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; (e) as a result of the method the subject has the frequency of bowel movement recommended by a medical authority for the age group of the subject; and/or (f) the starting aminosterol dose is determined by the severity of the constipation, wherein: (i) if the average complete spontaneous bowel movement (CSBM) or spontaneous bowel movement (SBM) is one or less per week, then the starting aminosterol dose is at least about 150 mg; and (ii) if the average CSBM or SBM is greater than one per week, then the starting aminosterol dose is about 75 mg or less.

In one embodiment, the CI-related symptom to be evaluated is neurodegeneration correlated with CI, and (a) treating the neurodegeneration prevents and/or delays the onset and/or progression of the CI; (b) the method results in treating, preventing, and/or delaying the progression and/or onset of neurodegeneration in the subject; (c) progression or onset of the neurodegeneration is slowed, halted, or reversed over a defined period of time following administration of the fixed escalated dose of the aminosterol or a salt or derivative thereof, as measured by a medically-recognized technique; and/or (d) the neurodegeneration is positively impacted by the fixed escalated dose of the aminosterol or a salt or derivative thereof, as measured by a medically-recognized technique. For example, the positive impact and/or progression of neurodegeneration can be measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis. In addition, the progression or onset of neurodegeneration can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In one embodiment, the CI-related symptom to be evaluated is a sleep problem, sleep disorder, or sleep disturbance and (a) the sleep problem, sleep disorder, or sleep disturbance comprises a delay in sleep onset, sleep fragmentation, REM-behavior disorder, sleep-disordered breathing including snoring and apnea, day-time sleepiness, micro-sleep episodes, narcolepsy, circadian rhythm dysfunction, REM disturbed sleep, or any combination thereof; (b) the sleep problem, sleep disorder, or sleep disturbance comprises REM-behavior disorder, which comprises vivid dreams, nightmares, and acting out the dreams by speaking or screaming, or fidgeting or thrashing of arms or legs during sleep; (c) treating the sleep problem, sleep disorder, or sleep disturbance prevents or delays the onset and/or progression of the CI; (d) the method results in a positive change in the sleeping pattern of the subject; wherein the positive change is defined as: (i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (f) as a result of the method the subject obtains the total number of hours of sleep recommended by a medical authority for the age group of the subject.

In another embodiment, the aminosterol or a salt or derivative thereof is administered in combination with at least one additional active agent to achieve either an additive or synergistic effect. For example, the additional active agent can be administered via a method selected from the group consisting of (a) concomitantly; (b) as an admixture; (c) separately and simultaneously or concurrently; or (d) separately and sequentially. In another embodiment, the additional active agent is a different aminosterol from that administered in primary method. In yet a further embodiment, the method of the invention comprises administering a first aminosterol which is aminosterol 1436 or a salt or derivative thereof intranasally and administering a second aminosterol which is squalamine or a salt or derivative thereof orally.

In another embodiment, the subject to be treated according to the methods of the invention can be a member of a patient population at risk for being diagnosed with CI.

8. Depression

In one aspect, encompassed is a method of treating, preventing, and/or slowing the onset or progression of depression and/or a related symptom in a subject in need comprising administering to the subject a therapeutically effective amount of at least one aminosterol, or a salt or derivative thereof, provided that the administering does not comprise oral administration. For example, the method of administration can comprise nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof.

In another aspect, encompassed is a method of treating, preventing, and/or slowing the onset or progression of depression and/or a related symptom in a subject in need comprising: (a) determining a dose of an aminosterol or a salt or derivative thereof for the subject, wherein the aminosterol dose is determined based on the effectiveness of the aminosterol dose in improving or resolving a depression symptom being evaluated, (b) followed by administering the dose of the aminosterol or a salt or derivative thereof to the subject for a defined period of time, wherein the method comprises: (i) identifying a depression symptom to be evaluated; (ii) identifying a starting dose of an aminosterol or a salt or derivative thereof for the subject; and (iii) administering an escalating dose of the aminosterol or a salt or derivative thereof to the subject over a defined period of time until an effective dose for the depression symptom being evaluated is identified, wherein the effective dose is the aminosterol dose where improvement or resolution of the depression symptom is observed, and fixing the aminosterol dose at that level for that particular depression symptom in that particular subject; and (c) optionally wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

Clinical depression is characterized by a sad, blue mood that goes above and beyond normal sadness or grief. Major depression is an episode of sadness or apathy along with other symptoms that lasts at least two consecutive weeks and is severe enough to interrupt daily activities. Depressive events feature not only negative thoughts, moods, and behaviors but also specific changes in bodily functions (like, eating, sleeping, energy and sexual activity, as well as potentially developing aches or pains). One in 10 people will have a depression in their lifetime. Doctors clinically diagnose depression; there is no laboratory test or X-ray for depression.

Increasingly sophisticated forms of brain imaging, such as positron emission tomography (PET), single-photon emission computed tomography (SPECT), and functional magnetic resonance imaging (fMRI), permit a much closer look at the worki6yng brain than was possible in the past. An fMRI scan, for example, can track changes that take place when a region of the brain responds during various tasks. A PET or SPECT scan can map the brain by measuring the distribution and density of neurotransmitter receptors in certain areas. Use of this technology has led to a better understanding of which brain regions regulate mood and how other functions, such as memory, may be affected by depression. Areas that play a significant role in depression are the amygdala, the thalamus, and the hippocampus.

Research shows that the hippocampus is smaller in some depressed people. For example, in one fMRI study published in The Journal of Neuroscience, investigators studied 24 women who had a history of depression. On average, the hippocampus was 9% to 13% smaller in depressed women as compared with those who were not depressed. The more bouts of depression a woman had, the smaller the hippocampus. Stress, which plays a role in depression, may be a key factor, since experts believe stress can suppress the production of new neurons (nerve cells) in the hippocampus.

Researchers are exploring possible links between sluggish production of new neurons in the hippocampus and low moods. An interesting fact about antidepressants supports this theory. These medications immediately boost the concentration of chemical messengers in the brain (neurotransmitters). Yet people typically don't begin to feel better for several weeks or longer. Experts have long wondered why, if depression were primarily the result of low levels of neurotransmitters, people don't feel better as soon as levels of neurotransmitters increase. The answer may be that mood only improves as nerves grow and form new connections, a process that takes weeks. In fact, animal studies have shown that antidepressants do spur the growth and enhanced branching of nerve cells in the hippocampus. So, the theory holds, the real value of these medications may be in generating new neurons (a process called neurogenesis), strengthening nerve cell connections, and improving the exchange of information between nerve circuits.

Thus, in one embodiment of the invention, encompassed are methods of treating and/or preventing depression comprising administering therapeutically effective fixed dose of an aminosterol composition according to the invention. While not wishing to be bound by theory, it is theorized that the aminosterol compositions of the invention trigger neurogenesis, which functions to combat depression.

In some embodiments, the methods of the invention produce an improvement in a subject's clinical depression. An improvement in a subject's depression can be measured using any clinically-recognized measurement. For example, improvement can be measured using a depression rating scale. In one embodiment of the invention, following treatment a subject experiences an about 5, about 10, about 15, about 20, about 25, about 30, about 35, about 40, about 45, about 50, about 55, about 60, about 65, about 70, about 75, about 80, about 85, about 90, about 95 or an about 100% improvement. The improvement can be measured using any clinically recognized tool or assessment.

As detailed in Example 1, depression and/or mood and the improvement following aminosterol treatment were assessed using several tools:

(1) Beck Depression Inventory (BDI-II);

(2) Unified Parkinson's Disease Rating Scale (UPDRS), sections 1.3 (depressed mood), 1.4 (anxious mood), 1.5 (apathy), and 1.13 (fatigue); and

(3) Parkinson's Disease Fatigue Scale (PFS-16).

Assessments were made at baseline and at the end of the fixed dose and washout periods. An analysis was done with respect to depression and mood scores. Total UPDRS score was 64.4 at baseline, 60.6 at the end of the fixed dose period and 55.7 at the end of the wash-out period, demonstrating a 13.5% improvement, and Part 1 of the UPDRS (which includes mood and depression scores) went from a mean score of 11.6 at baseline, to a mean of 10.6 during the fixed aminosterol dose period, with a mean score of 9.5 during the washout period, demonstrating an improvement of 18%. In addition, BDI-II scores decreased from 10.9 at baseline to 9.9 during treatment and 8.7 at wash-out, showing an improvement in depression scoring of 20%. Unlike stool-related indices, the improvement in many CNS symptoms persisted during wash-out.

In one embodiment, the invention encompasses a method of treating, preventing and/or slowing the onset or progression of depression and/or a related symptom in a subject in need comprising administering to the subject a therapeutically effective amount of at least one aminosterol or a salt or derivative thereof. In some embodiments, administration of the aminosterol is via non-oral means.

In another embodiment, the invention encompasses a method of treating, preventing and/or slowing the onset or progression of depression and/or a related symptom in a subject in need comprising (a) determining a dose of an aminosterol or a salt or derivative thereof for the subject, wherein the aminosterol dose is determined based on the effectiveness of the aminosterol dose in improving or resolving a depression symptom being evaluated, (b) followed by administering the aminosterol dose to the subject for a period of time, wherein the method comprises (i) identifying a depression symptom to be evaluated; (ii) identifying a starting aminosterol dose for the subject; and (iii) administering an escalating dose of the aminosterol to the subject over a period of time until an effective dose for the depression symptom being evaluated is identified, wherein the effective dose is the aminosterol dose where improvement or resolution of the depression symptom is observed, and fixing the aminosterol dose at that level for that particular depression symptom in that particular subject.

In another embodiment, the starting aminosterol or a salt or derivative thereof dose is higher if the depression symptom being evaluated is severe.

In one aspect, where severity of the depression is reduced over a defined period of time, the reduction in severity can be measured from one or more medically-recognized techniques selected from the group consisting of the Patient Health Questionnaire-9 (PHQ-9); the Beck Depression Inventory (BDI); Zung Self-Rating Depression Scale; Center for Epidemiologic Studies-Depression Scale (CES-D); and the Hamilton Rating Scale for Depression (HRSD). The defined period of time during which the severity of the depression is reduced can be about 1 day to about 10 days, about 10 days to about 30 days, about 30 days to about 3 months, about 3 months to about 6 months, about 6 months to about 12 months, or about greater than 12 months.

In one embodiment, the method results in slowing, halting, or reversing progression or onset of depression over a defined period of time following administration of the fixed escalated dose of the aminosterol or a salt or derivative thereof, as measured by a medically-recognized technique. For example, the progression or onset of depression may be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique. In addition, the method of the invention can result in positively impacting the depression, as measured by a medically-recognized technique. The positive impact on and/or progression of depression can be measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis. In addition, the progression or onset of depression can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In one embodiment, the fixed escalated aminosterol dose reverses dysfunction caused by the depression and treats, prevents, improves, and/or resolves the depression symptom being evaluated. In one aspect, the improvement or resolution of the depression symptom is measured using a clinically recognized scale or tool. For example, the improvement in the depression symptom can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale.

In yet another embodiment, the depression symptom to be evaluated can be selected from the group consisting of (a) a symptom from the Hamilton Depression Rating Scale (HAM-D) selected from the group consisting of depressed mood, feelings of guilt, suicide, initial insomnia, middle of night insomnia, delayed insomnia, work and interests, retardation, agitation, psychic anxiety, somatic anxiety, gastrointestinal symptoms, general somatic symptoms, genital symptoms, hypochondriasis, weight loss, insight, diurnal variation, depersonalization and derealization, paranoid symptoms, and obsessional symptoms; (b) a symptom from the Montgomery-Asberg Depression Scale (MADRS) selected from the group consisting of apparent sadness, reported sadness, inner tension, reduced sleep, concentration difficulties, lassitude, inability to feel, pessimistic thoughts, and suicidal thoughts; (c) a symptom from Beck's Depression Inventory (BDI) selected from the group consisting of sadness, outlook on the future, feelings of failure, satisfaction, guilt, feelings of being punished, disappointment with self, self-blame, suicidal ideation, crying frequency, prevalence of irritation, interest in others, ease of decision-making, self-image, ability to work, ease of sleep, tiredness, appetite, weight loss, preoccupation with health, and lack of libido; (d) apathy; (e) hopelessness; (f) loss of interest in hobbies; (g) sleep problem, sleep disorder, or sleep disturbance; (h) excessive hunger; (i) lack of appetite; (j) restlessness; (k) social isolation; (l) cognitive impairment; (m) weight loss; (n) weight gain; and (o) constipation.

In one aspect, the sleep problem, sleep disorder, or sleep disturbance comprises a delay in sleep onset, sleep fragmentation, REM-behavior disorder, sleep-disordered breathing including snoring and apnea, day-time sleepiness, micro-sleep episodes, narcolepsy, early awakening, insomnia, hallucinations, or any combination thereof; In another aspect, the REM-behavior disorder comprises vivid dreams, nightmares, and acting out the dreams by speaking or screaming, or fidgeting or thrashing of arms or legs during sleep. Finally, the hallucination can comprise a visual, auditory, tactile, gustatory or olfactory hallucination.

In one embodiment, where the depression symptom to be evaluated comprises a sleep problem, sleep disorder, sleep disturbance, circadian rhythm dysfunction, REM disturbed sleep, or REM behavior disorder, then (a) the method results in a positive change in the sleeping pattern of the subject; (b) the method results in a positive change in the sleeping pattern of the subject, wherein the positive change is defined as (i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (c) as a result of the method the subject obtains the total number of hours of sleep recommended by a medical authority for the age group of the subject.

In one embodiment, the depression symptom to be evaluated comprises suicidal thoughts and (a) the method results in a decreased number or severity of suicidal thoughts of the subject; (b) the method results in a decreased number or severity of suicidal thoughts of the subject and the decrease in number or severity in suicidal thoughts is defined as a reduction in occurrences or severity of suicidal thoughts selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (c) the method results in the subject being free of suicidal thoughts.

In another embodiment, the depression symptom to be evaluated is sadness and (a) the method results in improvement in the subject's sadness, as measured by one or more clinically-recognized depression rating scale; and/or (b) the method results in improvement in the subject's sadness, as measured by one or more clinically-recognized depression rating scale, and the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%.

In one embodiment, the depression symptom to be evaluated comprises cognitive impairment, and (a) progression or onset of the cognitive impairment is slowed, halted, or reversed over a defined period of time following administration of the fixed escalated dose of the aminosterol or a salt or derivative thereof, as measured by a medically-recognized technique; and/or (b) the cognitive impairment is positively impacted by the fixed escalated dose of the aminosterol or a salt or derivative thereof, as measured by a medically-recognized technique; (c) the cognitive impairment is positively impacted by the fixed escalated dose of the aminosterol or a salt or derivative thereof, as measured by a medically-recognized technique and the positive impact on and/or progression of cognitive impairment is measured quantitatively or qualitatively by one or more techniques selected from the group consisting of ADASCog, Mini-Mental State Exam (MMSE), Mini-cog test, Woodcock-Johnson Tests of Cognitive Abilities, Leiter International Performance Scale, Miller Analogies Test, Raven's Progressive Matrices, Wonderlic Personnel Test, IQ tests, and a computerized tested selected from Cantab Mobile, Cognigram, Cognivue, Cognision, or Automated Neuropsychological Assessment Metrics Cognitive Performance Test (CPT); and/or (d) the progression or onset of cognitive impairment is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In some embodiments, the depression symptom to be evaluated is constipation, and (a) treating the constipation prevents and/or delays the onset and/or progression of the depression; (b) the fixed escalated aminosterol dose causes the subject to have a bowel movement; (c) the method results in an increase in the frequency of bowel movement in the subject; (d) the method results in an increase in the frequency of bowel movement in the subject and the increase in the frequency of bowel movement is defined as: (i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; (e) as a result of the method the subject has the frequency of bowel movement recommended by a medical authority for the age group of the subject; and/or (f) the starting aminosterol dose is determined by the severity of the constipation, wherein: (i) if the average complete spontaneous bowel movement (CSBM) or spontaneous bowel movement (SBM) is one or less per week, then the starting aminosterol dose is at least about 150 mg; and (ii) if the average CSBM or SBM is greater than one per week, then the starting aminosterol dose is about 75 mg or less.

In one embodiment, the depression symptom to be evaluated comprises lack of libido, and (a) the method results in treating, preventing, and/or delaying the progression and/or onset of lack of libido in the subject; (b) progression or onset of the lack of libido is slowed, halted, or reversed over a defined period of time following administration of the fixed escalated dose of the aminosterol or a salt or derivative thereof, as measured by a medically-recognized technique; (c) the lack of libido is positively impacted by the fixed escalated dose of the aminosterol or a salt or derivative thereof, as measured by a medically-recognized technique; (d) the progression of (b) and/or the positive impact of (c) is measured quantitatively or qualitatively by one or more techniques selected from the group consisting of the Sexual Desire Inventory-2 (SDI-2), Brief Index for SF Form Women, Brief Sexual Function Questionnaire for Men, Deragotis Sexual Function Inventory (DSFI), Derogatis interview for Sexual Function, Female Sexual Arousability Index, Florida Sexual History Questionnaire (FSHD), General Information Form (GIF), Golombok Rust Inventory of Sexual Satisfaction (GRISS), Hanson Assessment of Sexual Health, Heterosexual Behavior Assessment Females, Heterosexual Behavior Assessment Males, Heterosexual Zuckerman, Homosexual Zuckerman, Hypogonadism and Sexual Function, Index of Sexual Satisfaction (ISS), International Index of Erectile Function, Jewish General Hospital Sexual Self-Monitoring Form, Leiden Impotence Questionnaire, McCoy Female Sexuality Questionnaire, Multiaxial Problem-oriented Diagnostic System of SF, Potency and Prostatectomy, Radical Prostatectomy Questionnaire, Sabbastberg Sexual Rating Scale (revised), Scalability of Sexual Experience, Segraves Sexual Symptomatology Interview, Sexual Activity of Men presenting Prostatism and Prostatectomy, Sexual Adjustment Questionnaire (SAQ), Sexual Dysfunction (Silence Hurts), Sexual Dysfunction in HIV+Men (assoc w/neuropathy/CD4 count), Sexual Dysfunction in HIV+Men, Sexual Dysfunction in Schizophrenic Patients, Sexual Function Scale, Sexual Interaction Inventory (SII), Sexual Interaction System Scale, Sexual Interest and Satisfaction Scale, Sexual Interest Questionnaire (SIQ), Sexual Inventory (SI), Sexual Orientation Method and Anxiety (SOMA), Sexual Self-Efficacy Scale for Erectile Disorder (SSES-E), Sexual Symptom Distress Scale, Sexuality Experience Scale, The Clark Sexual History Questionnaire, Urge-incontinence Impact Questionnaire, Vaginal Changes and Sexuality in Women with Cervical CA, and Watts Sexual Function Questionnaire; and/or (e) the progression or onset of (b) is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In another embodiment, the aminosterol or a salt or derivative thereof is administered in combination with at least one additional active agent to achieve either an additive or synergistic effect. For example, the additional active agent can be administered via a method selected from the group consisting of (a) concomitantly; (b) as an admixture; (c) separately and simultaneously or concurrently; or (d) separately and sequentially. In another embodiment, the additional active agent is a different aminosterol from that administered in primary method. In yet a further embodiment, the method of the invention comprises administering a first aminosterol which is aminosterol 1436 or a salt or derivative thereof intranasally and administering a second aminosterol which is squalamine or a salt or derivative thereof orally.

In another embodiment, the at least one additional active agent is an active agent used to treat depression or a symptom thereof. In some embodiments, the active agent is selected from the group consisting of selective serotonin reuptake inhibitors (SSRIs) such as citalopram (Celexa®, Cipramil®), escitalopram (Lexapro®, Cipralex®), paroxetine (Paxil®, Seroxat®), fluoxetine (Prozac®), fluvoxamine (Luvox®, Faverin®), sertraline (Zoloft®, Lustral®), indalpine (Upstene®), zimelidine (Normud®, Zelmid®); serotonin-norepinephrine reuptake inhibitors (SNRIs) such as desvenlafaxine (Pristiq®), duloxetine (Cymbalta®), levomilnacipran (Fetzima®), milnacipran (Ixel®, Savella®), venlafaxine (Effexor®); serotonin modulators and stimulators (SMSs) such as vilazodone (Viibryd®), vortioxetine (Trintellix®); serotonin antagonists and reuptake inhibitors such as nefazodone (Dutonin®, Nefadar®, Serzone®), trazodone (Desyrel®), etoperidone; norepinephrine reuptake inhibitors (NRIs) such as reboxetine (Edronax®), teniloxazine (Lucelan®, Metatone®), viloxazine (Vivalan®), atomoxetine (Strattera®); norepinephrine-dopamine reuptake inhibitors such as bupropion (Wellbutrin®), amineptine (Survector®, Maneon®), nomifensine (Merital®, Alival®), methylphenidate (Ritalin®, Concerta®), lisdexamfetamine (Vyvanse®); tricyclic antidepressants such asamitriptyline (Elavil®, Endep®), amitriptylinoxide (Amioxid®, Ambivalon®, Equilibrin®), clomipramine (Anafranil®), desipramine (Norpramin®, Pertofrane®), dibenzepin (Noveril®, Victoril®), dimetacrine (Istonil®), dosulepin (Prothiaden®), doxepin (Adapin®, Sinequan®), imipramine (Tofranil®), lofepramine (Lomont®, Gamanil®), melitracen (Dixeran®, Melixeran®, Trausabun®), nitroxazepine (Sintamil®), nortriptyline (Pamelor®, Aventyl®), noxiptiline (Agedal®, Elronon®, Nogedal®), opipramol (Insidon®), pipofezine (Azafen®/Azaphen®), protriptyline (Vivactil®), trimipramine (Surmontil®), butriptyline (Evadyne®), demexiptiline (Deparon®, Tinoran®), fluacizine (Phtorazisin®), imipraminoxide (Imiprex®, Elepsin®), iprindole (Prondol®, Galatur®, Tertran®), metapramine (Timaxel®), propizepine (Depressing, Vagran®), quinupramine (Kinupril®, Kevopril®), tiazesim (Altinil®), tofenacin (Elamol®, Tofacine®), amineptine (Survector®, Maneon®), tianeptine (Stablon®, Coaxil®); tetracyclic antidepressants such as amoxapine (Asendin®), maprotiline (Ludiomil®), mianserin (Bolvidon®, Norval®, Tolvon®), mirtazapine (Remeron®), setiptiline (Tecipul®), mianserin, mirtazapine, setiptiline; monoamine oxidase inhibitors (MAOIs) such as isocarboxazid (Marplan®), phenelzine (Nardil®), tranylcypromine (Parnate®), benmoxin (Neuralex®), iproclozide (Sursum®), iproniazid (Marsilid®), mebanazine (Actomol®), nialamide (Niamid®), octamoxin (Ximaol®), pheniprazine (Catron®), phenoxypropazine (Drazine®), pivhydrazine (Tersavid®), safrazine (Safra®), selegiline (Eldepryl®, Zelapar®, Emsam®), caroxazone (Surodil®, Timostenil®), metralindole (Inkazan®), moclobemide (Aurorix®, Manerix®), pirlindole (Pirazidol®), toloxatone (Humoryl®), eprobemide (Befol®), minaprine (Brantur®, Cantor®), bifemelane (Alnert®, Celeport®); atypical antipsychotics such as amisulpride (Solian®), lurasidone (Latuda®), quetiapine (Seroquel®); or N-methyl D-aspartate (NMDA) antagonists such ketamine (Ketalar®).

In another embodiment, the subject to be treated according to the methods of the invention can be a member of a patient population at risk for being diagnosed with depression.

9. Alpha-Synuclein Aggregation

Alpha-synuclein is a potent pro-inflammatory hormone. Inflammation can be blocked by either of two strategies. First, inflammation can be blocked by reducing the tissue concentration of alpha-synuclein by decreasing or stopping production of alpha-synuclein. Alternatively, inflammation can be blocked by interrupting the signaling between alpha-synuclein and inflammatory cells that express CD11b. The subject of the methods of the invention can be any mammal, including a human.

The inflammatory disease or condition caused by excessive expression of neuronal alpha synuclein can be a neurodegenerative disorder (NDD), such as an alpha-synucleinopathy. Exemplary alpha-synucleinopathies include, but are not limited to, PD, Lewy body dementia, multiple system atrophy, amytrophic lateral sclerosis, Huntington's chorea, multiple sclerosis or schizophrenia. In other embodiments, the inflammatory disease or condition caused by excessive expression of neuronal alpha synuclein can be an autoimmune disease, a chronic inflammatory disease, or an autoinflammatory disease. In other embodiments, the inflammatory disease or condition caused by excessive expression of neuronal alpha synuclein can be selected from the group consisting of asthma, chronic peptic ulcer, tuberculosis, chronic periodontitis, chronic sinusitis, chronic active hepatitis, psoriatic arthritis, gouty arthritis, acne vulgaris, osteoarthritis, rheumatoid arthritis, lupus, systemic lupus erythematosus, multiple sclerosis, ankylosing spondylitis, Crohn's disease, psoriasis, primary sclerosing cholangitis, ulcerative colitis, allergies, inflammatory bowel diseases, Celiac disease, Chronic prostatitis, diverticulitis, dermatomyositis, polymyositis, systemic sclerosis, glomerulonephritis, hidradenitis suppurativa, hypersensitivities, interstitial cystitis, otitis, pelvic inflammatory disease, reperfusion injury, rheumatic fever, sarcoidosis, transplant rejection, and vasculitis.

In some embodiments of the invention, patient populations particularly susceptible to excessive production or secretion of alpha-synuclein can benefit from the methods of the invention and are targeted for therapy, including for example preventative therapy. For example, a patient population having a mutated form of alpha-synuclein resulting in increased amounts of alpha-synuclein in tissues can be treated using the methods of the invention. Another example of a patient population susceptible for high levels of alpha-synuclein are patients having chronic inflammatory conditions or diseases.

The methods of the invention can result in a decrease in intensity of inflammation, blood levels of inflammatory markers, inflammatory markers in tissue, or number of inflammatory cells in tissue, or a combination thereof, as compared to a control or as compared to the qualitative or quantitative amount from the same patient or subject prior to treatment. For example, the decrease can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The improvement can be measured using any clinically recognized tool or assessment.

In some embodiments of the invention, patient populations particularly susceptible to excessive production or secretion of alpha-synuclein can benefit from the methods of the invention and are targeted for therapy, including for example preventative therapy. For example, a patient population having a mutated form of alpha-synuclein resulting in increased amounts of alpha-synuclein in tissues can be treated using the methods of the invention. Another example of a patient population susceptible for high levels of alpha-synuclein are patients having chronic inflammatory conditions or diseases. A still further example is a patient population having elevated levels of alpha-synuclein aggregation in their enteric nerve cells, manifesting as a constipation.

In addition, it follows from the present invention that an individual with an inflammatory condition appropriate for treatment or prophylaxis with the methods targeting alpha-synuclein described herein can be identified by determination of the tissue concentrations of alpha synuclein at sites of inflammation, with high levels of alpha-synuclein, as compared to a control or healthy subject, correlating with patients appropriate for treatment with a method of the invention.

Based on the data detailed in Example 1, it is believed that administration of an aminosterol reduces the formation of neurotoxic αS aggregates in vivo, and stimulates gastrointestinal motility in patients with neurodiseases such as PD and constipation. The observation that the dose required to achieve a prokinetic response increases with constipation severity supports the hypothesis that the greater the burden of αS impeding neuronal function, the higher the dose of aminosterol required to restore normal bowel function as well as address other symptoms of alpha-synuclein aggregation. The data detailed in Example 1 is the first proof of concept demonstration that directly targeting αS pharmacologically can achieve beneficial GI, autonomic and CNS responses.

This data in Example 1 supports the hypothesis that gastrointestinal dysmotility in neurodiseases such as PD results from the progressive accumulation of αS in the ENS, and that aminosterols can restore neuronal function by displacing αS and stimulating enteric neurons. Improvements were also seen in cognitive function (MMSE scores) (see FIG. 25), hallucinations, REM-behavior disorder (RBD) and sleep. These improvements are unlikely to be due to improved gastric motility and increased absorption of dopaminergic medications, since improvement persisted during the 2-week wash-out period, i.e., in the absence of study drug, thus indicating the likely improvement based upon aminosterol treatment restoring neuronal function by displacing αS and stimulating enteric neurons. These results demonstrate that the ENS in neurodisease such as PD is not irreversibly damaged and can be restored to normal function using the methods of the invention.

B. Disorders

1. Neurological Disorders or Diseases

The methods and aminosterol compositions of the invention can be used to treat and/or prevent neurodiseases such as Alzheimer's disease (AD), Huntington's Disease, Multiple Sclerosis, Amyotorphic Lateral Sclerosis (ALS), multiple system atrophy (MSA), schizophrenia, Friedreich's ataxia, vascular dementia, Lewy Body dementia or disease, spinal muscular atrophy, supranuclear palsy, fronto temperal dementia, progressive nuclear palsy, Guadeloupian Parkinsonism, spinocerebellar ataxia, and autism.

i. Parkinson's Disease

In one aspect encompassed is a method of treating, preventing, and/or slowing the onset or progression of Parkinson's disease (PD) and/or a related symptom in a subject in need comprising administering to the subject a therapeutically effective amount of at least one aminosterol, or a salt or derivative thereof, provided that the administering does not comprise oral administration. For example, the administration can comprise nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof. In another aspect, the administration comprises nasal administration.

In another aspect, encompassed is a method of treating, preventing, and/or slowing the onset or progression of Parkinson's disease (PD) and/or a related symptom in a subject in need comprising: (a) determining a dose of an aminosterol or a salt or derivative thereof for the subject, wherein the aminosterol dose is determined based on the effectiveness of the aminosterol dose in improving or resolving a PD symptom being evaluated; (b) followed by administering the dose of the aminosterol or a salt or derivative thereof to the subject for a defined period of time, wherein the method comprises: (i) identifying a PD symptom to be evaluated; (ii) identifying a starting dose of an aminosterol or a salt or derivative thereof for the subject; and (iii) administering an escalating dose of the aminosterol or a salt or derivative thereof to the subject over a period of time until an effective dose for the PD symptom being evaluated is identified, wherein the effective dose is the aminosterol dose where improvement or resolution of the PD symptom is observed, and fixing the aminosterol dose at that level for that particular PD symptom in that particular subject; and (c) optionally wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

Parkinson's disease (PD) is a progressive neurodegenerative disorder caused by accumulation of the protein α-synuclein (αS) within the enteric nervous system (ENS), autonomic nerves and brain (Braak et al. 2003). While motor symptoms are still required for a diagnosis of Parkinson's disease (Hughes et al. 1992), non-motor symptoms represent a greater therapeutic challenge (Zahodne et al. 2012). These symptoms include constipation (Ondo et al. 2012; Lin et al. 2014), disturbances in sleep architecture (Ondo et al. 2001; Gjerstad et al. 2006), cognitive dysfunction (Auyeung et al. 2012), hallucinations (Friedman et al. 2016; Diederich et al. 2009), REM behavior disorder (RBD) and depression (Aarsland et al. 2007), all of which result from impaired function of neural pathways not restored by replacement of dopamine. In fact, long-term institutionalization, caregiver burden and decrease in life expectancy correlate more significantly with the severity of these symptoms than with motor symptoms (Goetz et al. 1995).

Parkinson's Disease (PD) is the second most common age-related neurodegenerative disease after AD. PD affects over 1% of the population over the age of 60, which in the US equates to over 500,000 individuals, while in individuals over the age of 85 this prevalence reaches 5%, highlighting the impact that advancing age has on the risk of developing this condition.

Parkinson's disease (PD) is a progressive neurodegenerative disease associated with the accumulation of the protein α-synuclein within the peripheral and central nervous system (CNS). Whilst diagnosis of PD is primarily based on the presence of a combination of motor symptoms, non-motor symptoms, including neuropathic constipation, present an common important therapeutic challenge. In 2003, Braak proposed that PD begins within the GI tract caused when neurotoxic aggregates of α-synuclein form within the ENS, evidenced clinically by the appearance of constipation in a majority of people with PD many years before the onset of motor symptoms. A recent study in rats has demonstrated movement of aggregates of α-synuclein from the ENS to the CNS via the vagus and other afferent nerves. Neurotoxic aggregates accumulated progressively within the brainstem and then dispersed rostrally to structures within the diencephalon, eventually reaching the cerebral hemispheres.

Parkinson's disease (PD) is divided into three stages: preclinical (in which neurodegenerative process is started without evident symptoms or signs); prodromal (in which symptoms and signs are present but insufficient to define a full clinical PD diagnosis); and clinical (in which the diagnosis is achieved based on the presence of classical motor signs).

The so-called gold standard for PD diagnosis entails expert diagnosis based on patient symptoms. PD and prodromal PD diagnosis is probabilistic, made on the basis of the presence of particular motor and non-motor symptoms, physiological pathologies, genetic characteristics, and environmental factors. Diagnosis may include a combination of markers (any disease indicator, whether a symptom, sign, or biomarker) ranging from mild motor symptoms [i.e., UPDRS−1987 version score≥3 excluding action tremor; or MDS-UPDRS score>6 excluding postural-action tremor; slowness, loss of muscle movements, tremor, rigidity, imbalance, abnormal posture], non-motor symptoms (i.e., REM SBD, olfactory dysfunction, constipation, excessive daytime somnolence, symptomatic hypotension, erectile/urinary dysfunction, depression, cognition), and ancillary diagnostic tests (i.e., abnormal tracer uptake of the presynaptic dopaminergic system: SPECT or positron emission tomography).

Longitudinal studies show reasonable specificity and sensitivity in applying Movement Disorders Society criteria (i.e., research criteria and probability methodology defined by Berg et al., further defining “probable prodromal PD” as 80% certainty) (Berg et al. 2015) for prodromal PD (55% sensitivity; 99% specificity). Indicative signs in these studies include non-motor (in approximate order: REM SBD, hyposmia, constipation, depression, anxiety, executive dysfunction, fatigue, orthostatic hypertension, urinary dysfunction, apathy, pain, sleep problems, dementia, psychosis) and motor (in approximate order: early motor impairments, bradykinesia, tremor, rigidity, fluctuations, freezing, dyskinesias, falls, postural instability, dysphagia) signs that appear at various progressive stages of prodromal and clinical PD.

Parkinson's disease is defined as a synucleinopathy, and synuclein deposition remains the main final arbiter of diagnosis. Additionally, patients with dementia and Lewy bodies are considered as having PD if they meet clinical disease criteria. Imaging (e.g., MRI, single photon emission computed tomography [SPECT], and positron emission tomography [PET]) allows in vivo brain imaging of structural, functional, and molecular changes in PD patients.

There has been research in the last few years identifying particular markers or combinations of markers that are used for probabilistic estimates of prodromal PD. Researchers have identified a timeline of symptoms indicative of prodromal PD and predictive of PD. The presence of each contributes to an estimate of the likelihood of prodromal PD. Some have been adopted for identification of prodromal PD. Other studies use a combination of symptoms and imaging (e.g., hyposmia combined with dopamine receptor imaging has been found to have a high predictive value). In another example, REM sleep behavior disorder (SBD), constipation, and hyposmia were found to be individually common but to rarely co-occur in individuals without PD, leading to a high predictive value for PD.

PD may also be assessed using the Unified Parkinson's Disease Rating Scale (UPDRS) which consists of 42 items in four subscales: (1) Part I, Non-Motor Aspects of Experiences of Daily Living (nM-EDL): cognitive impairment (section 1.1), hallucinations and psychosis (section 1.2), depressed mood (section 1.3), anxious mood (section 1.4), apathy (section 1.5), features of dopamine dysregulation syndrome (section 1.6), sleep problems (section 1.7), daytime sleepiness (section 1.8), pain and other sensations (section 1.9), urinary problems (section 1.10), constipation problems (section 1.11), light headedness on standing (section 1.12), and fatigue (section 1.13); (2) Part II, Motor Aspects of Experiences of Daily Living (M-EDL): speech (section 2.1), saliva & drooling (section 2.2), chewing and swallowing (section 2.3), eating tasks (section 2.4), dressing (section 2.5), hygiene (section 2.6), handwriting (section 2.7), doing hobbies and other activities (section 2.8), turning in bed (section 2.9), tremor (section 2.10), getting out of bed, a car, or a deep chair (section 2.11), walking and balance (section 2.12), and freezing (section 2.13); Part III, Motor Examination: speech (section 3.1), facial expression (section 3.2), rigidity (section 3.3), finger tapping (section 3.4), hand movements (section 3.5), pronation-supination movements of hands (section 3.6), toe tapping (section 3.7), leg agility (section 3.8), arising from chair (section 3.9), gait (3.10), freezing of gait (section 3.11), postural stability (section 3.12), posture (section 3.13), global spontaneity of movement (body bradykinesia) (section 3.14), postural tremor of the hands (section 3.15), kinetic tremor of the hands (section 3.16), rest tremor amplitude (section 3.17), and constancy of rest tremor (section 3.18); Part IV, Motor Complications: time spent with dyskinesias (section 4.1), functional impact of dyskinesias (section 4.2), time spent in the off state (section 4.3), functional impact of fluctuations (section 4.4), complexity of motor fluctuations (section 4.5), and painful off-state dystonia (section 4.6).

Further, symptom-based endpoints can be assessed using known scales. For example, (1) depression can be assessed using the Beck Depression Inventory (BDI-II) (Steer et al. 2000), cognition can be assessed using the Mini Mental State Examination (MMSE) (Palsteia et al. 2018) (see FIG. 25), sleep and REM-behavior disorder (RBD) can be assessed using a daily diary and an RBD questionnaire (RBDQ) (Stiasny-Kolster et al. 2007), and hallucinations can be assessed using the PD hallucinations questionnaire (PDHQ) (Papapetropoulos et al. 2008) and direct questioning. Circadian system status can also be assessed by continuously monitoring wrist skin temperature (Thermochron iButton DS1921H; Maxim, Dallas) following published procedures (Sarabia et al. 2008).

In another embodiment, administration of a therapeutically effective fixed dose of an aminosterol composition to a PD patient results in improvement of one or more symptoms of Parkinson's disease or on one or more clinically accepted scoring metrics, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The improvement can be measured using any clinically recognized tool or assessment.

PD progression and treatment is particularly difficult in view of patients' development of resistance to dopamine and subsequent dose escalation until no response can be elicited. As explained in Example 1, the data disclosed herein relates to non-dopamine related symptoms. Thus, not to be bound by theory, it is believed that prior or co-administration of an aminosterol composition according to the invention may reduce the dopamine dosage required to elicit a therapeutic effect for Parkinson's symptoms and/or increase the period during which the patient is sensitive to dopamine. It is also theorized that prior or co-administration of an aminosterol composition according to the invention may delay the time period when a patient is advised to begin dopamine therapy. This is significant, as currently patients are encouraged to delay initiation of dopamine treatment as long as possible, as after a period of time subjects become resistant to dopamine.

Data described in Example 1 shows remarkable improvement in a wide variety of symptoms correlated with PD, including a significant and positive effect on bowel function and neurologic symptoms of PD. The study is the first proof of concept demonstration that directly targeting αS pharmacologically can achieve beneficial GI, autonomic and CNS responses in neurodiseases such as PD.

For example, regarding the effect on bowel function, in Stage 1 (single dose), cumulative response rate increased in a dose-dependent fashion from 25% at 25 mg to a maximum of 80% at 200 mg (FIG. 1A). In Stage 2 (daily dosing), the response rate increased in a dose-dependent fashion from 26% at 75 mg to 85.3% at 250 mg (FIG. 1A). The dose required for a bowel response was patient-specific and varied from 75 mg to 250 mg. Median efficacious dose was 100 mg. Average CSBM/week increased from 1.2 at baseline to 3.8 at fixed dose and SBM increased from 2.6 at baseline to 4.5 at fixed dose (Table 7). Use of rescue medication decreased from 1.8/week at baseline to 0.3 at fixed dose. Consistency based on the Bristol stool scale also improved, increasing from mean 2.7 to 4.1 and ease of passage increased from 3.2 to 3.7. Subjective indices of wellbeing (PAC-QOL) and constipation symptoms (PAC-SYM) also improved during treatment. While the improvement in most stool-related indices did not persist beyond the treatment period, CSBM frequency remained significantly above baseline value (Table 8).

CNS Symptoms:

Example 1 also describes an analysis with respect to the sleep data, the body temperature data, mood, fatigue, hallucinations, cognition and other motor and non-motor symptoms of PD. CNS symptoms were evaluated at baseline and at the end of the fixed dose period and the wash-out period (Table 12). Moreover, unlike stool-related indices, the improvement in many CNS symptoms persisted during wash-out. The results of treatment were dramatic:

(1) Total UPDRS score was 64.4 at baseline, 60.6 at the end of the fixed dose period and 55.7 at the end of the wash-out period; similarly, the motor component of the UPDRS improved from 35.3 at baseline to 33.3 at the end of fixed dose to 30.2 at the end of wash-out. The UPDRS score, a global assessment of motor and non-motor symptoms, showed significant improvement. Improvement was also seen in the motor component. The improvement in the motor component is unlikely to be due to improved gastric motility and increased absorption of dopaminergic medications, since improvement persisted during the 2-week wash-out period, i.e., in the absence of study drug (Table 12).

(2) MMSE (cognitive ability) improved from 28.4 at baseline to 28.7 during treatment and to 29.3 during wash-out.

(3) BDI-II (depression) decreased from 10.9 at baseline to 9.9 during treatment and 8.7 at wash-out.

(4) PDHQ (hallucinations) improved from 1.3 at baseline to 1.8 during treatment and 0.9 during wash-out. Hallucinations were reported by 5 patients at baseline and delusions in 1 patient. Both hallucinations and delusions improved or disappeared in 5 of 6 patients during treatment and did not return for 4 weeks following discontinuation of aminosterol treatment in 1 patient and 2 weeks in another. In one patient the hallucinations disappeared at 100 mg, despite not having reached the colonic prokinetic dose at 175 mg.

(5) Improvements were seen in REM-behavior disorder (RBD) and sleep. RBD and total sleep time also improved progressively in a dose-dependent manner. The frequency of arm or leg thrashing reported in the sleep diary diminished progressively from 2.2 episodes/week at baseline to 0 at maximal dose. Total sleep time increased progressively from 7.1 hours at baseline to 8.4 hours at 250 mg and was consistently higher than baseline beyond 125 mg (FIG. 4).

The data detailed in Example 1 is consistent with the hypothesis that gastrointestinal dysmotility in PD results from the progressive accumulation of αS in the ENS, and that aminosterols can restore neuronal function by displacing αS and stimulating enteric neurons. These results demonstrate that the ENS in PD is not irreversibly damaged and can be restored to normal function.

In one embodiment, the invention encompasses a method of treating, preventing and/or slowing the onset or progression of PD and/or a related symptom in a subject in need comprising administering to the subject a therapeutically effective amount of at least one aminosterol or a salt or derivative thereof via non-oral administration.

In another embodiment, the invention comprises method of treating and/or preventing Parkinson's disease (PD) and/or a related symptom in a subject in need comprising: (a) determining a dose of an aminosterol or a salt or derivative thereof for the subject, wherein the aminosterol dose is determined based on the effectiveness of the aminosterol dose in improving or resolving a PD symptom being evaluated; (b) followed by administering the aminosterol dose to the subject for a period of time, wherein the method comprises: (i) identifying a PD symptom to be evaluated; (ii) identifying a starting aminosterol dose for the subject; and (iii) administering an escalating dose of the aminosterol to the subject over a period of time until an effective dose for the PD symptom being evaluated is identified, wherein the effective dose is the aminosterol dose where improvement or resolution of the PD symptom is observed, and fixing the aminosterol dose at that level for that particular PD symptom in that particular subject.

In the methods of the invention, and in particular methods comprising aminosterol dose optimization, the aminosterol or a salt or derivative thereof can be administered orally, intranasally, or a combination thereof. For example, the aminosterol or a salt or derivative thereof can be administered orally, intranasally, by injection (IV, IP, or IM) or any combination thereof. In some embodiments, the dosage of the aminosterol or a salt or derivative thereof can be escalated every about 3 to about 5 days. In some embodiments, the dose of the aminosterol or a salt or derivative thereof can be escalated every about 1, about 2, about 3, about 4, about 5, about 6, about 7, about 8, about 9, about 10, about 11, about 12, about 13, or about 14 days. In some embodiments, the dose of the aminosterol or a salt or derivative thereof can be escalated about 1×/week, about 2×/week, about every other week, or about 1×/month. In some embodiments, the fixed dose of the aminosterol or a salt or derivative thereof can be administered once per day, every other day, once per week, twice per week, three times per week, four times per week, five times per week, six times per week, every other week, or every few days. In some embodiments, the fixed dose of the aminosterol or a salt or derivative thereof can be administered for a first period of time of administration, followed by a cessation of administration for a second period of time, followed by resuming administration upon recurrence of PD or a symptom of PD. In some embodiments, the fixed aminosterol dose can be incrementally reduced after the fixed dose of aminosterol or a salt or derivative thereof has been administered to the subject for a period of time. In some embodiments, the fixed aminosterol dose can be varied plus or minus a defined amount to enable a modest reduction or increase in the fixed dose. In some embodiments, the fixed aminosterol dose can be increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%. In some embodiments, the starting aminosterol dose can be higher if the symptom being evaluated is severe.

In the methods of the invention, progression or onset of PD can be slowed, halted, delayed, or reversed over a defined period of time following administration of the fixed escalated dose of the aminosterol or a salt or derivative thereof, as measured by a medically-recognized technique. In other embodiments, the PD can be positively impacted by the fixed escalated dose of the aminosterol or a salt or derivative thereof, as measured by a medically-recognized technique. In further embodiments, the positive impact and/or progression of PD can be measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis. In other embodiments, the progression or onset of PD can be slowed, halted, delayed or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In some aspects of the methods of the invention, (a) the method prolongs the period of time the subject can be sensitive to dopamine; (b) the method may delay the need for the subject to begin dopamine treatment; and/or (c) any combination thereof.

In the methods of the invention, the fixed escalated aminosterol dose can reverse dysfunction caused by the PD and may treat, prevent, improve, and/or resolve the symptom being evaluated. In further embodiments, the improvement or resolution of the PD symptom can be measured using a clinically recognized scale or tool. In still further embodiments, the improvement in the PD symptom can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale.

Non-limiting exemplary PD symptoms include but are not limited to (a) at least one non-motor aspect of experiences of daily living as defined by Part I of the Unified Parkinson's Disease Rating Scale selected from the group consisting of cognitive impairment, hallucinations and psychosis, depressed mood, anxious mood, apathy, features of dopamine dysregulation syndrome, sleep problems, daytime sleepiness, pain, urinary problems, constipation problems, lightheadedness on standing, and fatigue; (b) at least one motor aspect of experiences of daily living as defined by Part II of the Unified Parkinson's Disease Rating Scale selected from the group consisting of speech, saliva and drooling, chewing and swallowing, eating tasks, dressing, hygiene, handwriting, turning in bed, tremors, getting out of a bed, a car, or a deep chair, walking and balance, and freezing; (c) at least one motor symptom identified in Part III of the Unified Parkinson's Disease Rating Scale selected from the group consisting of speech, facial expression, rigidity, finger tapping, hand movements, pronation-supination movements of hands, toe tapping, leg agility, arising from chair, gait, freezing of gait, postural stability, posture, body bradykinesia, postural tremor of the hands, kinetic tremor of the hands, rest tremor amplitude, and constancy of rest tremor; (d) at least one motor complication identified in Part IV of the Unified Parkinson's Disease Rating Scale selected from the group consisting of time spent with dyskinesias, functional impact of dyskinesias, time spent in the off state, functional impact of fluctuations, complexity of motor fluctuations, and painful off-state dystonia; (e) constipation; (f) depression; (g) cognitive impairment; (h) short or long term memory impairment; (i) concentration impairment; (j) coordination impairment; (k) mobility impairment; (l) speech impairment; (m) mental confusion; (n) sleep problem, sleep disorder, or sleep disturbance; (o) circadian rhythm dysfunction; (p) hallucinations; (q) fatigue; (r) REM disturbed sleep; (s) REM behavior disorder; (t) erectile dysfunction; (u) postural hypotension; (v) correction of blood pressure or orthostatic hypotension; (w) nocturnal hypertension; (x) regulation of temperature; (y) improvement in breathing or apnea; (z) correction of cardiac conduction defect; (aa) amelioration of pain; (bb) urinary incontinence, or restoration of bladder sensation and urination; (cc) mood swings; (dd) apathy; (ee) control of nocturia; and/or (ff) neurodegeneration. In some embodiments, (a) the sleep disorder or sleep disturbance comprises a delay in sleep onset, sleep fragmentation, REM-behavior disorder, sleep-disordered breathing including snoring and apnea, day-time sleepiness, micro-sleep episodes, narcolepsy, hallucinations, or any combination thereof; (b) the REM-behavior disorder comprises vivid dreams, nightmares, and acting out the dreams by speaking or screaming, or fidgeting or thrashing of arms or legs during sleep; or (c) the hallucination comprises a visual, auditory, tactile, gustatory or olfactory hallucination.

In embodiments where the PD symptom to be evaluated is a sleep problem, sleep disorder, sleep disturbance, circadian rhythm dysfunction, REM disturbed sleep, or REM behavior disorder, (a) treating the sleep problem, sleep disorder, sleep disturbance may prevent or delay the onset and/or progression of the PD; (b) the sleep problem, sleep disorder or sleep disturbance may comprise a delay in sleep onset, sleep fragmentation, REM-behavior disorder, sleep-disordered breathing including snoring and apnea, day-time sleepiness, micro-sleep episodes, narcolepsy, hallucinations, or any combination thereof; (c) the REM-behavior disorder may comprise vivid dreams, nightmares, and acting out the dreams by speaking or screaming, or fidgeting or thrashing of arms or legs during sleep; (d) the method may result in a positive change in the sleeping pattern of the subject; (e) the method may result in a positive change in the sleeping pattern of the subject, wherein the positive change can be defined as: (i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (f) as a result of the method the subject may obtain the total number of hours of sleep recommended by a medical authority for the age group of the subject.

In embodiments where the PD symptom to be evaluated is hallucinations, (a) the hallucination may comprise a visual, auditory, tactile, gustatory or olfactory hallucination; (b) treating the hallucination may prevent and/or delay the onset and/or progression of the Parkinson's disease; (c) the method results in a decreased number or severity of hallucinations of the subject; (d) the method may result in a decreased number or severity of hallucinations of the subject and the decrease in number or severity in hallucinations can be defined as a reduction in occurrences or severity of hallucinations selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (e) the method may result in the subject being hallucination-free.

In embodiments where the PD symptom to be evaluated is depression, (a) treating the depression may prevent and/or delay the onset and/or progression of the Parkinson's disease; (b) the method may result in improvement in a subject's depression, as measured by one or more clinically-recognized depression rating scale; (c) the method may result in improvement in a subject's depression, as measured by one or more clinically-recognized depression rating scale and the improvement can be in one or more depression characteristics selected from the group consisting of mood, behavior, bodily functions such as eating, sleeping, energy, and sexual activity, and/or episodes of sadness or apathy; and/or (d) the method may result in improvement in a subject's depression, as measured by one or more clinically-recognized depression rating scale, and the improvement a subject experiences following treatment can be about 5, about 10, about 15, about 20, about 25, about 30, about 35, about 40, about 45, about 50, about 55, about 60, about 65, about 70, about 75, about 80, about 85, about 90, about 95 or about 100%.

In embodiments where the PD symptom to be evaluated is cognitive impairment, (a) treating the cognitive impairment may prevent and/or delay the onset and/or progression of the Parkinson's disease; (b) progression or onset of the cognitive impairment can be slowed, halted, or reversed over a defined period of time following administration of the fixed escalated dose of the aminosterol or a salt or derivative thereof, as measured by a medically-recognized technique; and/or (c) the cognitive impairment can be positively impacted by the fixed escalated dose of the aminosterol or a salt or derivative thereof, as measured by a medically-recognized technique; (d) the cognitive impairment can be positively impacted by the fixed escalated dose of the aminosterol or a salt or derivative thereof, as measured by a medically-recognized technique and the positive impact on and/or progression of cognitive decline can be measured quantitatively or qualitatively by one or more techniques selected from the group consisting of Mini-Mental State Exam (MMSE), Mini-cog test, and a computerized tested selected from Cantab Mobile, Cognigram, Cognivue, Cognision, or Automated Neuropsychological Assessment Metrics; and/or (e) the progression or onset of cognitive impairment can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In embodiments where the PD symptom to be evaluated is constipation, (a) treating the constipation may prevent and/or delay the onset and/or progression of the Parkinson's disease; (b) the fixed escalated aminosterol dose may cause the subject to have a bowel movement; (c) the method may result in an increase in the frequency of bowel movement in the subject; (d) the method may result in an increase in the frequency of bowel movement in the subject and the increase in the frequency of bowel movement can be defined as: (i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; (e) as a result of the method the subject may have the frequency of bowel movement recommended by a medical authority for the age group of the subject; and/or (f) the starting aminosterol dose can be determined by the severity of the constipation, wherein: (i) if the average complete spontaneous bowel movement (CSBM) or spontaneous bowel movement (SBM) is one or less per week, then the starting aminosterol dose is at least about 150 mg; and (ii) if the average CSBM or SBM is greater than one per week, then the starting aminosterol dose is about 75 mg or less.

In embodiments where the PD symptom to be evaluated is neurodegeneration correlated with PD, (a) treating the neurodegeneration may prevent and/or delay the onset and/or progression of the Parkinson's disease; (b) the method may result in treating, preventing, and/or delaying the progression and/or onset of neurodegeneration in the subject; (c) progression or onset of the neurodegeneration can be slowed, halted, or reversed over a defined period of time following administration of the fixed escalated dose of the aminosterol or a salt or derivative thereof, as measured by a medically-recognized technique; and/or (d) the neurodegeneration can be positively impacted by the fixed escalated dose of the aminosterol or a salt or derivative thereof, as measured by a medically-recognized technique. In further embodiments, (a) the positive impact and/or progression of neurodegeneration can be measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis; and/or (b) the progression or onset of neurodegeneration can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

ii. Alzheimer's Disease

In one aspect encompassed is a method of treating, preventing, and/or slowing the onset or progression of Alzheimer's Disease (AD) and/or a related symptom in a subject in need comprising administering to the subject a therapeutically effective amount of at least one aminosterol, or a salt or derivative thereof, provided that the method of administration does not comprise oral administration. For example, the method of administration can comprise nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof. In a preferred aspect, the method of administration comprises nasal administration.

In another aspect, encompassed is a method of treating, preventing and/or slowing the onset or progression of Alzheimer's Disease (AD) and/or a related symptom in a subject in need comprising: (a) determining a dose of an aminosterol or a salt or derivative thereof for the subject, wherein the aminosterol dose is determined based on the effectiveness of the aminosterol dose in improving or resolving an AD symptom being evaluated, (b) followed by administering the dose of the aminosterol or a salt or derivative thereof to the subject for a defined period of time, wherein the method comprises: (i) identifying an AD symptom to be evaluated; (ii) identifying a starting dose of an aminosterol or a salt or derivative thereof for the subject; and (iii) administering an escalating dose of the aminosterol or a salt or derivative thereof to the subject over a defined period of time until an effective dose for the AD symptom being evaluated is identified, wherein the effective dose is the aminosterol dose where improvement or resolution of the AD symptom is observed, and fixing the aminosterol dose at that level for that particular AD symptom in that particular subject; and (c) optionally wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

Alzheimer's disease (AD) is a chronic neurodegenerative disease that usually starts slowly and worsens over time. It is the cause of 60-70% of cases of dementia. As the disease advances, symptoms can include problems with language, disorientation, mood swings, loss of motivation, not managing self care, and behavioral issues. As a person's condition declines, they often withdraw from family and society. Gradually, bodily functions are lost, ultimately leading to death. Although the speed of progression can vary, the typical life expectancy following diagnosis is 3 to 9 years. In 2015, there were approximately 29.8 million people worldwide with AD. It most often begins in people over 65 years of age, although 4% to 5% of cases are early-onset Alzheimer's. It affects about 6% of people 65 years and older. In 2015, dementia resulted in about 1.9 million deaths.

The World Health Organization looked at 23 low-to middle-income nations and estimated that their combined loss in economic output between 2006 and 2015 due to age-related diseases was USD84 billion, and the global cost of AD alone in 2010 was estimated at USD604 billion. Wimo et al. 2013.

The symptoms of Alzheimer's disease are primarily marked by cognitive deficits including memory impairment, language dysfunction, and visuospatial skills; functional impairment that may span occupational and social issues (e.g., activities of daily living); and behavioral symptoms including depression, anxiety, aggression and psychosis may also appear as the disease progresses in severity.

At this time, unambiguous diagnosis of AD requires clinical findings of cognitive deficits consistent with AD and post-mortem identification of brain pathologies consistent with AD. The term AD dementia is used to describe dementia that is due to the pathophysiologies of AD. The term “probable Alzheimer's disease” is used in life when a subject demonstrates clinical characteristics of AD and when other possible biological causes of dementia (e.g. PD or stroke) are excluded.

There are currently a variety of art-accepted methods for diagnosing probable AD. Typically, these methods are used in combination. These methods include determining an individual's ability to carry out daily activities and identifying changes in behavior and personality. Dementia of the AD type is also typically characterized by an amnestic presentation (memory deficit) or language, visuospatial or executive function deficits. Cognitive ability/impairment may be determined by art-accepted methods, including, but not limited to, validated instruments that assess global cognition (e.g., the Modified Mini Mental State Examination (3MS-E)), and specific domains such as visual and verbal memory (e.g., the Brief Visuospatial Memory Test (Revised) (BVMT-R) and the Hopkins Verbal Learning Test (Revised) (HVLT-R), respectively), language (e.g., the Generative Verbal Fluency Test (GVFT)) and executive function and attention (e.g., the Digit Span Test (DST)). Dementia due to AD is also defined by insidious onset and a history of worsening cognitive performance.

The criteria for ‘probable Alzheimer's disease’ are described a National Institute of Aging-Alzheimer's Association workgroup (McKhann et al. 2011 Alzheimers Dement, 7: 263-269). According to this workgroup, for people who first exhibit the core clinical characteristics of Alzheimer's disease dementia, evidence of biomarkers associated with the disease may enhance the certainty of the diagnosis.

In another embodiment, administration of a therapeutically effective fixed dose of an aminosterol composition to an AD results in improvement of one or more symptoms of AD or on one or more clinically accepted scoring metrics, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In one embodiment, a method of treating, preventing, and/or slowing the onset or progression of AD and/or a related symptom in a subject in need is provided, the method comprising administering to the subject a therapeutically effective amount of at least one aminosterol, or a salt or derivative thereof.

In one embodiment, the invention encompasses a method of treating, preventing and/or slowing the onset of AD and/or a related symptom in a subject in need comprising (a) determining a dose of an aminosterol or a salt or derivative thereof for the subject, wherein the aminosterol dose is determined based on the effectiveness of the aminosterol dose in improving or resolving an AD symptom being evaluated, (b) followed by administering the aminosterol dose to the subject for a period of time, wherein the method comprises (i) identifying an AD symptom to be evaluated; (ii) identifying a starting aminosterol dose for the subject; and (iii) administering an escalating dose of the aminosterol to the subject over a period of time until an effective dose for the AD symptom being evaluated is identified, wherein the effective dose is the aminosterol dose where improvement or resolution of the AD symptom is observed, and fixing the aminosterol dose at that level for that particular AD symptom in that particular subject.

In another embodiment, the starting aminosterol or a salt or derivative thereof dose is higher if the AD symptom being evaluated is severe.

In one embodiment, the method of the invention results in slowing, halting, or reversing progression or onset of AD over a defined time period following administration of the fixed escalated dose of the aminosterol or a salt or derivative thereof, as measured by a medically-recognized technique. For example, the progression or onset of AD may be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique. In addition, the method of the invention can result in positively impacting the AD, as measured by a medically-recognized technique. The positive impact and/or progression of AD may be measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis.

In one embodiment, the fixed escalated aminosterol dose reverses dysfunction caused by the AD and treats, prevents, improves, and/or resolves the symptom being evaluated. The improvement or resolution of the AD symptom can be measured using a clinically recognized scale or tool. For example, the improvement in the AD symptom can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale.

In yet another embodiment, the symptom to be evaluated can be selected from the group consisting of (a) a symptom from the Integrated Alzheimer's Disease Rating Scale (iADRS) selected from the group consisting of, personal belonging management, selection of clothes, ability to dress self, ability to clean habitation, financial management ability, writing ability, ability to keep appointments, ability to use telephone, ability to prepare food for self, travel ability, awareness of current events, reading ability, interest in television, ability to shop for self, ability to remain alone, ability to perform chores, ability to perform a hobby or game, driving ability, self-management of medications, ability to initiate and finish complex tasks, and ability to initiate and finish simple tasks; (b) a symptom from the Alzheimer's Disease Assessment Scale-Cognitive subscale (ADAS-Cog) selected from the group consisting of learning, naming, command following, ideational praxis, constructional praxis, orientation, and recognition memory; (c) a symptom from the Alzheimer's Disease Cooperative Study—instrumental Activities of Daily Living (ADCS-iADL) wherein the symptom is any of the symptoms recited in (a) or (b); (d) constipation; (e) depression; (f) cognitive impairment; (g) short or long term memory impairment; (h) concentration impairment; (i) coordination impairment; (j) mobility impairment; (k) speech impairment; (l) mental confusion; (m) sleep problem, sleep disorder, or sleep disturbance; (n) circadian rhythm dysfunction; (o) REM disturbed sleep; (p) REM behavior disorder; (q) hallucinations; (r) fatigue; (s) apathy; (t) erectile dysfunction; (u) mood swings; (v) urinary incontinence; or (w) neurodegeneration.

In one embodiment, the AD symptom to be evaluated is a sleep problem, sleep disorder, or sleep disturbance associated with AD. The sleep problem, sleep disorder, or sleep disturbance can comprise a delay in sleep onset, sleep fragmentation, REM-behavior disorder, sleep-disordered breathing including snoring and apnea, day-time sleepiness, micro-sleep episodes, narcolepsy, hallucinations, or any combination thereof. Further, the REM-behavior disorder can comprise vivid dreams, nightmares, and acting out the dreams by speaking or screaming, or fidgeting or thrashing of arms or legs during sleep.

In one embodiment, the AD symptom to be evaluated is a sleep problem, sleep disorder, sleep disturbance, circadian rhythm dysfunction, REM disturbed sleep, or REM behavior disorder, and (a) the method results in a positive change in the sleeping pattern of the subject; (b) the method results in a positive change in the sleeping pattern of the subject, wherein the positive change is defined as: (i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (c) as a result of the method the subject obtains the total number of hours of sleep recommended by a medical authority for the age group of the subject.

In another embodiment, the AD symptom to be evaluated is hallucinations. The hallucination can comprise, for example, a visual, auditory, tactile, gustatory or olfactory hallucination. For example, the method can (a) result in a decreased number or severity of hallucinations of the subject; (b) the method can result in a decreased number or severity of hallucinations of the subject and the decrease in number or severity in hallucinations is defined as a reduction in occurrences or severity of hallucinations selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (c) the method can result in the subject being hallucination-free. In one embodiment, the fixed escalated aminosterol dose reverses dysfunction caused by the Alzheimer's disease and treats and/or prevents the hallucination.

In another embodiment, the AD symptom to be evaluated is depression. In an exemplary embodiment, the method results in improvement in a subject's depression, as measured by one or more clinically-recognized depression rating scales. For example, the improvement can be in one or more depression characteristics selected from the group consisting of mood, behavior, bodily functions such as eating, sleeping, energy, and sexual activity, and/or episodes of sadness or apathy. In another embodiment, the improvement a subject experiences following treatment can be about 5, about 10, about 15, about 20, about 25, about 30, about 35, about 40, about 45, about 50, about 55, about 60, about 65, about 70, about 75, about 80, about 85, about 90, about 95 or about 100%.

In some embodiments, the AD symptom to be evaluated is cognitive impairment, and (a) progression or onset of the cognitive impairment is slowed, halted, or reversed over a defined time period following administration of the fixed escalated dose of the aminosterol or a salt or derivative thereof, as measured by a medically-recognized technique; (b) the cognitive impairment is positively impacted by the fixed escalated dose of the aminosterol or a salt or derivative thereof, as measured by a medically-recognized technique; (c) the cognitive impairment is positively impacted by the fixed escalated dose of the aminosterol or a salt or derivative thereof, as measured by a medically-recognized technique and the positive impact on and/or progression of cognitive decline is measured quantitatively or qualitatively by one or more techniques selected from the group consisting of Mini-Mental State Exam (MMSE), Mini-cog test, and a computerized tested selected from Cantab Mobile, Cognigram, Cognivue, Cognision, or Automated Neuropsychological Assessment Metrics; and/or (d) the progression or onset of cognitive impairment is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In some embodiments, the AD symptom to be evaluated is constipation and: (a) the fixed escalated aminosterol dose causes the subject to have a bowel movement; (b) the method results in an increase in the frequency of bowel movements in the subject; (c) the method results in an increase in the frequency of bowel movements in the subject and the increase in the frequency of bowel movements is defined as: (i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; (d) as a result of the method the subject has the frequency of bowel movements recommended by a medical authority for the age group of the subject; and/or (e) the starting aminosterol dose is determined by the severity of the constipation, wherein: (i) if the average complete spontaneous bowel movement (CSBM) or spontaneous bowel movement (SBM) is one or less per week, then the starting aminosterol dose is at least about 150 mg; and (ii) if the average CSBM or SBM is greater than one per week, then the starting aminosterol dose is about 75 mg or less.

In one embodiment, the AD symptom to be evaluated is neurodegeneration, and the method results in treating, preventing, and/or delaying the progression and/or onset of neurodegeneration in the subject. In an exemplary embodiment (a) progression or onset of the neurodegeneration is slowed, halted, or reversed over a defined time period following administration of the fixed escalated dose of the aminosterol or a salt or derivative thereof, as measured by a medically-recognized technique; and/or (b) the neurodegeneration is positively impacted by the fixed escalated dose of the aminosterol or a salt or derivative thereof, as measured by a medically-recognized technique. The positive impact and/or progression of neurodegeneration can be measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis. In addition, the progression or onset of neurodegeneration can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In yet another embodiment of the invention, the administered aminosterol or a salt or derivative thereof functions to do one or more of the following: (a) binds luminal and/or intraepithelial lipopolysaccharide (LPS); (b) displaces LPS bound to amyloid-beta (Abeta); (c) prevents or reduces amyloid precursor protein (APP) and/or Abeta synthesis; (d) reduces circulating LPS; (e) reduces chylomicron Abeta-LPS content; (f) reduces amyloid trafficking to the subject's brain; (g) reduces amyloid trafficking to the portions of the subject's brain outside of the blood brain barrier; (h) reduces deposition of amyloid in vascular structures; and/or (i) reduces microglial inflammation in the subject's brain. In an exemplary embodiment for these aspects of the invention, the aminosterol is administered orally.

For example, in one embodiment, the aminosterol or a salt or derivative thereof is administered orally and the administered aminosterol or a salt or derivative thereof binds luminal and/or intraepithelial LPS. In another embodiment the aminosterol or a salt or derivative thereof is administered orally and the administered aminosterol or a salt or derivative thereof displaces LPS bound to Abeta.

In a further embodiment the aminosterol or a salt or derivative thereof is administered orally and the administered aminosterol or a salt or derivative thereof prevents or reduces amyloid precursor protein (APP) and/or Abeta synthesis. For example, the method can result in reducing the synthesis of APP and/or Abeta by about 5%, about 10%, about 15%, about 20%, about 25%, about 30%, about 35%, about 40%, about 45%, about 50%, about 55%, about 60%, about 65%, about 70%, about 75%, about 80%, about 90%, about 95%, or about 100%.

In yet another embodiment the aminosterol or a salt or derivative thereof is administered orally and the administered aminosterol or a salt or derivative thereof reduces circulating LPS. For example, the method can result in reducing circulating LPS by about 5%, about 10%, about 15%, about 20%, about 25%, about 30%, about 35%, about 40%, about 45%, about 50%, about 55%, about 60%, about 65%, about 70%, about 75%, about 80%, about 90%, about 95%, or about 100%.

In another embodiment, the aminosterol or a salt or derivative thereof is administered orally and the administered aminosterol or a salt or derivative thereof reduces chylomicron Abeta-LPS content. For example, the method can result in reducing the chylomicron Abeta-LPS content by about 5%, about 10%, about 15%, about 20%, about 25%, about 30%, about 35%, about 40%, about 45%, about 50%, about 55%, about 60%, about 65%, about 70%, about 75%, about 80%, about 90%, about 95%, or about 100%.

In another embodiment, the aminosterol or a salt or derivative thereof is administered orally and the administered aminosterol or a salt or derivative thereof reduces amyloid trafficking to the subject's brain. For example, the method results in reducing amyloid trafficking to the subject's brain by about 5%, about 10%, about 15%, about 20%, about 25%, about 30%, about 35%, about 40%, about 45%, about 50%, about 55%, about 60%, about 65%, about 70%, about 75%, about 80%, about 90%, about 95%, or about 100%.

In yet another embodiment, the aminosterol or a salt or derivative thereof is administered orally and the administered aminosterol or a salt or derivative thereof reduces amyloid trafficking to the portions of the subject's brain outside of the blood brain barrier. For example, the method results in reducing amyloid trafficking to the portions of the subject's brain outside of the blood brain barrier by about 5%, about 10%, about 15%, about 20%, about 25%, about 30%, about 35%, about 40%, about 45%, about 50%, about 55%, about 60%, about 65%, about 70%, about 75%, about 80%, about 90%, about 95%, or about 100%.

In another embodiment, the aminosterol or a salt or derivative thereof is administered orally and the administered aminosterol or a salt or derivative thereof reduces deposition of amyloid in vascular structures. For example, the method results in reducing deposition of amyloid in vascular structures by about 5%, about 10%, about 15%, about 20%, about 25%, about 30%, about 35%, about 40%, about 45%, about 50%, about 55%, about 60%, about 65%, about 70%, about 75%, about 80%, about 90%, about 95%, or about 100%.

In another embodiment, the aminosterol or a salt or derivative thereof is administered orally and the administered aminosterol or a salt or derivative thereof reduces microglial inflammation in the subject's brain. For example, the method results in reducing microglial inflammation in the subject's brain by about 5%, about 10%, about 15%, about 20%, about 25%, about 30%, about 35%, about 40%, about 45%, about 50%, about 55%, about 60%, about 65%, about 70%, about 75%, about 80%, about 90%, about 95%, or about 100%.

In another embodiment, the aminosterol or a salt or derivative thereof is administered in combination with at least one additional active agent to achieve either an additive or synergistic effect. For example, the additional active agent can be administered via a method selected from the group consisting of (a) concomitantly; (b) as an admixture; (c) separately and simultaneously or concurrently; or (d) separately and sequentially. In another embodiment, the additional active agent is a different aminosterol from that administered in primary method. In yet a further embodiment, the method of the invention comprises administering a first aminosterol which is aminosterol 1436 or a salt or derivative thereof intranasally and administering a second aminosterol which is squalamine or a salt or derivative thereof orally.

In another embodiment, the at least one additional active agent is an active agent used to treat AD or a symptom thereof. In some embodiments, the active agent is selected from the group consisting of cholinesterase inhibitors such as donepezil (Aricept®), galantamine (Razadyne®), rivastigmine (Exelon®), and tacrine (Cognex®); N-methyl D-aspartate (NMDA) antagonists such as memantine (Namenda®); and Namzaric®, a combination of Namenda® and Aricept®.

The methods of the invention also encompass methods where the subject suffers from, is or at risk of developing, an inflammatory disease or condition caused by excessive expression or concentration of alpha synuclein in the subject. In one embodiment, the excessive expression of alpha synuclein is associated with AD. In one embodiment, the method results in a decrease in intensity of inflammation, blood levels of inflammatory markers, inflammatory markers in tissue, number of inflammatory cells in tissue, or any combination thereof, as compared to a control or as compared to the qualitative or quantitative amount from the same patient or subject prior to treatment.

In yet another embodiment, the method results in a decrease in concentration of alpha synuclein in the subject. The decrease in alpha-synuclein concentration can be measured, for example, qualitatively, quantitatively, or semi-quantitatively by one or more methods. Such methods include for example (a) first determining the concentration of alpha-synuclein in a tissue sample from the subject prior to treatment, followed by: (i) after treatment determining the alpha-synuclein concentration in the same tissue type from the same subject; or (ii) after treatment comparing the alpha-synuclein concentration in the same tissue type to a control; (b) measuring the intensity of inflammation over time; (c) measuring the amount of inflammatory markers over time; (d) measuring the amount of inflammatory markers in blood, plasma, or tissue over time, either qualitatively or quantitatively; (e) measuring the amount of one or more inflammatory marker cytokines in blood, plasma, or tissue over time, either qualitatively or quantitatively; (f) measuring the amount of one or more plasma markers of inflammation such as TNF, IL-8, or CRP in blood, plasma, or tissue over time, either qualitatively or quantitatively; or (g) measuring the amount of inflammatory cells in blood, plasma, or tissue over time, either qualitatively or quantitatively. The decrease can be, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In one embodiment, the method is applied to a patient population susceptible to excessive expression of alpha-synuclein, resulting in an excessive or high concentration of alpha-synuclein.

In one embodiment, the invention encompasses a method of treating, preventing and/or slowing the onset or progression of AD and/or a related symptom in a subject in need comprising administering to the subject a therapeutically effective amount of at least one aminosterol or a salt or derivative thereof, preferably where the administration of the aminosterol is via non-oral means. In one aspect, the at least one aminosterol or a salt or derivative thereof is administered via nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof. In another aspect, the at least one aminosterol or a salt or derivative thereof is administered nasally. In another aspect, administration of the at least one aminosterol or a salt or derivative thereof comprises non-oral administration.

In another embodiment, the invention encompasses a method of treating, preventing and/or slowing the onset or progression of AD and/or a related symptom in a subject in need comprising (a) determining a dose of an aminosterol or a salt or derivative thereof for the subject, wherein the aminosterol dose is determined based on the effectiveness of the aminosterol dose in improving or resolving a AD symptom being evaluated, (b) followed by administering the aminosterol dose to the subject for a period of time, wherein the method comprises (i) identifying a AD symptom to be evaluated; (ii) identifying a starting aminosterol dose for the subject; and (iii) administering an escalating dose of the aminosterol to the subject over a period of time until an effective dose for the AD symptom being evaluated is identified, wherein the effective dose is the aminosterol dose where improvement or resolution of the AD symptom is observed, and fixing the aminosterol dose at that level for that particular AD symptom in that particular subject.

iii. Multiple System Atrophy

In one aspect, encompassed is a method of treating, preventing, and/or slowing the onset or progression of multiple system atrophy (MSA) and/or a related symptom in a subject in need comprising administering to the subject a therapeutically effective amount of at least one aminosterol or a salt or derivative thereof. In one aspect, the method of administration can comprise oral, nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof. In another aspect, the method of administration comprises non-oral administration and optionally nasal administration.

In another aspect, the disclosure encompasses a method of treating or preventing MSA and/or a related symptom in a subject in need comprising: (a) determining a dose of an aminosterol or a pharmaceutically acceptable salt or derivative thereof for the subject, wherein the aminosterol dose is determined based on the effectiveness of the aminosterol dose in improving or resolving an MSA symptom being evaluated, (b) followed by administering the dose of the aminosterol or a pharmaceutically acceptable salt or derivative thereof to the subject for a defined period of time, wherein the method comprises: (i) identifying an MSA symptom to be evaluated; (ii) identifying a starting dose of an aminosterol or a pharmaceutically acceptable salt or derivative thereof for the subject; and (iii) administering an escalating dose of the aminosterol or a pharmaceutically acceptable salt or derivative thereof to the subject over a defined period of time until an effective dose for the MSA symptom being evaluated is identified, wherein the effective dose is the dose of the aminosterol or a pharmaceutically acceptable salt or derivative thereof where improvement or resolution of the MSA symptom is observed, and fixing the dose of the aminosterol or a pharmaceutically acceptable salt or derivative thereof at that level for that particular MSA symptom in that particular subject; and (c) optionally wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

Multiple system atrophy (MSA) is a progressive neurodegenerative disorder characterized by a combination of symptoms that affect both the autonomic nervous system (the part of the nervous system that controls involuntary action such as blood pressure or digestion) and movement. MSA, also known as Shy-Drager syndrome, is a neurodegenerative disorder characterized by tremors, slow movement, muscle rigidity, and postural instability (collectively known as parkinsonism) due to dysfunction of the autonomic nervous system, and ataxia. This is caused by progressive degeneration of neurons in several parts of the brain including the substantia nigra, striatum, inferior olivary nucleus, and cerebellum. There is no known cure for MSA and management is primarily supportive.

Progression of neurodegeneration can be measured using well known techniques. For example, an electroencephalogram (EEG) can be used as a biomarker for the presence and progression of a neurodegenerative disease. S. Morairty, 2013. Another exemplary technique that can be used to measure progression of neurodegeneration of MRI. Rocca et al. 2017.

A variety of neuroimaging techniques may be useful for the early diagnosis and/or measurement of progression of MSA. Examples of such techniques include but are not limited to neuroimaging, functional MRI, structural MRI, diffusion tensor imaging (DTI) (including for example diffusion tensor measures of anatomical connectivity), [18F]fluorodeoxyglucose (FDG) PET, agents that label amyloid, [18F]F-dopa PET, radiotracer imaging, volumetric analysis of regional tissue loss, specific imaging markers of abnormal protein deposition (e.g., for AD progression), multimodal imaging, and biomarker analysis. Jon Stoessl, 2012. Combinations of these techniques can also be used to measure disease progression.

For example, structural MRI can be used to measure atrophy of the hippocampus and entorhinal cortex in AD, as well as involvement of the lateral parietal, posterior superior temporal and medial posterior cingulate cortices. In frontotemporal dementias (FTD), structural MRI can show atrophy in frontal or temporal poles.

In another embodiment, administration of a therapeutically effective fixed dose of an aminosterol composition to an MSA patient results in improvement of one or more symptoms of MSA, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. Improvement can be measured using any clinically recognized tool or assessment.

In a first embodiment, the invention encompasses a method of treating, preventing and/or slowing the onset or progression of MSA and/or a related symptom in a subject in need comprising administering to the subject a therapeutically effective amount of at least one aminosterol or a salt or derivative thereof.

In another embodiment, the invention encompasses a method of treating, preventing and/or slowing the onset or progression of MSA and/or a related symptom in a subject in need comprising (a) determining a dose of an aminosterol or a salt or derivative thereof for the subject, wherein the aminosterol dose is determined based on the effectiveness of the aminosterol dose in improving or resolving a MSA symptom being evaluated, (b) followed by administering the aminosterol dose to the subject for a period of time, wherein the method comprises (i) identifying a MSA symptom to be evaluated; (ii) identifying a starting aminosterol dose for the subject; and (iii) administering an escalating dose of the aminosterol to the subject over a period of time until an effective dose for the MSA symptom being evaluated is identified, wherein the effective dose is the aminosterol dose where improvement or resolution of the MSA symptom is observed, and fixing the aminosterol dose at that level for that particular MSA symptom in that particular subject.

In another embodiment, the starting aminosterol or a salt or derivative thereof dose is higher if the MSA symptom being evaluated is severe.

In one embodiment, the method results in slowing, halting, or reversing progression or onset of MSA over a defined period of time following administration of the fixed escalated dose of the aminosterol or a salt or derivative thereof, as measured by a medically-recognized technique. For example, the progression or onset of schizophrenia may be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique. In addition, the method of the invention can result in positively impacting the SZ, as measured by a medically-recognized technique.

In another embodiment, the fixed escalated aminosterol dose reverses dysfunction caused by the MSA and treats, prevents, improves, and/or resolves the MSA symptom being evaluated. Optionally, the improvement or resolution of the MSA and/or MSA symptom can be measured using a clinically recognized scale or tool. The improvement in the MSA symptom can be, for example, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale or tool.

In another embodiment, progression or onset of MSA is slowed, halted, or reversed over a defined period of time following administration of the fixed escalated dose of the aminosterol or a pharmaceutically acceptable salt or derivative thereof, as measured by a medically-recognized technique; and/or the MSA is positively impacted by the fixed escalated dose of the aminosterol or a pharmaceutically acceptable salt or derivative thereof, as measured by a medically-recognized technique. The positive impact and/or progression of MSA can be measured quantitatively or qualitatively by one or more techniques selected from the group consisting of conventional MRI (cMRI), susceptibility weighted imaging (SWI), magnetic resonance volumetry, diffusion weighted imaging, magnetic resonance spectroscopy, positron emission tomography (PET), single-photon emission computed tomography (SPECT), and [¹²³I] metaiodobenzylguanidine (MIBG) cardiac scintigraphy. In addition, the progression or onset of MSA can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In the methods of the invention utilizing an MSA symptom to be evaluated, the MSA symptom can be, for example, selected from the group consisting of: (a) a symptom from the Integrated Unified MSA Rating Scale (UMSARS) selected from the group consisting of speech impairment; swallowing impairment, handwriting impairment, difficulty using eating utensils, difficulty dressing, difficulty maintaining personal hygiene, walking impairment, falling, orthostatic impairment, urinary urgency, urinary frequency, urinary incontinence, sexual dysfunction, constipation, hypomania, and slowed speech; (b) a symptom from the Assessment and Rating of Ataxia (SARA) selected from the group consisting of abnormal gait, staggering when walking, inability to stand still, inability to sit still, speech impairment, dysmetria, tremor, difficulty with propination and supination of hand, and difficulty with heel-shin slide; (c) a symptom from the Berg Balance Scale (BBS) selected from the group consisting of difficulty standing from sitting, difficulty standing unsupported, difficulty sitting unsupported difficulty transferring from one seat to another, difficulty standing unsupported with eyes closed, difficulty standing with feet together, difficulty reaching with arm, difficulty retrieving objects from floor, difficulty looking behind oneself, difficulty turning 360 degrees, difficulty placing an alternate foot on a stool, difficulty standing with one foot in front of the other, and difficulty standing on one foot; (d) parkinsonism; (e) muscle rigidity; (f) difficulty bending arms and/or legs; (g) slow movement (bradykinesia); (h) tremors; (i) problems with posture and balance; (j) impaired movement and coordination, such as unsteady gait and loss of balance; (k) slurred, slow or low-volume speech (dysarthria); (l) visual disturbances, such as blurred or double vision and/or difficulty focusing eyes; (m) difficulty swallowing (dysphagia) or chewing; (m) ataxia; (o) orthostatic hypotension; (p) high blood pressure; (q) urinary and/or bowel dysfunction; (r) constipation; (s) loss of bladder and/or bowel control (incontinence); (t) reduced production of sweat, tears, and/or saliva (dry mouth); (u) heat intolerance due to reduced sweating; (v) impaired body temperature control, often causing cold hands and/or feet; (w) sleep disturbance and/or sleep disorder; (x) sexual dysfunction, such as impotence and/or loss of libido; (y) cardiovascular problems, such as irregular heartbeat; (z) vocal cord palsy; (aa) cognitive impairment; (bb) depression; (cc) psychiatric problems, such as difficulty controlling emotions; and (dd) neurodegeneration.

In one embodiment, the MSA symptom to be evaluated is ataxia, and (a) the ataxia comprises a lack of coordinated muscle movement, gait abnormality, speech abnormality, abnormal eye movement, hemiataxia, cerebellar ataxia, sensory ataxia, vestibular ataxia, or any combination thereof; (b) the method results in a positive change in the ataxia of the subject; and/or (c) the method results in a positive change in the ataxia of the subject, wherein the positive change is defined as: (i) an increase in the total amount of words a subject can recite without error of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100% over a defied period of time; and/or (ii) a percent decrease over a defined period of time, in the distance between an object the subject intends to reach for and the location the subject actually reaches to of 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In another embodiment, the MSA symptom to be evaluated is falling and (a) the method results in a decreased number of falls by the subject over a defined period of time; (b) the method results in a decreased number of falls by the subject over a defined period of time by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (c) the method results in the subject being free of instances of falling.

In yet another embodiment, the MSA symptom to be evaluated is falling and (a) the method results in a decreased likelihood of falling by the subject over a defined period of time, wherein the decreased likelihood is measured using one or more medically recognized technique; and/or (b) the method results in a decreased likelihood of falling by the subject over a defined period of time of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%, wherein the decreased likelihood is measured using one or more medically recognized technique. The one or more medically recognized techniques can be, for example, the Morse Scale, the Conley Scale, the STRATIFY Scale, The Hendrich II Fall Risk Model, Johns Hopkins Fall Risk Assessment Tool, and Stopping Elderly Accidents Deaths and Injuries (STEADI).

In one embodiment, the MSA symptom to be evaluated is urinary frequency, and/or urinary and/or bowel incontinence, and (a) the method results in a positive change in the urinary frequency, and/or urinary and/or bowel incontinence, of the subject; (b) the method results in a positive change in the urinary frequency, and/or urinary and/or bowel incontinence, of the subject and the positive change is defined as: (i) an increase in the amount of time between urinations and/or incontinence by the subject of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of urinations per day and/or incontinence by the subject selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; (c) as a result of the method the subject urinates the amount of times per day recommended by a medical authority for the age group of the subject; and/or (d) as a result of the method the subject does not experience any episodes of urinary and/or bowel incontinence.

In one embodiment, the MSA symptom to be evaluated is parkinsonism and (a) the parkinsonism comprises tremor, bradykinesia, rigidity, postural instability, or any combination thereof; (b) the method results in improvement in a subject's parkinsonism, as measured by one or more clinically-recognized parkinsonism rating scale; (c) the method results in improvement in a subject's parkinsonism, as measured by one or more clinically-recognized parkinsonism rating scale and the improvement is in one or more parkinsonism characteristics selected from the group consisting of tremor, bradykinesia, rigidity, and postural instability; and/or (d) the method results in improvement in a subject's parkinsonism, as measured by one or more clinically-recognized parkinsonism rating scale, and the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%.

In another embodiment, the MSA symptom to be evaluated is constipation, and (a) the fixed escalated aminosterol dose causes the subject to have a bowel movement; (b) the method results in an increase in the frequency of bowel movement in the subject; (c) the method results in an increase in the frequency of bowel movement in the subject and the increase in the frequency of bowel movement is defined as: (i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; (d) as a result of the method the subject has the frequency of bowel movement recommended by a medical authority for the age group of the subject; and/or (e) the starting aminosterol dose is determined by the severity of the constipation, wherein: (i) if the average complete spontaneous bowel movement (CSBM) or spontaneous bowel movement (SBM) is one or less per week, then the starting aminosterol dose is at least about 150 mg; and (ii) if the average CSBM or SBM is greater than one per week, then the starting aminosterol dose is about 75 mg or less.

In yet another embodiment, the MSA symptom to be evaluated is cognitive impairment, and (a) progression or onset of the cognitive impairment is slowed, halted, or reversed over a defined period of time following administration of the fixed escalated dose of the aminosterol or a salt or derivative thereof, as measured by a medically-recognized technique; and/or (b) the cognitive impairment is positively impacted by the fixed escalated dose of the aminosterol or a salt or derivative thereof, as measured by a medically-recognized technique; (c) the cognitive impairment is positively impacted by the fixed escalated dose of the aminosterol or a salt or derivative thereof, as measured by a medically-recognized technique and the positive impact on and/or progression of cognitive impairment is measured quantitatively or qualitatively by one or more techniques selected from the group consisting of ADASCog, Mini-Mental State Exam (MMSE), Mini-cog test, Woodcock-Johnson Tests of Cognitive Abilities, Leiter International Performance Scale, Miller Analogies Test, Raven's Progressive Matrices, Wonderlic Personnel Test, IQ tests, and a computerized tested selected from Cantab Mobile, Cognigram, Cognivue, Cognision, or Automated Neuropsychological Assessment Metrics Cognitive Performance Test (CPT); and/or (d) the progression or onset of cognitive impairment is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In yet another embodiment, the MSA symptom to be evaluated is a sleep disorder or sleep disturbance and (a) the sleep disorder or sleep disturbance comprises reduced sleep, excessive daytime sleepiness, REM sleep behavior disorder (RBD), sleep-disordered breathing, circadian rhythm dysfunction, a delay in sleep onset, sleep fragmentation, sleep-disordered breathing including snoring and apnea, micro-sleep episodes, narcolepsy, REM disturbed sleep, agitated sleep due to “acting out” dreams, inspiratory stridor during sleep, or any combination thereof; (b) the sleep disorder or sleep disturbance comprises REM-behavior disorder, and the REM-behavior disorder comprises vivid dreams, nightmares, and acting out the dreams by speaking or screaming, or fidgeting or thrashing of arms or legs during sleep; (c) the method results in a positive change in the sleeping pattern of the subject; (d) the method results in a positive change in the sleeping pattern of the subject, wherein the positive change is defined as (i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (e) as a result of the method the subject obtains the total number of hours of sleep recommended by a medical authority for the age group of the subject.

In one embodiment, the MSA symptom to be evaluated is depression and (a) the method results in improvement in a subject's depression, as measured by one or more clinically-recognized depression rating scale; and/or (b) the improvement is in one or more depression characteristics selected from the group consisting of mood, behavior, bodily functions such as eating, sleeping, energy, and sexual activity, and/or episodes of sadness or apathy; and/or (c) the improvement in (a) or (b) a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%, as measured by one or more clinically-recognized depression rating scale. The one or more clinically-recognized depression rating scales can be, for example, the Patient Health Questionnaire-9 (PHQ-9); the Beck Depression Inventory (BDI); Zung Self-Rating Depression Scale; Center for Epidemiologic Studies-Depression Scale (CES-D); and the Hamilton Rating Scale for Depression (HRSD).

In one embodiment, the MSA symptom to be evaluated is neurodegeneration correlated with MSA and (a) treating the neurodegeneration prevents and/or delays the onset and/or progression of the MSA; (b) progression or onset of the neurodegeneration is slowed, halted, or reversed over a defined period of time following administration of the fixed escalated dose of the aminosterol or a salt or derivative thereof, as measured by a medically-recognized technique; (c) the neurodegeneration is positively impacted by the fixed escalated dose of the aminosterol or a salt or derivative thereof, as measured by a medically-recognized technique; (d) the neurodegeneration is positively impacted by the fixed escalated dose of the aminosterol or a salt or derivative thereof, as measured by a medically-recognized technique and wherein: (i) the positive impact and/or progression of neurodegeneration is measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis; and/or (ii) the progression or onset of neurodegeneration is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In another embodiment, the aminosterol or a salt or derivative thereof is administered in combination with at least one additional active agent to achieve either an additive or synergistic effect. For example, the additional active agent can be administered via a method selected from the group consisting of (a) concomitantly; (b) as an admixture; (c) separately and simultaneously or concurrently; or (d) separately and sequentially. In another embodiment, the additional active agent is a different aminosterol from that administered in primary method. In yet a further embodiment, the method of the invention comprises administering a first aminosterol which is aminosterol 1436 or a salt or derivative thereof intranasally and administering a second aminosterol which is squalamine or a salt or derivative thereof orally.

In another embodiment, the at least one additional active agent is an active agent used to treat MSA or a symptom thereof. In some embodiments, the active agent is selected from the group consisting of antihypotensive agents such as fludrocortisone, pyridostigmine (Mestinon®), mitrodrine (Amatine®), or droxidopa (Northera®); vasodilators such as tadalafil (Cialis®) or sildenafil (Viagra®); dopamine receptor agonists such as pramipexole (Mirapex®) and apomorphine; antivirals such as amantadine (Symmetrel®); selective serotonin reuptake inhibitors such as paroxetine (Paxil®); and levodopa and carbidopa (Sinemet®).

iv. Schizophrenia

In one aspect, encompassed is a method of treating, preventing, and/or slowing the onset or progression of schizophrenia (SZ) and/or a related symptom in a subject in need comprising administering to the subject a therapeutically effective amount of at least one aminosterol or a salt or derivative thereof. In one aspect, the method of administration comprises oral, nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof. In another aspect, encompassed is a non-oral method of administration, as well as nasal administration.

In another aspect, encompassed is a method of treating, preventing and/or slowing the onset or progression of schizophrenia (SZ) and/or a related symptom in a subject in need comprising: (a) determining a dose of an aminosterol or a salt or derivative thereof for the subject, wherein the aminosterol dose is determined based on the effectiveness of the aminosterol dose in improving or resolving a SZ symptom being evaluated, (b) followed by administering the aminosterol dose to the subject for a defined period of time, wherein the method comprises: (i) identifying a SZ symptom to be evaluated; (ii) identifying a starting aminosterol dose for the subject; and (iii) administering an escalating dose of the aminosterol to the subject over a defined period of time until an effective dose for the SZ symptom being evaluated is identified, wherein the effective dose is the aminosterol dose where improvement or resolution of the SZ symptom is observed, and fixing the aminosterol dose at that level for that particular SZ symptom in that particular subject; and optionally (c) wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

Schizophrenia is a chronic progressive disorder that has at its origin structural brain changes in both white and gray matter. It is likely that these changes begin prior to the onset of clinical symptoms in cortical regions, particularly those concerned with language processing. Later, they can be detected by progressive ventricular enlargement. Current magnetic resonance imaging (MRI) technology can provide a valuable tool for detecting early changes in cortical atrophy and anomalous language processing, which may be predictive of who will develop schizophrenia.

A 2013 study of schizophrenia patients documented brain changes seen in MRI scans from more than 200 patients beginning with their first episode and continuing with scans at regular intervals for up to 15 years. The scans showed that people at their first episode had less brain tissue than healthy individuals. The findings suggest that those who have schizophrenia are being affected by something before they show outward signs of the disease.

The mainstay of treatment is antipsychotic medication, along with counselling, job training and social rehabilitation. However, the 2013 study found that in general, the higher the anti-psychotic medication doses, the greater the loss of brain tissue.

About 0.3-0.7% of people are affected by schizophrenia during their lifetimes. In 2013 there were an estimated 23.6 million cases globally. Males are more often affected, and on average experience more severe symptoms. About 20% of people do well and a few recover completely. About 50% have lifelong impairment. Social problems, such as long-term unemployment, poverty and homelessness are common. The average life expectancy of people with the disorder is ten to twenty-five years less than for the general population. This is the result of increased physical health problems and a higher suicide rate (about 5%). In 2015 an estimated 17,000 people worldwide died from behavior related to, or caused by, schizophrenia.

While not wished to be bound by theory, it is theorized that administration of a therapeutically effective fixed dose of an aminosterol composition to a schizophrenia patient may treat and/or prevent schizophrenia or any one or more symptoms thereof. In some embodiments, the administration may be oral—resulting in absorption in the ENS. In some embodiments, the administration may be intranasal—resulting in stimulation of neurogenesis, which has a positive impact on the loss of brain tissue characteristic of schizophrenia subjects.

In one embodiment of the invention, administration of a therapeutically effective fixed dose of an aminosterol composition to a schizophrenia patient results in improvement of one or more symptoms as determined by a clinically recognized psychiatric symptom rating scale. Examples of such rating scales include for example, the Positive and Negative Syndrome Scale (PANSS), the Psychotic Symptom Rating Scales (PSYRATS), the Quality of Life Scale (QLS), the Schizophrenia Cognition Rating Scale (SCoRS), the Drug Attitude Inventory (DAI), and the Abnormal Involuntary Movement Scale (AIMS).

In another embodiment, administration of a therapeutically effective fixed dose of an aminosterol composition to a schizophrenia patient results in improvement of one or more symptoms as determined by a clinically recognized psychiatric symptom rating scale, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. Improvement can be measured using any clinically recognized tool or assessment.

In one embodiment, the invention encompasses a method of treating, preventing and/or slowing the onset or progression of schizophrenia and/or a related symptom in a subject in need comprising administering to the subject a therapeutically effective amount of at least one aminosterol or a salt or derivative thereof.

In another embodiment, the invention encompasses a method of treating, preventing and/or slowing the onset or progression of schizophrenia and/or a related symptom in a subject in need comprising (a) determining a dose of an aminosterol or a salt or derivative thereof for the subject, wherein the aminosterol dose is determined based on the effectiveness of the aminosterol dose in improving or resolving a SZ symptom being evaluated, (b) followed by administering the aminosterol dose to the subject for a period of time, wherein the method comprises (i) identifying a SZ symptom to be evaluated; (ii) identifying a starting aminosterol dose for the subject; and (iii) administering an escalating dose of the aminosterol to the subject over a period of time until an effective dose for the SZ symptom being evaluated is identified, wherein the effective dose is the aminosterol dose where improvement or resolution of the SZ symptom is observed, and fixing the aminosterol dose at that level for that particular SZ symptom in that particular subject.

In another embodiment, the starting aminosterol or a salt or derivative thereof dose is higher if the SZ symptom being evaluated is severe.

In one embodiment, the method results in slowing, halting, or reversing progression or onset of SZ over a defined period of time following administration of the fixed escalated dose of the aminosterol or a salt or derivative thereof, as measured by a medically-recognized technique. For example, the progression or onset of schizophrenia may be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique. In addition, the method of the invention can result in positively impacting the SZ, as measured by a medically-recognized technique.

The positive impact and/or progression of SZ, and/or improvement or resolution of the SZ symptom being evaluated, may be measured quantitatively or qualitatively by one or more clinically recognized scales, tools, or techniques selected from the group consisting of The Clinical Assessment Interview for Negative Symptoms (CAINS), The Brief Negative Symptom Scale (BNSS), Scale for the Assessment of Positive Symptoms (SAPS), the Scale for the Assessment of Negative Symptoms (SANS), the Positive and Negative Symptoms Scale (PANSS), the Negative Symptom Assessment (NSA-16), the Clinical Global Impression Schizophrenia (CGI-SCH), computed tomography (CT), magnetic resonance imaging (MRI), magnetic resonance spectroscopy, functional MRI (fMRI), diffusion tensor imaging, single photon emission computed tomography (SPECT), and positron emission tomography (PET).

In one embodiment, the fixed escalated aminosterol dose reverses dysfunction caused by the SZ and treats, prevents, improves, and/or resolves the schizophrenia symptom being evaluated.

In another embodiment, the fixed escalated aminosterol dose reverses dysfunction caused by the SZ and treats, prevents, improves, and/or resolves the schizophrenia symptom being evaluated. The improvement or resolution of the SZ symptom can be measured using a clinically recognized scale or tool. For example, the improvement in the schizophrenia symptom can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale.

In yet another embodiment, the SZ symptom to be evaluated can be selected from the group consisting of (a) reduced social engagement, social withdrawal, and/or social isolation; (b) reduced emotional expression; (c) disorganized or irrational behavior; (d) disorganized or irrational thinking; (e) disorganized or irrational speech; (f) aggression or anger; (g) anxiety; (h) compulsive behavior; (i) excitability; (j) repetitive movements; (k) self-harm; (l) delusions; (m) amnesia; (n) emotional instability, including difficulty controlling emotions; (o) hallucinations; (p) depression; (q) constipation; (r) neurodegeneration associated with schizophrenia; (s) sleep problem, sleep disorder, and/or sleep disturbance; (t) cognitive impairment; (u) feelings of fright and/or paranoia; (v) false beliefs; (w) distorted thoughts; (x) lack of emotion or a very limited range of emotions; (y) catatonia; (z) impaired motor behavior and coordination; (aa) inability to make decisions; (bb) forgetting or losing things; (cc) poor executive functioning; (dd) ADHD, trouble focusing, paying attention and/or difficulty concentrating; (ee) difficulty with working memory; (ff) lack of motivation; (gg) reduced energy or apathy; (hh) reduced speech; (ii) loss of pleasure or interest in life; (jj) poor hygiene and grooming habits; (kk) hypertension; (ll) hypotension; (mm) sexual dysfunction, such as impotence and/or loss of libido, and/or (nn) cardiovascular disease.

In one embodiment, the schizophrenia symptom to be evaluated is a sleep problem, sleep disorder, or sleep disturbance associated with schizophrenia, and the sleep problem, sleep disorder, or sleep disturbance comprises a delay in sleep onset, sleep fragmentation, REM-behavior disorder, sleep-disordered breathing including snoring and apnea, day-time sleepiness, micro-sleep episodes, narcolepsy, hallucinations, or any combination thereof. Further, the REM-behavior disorder can comprise vivid dreams, nightmares, and acting out the dreams by speaking or screaming, or fidgeting or thrashing of arms or legs during sleep. Treating the sleep problem, sleep disorder, or sleep disturbance prevents or delays the onset and/or progression of the schizophrenia.

In one embodiment, the schizophrenia symptom to be evaluated is a sleep problem, sleep disorder, sleep disturbance, circadian rhythm dysfunction, REM disturbed sleep, or REM behavior disorder, and (a) the method results in a positive change in the sleeping pattern of the subject; (b) the method results in a positive change in the sleeping pattern of the subject, wherein the positive change is defined as: (i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (c) as a result of the method the subject obtains the total number of hours of sleep recommended by a medical authority for the age group of the subject.

In another embodiment, the SZ symptom to be evaluated is hallucinations and wherein: (a) the hallucinations comprise a visual, auditory, tactile, gustatory or olfactory hallucinations (b) the method results in a decreased number of hallucinations over a defined period of time in the subject; (c) the method results in a decreased number of hallucinations over a defined period of time in the subject selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (d) the method results in the subject being hallucination-free. In one embodiment, the fixed escalated aminosterol dose reverses dysfunction caused by the schizophrenia and treats and/or prevents the hallucination.

In another embodiment, the SZ symptom to be evaluated is hallucinations and wherein: (a) the hallucinations comprise a visual, auditory, tactile, gustatory or olfactory hallucinations; (b) the method results in a decreased severity of hallucinations in the subject over a defined period of time, wherein the decrease in severity is measured by one or more medically-recognized techniques; (c) the method results in a decreased severity of hallucinations in the subject over a defined period of time, wherein the decrease in severity is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%, as measured by one or more medically recognized techniques; and/or (d) the method results in the subject being hallucination-free. The one or more medically recognized techniques may be selected from the group consisting of Chicago Hallucination Assessment Tool (CHAT), The Psychotic Symptom Rating Scales (PSYRATS), Auditory Hallucinations Rating Scale (AHRS), Hamilton Program for Schizophrenia Voices Questionnaire (HPSVQ), Characteristics of Auditory Hallucinations Questionnaire (CAHQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA).

In another embodiment, the schizophrenia symptom to be evaluated is depression. In an exemplary embodiment, the method results in improvement in a subject's depression, as measured by one or more clinically-recognized depression rating scales. For example, the improvement can be in one or more depression characteristics selected from the group consisting of mood, behavior, bodily functions such as eating, sleeping, energy, and sexual activity, and/or episodes of sadness or apathy. In another embodiment, the improvement a subject experiences following treatment can be about 5, about 10, about 15, about 20, about 25, about 30, about 35, about 40, about 45, about 50, about 55, about 60, about 65, about 70, about 75, about 80, about 85, about 90, about 95 or about 100%.

In some embodiments, the schizophrenia symptom to be evaluated is cognitive impairment, and (a) progression or onset of the cognitive impairment is slowed, halted, or reversed over a defined period of time following administration of the fixed escalated dose of the aminosterol or a salt or derivative thereof, as measured by a medically-recognized technique; (b) the cognitive impairment is positively impacted by the fixed escalated dose of the aminosterol or a salt or derivative thereof, as measured by a medically-recognized technique; (c) the cognitive impairment is positively impacted by the fixed escalated dose of the aminosterol or a salt or derivative thereof, as measured by a medically-recognized technique and the positive impact on and/or progression of cognitive impairment is measured quantitatively or qualitatively by one or more techniques selected from the group consisting of ADASCog, Mini-Mental State Exam (MMSE), Mini-cog test, Woodcock-Johnson Tests of Cognitive Abilities, Leiter International Performance Scale, Miller Analogies Test, Raven's Progressive Matrices, Wonderlic Personnel Test, IQ tests, or a computerized tested selected from Cantab Mobile, Cognigram, Cognivue, Cognision, and Automated Neuropsychological Assessment Metrics Cognitive Performance Test (CPT); and/or (d) the progression or onset of cognitive impairment is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In some embodiments, the schizophrenia symptom to be evaluated is constipation, and (a) treating the constipation prevents and/or delays the onset and/or progression of the schizophrenia; (b) the fixed escalated aminosterol dose causes the subject to have a bowel movement; (c) the method results in an increase in the frequency of bowel movement in the subject; (d) the method results in an increase in the frequency of bowel movement in the subject and the increase in the frequency of bowel movement is defined as: (i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; (e) as a result of the method the subject has the frequency of bowel movement recommended by a medical authority for the age group of the subject; and/or (f) the starting aminosterol dose is determined by the severity of the constipation, wherein: (i) if the average complete spontaneous bowel movement (CSBM) or spontaneous bowel movement (SBM) is one or less per week, then the starting aminosterol dose is at least about 150 mg; and (ii) if the average CSBM or SBM is greater than one per week, then the starting aminosterol dose is about 75 mg or less.

In one embodiment, the schizophrenia symptom to be evaluated is neurodegeneration, and (a) treating the neurodegeneration prevents and/or delays the onset and/or progression of the schizophrenia; and/or (b) the method results in treating, preventing, and/or delaying the progression and/or onset of neurodegeneration in the subject. In an exemplary embodiment (a) progression or onset of the neurodegeneration is slowed, halted, or reversed over a defined period of time following administration of the fixed escalated dose of the aminosterol or a salt or derivative thereof, as measured by a medically-recognized technique; and/or (b) the neurodegeneration is positively impacted by the fixed escalated dose of the aminosterol or a salt or derivative thereof, as measured by a medically-recognized technique. The positive impact and/or progression of neurodegeneration can be measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis. In addition, the progression or onset of neurodegeneration can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In another embodiment, the aminosterol or a salt or derivative thereof is administered in combination with at least one additional active agent to achieve either an additive or synergistic effect. For example, the additional active agent can be administered via a method selected from the group consisting of (a) concomitantly; (b) as an admixture; (c) separately and simultaneously or concurrently; or (d) separately and sequentially. In another embodiment, the additional active agent is a different aminosterol from that administered in primary method. In yet a further embodiment, the method of the invention comprises administering a first aminosterol which is aminosterol 1436 or a salt or derivative thereof intranasally and administering a second aminosterol which is squalamine or a salt or derivative thereof orally.

In another embodiment, the at least one additional active agent is an active agent used to treat schizophrenia or a symptom thereof. In some embodiments, the active agent is selected from the group consisting of first-generation antipsychotics such as chlorpromazine (Thorazine®), fluphenazine (Prolixin®), haloperidol (Haldol®), perphenazine (Trilafon®), thioridazine thiothixene (Navane®), and trifluoperazine (Stelazine®); atypical antipsychotics such as aripiprazole (Abilify®), aripiprazole lauroxil (Aristada®), asenapine (Saphris®), clozapine (Clozaril®), iloperidone (Fanapt®), lurasidone (Latuda®), olanzapine (Zyprexa®), paliperidone (Invega Sustenna®), paliperidone palmitate (Invega Trinza®), quetiapine (Seroquel®), risperidone (Risperdal®), and ziprasidone (Geodon®).

In another embodiment, the subject to be treated according to the methods of the invention can be a member of a patient population at risk for being diagnosed with SZ.

v. Autism

In one aspect, encompassed is a method of treating, preventing, and/or slowing the onset or progression of autism spectrum disorder (ASD) and/or a related symptom in a subject in need comprising administering to the subject a therapeutically effective amount of at least one aminosterol, or a salt or derivative thereof, provided that the administering does not comprise oral administration. The method of administration can comprise, for example, nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof. In a preferred aspect, the method of claim 1, wherein the method of administration comprises nasal administration.

In another aspect, a method of treating, preventing, and/or slowing the onset or progression of autism spectrum disorder (ASD) and/or a related symptom in a subject in need comprising: (a) determining a dose of an aminosterol or a salt or derivative thereof for the subject, wherein the aminosterol dose is determined based on the effectiveness of the aminosterol dose in improving or resolving an ASD symptom being evaluated, (b) followed by administering the aminosterol dose to the subject for a defined period of time, wherein the method comprises: (i) identifying an ASD symptom to be evaluated; (ii) identifying a starting aminosterol dose for the subject; and (iii) administering an escalating dose of the aminosterol to the subject over a defined period of time until an effective dose for the ASD symptom being evaluated is identified, wherein the effective dose is the aminosterol dose where improvement or resolution of the ASD symptom is observed, and fixing the aminosterol dose at that level for that particular ASD symptom in that particular subject; and (c) optionally wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

Autism, or autism spectrum disorder, refers to a range of conditions characterized by challenges with social skills, repetitive behaviors, speech and nonverbal communication, as well as by unique strengths and differences. There are many types of autism, caused by different combinations of genetic and environmental influences.

Autism's most-obvious signs tend to appear between 2 and 3 years of age. In some cases, it can be diagnosed as early as 18 months. Some developmental delays associated with autism can be identified and addressed even earlier.

The Centers for Disease Control and Prevention (CDC) estimates autism's prevalence as 1 in 59 children in the United States. This includes 1 in 37 boys and 1 in 151 girls. Around one third of people with autism remain nonverbal. Around one third of people with autism have an intellectual disability. Certain medical and mental health issues frequently accompany autism. They include gastrointestinal (GI) disorders, seizures, sleep disturbances, attention deficit and hyperactivity disorder (ADHD), anxiety and phobias.

Experts are still uncertain about of all the causes of autism. In all likelihood, there are multiple causes. It appears that a number of different circumstances, including environmental, biologic, and genetic factors, set the stage for autism and make a child more likely to have the disorder. It is likely that genetics play a large factor in the development of autism. Identical twins are more likely to both be affected than twins who are fraternal (not genetically identical). In a family with one autistic child, the chance of having another child with autism is about 5 percent—or one in 20—which is much higher than in the normal population. Research also has found that some emotional disorders (such as manic depression) occur more often in families of a child with autism.

At least one group of researchers has found a link between an abnormal gene and autism. The gene may be just one of three to five or more genes that interact in some way to cause the condition. Scientists suspect that a faulty gene or genes might make a person more likely to develop autism when there are also other factors present, such as a chemical imbalance, viruses or chemicals, or a lack of oxygen at birth.

Other potential causes of autism are environmental toxins, including pesticides and heavy metals such as mercury. Heavy metals are certainly more commonly encountered in the environment now than they were in the past. It may be that people with autism or those at higher risk for developing it are more sensitive than others to these toxins.

A recent brain-tissue study suggests that children affected by autism have a surplus of synapses, or connections between brain cells. The excess is due to a slowdown in the normal pruning process that occurs during brain development. During normal brain development, a burst of synapse formation occurs in infancy. This is particularly pronounced in the cortex, which is central to thought and processing information from the senses. But by late adolescence, pruning eliminates about half of these cortical synapses. In addition, many genes linked to autism are known to affect the development or function of brain synapses. The study also found that the brain cells from individuals with autism were filled with damaged parts and deficient in signs of a normal breakdown pathway called “autophagy.” Tang et al. 2014.

Thus, one embodiment of the invention is directed to methods of treating autism comprising administering a therapeutically effective fixed dose of an aminosterol composition according to the invention. In one embodiment, treatment results in improvement in one or more characteristics of autism. Such characteristics can be, for example, communication skills, social interaction, sensory sensitivity, and behavior. Improvement can be measured using any clinically recognized tool or assessment.

For example, the methods of the invention may show an improvement in one or more characteristics of autism, such as behavior, communication, mood, etc., as measured by a medically recognized scale. The improvement may be, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In one embodiment, encompassed are methods of treating, preventing, and/or slowing the onset or progression of autism spectrum disorder (ASD) and/or a related symptom in a subject in need comprising administering to the subject a therapeutically effective amount of at least one aminosterol or a salt or derivative thereof.

Certain embodiments describe the determination and administration of a “fixed aminosterol dose” that is not age, size, or weight dependent but rather is individually calibrated. Thus, in another embodiment, encompassed is a method of treating, preventing and/or slowing progression ASD and/or a related symptom in a subject in need comprising (a) determining a dose of an aminosterol or a salt or derivative thereof for the subject, wherein the aminosterol dose is determined based on the effectiveness of the aminosterol dose in improving or resolving an ASD symptom being evaluated, (b) followed by administering the aminosterol dose to the subject for a period of time, wherein the method comprises (i) identifying an ASD symptom to be evaluated; (ii) identifying a starting dose of an aminosterol or a salt or derivative thereof for the subject; and (iii) administering an escalating dose of the aminosterol or a salt or derivative thereof to the subject over a period of time until an effective dose for the ASD symptom being evaluated is identified, wherein the effective dose is the aminosterol dose where improvement or resolution of the ASD symptom is observed, and fixing the dose of the aminosterol or a salt or derivative thereof at that level for that particular ASD symptom in that particular subject. In this aspect of the invention, the aminosterol or a salt or derivative thereof can be administered via any pharmaceutically acceptable means. For example, the aminosterol or a salt or derivative thereof can be administered via any pharmaceutically acceptable means, such as orally, intranasally, by injection (IV, IP, or IM) or any combination thereof. The aminosterol or a salt or derivative thereof can be formulated with one or more pharmaceutically acceptable carriers or excipients.

In another embodiment, the starting aminosterol or a salt or derivative thereof dose is higher if the ASD symptom being evaluated is severe. In one embodiment, the fixed escalated dose of the aminosterol or a salt or derivative thereof reverses dysfunction caused by the ASD and treats, prevents, improves, and/or resolves the symptom being evaluated.

In yet another embodiment, the ASD symptom to be evaluated can be selected from the group consisting of (a) a symptom from the Autism Spectrum Rating Scales (ASRS™) selected from the group consisting of social skills, communication skills, unusual behavior, self-regulation ability, peer socialization, adult socialization, atypical language, and stereotypy; (b) a symptom from the Autism Diagnostic Observation Schedule (ADOS) selected from the group consisting of performance in Module 1 (used with children who use little or no phrase speech), performance in Module 2 (used with subjects that use phrase speech but who do not speak fluently), performance in Module 3 (used with younger subjects who are verbally fluent), and performance in Module 4 (used with adolescents and adults who are verbally fluent); (c) a symptom from the bl Diagnostic Interview-Revised (ADI-R), wherein the symptom is selected from the group consisting of emotional sharing, offering and seeking comfort, social smiling, responding to other children, stereotyped utterances, pronoun reversal, social usage of language, preoccupation with unusual things, hand and finger mannerism, unusual sensory interests, self-injury, aggression, and overactivity; (d) failure to respond to name; (e) failure to point at objects of interest; (f) inability to role play; (g) avoidance of eye contact; (h) preference to be alone; (i) inability to understand feelings of others; (j) no speech or delayed development of speech; (k) echolalia and/or palilalia; (l) answering questions with unrelated answers; (m) upset by minor changes; (n) obsessive interests; (o) lining-up or stacking of objects; (p) repetitive motion; (q) avoidance of physical contact with others; (r) lack of awareness of danger; (s) sleep disorder or sleep disturbance; (r) constipation; (s) cognitive impairment; (t) gastrointestinal (GI) problems; (u) epilepsy; (v) feeding issues; (w) Attention-deficit/hyperactivity disorder (ADHD); (x) anxiety; (y) depression; (z) Obsessive compulsive disorder (OCD); (aa) schizophrenia; (bb) Bipolar Disorder; and (cc) neurodegeneration associated with ASD. Examples of GI issues/disorders include, but are not limited to, chronic constipation, abdominal pain, gastroesophageal reflux, and bowel inflammation.

In some embodiments, the ASD symptom is a sleep disorder or sleep disturbance and is selected from the group consisting of decreased quantity of REM sleep, increased undifferentiated sleep, immature organization of eye movements into discrete bursts during REM sleep, decreased time in bed, decreased total sleep time, decreased REM sleep latency, increased proportion of stage 1 sleep, circadian rhythm disruption, and any combination thereof.

In some embodiments, the symptom to be evaluated is a sleep disorder or sleep disturbance wherein (a) the method results in a positive change in the sleeping pattern of the subject; (b) the method results in a positive change in the sleeping pattern of the subject, wherein the positive change is defined as (i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (c) as a result of the method the subject obtains the total number of hours of sleep recommended by a medical authority for the age group of the subject.

In some embodiments, the ASD symptom to be evaluated is a avoidance of eye contact, wherein (a) the method results in a positive change in the amount of eye contact engaged in by the subject; (b) the method results in a positive change in the amount of eye contact engaged in by the subject, wherein the positive change is defined as (i) an increase in the amount of eye contact of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of instances in which the subject avoids eye contact selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the ASD symptom to be evaluated is echolalia (unsolicited repetition of vocalizations made by another person) and/or palilalia (repetition of vocalizations made by the same person), wherein (a) the method results in a decreased number or severity of instances in which the subject engages in echolalia and/or palilalia; (b) the method results in a decreased number or severity of instances in which the subject engages in echolalia and/or palilalia and the decrease in number or severity of instances in which the subject engages in echolalia and/or palilalia is defined as a reduction in engagement in echolalia and/or palilalia selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (c) the method results in the subject ceasing to engage in echolalia and/or palilalia.

In some embodiments, the ASD symptom to be evaluated is self-injury wherein (a) the method results in a decreased number or severity of instances in which the subject engages in self-injury; (b) the method results in a decreased number or severity of instances in which the subject engages in self-injury and the decrease is selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (c) the method results in the subject ceasing to engage in self-injury.

In some embodiments, the ASD symptom to be evaluated is repetitive motion wherein (a) the method results in a decreased number or severity of instances in which the subject engages in repetitive motion; (b) the method results in a decreased number or severity of instances in which the subject engages in repetitive motion and the decrease is selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (c) the method results in the subject ceasing to engage in repetitive motion.

In some embodiments, the ASD symptom to be evaluated is constipation, wherein (a) the fixed escalated aminosterol dose causes the subject to have a bowel movement; (b) the method results in an increase in the frequency of bowel movement in the subject; (c) the method results in an increase in the frequency of bowel movement in the subject and the increase in the frequency of bowel movement is defined as (i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; (d) as a result of the method the subject has the frequency of bowel movement recommended by a medical authority for the age group of the subject; and/or (e) the starting aminosterol dose is determined by the severity of the constipation, wherein (i) if the average complete spontaneous bowel movement (CSBM) or spontaneous bowel movement (SBM) is one or less per week, then the starting aminosterol dose is at least about 150 mg/day; and (ii) if the average CSBM or SBM is greater than one per week, then the starting aminosterol dose is about 75 mg/day or less.

In some embodiments, the ASD symptom to be evaluated is cognitive impairment, and (a) progression or onset of the cognitive impairment is slowed, halted, or reversed over a defined period of time following administration of the fixed escalated dose of the aminosterol or a salt or derivative thereof, as measured by a medically-recognized technique; and/or (b) the cognitive impairment is positively impacted by the fixed escalated dose of the aminosterol or a salt or derivative thereof, as measured by a medically-recognized technique; (c) the cognitive impairment is positively impacted by the fixed escalated dose of the aminosterol or a salt or derivative thereof, as measured by a medically-recognized technique and the positive impact on and/or progression of cognitive impairment is measured quantitatively or qualitatively by one or more techniques selected from the group consisting of ADASCog, Mini-Mental State Exam (MMSE), Mini-cog test, Woodcock-Johnson Tests of Cognitive Abilities, Leiter International Performance Scale, Miller Analogies Test, Raven's Progressive Matrices, Wonderlic Personnel Test, IQ tests, and a computerized tested selected from Cantab Mobile, Cognigram, Cognivue, Cognision, or Automated Neuropsychological Assessment Metrics Cognitive Performance Test (CPT); and/or (d) the progression or onset of cognitive impairment is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In embodiments where the ASD symptom to be evaluated is depression, (a) treating the depression may prevent and/or delay the onset and/or progression of ASD; (b) the method may result in improvement in a subject's depression, as measured by one or more clinically-recognized depression rating scale; (c) the method may result in improvement in a subject's depression, as measured by one or more clinically-recognized depression rating scale and the improvement can be in one or more depression characteristics selected from the group consisting of mood, behavior, bodily functions such as eating, sleeping, energy, and sexual activity, and/or episodes of sadness or apathy; and/or (d) the method may result in improvement in a subject's depression, as measured by one or more clinically-recognized depression rating scale, and the improvement a subject experiences following treatment can be about 5, about 10, about 15, about 20, about 25, about 30, about 35, about 40, about 45, about 50, about 55, about 60, about 65, about 70, about 75, about 80, about 85, about 90, about 95 or about 100%.

In embodiments where the ASD symptom to be evaluated is neurodegeneration correlated with ASD, (a) treating the neurodegeneration may prevent and/or delay the onset and/or progression of the ASD; (b) the method may result in treating, preventing, and/or delaying the progression and/or onset of neurodegeneration in the subject; (c) progression or onset of the neurodegeneration can be slowed, halted, or reversed over a defined period of time following administration of the fixed escalated dose of the aminosterol or a salt or derivative thereof, as measured by a medically-recognized technique; and/or (d) the neurodegeneration can be positively impacted by the fixed escalated dose of the aminosterol or a salt or derivative thereof, as measured by a medically-recognized technique. In further embodiments, (a) the positive impact and/or progression of neurodegeneration can be measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis; and/or (b) the progression or onset of neurodegeneration can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In some embodiments, the aminosterol or a salt or derivative thereof is administered in combination with at least one additional active agent to achieve either an additive or synergistic effect. In some embodiments, the additional active agent is administered via a method selected from the group consisting of (a) concomitantly; (b) as an admixture; (c) separately and simultaneously or concurrently; and (d) separately and sequentially.

In some embodiments, the additional active agent is a different aminosterol or a salt or derivative thereof from that administered in the method described herein.

In some embodiments, the method comprises administering a first aminosterol or a salt or derivative thereof which is aminosterol 1436 or a salt or derivative thereof administered intranasally and a second aminosterol or a salt or derivative thereof which is squalamine or a salt or derivative thereof administered orally. In some embodiments, the additional active agent is an active agent used to treat ASD or a symptom thereof.

In some embodiments, the active agent is selected from the group consisting of serotonin-norepinephrine reuptake inhibitors such as venlafaxine, (Effexor®); selective serotonin reuptake inhibitor such as fluoxetine (Prozac®) or citalopram (Celexa®); N-methyl D-aspartate (NMDA) antagonists such as memantine (Namenda®); dopamine receptor antagonists such as haloperidol (Haldol®); a loop diuretic such as bumetanide; an acetylcholinesterase inhibitor such as rivastigmine (Exelon®); a central nervous system stimulant such as methylphenidate (Ritalin®) or amphetamine (Adderall®); and/or atypical antipsychotics such as risperidone (Risperdol®), aripiprazole (Abilify®), ziprasidone (Geodon®), paliperidone (Invega®), or clozapine (Clozaril®).

vi. Other Neurodiseases

The methods and compositions of the invention may also be useful in treating and/or preventing a variety of other neurodiseases.

Huntington's disease (HD) is a fatal genetic disorder that causes the progressive breakdown of nerve cells in the brain. It deteriorates a person's physical and mental abilities during their prime working years and has no cure. Full-time care is required in the later stages of the disease. Symptoms of Huntington's disease most commonly become noticeable between the ages of 35 and 44 years, but they can begin at any age from infancy to old age. The most characteristic initial physical symptoms are jerky, random, and uncontrollable movements called chorea. Suicide is the cause of death in about 9% of cases. Death typically occurs 15 to 20 years from when the disease was first detected.

Progressive supranuclear palsy, also called Steele-Richardson-Olszewski syndrome, is an brain disorder that causes serious problems with walking, balance and eye movements. The disorder results from deterioration of cells in areas of the brain that control body movement and thinking. There is no known cure for PSP and management is primarily supportive.

Frontotemporal dementia (FTD) is a group of related conditions resulting from the progressive degeneration of the temporal and frontal lobes of the brain. These areas of the brain play a significant role in decision-making, behavioral control, emotion and language. The frontotemporal dementias (FTD) encompass six types of dementia involving the frontal or temporal lobes. They are: behavioral variant of FTD, semantic variant primary progressive aphasia, nonfluent agrammatic variant primary progressive aphasia, corticobasal syndrome, progressive supranuclear palsy, and FTD associated with motor neuron disease. Currently, there is no cure for FTD.

Vascular dementia, also known as multi-infarct dementia (MID) and vascular cognitive impairment (VCI), is dementia caused by problems in the supply of blood to the brain, typically a series of minor strokes, leading to worsening cognitive decline that occurs step by step. Risk factors for vascular dementia include age, hypertension, smoking, hypercholesterolemia, diabetes mellitus, cardiovascular disease, and cerebrovascular disease. Other risk factors include geographic origin, genetic predisposition, and prior strokes.

Amyotrophic lateral sclerosis (ALS), also known as motor neurone disease (MND), or Lou Gehrig's disease, is a specific disease which causes the death of neurons controlling voluntary muscles. ALS is characterized by stiff muscles, muscle twitching, and gradually worsening weakness due to muscles decreasing in size. This results in difficulty speaking, swallowing, and eventually breathing. The cause is not known in 90% to 95% of cases. The remaining 5-10% of cases are genetic. The underlying mechanism involves damage to both upper and lower motor neurons. No cure for ALS is known. The disease can affect people of any age, but usually starts around the age of 60 and in inherited cases around the age of 50. The average survival from onset to death is 2 to 4 years, although about 10% survive longer than 10 years.

Multiple sclerosis (MS) is a demyelinating disease in which the insulating covers of nerve cells in the brain and spinal cord are damaged. This damage disrupts the ability of parts of the nervous system to communicate, resulting in a range of signs and symptoms, including physical, mental, and sometimes psychiatric problems. Specific symptoms can include double vision, blindness in one eye, muscle weakness, trouble with sensation, or trouble with coordination. MS takes several forms, with new symptoms either occurring in isolated attacks (relapsing forms) or building up over time (progressive forms). Between attacks, symptoms may disappear completely; however, permanent neurological problems often remain, especially as the disease advances. While the cause is not clear, the underlying mechanism is thought to be either destruction by the immune system or failure of the myelin-producing cells. Proposed causes for this include genetics and environmental factors such as being triggered by a viral infection. There is no known cure for MS. Life expectancy is on average 5 to 10 years lower than that of an unaffected population. MS is the most common immune-mediated disorder affecting the central nervous system. In 2015, about 2.3 million people were affected globally, and in 2015 about 18,900 people died from MS, up from 12,000 in 1990.

Spinal muscular atrophy (SMA) is an inherited neuromuscular disorder characterized by loss of motor neurons and progressive muscle wasting, often leading to early death. The disorder is caused by a genetic defect in the SMN1 gene, which encodes SMN, a protein necessary for survival of motor neurons. Lower levels of the protein results in loss of function of neuronal cells in the anterior horn of the spinal cord and subsequent system-wide atrophy of skeletal muscles. SMA is the most common genetic cause of infant death. In December 2016, nusinersen became the first approved drug to treat SMA while several other compounds remain in clinical trials.

Friedreich's ataxia is an autosomal recessive inherited disease that causes progressive damage to the nervous system. It manifests in initial symptoms of poor coordination such as gait disturbance; it can also lead to scoliosis, heart disease and diabetes, but does not affect cognitive function. The ataxia of Friedreich's ataxia results from the degeneration of nervous tissue in the spinal cord, in particular sensory neurons essential (through connections with the cerebellum) for directing muscle movement of the arms and legs. The spinal cord becomes thinner and nerve cells lose some of their myelin sheath (the insulating covering on some nerve cells that helps conduct nerve impulses).

Progression of neurodegeneration can be measured using well known techniques. For example, an electroencephalogram (EEG) can be used as a biomarker for the presence and progression of a neurodegenerative disease. S. Morairty, “Detecting Neurodegenerative Diseases Before Damage Is Done,” SRI International (Jul. 26, 2013) (https://www.sri.com/blog/detecting-neurodegenerative-diseases). Another exemplary technique that can be used to measure progression of neurodegeneration of MRI. Rocca et al., “The Role of T1-Weighted Derived Measures of Neurodegeneration for Assessing Disability Progression in Multiple Sclerosis,” Front Neurol., 8:433 (Sep. 4, 2017).

A variety of neuroimaging techniques may be useful for the early diagnosis and/or measurement of progression of neurodegenerative disorders. Examples of such techniques include but are not limited to neuroimaging, functional MRI, structural MRI, diffusion tensor imaging (DTI) (including for example diffusion tensor measures of anatomical connectivity), [18F]fluorodeoxyglucose (FDG) PET, agents that label amyloid, [18F]F-dopa PET, radiotracer imaging, volumetric analysis of regional tissue loss, specific imaging markers of abnormal protein deposition (e.g., for AD progression), multimodal imaging, and biomarker analysis. Jon Stoessl, “Neuroimaging in the early diagnosis of neurodegenerative disease,” Transl. Neurodegener., 1: 5 (2012). Combinations of these techniques can also be used to measure disease progression.

For example, structural MRI can be used to measure atrophy of the hippocampus and entorhinal cortex in AD, as well as involvement of the lateral parietal, posterior superior temporal and medial posterior cingulate cortices. In frontotemporal dementias (FTD), structural MRI can show atrophy in frontal or temporal poles. DTI can be used to show abnormal white matter in the parietal lobes of patients with dementia with Lewy bodies (DLB) as compared to AD. Functional MRI may reveal reduced frontal but increased cerebellar activation during performance of a working memory task in FTD compared to AD. In another example, [18F]fluorodeoxyglucose (FDG) PET can show reduced glucose metabolism in parietotemporal cortex in AD. Id.

In one embodiment of the invention, the progression or onset of a neurodegenerative disorder is slowed or prevented over a defined time period, following administration of a fixed aminosterol dose according to the invention to a subject in need, as measured by a medically-recognized technique. For example, the progression or onset of a neurodegenerative disorder can be slowed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

The period of time over which the progression or onset of a neurodegenerative disorder is measured can be for example, one or more months or one or more years, e.g., about 6 months, about 1 year, about 18 months, about 2 years, about 36 months, about 3, about 4, about 5, about 6, about 7, about 8, about 9, about 10, about 11, about 12, about 13, about 14, about 15, about 16, about 17, about 18, about 19, or about 20 years, or any amount of months or years in between the values of about 6 months to about 20 years or more.

In another embodiment of the invention, a neurodegenerative disorder may be positively impacted by administration of a fixed aminosterol dose according to the invention. A “positive impact” includes for example slowing advancement of the condition, improving one or more symptoms, etc.

### V. Definitions

The following definitions are provided to facilitate understanding of certain terms used throughout this specification.

Technical and scientific terms used herein have the meanings commonly understood by one of ordinary skill in the art, unless otherwise defined. Any suitable materials and/or methodologies known to those of ordinary skill in the art can be utilized in carrying out the methods described herein.

As used in the description of the invention and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

As used herein the term “aminosterol” refers to an amino derivative of a sterol. Non-limiting examples of suitable aminosterols for use in the composition and methods disclosed herein are Aminosterol 1436, squalamine, aminosterols isolated from Squalus acanthias, and isomers, salts, and derivatives each thereof.

The term “administering” as used herein includes prescribing for administration as well as actually administering, and includes physically administering by the subject being treated or by another.

As used herein “subject,” “patient,” or “individual” refers to any subject, patient, or individual, and the terms are used interchangeably herein. In this regard, the terms “subject,” “patient,” and “individual” includes mammals, and, in particular humans. When used in conjunction with “in need thereof,” the term “subject,” “patient,” or “individual” intends any subject, patient, or individual having or at risk for a specified symptom or disorder.

As used herein, the phrase “therapeutically effective” or “effective” in context of a “dose” or “amount” means a dose or amount that provides the specific pharmacological effect for which the compound or compounds are being administered. It is emphasized that a therapeutically effective amount will not always be effective in achieving the intended effect in a given subject, even though such dose is deemed to be a therapeutically effective amount by those of skill in the art. For convenience only, exemplary dosages are provided herein. Those skilled in the art can adjust such amounts in accordance with the methods disclosed herein to treat a specific subject suffering from a specified symptom or disorder. The therapeutically effective amount may vary based on the route of administration and dosage form.

The terms “treatment,” “treating,” or any variation thereof includes reducing, ameliorating, or eliminating (i) one or more specified symptoms and/or (ii) one or more symptoms or effects of a specified disorder. The terms “prevention,” “preventing,” or any variation thereof includes reducing, ameliorating, or eliminating the risk of developing (i) one or more specified symptoms and/or (ii) one or more symptoms or effects of a specified disorder

### EXAMPLES

### Example 1—Clinical Study on Parkinson's Disease

This example describes an exemplary method of treating and/or preventing symptoms of Parkinson's disease (PD) in a clinical trial setting.

Overview:

The subjects of the trial all had PD and experienced constipation, which is a characteristic of PD. The primary objectives of the trial involving patients with PD and constipation were to evaluate the safety and pharmacokinetics of oral squalamine (ENT-01) and to identify the dose required to improve bowel function, which was used as a clinical endpoint.

Several non-constipation PD symptoms were also assessed as endpoints, including, for example, (1) sleep problems, including daytime sleepiness; (2) non-motor symptoms, such as (i) depression (including apathy, anxious mood, as well as depression), (ii) cognitive impairment (e.g., using trail making (see FIGS. 26 and 27) test and the UPDRS), (iii) hallucinations (e.g., using The University of Miami Parkinson's Disease Hallucinations Questionnaire (UM-PDHQ) and the UPDRS, (iv) dopamine dysregulation syndrome (UPDRS), (v) pain and other sensations, (vi) urinary problems, (vii) light headedness on standing, and (viii) fatigue (e.g., using Parkinson's Disease Fatigue Scale 9PFS-1t and the UPDRS); (3) motor aspects of experiences of daily living, such as (i) speech, (ii) saliva and drooling, (iii) chewing and swallowing, (iv) eating tasks, (v) dressing, (vi) hygiene, (vii) handwriting; (viii) doing hobbies and other activities, (ix) turning in bed, (x) tremor, (xi) getting out of bed, a car, or a deep chair, (xii) walking and balance, (xiii) freezing; (4) motor examination, such as (i) speech, (ii) facial expression, (iii) rigidity, (ix) finger tapping, (v) hand movements, (vi) pronation-supination movements of hands, (vii) toe tapping, (viii) leg agility, arising from chair, (ix) gait, (x) freezing of gait, (xi) postural stability, (xii) posture, (xiii) global spontaneity of movement (body bradykinesia), (xiv) postural tremor of the hands, (xv) kinetic tremor of the hands, (xvi) rest tremor amplitude, (xvii) constancy of rest tremor; (5) motor complications, such as (i) time spent with dyskinesias, (ii) functional impact of dyskinesias, (iii) time spent in the off state, (iv) functional impact of fluctuations, (v) complexity of motor fluctuations, and (vi) painful off-state dystonia.

Active Agent & Dosing:

Squlamaine (ENT-01; Enterin, Inc.) was formulated for oral administration in the trial. The active ion of ENT-01, squalamine, an aminosterol originally isolated from the dogfish shark, has been shown to reverse gastrointestinal dysmotility in several mouse models of PD. In addition, ENT-01 has been shown to inhibit the formation of aggregates of αS both in vitro, and in a C. elegans model of PD in vivo (Perni et al. 2017). In the C. elegans model, squalamine produced a complete reversal of muscle paralysis.

ENT-01 is the phosphate salt of squalamine. For this study it has been formulated as a small 25 mg coated tablet. Dosing ranged from 25 mg to 250 mg, with dosages greater than 25 mg requiring multiple pills (e.g., 50 mg=two 25 mg pills). Dosing instructions=take 60 mins before breakfast with 8 oz water. The dose was taken by each patient upon awakening on an empty stomach along with 8 oz. of water simultaneously to dopamine. The subject was not allowed to ingest any food for at least 60 minutes after study medication. The compound is highly charged and will adsorb to foodstuffs, so it was administered prior to feeding.

The phosphate salt of squalamine (ENT-01) is weakly soluble in water at neutral pH but readily dissolves at pH<3.5 (the pH of gastric fluid). Squalamine, as the highly water soluble dilactate salt has been extensively studied in over three Phase 1 and eight Phase 2 human clinical trials as an intravenous agent for the treatment of cancer and diabetic retinopathy. The compound is well tolerated in single and repeat intravenous administration, alone or in combination with other agents, to doses of at least 300 mg/m²).

In the current clinical trial, squalamine (ENT-01) was administered orally to subjects with PD who have long standing constipation. Although this trial was the first in man oral dosing study of ENT-01, humans have long been exposed to low doses of squalamine (milligram to microgram) in the various commercial dogfish shark liver extracts available as nutraceuticals (e.g., Squalamax). In addition, following systemic administration squalamine is cleared by the liver and excreted as the intact molecule (in mice) into the duodenum through the biliary tract. Drug related GI toxicology has not been reported in published clinical trials involving systemic administration of squalamine.

Squalamine (ENT-01) has limited bioavailability in rats and dogs. Based on measurement of portal blood concentrations following oral dosing of radioactive ENT-01 to rat's absorption of ENT-01 from the intestine is low. As a consequence, the principal focus of safety is on local effects on the gastrointestinal tract. However, squalamine (ENT-01) appears to be well tolerated in both rats and dogs.

The starting dose in the Stage 1 segment of the trial was 25 mg (0.33 mg/kg for a 75 kg subject). The maximum single dose in Stage 1 was 200 mg (2.7 mg/kg for a 75 kg subject). The maximum dose evaluated in Stage 2 of the trial was 250 mg/day (3.3 mg/kg/day for a 75 kg subject), and the total daily dosing exposure lasted no longer than 25 days.

The daily dosing range in the clinical trial was from 25 mg (14.7 mg/m²) to 250 mg (147 mg/m²). Oral dosing of squalamine (ENT-01), because of its low oral bioavailability, is not anticipated to reach significant plasma concentrations in human subjects. In preclinical studies, squalamine (ENT-01) exhibited an oral bioavailability of about 0.1% in both rats and dogs. In Stage 1 of this phase 2 study, oral dosing up to 200 mg (114 mg/m²) yielded an approximate oral bioavailability of about 0.1%, based on a comparison of a pharmacokinetic data of the oral dosing and the pharmacokinetic data measured during prior phase 1 studies of IV administration of squalamine.

Study Protocol:

The multicenter Phase 2 trial was conducted in two Stages: a dose-escalation toxicity study in Stage 1 and a dose range-seeking and proof of efficacy study in Stage 2.

PD symptoms were assessed using a number of different tools:

(1) Numeric Rating Scales for Pain and Swelling (scale of 0-10, with 0=no pain and 10=worst pain ever experienced);

(2) Rome-IV Criteria for Constipation (7 criteria, with constipation diagnosis requiring two or more of the following: (i) straining during at least 25% of defecations, (ii) lumpy or hard stools in at least 25% of defecations, (iii) sensation of incomplete evacuation for at least 25% of defecations, (iv) sensation of anorectal obstruction/blockage for at least 25% of defecations; (v) manual maneuvers to facilitate at least 25% of defecations; (vi) fewer than 3 defecations per week; and (vii) loose stools are rarely present without the use of laxatives;

(3) Constipation—Ease of Evacuation Scale (from 1-7, with 7=incontinent, 4=normal, and 1=manual disimpaction);

(4) Bristol Stool Chart, which is a patient-friendly means of categorizing stool characteristics (assessment of stool consistency is a validated surrogate of intestinal motility) and Stool Diary;

(5) Sleep Diary (participants completed a sleep diary on a daily basis throughout the study. The diaries included time into bed and estimated time to sleep as well as wake time and duration during the night.);

(6) I-Button Temperature Assessment. The I-Button is a small, rugged self-sufficient system that measures temperature and records the results in a protected memory section. The Thermochron I-Button DS1921H (Maxim Integrated, Dallas, Tex.) was used for skin temperature measurement. I-Buttons were programmed to sample every 10 mins., and attached to a double-sided cotton sport wrist band using Velcro, with the sensor face of the I-Button placed over the inside of the wrist, on the radial artery of the dominant hand. Subjects removed and replaced the data logger when necessary (i.e., to have a bath or shower). The value of skin temperature assessment in sleep research is that the endogenous skin warming resulting from increased skin blood flow is functionally linked to sleep propensity. From the collected data, the mesor, amplitude, acrophase (time of peak temperature), Rayleight test (an index of interdaily stability), mean waveforms are calculated.);

(7) Non-motor Symptoms Questionnaire (NMSQ);

(8) Beck Depression Inventory (BDI-II);

(9) Unified Parkinson's Disease Rating Scale (UPDRS), which consists of 42 items in four subscales (Part I=Non-Motor Aspects of Experiences of Daily Living (nM-EDL) (1.1 cognitive impairment, 1.2 hallucinations and phychosis, 1.3 depressed mood, Part II=Motor Aspects of Experiences of Daily Living (M-EDL), Part III=Motor Examination, and Part IV=Motor Complications;

(10) Mini Mental State Examination (MMSE) (see FIG. 25);

(11) Trail Making Test (TMT) Parts A and B (see FIGS. 26 and 27);

(12) The University of Miami Parkinson's Disease Hallucinations Questionnaire (UM-PDHQ);

(13) Parkinson's Disease Fatigue Scale (PFS-16);

(14) Patient Assessment of Constipation Symptoms (PAC-SYM);

(15) Patient Assessment of Constipation Quality of Life (PAC-QOL);

(16) REM Sleep Behavior Disorder Screening Questionnaire; and

(17) Parkinson's Disease Sleep Scale.

Exploratory end-points, in addition to constipation, included for example, (i) depression assessed using the Beck Depression Inventory (BDI-II) (Steer et al. 2000) and Unified Parkinson's Disease Rating Scale (UPDRS); (ii) cognition assessed using the Mini Mental State Examination (MMSE) (Palsteia et al. 2018), Unified Parkinson's Disease Rating Scale (UPDRS), and Trail Making Test (TMT); (iii) sleep and REM-behavior disorder (RBD) using a daily sleep diary, I-Button Temperature Assessment, a REM sleep behavior disorder (RBD) questionnaire (RBDQ) (Stiasny-Kolster et al. 2007), and the UPDRS; (iv) hallucinations assessed using the PD hallucinations questionnaire (PDHQ) (Papapetropoulos et al. 2008), the UPDRS, and direct questioning; (v) fatigue using the Parkinson's Disease Fatigue Scale (PFS-16) and the UPDRS; (vi) motor functions using the UPDRS; and (vii) non-motor functions using the UPDRS.

Assessments were made at baseline and at the end of the fixed dose and washout periods. Circadian system status was evaluated by continuously monitoring wrist skin temperature (Thermochron iButton DS1921H; Maxim, Dallas) following published procedures (Sarabia et al. 2008).

Based on these data, it is believed that administration of squalamine (ENT-01), a compound that can displace αS from membranes in vitro, reduces the formation of neurotoxic αS aggregates in vivo, and stimulates gastrointestinal motility in patients with PD and constipation. The observation that the dose required to achieve a prokinetic response increases with constipation severity supports the hypothesis that the greater the burden of αS impeding neuronal function, the higher the dose of squalamine (ENT-01) required to restore normal bowel function.

Study Design:

A multicenter Phase 2 trial was conducted in two Stages: a dose-escalation toxicity study in Stage 1 and a dose range-seeking and proof of efficacy study in Stage 2. The protocol was reviewed and approved by the institutional review board for each participating center and patients provided written informed consent.

Following successful screening, all subjects underwent a 14-day run-in period where the degree of constipation was assessed through a validated daily log (Zinsmeister et al. 2013) establishing baseline CSBMs/week. Subjects with an average of <3 CSBMs/week proceeded to dosing.

In Stage 1, ten (10) PD patients received a single escalating dose of squalamine (ENT-01) every 3-7 days beginning at 25 mg and continuing up to 200 mg or the limit of tolerability, followed by 2-weeks of wash-out. Duration of this part of the trial was 22-57 days. The 10 subjects in the sentinel group were assigned to Cohort 1 and participated in 8 single dosing periods. Tolerability limits included diarrhea or vomiting. A given dose was considered efficacious in stimulating bowel function (prokinetic) if the patient had a complete spontaneous bowel movement (CSBM) within 24 hours of dosing.

Each dose period was staggered, so that subjects 1-2 were administered a single dose of the drug at the lowest dose of 25 mg. Once 24 hours have elapsed, and provided there are no safety concerns, the patient was sent home and brought back on day 4-8 for the next dose. During the days the subjects are home, they completed the daily diaries and e-mailed them to the study coordinators. Subjects 3-10 were dosed after the first 2 subjects have been observed for 72 hours, i.e. on Day 4. Subjects 1-2 were also brought back on Day 4-8 and given a single dose of 50 mg. Once another 24 hours have elapsed and provided there are no safety concerns, the patients were all sent home and instructed to return on Day 7 for the next dosing level. This single dosing regimen was continued until each subject was given a single dose of 200 mg or has reached a dose limiting toxicity (DLT). DLT was the dose which induces repeated vomiting, diarrhea, abdominal pain or symptomatic postural hypotension within 24 hours of dosing.

In Stage 2, 34 patients were evaluated. First, 15 new PD patients were administered squalamine (ENT-01) daily, beginning at 75 mg, escalating every 3 days by 25 mg to a dose that had a clear prokinetic effect (CSBM within 24 hours of dosing on at least 2 of 3 days at a given dose), or the maximum dose of 175 mg or the tolerability limit. This dose was then maintained (“fixed dose”) for an additional 3-5 days. After the “fixed dose”, these patients were randomly assigned to either continued treatment at that dose or to a matching placebo, for an additional 4-6 days prior to a 2-week wash-out.

A second cohort of 19 patients received squalamine (ENT-01) escalating from 100 mg/day to a maximum of 250 mg/day without subsequent randomization to squalamine (ENT-01) or placebo. Criteria for dose selection and efficacy were identical to those used in the previous cohort.

Patient Population:

Patients were between 18 and 86 years of age and diagnosed with PD by a clinician trained in movement disorders following the UK Parkinson's Disease Society Brain Bank criteria (Fahn et al. 1987). Patients were required to have a history of constipation as defined by <3 CSBMs/week and satisfy the Rome IV criteria for functional constipation (Mearin et al. 2016) at screening, which requires 2 or more of the following: Straining during at least 25% of defecations; lumpy or hard stools in at least 25% of defecations; sensation of incomplete evacuation in at least 25% of defecations; sensation of anorectal obstruction/blockage in at least 25% of defecations; and/or manual maneuvers to facilitate at least 25% of defecations.

Baseline characteristics of patients are shown in Table 2. Patients in Stage 2 had somewhat longer duration of Parkinson's disease and higher UPDRS scores than participants in Stage 1.

TABLE 2
Baseline Characteristics of Dosed Patients

| | | | |
|---|---|---|---|
| | Stage 1** | Stage 2*** | Total |
| Characteristic | (n = 10) | (n = 34) | (n = 44) |

Sex- no. (%)

| | | | |
|---|---|---|---|
| Male | 5 (50) | 25 (73.5) | 30 (68.1) |
| Female | 5 (50) | 9 (26.5) | 14 (31.8) |
| White race-no. (%) | 8 (80) | 34 (100) | 42 (95.54) |

Age-yr

| | | | |
|---|---|---|---|
| Mean | 65.0 | 74.5 | 72.5 |
| Range | 58-70.5 | 60.6-84.2 | 58-84.2 |

Age at PD diagnosis-yr

| | | | |
|---|---|---|---|
| Mean | 61.1 | 67.7 | 66.2 |
| Range | 54.2-69 | 50.6-82.5 | 50.6-82.5 |

Duration of PD-yr

| | | | |
|---|---|---|---|
| Mean | 4.2 | 6.8 | 6.2 |
| Range | 1-11 | 0.3-17.3 | 0.3-17.3 |

Duration of constipation-yr

| | | | |
|---|---|---|---|
| Mean | 25.8 | 16.8 | 18.9 |
| Range | 1-65 | 0.5-66.0 | 0.5-66.0 |

UPDRS score

| | | | |
|---|---|---|---|
| Mean | 53.4 | 63.2 | 61.3 |
| Range | 33-88 | 24-122 | 24.0-122.0 |

Hoehn and Yahr-Stage

| | | | |
|---|---|---|---|
| Mean | 2.0 | 2.4 | 2.3 |
| Range | 2.0 | 1.0-5.0 | 1.0-5.0 |

Constipation severity* - CSBM/wk- no. (%)

| | | | |
|---|---|---|---|
| 0-1 | 8(80) | 14(41.2) | 22 (50) |
| 1.1-2 | 2 (20) | 17(50) | 19 (43.2) |
| 2.1-3 | 0 | 3 (8.8) | 3 (6.8) |

*At baseline. Baseline value is the average number of CSBMs per week calculated at the end of the 2-week run-in period.
**In Stage 1, 10 patients received single escalating doses every 3-7 days starting at 25 mg and escalating up to dose limiting toxicity (DLT) or 200 mg, whichever came first, followed by a 2-week wash-out period.
***In Stage 2, 15 patients received daily doses starting at 75 mg and escalating every 3 days up to prokinetic dose (dose producing CSBMs on at least 2 of 3 days) or 175 mg, whichever came first, followed by an additional 2-4 days at that dose (“fixed dose” period) and were then randomized to treatment at the “fixed-dose” or placebo for 4-6 days. Wash-out lasted 2 weeks. The remaining 19 patients were escalated from 100 mg to prokinetic dose or 250 mg, whichever came first, followed by an additional 2-4 days at that dose and then a 2-week wash-out period.

Safety and Adverse Event (AE) Profile:

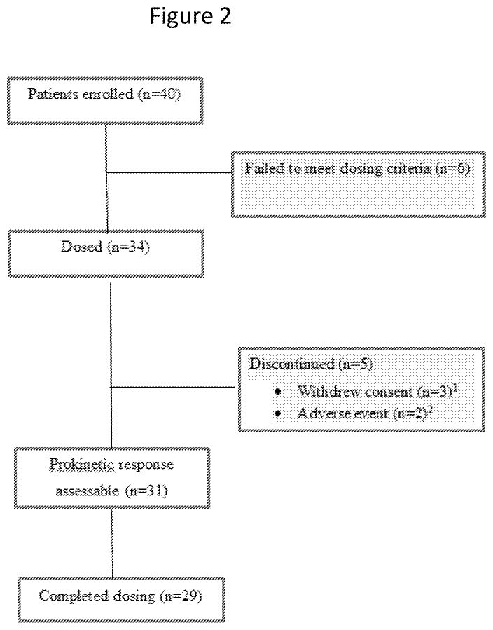

Fifty patients were enrolled and 44 were dosed. In Stage 1, 10 patients were dosed, 1 (10%) withdrew prior to completion and 9 (90%) completed dosing. In stage 2, 6 (15%) patients had ≥3 CSBM/week at the end of the run-in period and were excluded, 34 patients were dosed and bowel response was assessable in 31 (91%). Two patients (5.8%) were terminated prior to completion because of recurrent dizziness, and 3 others withdrew during dosing (8.8%): 2 because of diarrhea and 1 because of holiday. Fifteen patients were randomized. Study-drug assignments and patient disposition are shown in Table 3 and FIG. 2.

TABLE 3
Study drug assignments and adherence to treatment

| | Stage 1 | Stage 2 |
|---|---|---|
| Enrolled | 10 | 40 |
| Failed prior to dosing | 0 | 6 |
| Dosed | 10 | 34 |
| 25-200 mg | 10 | |
| 75-175 mg | | 19 |
| 100-250 mg | | 15 |
| Terminated (%) | 0 (0) | 2* (5.8) |
| Withdrew (%) | 1 (10) | 3 (8.8) |
| Completed dosing (%) | 9 (90) | 31** (91) |
| Randomized | | 15 |
| Treatment | | 6 |
| Placebo | | 9 |

The 2 patients who were terminated
**29 patients completed dosing but an additional 2 who withdrew had an assessable prokinetic end-point.

Most AEs were confined to the GI tract (88% in Stage 1 and 63% in Stage 2). The most common AE was nausea which occurred in 4/10 (40%) patients in Stage 1 and in 18/34 (52.9%) in Stage 2 (Table 2). Diarrhea occurred in 4/10 (40%) patients in Stage 1 and 15/34 (44%) in Stage 2. One patient withdrew because of recurrent diarrhea. Other GI related AEs included abdominal pain 11/44 (32%), flatulence 3/44 (6.8%), vomiting 3/44 (6.8%), worsening of acid reflux 2/44 (4.5%), and worsening of hemorrhoids 1/44 (2.2%). One patient had a lower GI bleed (Serious adverse event, SAE) during the withdrawal period. This patient was receiving aspirin, naproxen and clopidogrel at the time of the bleed, and colonoscopy revealed large areas of diverticulosis and polyps. This SAE was considered unrelated to study medication. The only other noteworthy AE was dizziness 8/44 (18%). Dizziness was graded as moderate in one patient who was receiving an alpha-adrenergic blocking agent (Terazosin). This patient was withdrawn from the study and recovered spontaneously. All other AEs resolved spontaneously without discontinuation of squalamine (ENT-01). The relationship between dose and AEs is shown in Table 4.

TABLE 4
All adverse events (n, %)

Enrolled

| | | |
|---|---|---|
| | Stage 1 (n = 10) | Stage 2 (n = 40) |
| Dosed | 10 | 34 |

GI:
Nausea

| | | |
|---|---|---|
| Mild | 4(40) | 18(52) |
| Moderate | 0 | 1(2.9) |

Diarrhea

| | | |
|---|---|---|
| Mild | 1(10) | 12(35) |
| Moderate | 3(30) | 2(5.8) |
| Severe | 0 | 1(2.9) |

Vomiting

| | | |
|---|---|---|
| Mild | 1(10) | 2(5.8) |
| Moderate | 0 | 0 |

Abdominal pain

| | | |
|---|---|---|
| Mild | 2(20) | 4(11.7) |
| Moderate | 3(30) | 2(5.8) |

Flatulence

| | | |
|---|---|---|
| Mild | 2(20) | 1(3) |
| Moderate | 0 | 0 |

Loss of appetite*

| | | |
|---|---|---|
| Mild | 1(10) | 0 |
| Moderate | 0 | 0 |

Worsening acid reflux

| | | |
|---|---|---|
| Mild | 0 | 4(11.7) |
| Moderate | 0 | 0 |

Worsening hemorrhoid

| | | |
|---|---|---|
| Mild | 0 | 1(3) |
| Moderate | 0 | 0 |

Lower GI bleed**

| | | |
|---|---|---|
| Severe | 0 | 1(2.5) |

Non-GI:
Dizziness

| | | |
|---|---|---|
| Mild | 0 | 7(20.5) |
| Moderate | 0 | 1(2.9) |

Blood in urine*

| | | |
|---|---|---|
| Mild | 1(10) | 0 |
| Moderate | 0 | 0 |

Headache

| | | |
|---|---|---|
| Mild | 1(10) | 3(8.8) |
| Moderate | 0 | 0 |

Urinary retention

| | | |
|---|---|---|
| Mild | 0 | 1(3) |
| Moderate | 0 | 0 |

Urinary tract infection

| | | |
|---|---|---|
| Mild | 0 | 1(3) |
| Moderate | 0 | 2(5.8) |

Increased urinary frequency

| | | |
|---|---|---|
| Mild | 0 | 2(5.8) |
| Moderate | 0 | 0 |

Skin lesions-rash

| | | |
|---|---|---|
| Mild | 0 | 3(8.8) |
| Moderate | 0 | 0 |

Eye infection

| | | |
|---|---|---|
| Mild | 0 | 1(3) |
| Moderate | 0 | 0 |

Difficulty falling asleep

| | | |
|---|---|---|
| Mild | 0 | 1(3) |
| Moderate | 0 | 0 |

*Unrelated to ENT-01
**colonic diverticulosis, polyp, patient on aspirin, Plavix and naproxen. Unrelated to ENT-01

TABLE 5
Common adverse events by dose

| Dose | Stage 1 | | | Stage 2 | | |
|---|---|---|---|---|---|---|
| (mg) | Diarrhea | Nausea | Vomiting | Diarrhea | Nausea | Dizziness* |
| 0 | 0 | 0 | 0 | 1 | 0 | 2 |
| 25 | 1 | 0 | 0 | — | — | — |
| 50 | 1 | 0 | 0 | — | — | — |
| 75 | 1 | 0 | 0 | 7 | 3 | 8 |
| 100 | 0 | 1 | 1 | 10 | 12 | 7 |
| 125 | 1 | 2 | 1 | 3 | 4 | 8 |
| 150 | 1 | 0 | 0 | 2 | 11 | 2 |
| 175 | 1 | 1 | 0 | 1 | 12 | 0 |
| 200 | 0 | 2 | 0 | 3 | 6 | — |
| 225 | — | — | — | 3 | 1 | |

TABLE 5
Common adverse events by dose

| Dose | Stage 1 | | | Stage 2 | | |
|---|---|---|---|---|---|---|
| (mg) | Diarrhea | Nausea | Vomiting | Diarrhea | Nausea | Dizziness* |
| 250 | — | — | — | 2 | — | |

*lightheadedness included

TABLE 6
Dose limiting toxicity criteria

| | |
|---|---|
| Diarrhea | Increase 4-6 stools/day over baseline |
| Vomiting | 3-5 episodes in 24 hours |
| Abdominal pain | Moderate pain limiting daily activities |
| Postural hypotension | Moderately symptomatic and limiting daily |
| | activities or BP <80/40 |

No formal sample size calculation was performed for Stage 1. The number of subjects (n=10) was based on feasibility and was considered sufficient to meet the objectives of the study; which was to determine the tolerability of the treatment across the range of tested doses. For Stage 2, assuming the highest proportion of spontaneous resolution of constipation with no treatment to be 0.10, 34 evaluable subjects who have measurements at both baseline and at the end of the fixed dose period provided 80% power to detect the difference between 0.10 (proportion expected if patients are not treated) and a squalamine (ENT-01) treated proportion of 0.29.

No randomization was performed for Stage 1. During the randomization period of Stage 2, subjects were randomly allocated in equal proportion (1:1) to 1 of 2 double-blind treatment groups in a block size of 4: (1) squalamine (ENT-01) at the identified fixed dose level, or (2) placebo at the identified fixed dose level.

Adverse events were coded using the current version of MedDRA. Severity of AEs were assessed by investigators according to CTCAE (v4.03): Grade 1 is labeled as Mild, Grade 2 as Moderate, and Grade 3 and above as Severe. AEs that have a possible, probable or definite relationship to study drug were defined to be related to the study drug while others were defined as “not related”. The number (percentage) of subjects who experienced an AE during escalation and fixed dosing periods were summarized by dose level and overall for each stage. The denominator for calculating the percentages were based on the number of subjects ever exposed to each dose and overall.

Effect on Bowel Function:

Cumulative responder rates of bowel function are shown in FIG. 1A. In Stage 1 (single dose), cumulative response rate increased in a dose-dependent fashion from 25% at 25 mg to a maximum of 80% at 200 mg.

In Stage 2 (daily dosing), the response rate increased in a dose-dependent fashion from 26% at 75 mg to 85.3% at 250 mg. The dose required for a bowel response was patient-specific and varied from 75 mg to 250 mg. Median efficacious dose was 100 mg. Average CSBM/week increased from 1.2 at baseline to 3.8 at fixed dose (p=2.3×10⁻⁸) and SBM increased from 2.6 at baseline to 4.5 at fixed dose (p=6.4×10⁻⁶) (Table 7). Use of rescue medication decreased from 1.8/week at baseline to 0.3 at fixed dose (p=1.33×10⁻⁵). Consistency based on the Bristol stool scale also improved, increasing from mean 2.7 to 4.1 (p=0.0001) and ease of passage increased from 3.2 to 3.7 (p=0.03). Subjective indices of wellbeing (PAC-QOL) and constipation symptoms (PAC-SYM) also improved during treatment (p=0.009 and p=0.03 respectively).

TABLE 7
Stool related indices Stage 2 (Dosed patients, n = 34)

| | Baseline | Fixed dose | |
|---|---|---|---|
| | (mean, SD) | (mean, SD) | P-value |
| CSBM* | 1.2 (0.90) | 3.8 (2.40) | 2.3 × 10⁻⁸ |
| SBM* | 2.6 (1.45) | 4.5 (2.21) | 6.4 × 10⁻⁶ |
| Suppository use* | 1.8 (1.92) | 0.3 (0.67) | 1.33 × 10⁻⁵ |
| Consistency*** | 2.7 (1.20) | 4.1 (2.13) | 0.0001 |
| Ease of passage** | 3.2 (0.73) | 3.7 (1.19) | 0.03 |
| PAC-QOL total | 1.4 (0.49) | 1.2 (0.59) | 0.009 |
| PAC-SYM | 1.3 (0.45) | 1.1 (0.49) | 0.03 |

*weekly average;
**Ease of evacuation scale, where 1-manual disimpaction and 7 = incontinent;
***Bristol stool scale 1-7, where 1 = separate hard lumps and 7 = liquid consistency

The dose that proved efficacious in inducing a bowel response was strongly related to constipation severity at baseline (p=0.00055) (FIG. 1B); patients with baseline constipation of <1 CSBM/week required higher doses for a response (mean 192 mg) than patients with ≥1 CSBM/week (mean 120 mg).

While the improvement in most stool-related indices did not persist beyond the treatment period, CSBM frequency remained significantly above baseline value (Table 8).

TABLE 8
Reversal of stool indices to baseline
during the wash-out period (Stage 2)

| | | | | P-value |
|---|---|---|---|---|
| | Baseline | Fixed dose | Wash-out | (wash-out vs. |
| | (Mean, SD) | (Mean, SD) | (Mean, SD) | baseline) |
| CSBM | 1.2 (0.90) | 3.8 (2.4) | 1.8 (1.19) | 0.01 |
| SBM | 2.6 (1.45) | 4.5 (2.21) | 3.2 (1.80) | 0.16 |
| Ease | 3.2 (0.73) | 3.7 (1.19) | 3.3 (0.81) | 0.78 |
| Consistency | 2.7 (1.20) | 4.1 (2.13) | 2.8 (1.39) | 0.85 |
| Rescue meds | 1.8 (1.92) | 0.3 (0.67) | 1.0 (1.40) | 0.13 |
| PAQ-QOL | 1.4 (0.49) | 1.2 (0.59 | 1.2 (0.63) | 0.04 |
| PAQ-SYM | 1.3 (0.45) | 1.1 (0.49) | 1.1 (0.60) | 0.11 |

The primary efficacy outcome variable was whether or not a subject was a “success” or “failure”. This is an endpoint based on subject diary entries for the “fixed dose” period prior to the endpoint assessment defined as average complete stool frequency increase by 1 or more over baseline, or 3 or more complete spontaneous stools/week. The subject was deemed a “success” if s/he met one or more of the criteria listed above, otherwise the subject was deemed a “failure”. The primary analysis was based on all subjects with a baseline assessment and an assessment at the end of the “fixed-dose” period and was a comparison of the proportion of successes with 0.10 (the null hypothesis corresponding to no treatment effect).

The proportion of subjects for whom the drug was a success was estimated with a binomial point estimate and corresponding 95% confidence interval. A secondary analysis compared the proportions of subjects who are deemed a success at the end of the randomized fixed-dose period between those randomized to the squalamine (ENT-01) arm and those randomized to the placebo arm. A Fisher's exact test was used to compare the proportions of subjects who were deemed a success at the end of randomization period between the two randomized arms

Subgroup Analysis:

Fifteen patients were randomized to treatment (n=6) or placebo (n=9) after the fixed dose period. During the 4-6 days of randomized treatment, the mean CSBM frequency in the treatment group remained higher than baseline as compared to those receiving placebo who returned to their baseline values (Table 9).

TABLE 9
CSBM frequency in the randomized cohort

| CSBM/week | Baseline | Fixed dose | Randomized | Washout |
|---|---|---|---|---|
| Treatment (n = 6) | 0.8 | 3.2 | 2.4 | 0.9 |
| Placebo (n = 9) | 1.6 | 3.3 | 1.4 | 1.6 |

CSBM increased in both groups during the treatment period and remained high in the treatment group during the randomized period but fell to baseline values in the placebo group.

Pharmakokinetics:

PK data were collected on the 10 patients enrolled in Stage 1 and 10 patients enrolled in Stage 2 to determine the extent of systemic absorption. In Stage 1, PK data were obtained at each visit, pre-medication, at 1, 2, 4, 8 and 24 hours (Table 10). In Stage 2, PK was measured on days 1 and 6 of the randomization period pre-medication, at 1, 2, 4 and 8 hours (Table 11). Based on the pharmacokinetic behavior of intravenously administered squalamine determined in prior clinical studies it is estimated that squalamine (ENT-01) exhibited oral bioavailability of less than 0.3% (Bhargava et al. 2001; Hao et al. 2003).

TABLE 10
Pharmacokinetics of orally administered squalamine (ENT-01) in Stage 1.
Stage 1

| | | | Tₘₐₓ | | | |
|---|---|---|---|---|---|---|
| | | | (hour) | T_{1/2} | | |
| Dose | # of | Cₘₐₓ | (Median | (hours) | AUC_{0-8 hr} | AUC_{0-16 hr} |
| (mg) | patients | (ng/ml) | Value) | (n) | (ng*hour/ml | (ng*hour/ml |
| 25 | 9 | 2.84 | 1.0 | 2.6 (3) | 10.8 | 19.6 |
| 50 | 10 | 3.73 | 2.0 | 3.4 (3) | 18.5 | 33.1 |
| 75 | 9 | 4.33 | 2.0 | 2.8 (2) | 18.4 | 29.8 |
| 100 | 9 | 6.18 | 2.0 | 3.9 (5) | 29.6 | 51.5 |
| 125 | 9 | 9.63 | 2.0 | 3.9 (4) | 43.1 | 77.7 |
| 150 | 7 | 6.27 | 2.0 | 5.6 (4) | 31.5 | 64.0 |
| 175 | 7 | 10.3 | 2.0 | 9.1 (6) | 49.7 | 91.2 |
| 200 | 6 | 15.1 | 2.0 | 9 0 (5) | 78.3 | 157 |

TABLE 11
Pharmacokinetics of orally administered squalamine (ENT-01) in Stage 2.
Stage 2

| | # of | | Tₘₐₓ | | |
|---|---|---|---|---|---|
| | patients | | (hour) | T_{1/2} | |
| Dose | (2 visits | Cₘₐₓ | (Median | (hours) | AUC_{0-8 hr} |
| (mg) | each) | (ng/ml) | Value) | (n) | (ng*hour/ml |
| 75 | 1 | 10.0 | 3.0 | 5.5 (1) | 59.0 |
| 100 | 4 | 17.7 | 1.0 | 4.8 (5) | 70.3 |
| 125 | | | | | |
| 150 | | | | | |
| 175 | 5 | 11.8 | 2.0 | 10 (6) | 66.8 |

The mean Cₘₐₓ, Tₘₐₓ and T_{1/2} and AUC of the squalamine ion following squalamine (ENT-01) oral dosing for Stage 1 patients. The PK analyses are only approximate, as the lower limit of the validated concentration range was 10 ng/ml; most of the measured concentrations fell below that value. The mean Cₘₐₓ, Tₘₐₓ and T½ and AUC of the squalamine ion following squalamine (ENT-01) oral dosing for Stage 2 patients. The PK analyses are only approximate, as the lower limit of the validated concentration range was 0.5 ng/ml.

CNS Symptoms in Stage 2:

An exploratory analysis was done with respect to the sleep data, the body temperature data, mood, fatigue, hallucinations, cognition and other motor and non-motor symptoms of PD. Continuous measurements within a subject were compared with a paired t-test and continuous measurements between subject groups were compared with a two-group t-test. Categorical data were compared with a chi-squared test or a Fisher's exact test if the expected cell counts are too small for a chi-squared test.

CNS Symptoms:

CNS symptoms were evaluated at baseline and at the end of the fixed dose period and the wash-out period (Table 12). Total UPDRS score was 64.4 at baseline, 60.6 at the end of the fixed dose period and 55.7 at the end of the wash-out period (p=0.002); similarly, the motor component of the UPDRS improved from 35.3 at baseline to 33.3 at the end of fixed dose to 30.2 at the end of wash-out (p=0.006). MIVISE improved from 28.4 at baseline to 28.7 during treatment and to 29.3 during wash-out (p=0.0006). BDI-II decreased from 10.9 at baseline to 9.9 during treatment and 8.7 at wash-out (p=0.10). PDHQ improved from 1.3 at baseline to 1.8 during treatment and 0.9 during wash-out (p=0.03). Hallucinations were reported by 5 patients at baseline and delusions in 1 patient. Both hallucinations and delusions improved or disappeared in 5 of 6 patients during treatment and did not return for 4 weeks following discontinuation of squalamine (ENT-01) in 1 patient and 2 weeks in another. The frequency of arm or leg thrashing reported in the sleep diary diminished progressively from 2.2 episodes/week at baseline to 0 at maximal dose. Total sleep time increased progressively from 7.1 hours at baseline to 8.4 hours at 250 mg and was consistently higher than baseline beyond 125 mg (FIG. 4). Unlike stool-related indices, the improvement in many CNS symptoms persisted during wash-out.

TABLE 12
Effect of Squalamine (ENT-01) on neurological symptoms (n = 34)

| | Baseline | Fixed dose | | Wash-out | |
|---|---|---|---|---|---|
| UPDRS | (Mean, SD) | (Mean, SD) | P-value | (Mean, SD) | P-value |
| Part 1 | 11.6 (6.51) | 10.6 (6.18)) | 0.28 | 9.5 (5.27) | 0.06 |
| (NMS) | | | | | |
| Part 2 | 14.9 (8.11) | 14.7 (9.02) | 0.77 | 14.1 (8.21) | 0.40 |
| (Daily | | | | | |
| living) | | | | | |
| Part 3 | 35.3 (14.35) | 33.3 (15.20) | 0.13 | 30.2 (13.23) | 0.005 |
| (Motor) | | | | | |
| Total | 64.4 (23.72) | 60.6 (25.60) | 0.09 | 55.7 (23.69) | 0.002 |
| MMSE | 28.4 (1.75) | 28.7 (1.9) | 0.21 | 29.3 (1.06) | 0.0006 |
| PDHQ | 1.3 (2.99) | 1.8 (3.34) | 0.45 | 0.9 (2.33) | 0.03 |
| BDI-II | 10.9 (7.12) | 9.9 (6.45) | 0.14 | 8.7 (5.19) | 0.10 |

UPDRS: Unified Parkinson's Disease Severity Score;
NMS: Non-motor symptoms;
BDI: Beck Depression Index-II;
MMSE: Mini-mental State exam.
PDHQ: Parkinson's Disease Hallucination Questionnaire

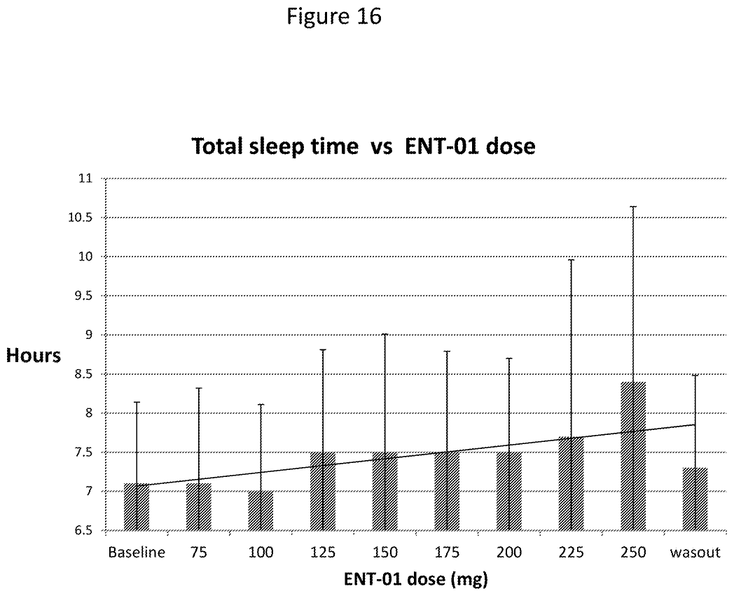

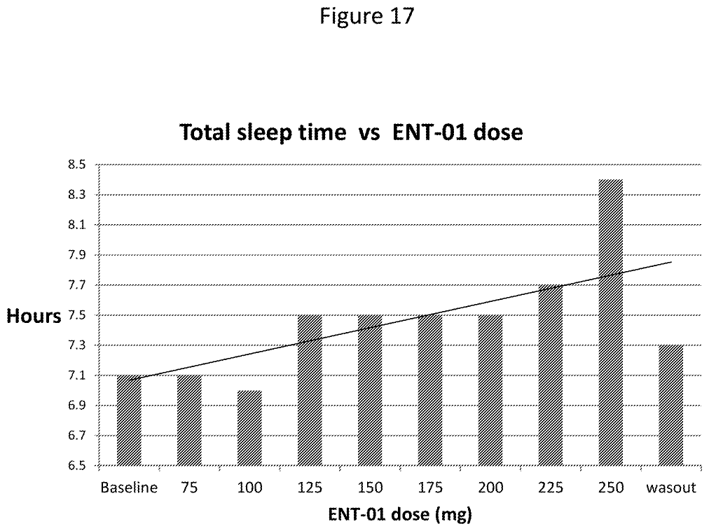

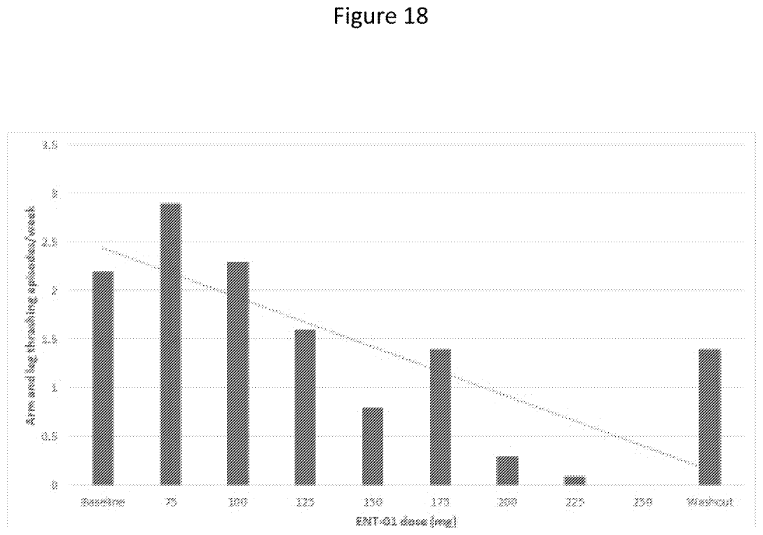

Circadian rhythm of skin temperature was evaluable in 12 patients (i.e., those who had recordings that extended from baseline through washout). Circadian system functionality was evaluated by continuously monitoring wrist skin temperature using a temperature sensor (Thermochron iButton DS1921H; Maxim, Dallas, Tex.) (Sarabia et al. 2008). A nonparametric analysis was performed for each participant to characterize DST as previously described (Sarabia et al. 2008; Ortiz-Tudela et al. 2010). Further, an analysis was done with respect to the sleep data, the body temperature data, and fatigue data. The frequency of arm or leg thrashing reported in the sleep diary diminished progressively from 2.2 episodes/week at baseline to 0 at maximal dose (100% improvement). Total sleep time increased progressively from 7.1 hours at baseline to 8.4 hours at 250 mg (an 18% increase) and was consistently higher than baseline beyond 125 mg (FIGS. 16 and 17). FIG. 18 shows REM-behavior disorder in relation to squalamine (ENT-01) dose, with arm and leg thrashing episodes (mean values) calculated using sleep diaries. The frequency of arm or leg thrashing reported in the sleep diary diminished progressively from 2.2 episodes/week at baseline to 0 at maximal dose. Unlike stool-related indices, the improvement in many CNS symptoms persisted during wash-out.

Briefly, this analysis includes the following parameters: (i) the inter-daily stability (the constancy of 24-hour rhythmic pattern over days, IS); (ii) intra-daily variability (rhythm fragmentation, IV); (iii) average of 10-minute intervals for the 10 hours with the minimum temperature (L10); (iv) average of 10-minute intervals for the 5 hours with the maximum temperature (M5) and the relative amplitude (RA), which was determined by the difference between M5 and L10, divided by the sum of both. Finally, the Circadian Function Index (CFI) was calculated by integrating IS, IV, and RA. Consequently, CFI is a global measure that oscillates between 0 for the absence of circadian rhythmicity and 1 for a robust circadian rhythm (Ortiz-Tudela et al. 2010).

A comparison was performed of circadian rhythm parameters during the baseline, fixed dose and washout periods. ENT-01 administration improved all markers of healthy circadian function, increasing rhythm stability (IS, p=0.026), relative amplitude (RA, p=0.001) and circadian function index (CFI, p=0.016), while reducing rhythm fragmentation (IV, p=0.031). The improvement persisted for several of these circadian parameters during wash-out period (IS, p=0.008 and CFI, p=0.004). (FIG. 5).

Conclusions:

This Phase 2 trial involving 50 patients with PD assessed the safety of orally administered ENT-01, and the effect on bowel function and neurologic symptoms of PD. In addition, the study aimed to identify a dose of ENT-01 that normalizes bowel function in each patient. The study achieved the objectives of identifying safety and pharmacodynamic responses of ENT-01 in PD. In addition, the study is the first proof of concept demonstration that directly targeting αS pharmacologically can achieve beneficial GI, autonomic and CNS responses.

The effective dose ranged between 75 mg and 250 mg, with 85% of patients responding within this range. This dose correlated positively with constipation severity at baseline consistent with the hypothesis that gastrointestinal dysmotility in PD results from the progressive accumulation of αS in the ENS, and that squalamine (ENT-01) can restore neuronal function by displacing αS and stimulating enteric neurons. These results demonstrate that the ENS in PD is not irreversibly damaged and can be restored to normal function.

Several exploratory endpoints were incorporated into the trial to evaluate the impact of ENT-01 on neurologic symptoms associated with PD. The UPDRS score, a global assessment of motor and non-motor symptoms, showed significant improvement. Improvement was also seen in the motor component. The improvement in the motor component is unlikely to be due to improved gastric motility and increased absorption of dopaminergic medications, since improvement persisted during the 2-week wash-out period, i.e., in the absence of study drug (Table 12).

Improvements were also seen in cognitive function (MMSE scores), hallucinations, REM-behavior disorder (RBD) and sleep. Six of the patients enrolled had daily hallucinations or delusions and these improved or disappeared during treatment in five. In one patient the hallucinations disappeared at 100 mg, despite not having reached the colonic prokinetic dose at 175 mg. The patient remained free of hallucinations for 1 month following cessation of dosing. RBD and total sleep time also improved progressively in a dose-dependent manner.

The prokinetic effect of the aminosterol squalamine appears to occur through local action of the compound on the ENS, since squalamine, the active zwitterion, is not significantly absorbed into the systemic circulation.

### Example 2—Pharmacokinetics of Intracerebroventricular (ICV) and Intravenous (IV) Administration of Aminosterol

The purpose of this example was to evaluate the in vivo distribution of Aminosterol 1436 following intracerebroventricular (ICV) and intravenous (IV) administration to rats. ICV injection is an invasive injection technique of substances directly into the cerebrospinal fluid in cerebral ventricles to bypass the blood brain barrier. The results described below detail how aminosterols such as Aminosterol 1436 localize in the brain following in vivo administration, regardless of the route of administration.

Radiolabeled Aminosterol 1436 was injected into rats by two different forms of administration: ICV and IV administration. Surprisingly, it was found that following both forms of administration, Aminosterol 1436 localized to the same portion of the brain.

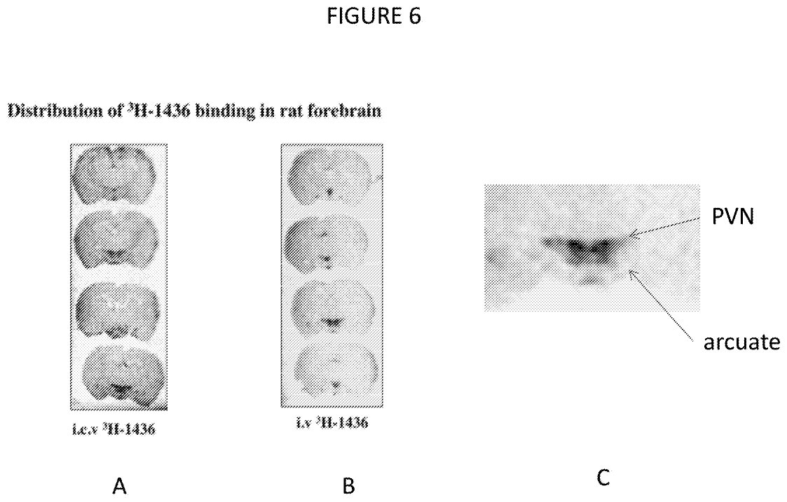

Intravenously administered Aminosterol 1436 localized in the hypothalamus. See FIG. 6B. In particular, FIG. 6B shows two panels of the distribution of ³H-Aminosterol 1436 in rat forebrain following IV administration to rats. The specific areas of ³H-Aminosterol 1436 localization include the regions below the third ventricle, in the mesiobasal hypothalamus, periventricular (PVN) and arcuate nuclei (FIG. 6C); these parts of the brain control feeding behavior and appetite and have significant involvement with neurogenesis.

Intracerebroventricularly (ICV) administered Aminosterol 1436 (ICV) localized to the same regions of the brain. See FIG. 6A. From the ventricular cerebrospinal fluid, Aminosterol 1436 is absorbed through the choroid plexus of the ventricles and vascularly transported to the same regions. In particular, FIG. 6A shows two panels of the distribution of ³H-Aminosterol 1436 binding in rat forebrain following ICV administration. The drug distribution parallels that seen with IV administration.

This example clearly demonstrates that the route of administration does not impact the site of in vivo localization of an aminosterol such as Aminosterol 1436.

### Example 3—Pharmacokinetics of Intraperitoneal Administration of Aminosterol

The purpose of this example was to evaluate the in vivo distribution of the aminosterol Aminosterol 1436 following intraperitoneal administration (IP) and ICV administration, and to determine the impact the drug has on food intake and body weight when administered IP and ICV.

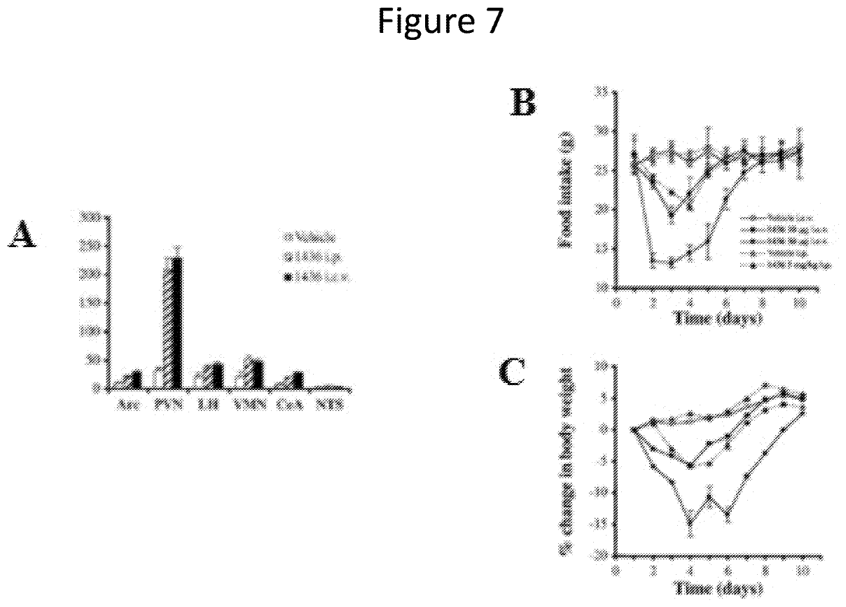

Additional data regarding in vivo distribution and the effect of the route of administration for an aminosterol is shown in FIG. 7. In particular, FIG. 7A shows the in vivo distribution of the aminosterol Aminosterol 1436 administered IP or ICV as compared to vehicle (administered IP) in the Arc (arcuate nucleus of the hypothalamus), PVN (paraventricular nucleus of the hypothalamus), LH (lateral hypothalamus), VMN (ventromedial nucleus of the hypothalamus), CcA (central amygdala), and NTS (Nucleus Tractus Solitarius, a longitudinal structure in the medulla). The data in FIG. 7A clearly show similar in vivo distribution for all areas of the brain evaluated for Aminosterol 1436 administered IP or ICV.

FIG. 7B shows the effect on food intake over a 10 day period for animals administered vehicle ICV, vehicle IP, Aminosterol 1436 at 10 and 40 μg ICV, and Aminosterol 1436 at 5 mg/kg intraperitoneal injection (IP). This experiment is relevant as the areas of the brain where the aminosterol Aminosterol 1436 localized upon administration are known to control feeding behavior and appetite. The results shown in FIG. 7B demonstrate that an aminosterol such as Aminosterol 1436 administered either IP or ICV has a significant impact on food intake, which is consistent with the area of localization of the drug in the brain.

Finally, FIG. 7C shows the percent change in body weight for the experiment detailed in FIG. 7B, with a decrease in body weight correlating with a decrease in food intake shown in FIG. 7B.

This example clearly demonstrates that the route of administration does not impact the site of in vivo localization of an aminosterol such as Aminosterol 1436.

### Example 4—Pharmacokinetics of Intranasal Aminosterol

The purpose of this example was to evaluate the in vivo distribution and function of aminosterols, such as Aminosterol 1436 and squalamine, following intranasal administration. This experiment relates to the amount of drug needed to obtain a therapeutic result, based on an IN route of administration. The results described below detail that aminosterols such as Aminosterol 1436 act at the level of the hypothalamus following in vivo administration, regardless of the route of administration.

Prior to the present invention, it was assumed that intranasal administration of an aminosterol such as Aminosterol 1436 would result in first systemic drug circulation, followed by dilution in various organs around the body, and that some small fraction of the aminosterol would eventually reach the hypothalamus. Alternatively, it was also believed that an intranasally administered aminosterol would be transported across the olfactory epithelium, enter the perineural space and track along the olfactory nerves and find its way into the cerebrospinal fluid (CSF), and subsequently to the hypothalamus.

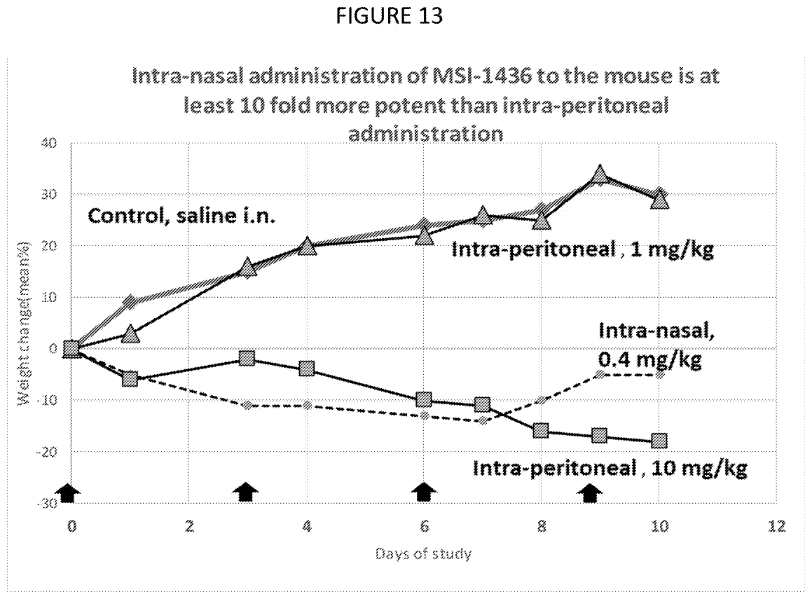

Instead, it was surprisingly found that an intranasally administered aminosterol, such as Aminosterol 1436, was not absorbed into the CSF, but rather was readily absorbed into the blood. In fact, it was unexpectedly found that intranasal administration of an aminosterol such as Aminosterol 1436 produced 10 times higher blood levels of Aminosterol 1436 than peripherally injected Aminosterol 1436. See e.g., FIG. 13. In addition, administration of the aminosterol squalamine was also found to result in a similar distribution pattern.

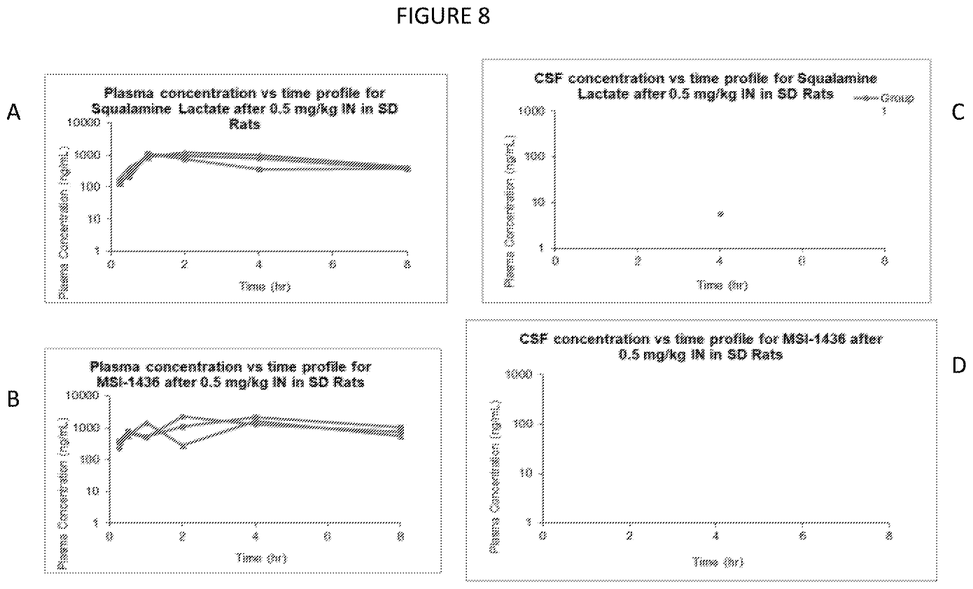

Specifically, FIG. 8A shows the plasma concentration (ng/mL) vs time for squalamine lactate after 0.5 mg/kg administered intranasally (IN) in Sprague Dawley® (SD) rats, and FIG. 8C shows the CSF concentration (ng/mL) vs time profile for squalamine lactate following 0.5 mg/kg administered IN to SD rats. Similarly, FIG. 8B shows the plasma concentration (ng/mL) vs time for Aminosterol −1436 (“MSI-1436”) after 0.5 mg/kg administered IN in SD rats, and FIG. 8D shows the CSF concentration (ng/mL) vs time profile for Aminosterol 1436 following 0.5 mg/kg administered IN to SD rats. No squalamine lactate or Aminosterol 1436 was found in CSF following intranasal administration.

Pharmacokinetic information for these tests can be found in the tables below.

TABLE 13
Summary of Aminosterol 1436 IN pharmacokinetic parameters
in plasma following 0.5 mg/kg IN dose

| PK parameters | Unit | Mean |
|---|---|---|
| T_{1/2} | h | 7.59 |
| Tₘₐₓ | h | 4.00 |
| Cₘₐₓ | ng/mL | 1677 |
| AUCₗₐₛₜ | h*ng/mL | 9371 |
| AUC_{Inf} | h*ng/mL | 17898 |
| AUC_{_{—}}_{% Extrap}_{_{—}}obs | % | 47.6 |
| MRT_{Inf}_{_{—}}obs | h | 11.1 |
| AUCₗₐₛₜ/D | h*mg/mL | 18742 |
| F | % | NA |

Data for Table 13 is also depicted in FIG. 8B.

TABLE 14
Summary of Aminosterol 1436 pharmacokinetic
parameters in CSF following 0.5 mg/kg IN dose

| PK parameters | Unit | Mean |
|---|---|---|
| T_{1/2} | h | NA |
| Tₘₐₓ | h | NA |
| Cₘₐₓ | ng/mL | NA |
| AUCₗₐₛₜ | h*ng/mL | NA |
| AUC_{Inf} | h*ng/mL | NA |
| AUC_{_{—}}_{% Extrap}_{_{—}}obs | % | NA |
| MRT_{Inf}_{_{—}}obs | h | NA |
| AUCₗₐₛₜ/D | h*mg/mL | NA |
| F | % | NA |

Data for Table 14 is also depicted in FIG. 8D.

TABLE 15
Summary of Squalamine Lactate pharmacokinetic parameters
in plasma following 0.5 mg/kg IN dose

| PK parameters | Unit | Mean |
|---|---|---|
| T_{1/2} | H | 4.55 |
| Tₘₐₓ | H | 1.00 |
| Cₘₐₓ | ng/mL | 1001 |
| AUCₗₐₛₜ | h*ng/mL | 5296 |
| AUC_{Inf} | h*ng/mL | 7863 |
| AUC_{_{—}}_{% Extrap}_{_{—}}obs | % | 32.7 |
| MRT_{Inf}_{_{—}}obs | H | 7.12 |
| AUCₗₐₛₜ/D | h*mg/mL | 10592 |
| F | % | NA |

Data for Table 15 is also depicted in FIG. 8A.

TABLE 16
Summary of Squalamine Lactate pharmacokinetic
parameters in CSF following 0.5 mg/kg IN dose

| PK parameters | Unit | Mean |
|---|---|---|
| T_{1/2} | h | NA |
| Tₘₐₓ | h | NA |
| Cₘₐₓ | ng/mL | NA |
| AUCₗₐₛₜ | h*ng/mL | NA |
| AUC_{Inf} | h*ng/mL | NA |
| AUC_{_{—}}_{% Extrap}_{_{—}}obs | % | NA |
| MRT_{Inf}_{_{—}}obs | h | NA |
| AUCₗₐₛₜ/D | h*mg/mL | NA |
| F | % | NA |

Data for Table 16 is also depicted in FIG. 8C.

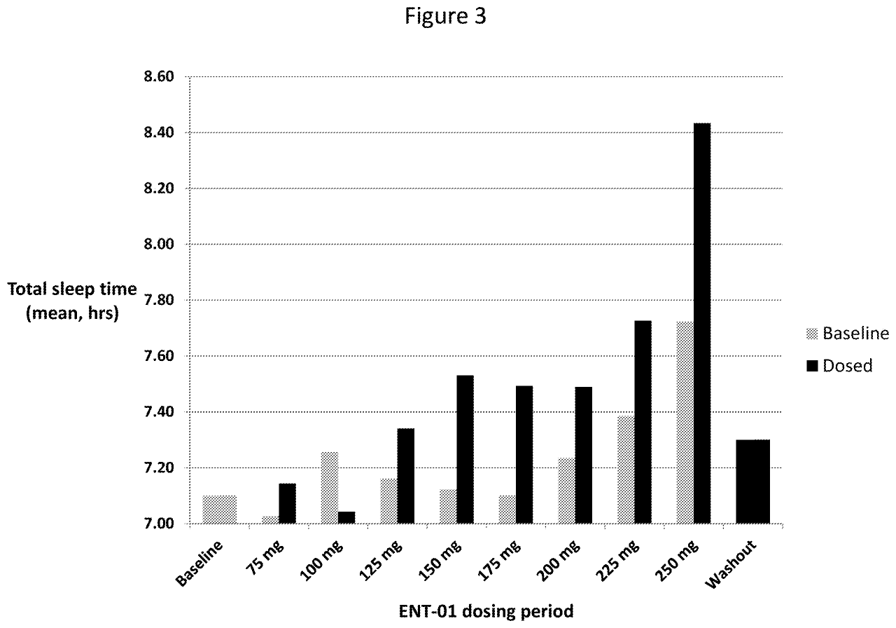

Based on the data detailed in FIGS. 3A-D, and Tables 13-16, it was concluded that the administration of aminosterols, such as Aminosterol 1436 and squalamine, results in the drug crossing the nasal epithelium and being absorbed into a very rich submucosal capillary network, from which the drug then drained into the cavernous sinus. Within the cavernous sinus, arterial and venous blood are admixed. Blood from the cavernous sinus is pumped by the internal carotid artery passing through it into the microvasculature of the brain, specifically the microvasculature of the hypothalamus, and more specifically the mesiobasal hypothalamus.

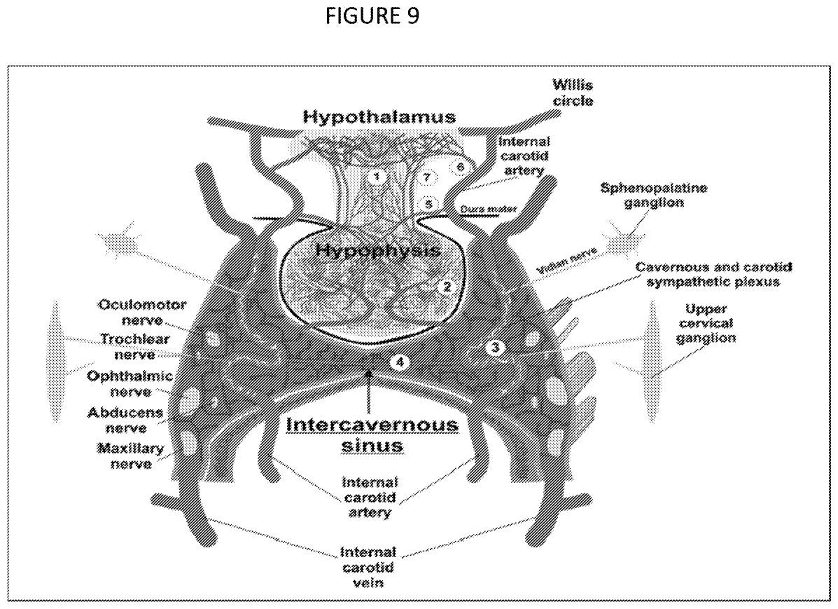

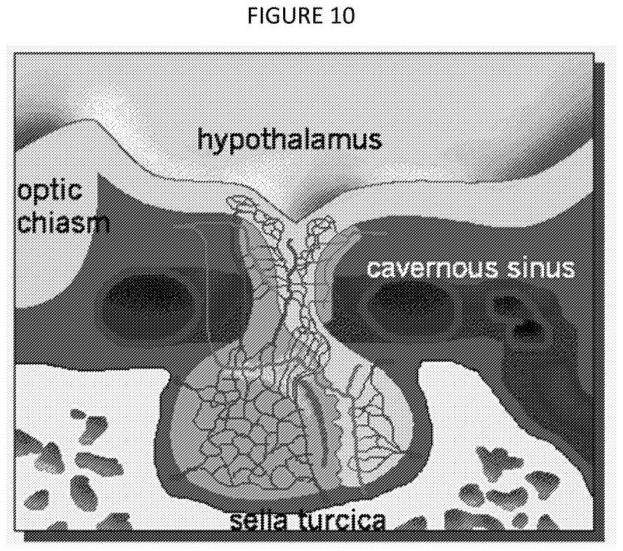

It so happens that the vascular network in the nasal cavity, the cavernous sinuses right behind the nasal cavity and the mesiobasal hypothalamus, are all incredibly close to each other (e.g., no more than 1-2 cm apart). See FIG. 9, which shows the structure of the hypothalamus, including (1) the hypophysis and (2) intercavernous sinus, (3) the internal carotid artery and internal carotid vein, (4) specific nerves, including the oculomotor nerve, trochlear nerve, ophthalmic nerve, abducens nerve, and maxillary nerve, (5) ganglions, including the sphenopalatine ganglion and upper cervical ganglion, and (6) the cavernous and carotid sympathetic plexus. FIG. 9 clearly shows that the hypothalamus is located very close to the cavernous sinus. A close up of this structure is shown in FIG. 10.

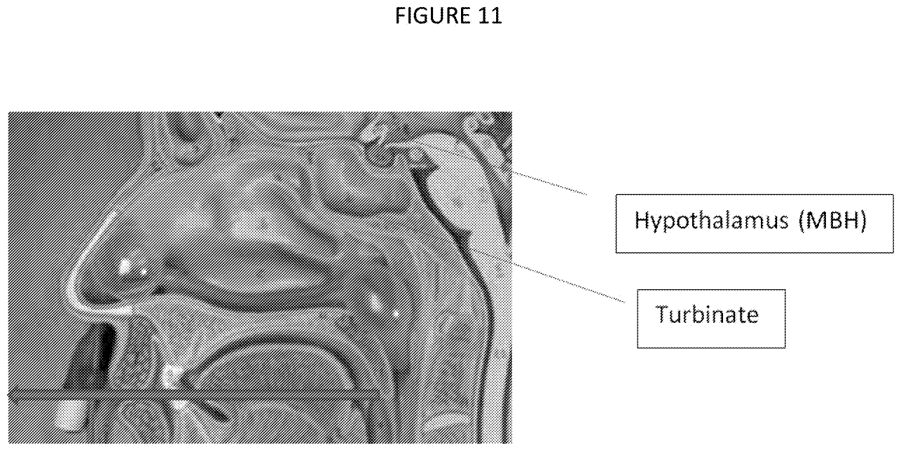

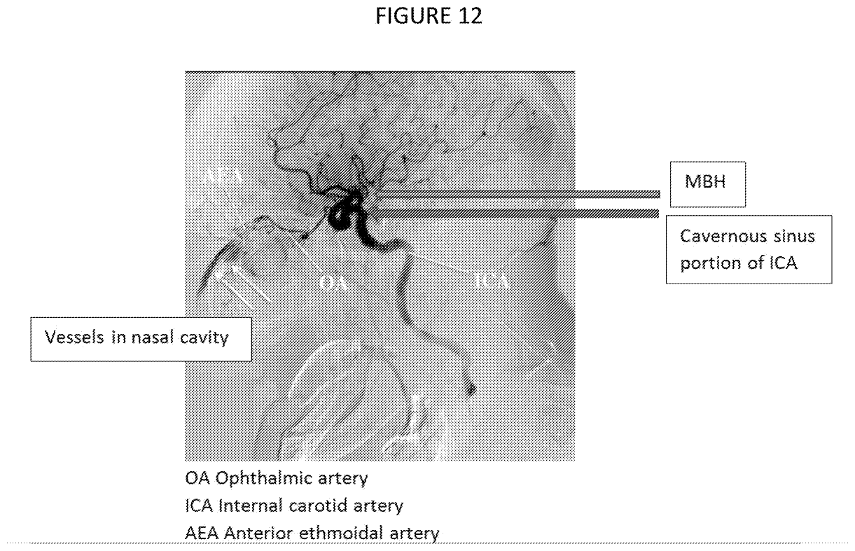

FIG. 11 shows a side-on picture through the nasal cavity showing the turbinates which are highly vascularized. This Fig. also shows how close the mesial basal hypothalamus is to this large cavity. Similarly, FIG. 12 shows the vessels in the nasal cavity, with the cavernous sinus portion of the internal carotid artery (ICA) and the medial basal hypothalamus (MBH), ophthalmic artery (OA), internal carotid artery (ICA), and anterior ethmoidal artery (AEA) identified on the figure.

This structure of the brain provides for incredibly rapid transport of an aminosterol directly into the site at which it acts, e.g., the hypothalamus. Thus, minute amounts of an aminosterol compound administered intranasally are sufficient to produce a pharmacologic effect because they are directly delivered into a tiny compartment very close to the hypothalamus. This was not known prior to the present invention.

### Example 5—Aminosterol Administration Route and Weight Loss

The purpose of this example was to evaluate and compare the impact on weight following administration of an aminosterol such as Aminosterol 1436 to mice via IP or IN.

Mice were administered: (i) intraperitoneally 1 mg/kg or 10 mg/kg of Aminosterol 1436, (ii) intranasally 0.4 mg/kg of Aminosterol 1436, or (iii) a saline control administered IN. See FIG. 13. Weight of the mice was then measured for 10 days post-administration.

The results shown in FIG. 13 demonstrate that 1 mg/kg of intraperitoneally administered Aminosterol 1436 compared similarly to the intranasally administered saline control. However, surprisingly, the IN administered Aminosterol 1436 in the amount of 0.4 mg/kg resulted in a decrease in the weight of the mice in an amount comparable to 10 mg/kg of intraperitoneally administered Aminosterol 1436. See FIG. 13.

Thus, surprisingly, intranasal administration of an aminosterol in an animal model was found to be at least 10 fold more potent than intraperitoneal administration of the same aminosterol.

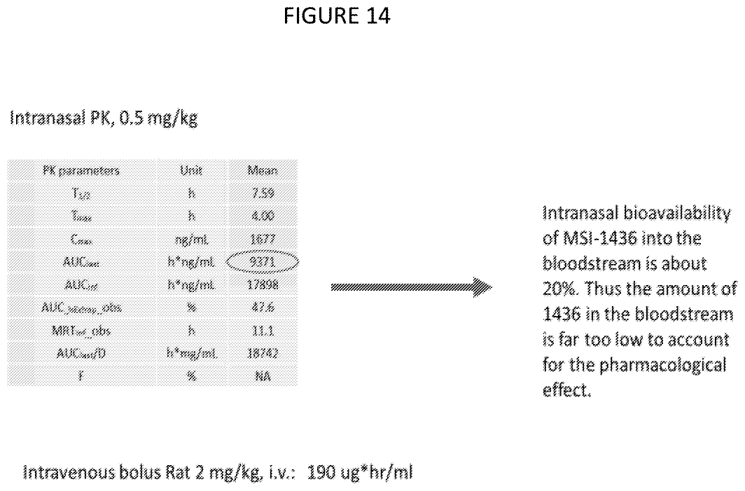

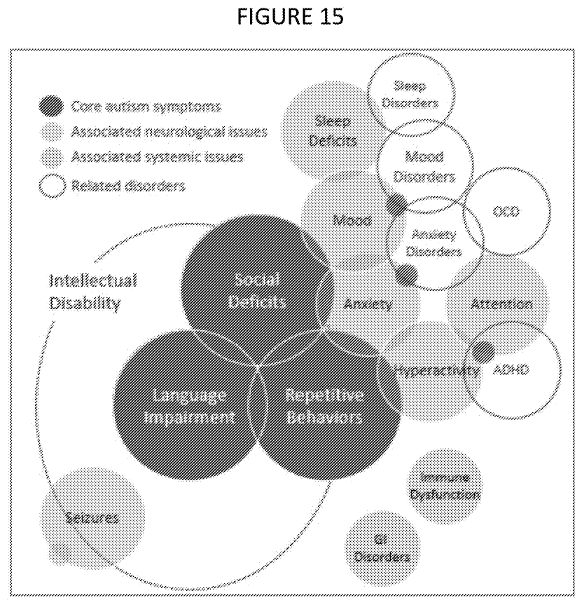

However, as pharmacokinetic testing indicates, the intranasal bioavailability of the aminosterol Aminosterol 1436 in the bloodstream is about 20%. In particular, FIG. 14 shows the PK profile in a rat following IV bolus injection of 2 mg/kg, 190 μg hr/ml of Aminosterol 1436, as compared to IN administration of 0.5 mg/kg Aminosterol 1436. The pK profile shows that IN bioavailability of Aminosterol 1436 is about 20%. Thus, the amount of aminosterol in the bloodstream is too low to account for the observed pharmacological effect. This means that when administered IN, an aminosterol is having a pharmacological effect via a mechanism other than via plasma concentration of the drug.

### Example 6—Studies in Mice

This example describes mouse studies in a PD model to elucidate details of the mechanism of action of squalamine.

Overview: Orally administered squalamine has been shown to reverse constipation in PD patients (Example 1) and inhibit α-synuclein aggregate formation in a C. elegans PD model. This Example explores the prokinetic effect of squalamine on GI motility and ENS function in wild type and velocity of colonic propagating contractile clusters (PCCs), which has improved by intraluminal squalamine treatment.

Feeding squalamine (40 mg/kg/d) to PD and wild type mice for 5 days increased their fecal pellet output. Whole cell patch clamp of single neurons in the myenteric plexus of PD mice was used to elucidate the mechanisms of prokinetic action of squalamine. PD had reduced intrinsic primary afferent neuron (IPAN) excitability; activation of these neurons produces colonic PCCs that drive peristalsis. Squalamine in turn increased IPAN excitability, which supports the local, prokinetic action of squalamine on the ENS and provides pharmacological support for the use of squalamine in the treatment of human PD, particularly in relation to constipation.

Study design: This study was designed to investigate the pharmacological activity of squalamine (10-30 μM) on the GI tract in vivo and ex vivo in several mouse models, providing physiological evidence for the therapeutic effects seen in clinical trials of PD patients with constipation and other non-motor symptoms (Hauser et al submitted for publication). All animal studies were approved by and performed in accordance with the Animal Research Ethics Board (AREB) of McMaster University and of the Florey Institute of Neuroscience and Mental Health (approval 16-029).

The short-latency direct prokinetic effects of squalamine were tested on ex vivo colon segments from commonly used control (WT) mouse strains and A53T human α-synuclein overexpressing transgenic mice, these experiments were performed and replicated in two separate laboratories. The prokinetic effect of daily oral dosing of squalamine in treating PD-related constipation was evaluated in vivo using the fecal pellet output test in WT and A53T mice. Lastly, whole cell patch clamp and the hemi-dissection protocol were used to identify differences in IPAN electrophysiology between the PD model and control and following application of squalamine. Investigators were blinded to PD model and PD control groups for IPAN experiments, but not in other studies where different strains were easily identified.

Animals:

6-8 week old male Swiss Webster, C57BL/6, and CD-1 mice (20-35 g) from Charles River Laboratories (Quebec, Canada) were used in the first portion of this study. A total of 27 mice were used for this study. In the second portion of the study, 7-month old male and female A53T human α-synuclein overexpressing transgenic mice and their WT littermate controls (25-35 g) were used. A total of 30 mice were used for this study. For the dose ranging in vivo portion of the study, a total of 100 male mice, or 5 sets of non-Tg (WT, N=10) and A53T (N=10) mice aged 7-months were used. Mice used for electrophysiological recordings were obtained from Jackson Laboratories (Maine, USA). 13-16 male PAC-Tg (SNCA^{WT}) (Stock No. 010710; FVB control) and (dbl-PAC-Tg(SNCA^{A53T}) (Stock No. 010799; FVB PD) were aged 8-9 months prior to experiments. All mice were housed 3-5 per cage on a 12 h light/dark cycle with food and water provided ad libitum and allowed a 1-week acclimation period after arrival.

Ex Vivo Colon Motility:

For the ex vivo colonic motility experiments, the colon was excised and placed within an organ-bath perfusion chamber filled with warmed, oxygenated Krebs buffer or physiological saline (35° C., 95% 02, 5% CO₂). The colon was flushed and cannulated at the oral and anal ends to a manifold and syringe to allow inflow of oxygenated Krebs buffer (or physiological saline) or Krebs and squalamine and to maintain intraluminal pressure. The height of the inflow tube at baseline measurements was parallel to the height of the colon in the organ bath (1.1 cm). Mechanical threshold defined an inflow pressure great enough to generate a contraction in under 30 sec (1.8 cm). Recordings in the first portion of the study were measured at a mechanical threshold causing a pressure differential of 2 hPa (cm H₂O). Inflow was raised 2-3 hPa above baseline and outflow was raised a minimum of 0.2 hPa above inflow. Motor patterns were recorded using a Microsoft LifeCam 3000 web camera or a Logitech Quickcam Pro camera positioned 7-8 cm above the tissue. Videos were recorded during a 20-minute Krebs control and a 20-minute Krebs+squalamine period in which solutions were added to the inflow syringe.

Spatiotemporal Maps:

Video recordings were used to construct spatiotemporal maps (STmaps) using edge detection software. STmaps are presented as heat maps showing the oral to anal direction across the y-axis and time across the x-axis (FIGS. 19A-C). Color corresponds to the changing diameter of the colon during periods of relaxation (green-yellow) and contraction (red) as contractile motor patterns occurred. ENS-dependent PCCs were defined as broad bands directed from the oral to anal ends that spanned more than 50% of the colon length. Parameters of motor patterns including, velocity, amplitude, and frequency were measured using ImageJ and Matlab (Version 12) software.

In Vivo Fecal Pellet Output:

Mice were subjected to the FPO test 1 day prior to the start of dosing with squalamine or vehicle (sterile water) (day 0). Mice were fasted for one hour and then given access to food one hour before FPO testing. On days 1-5 mice were fasted for one hour prior to oral gavage with vehicle or 20, 40, 80, or 120 mg/kg squalamine. Oral gavage occurred between 10:00 to 11:00 am daily. On day 5, the FPO test was performed 1 hour after the final dose was administered. Total number of stool pellets produced in the first 15 min and over a 60 min period was measured in each group. Stool water content was measured by comparing wet and dry weights of the stool.

Whole-Cell Patch Clamp:

Whole-cell patch clamp was performed on a hemi-dissected myenteric plexus preparation as previously described.

Statistical Analysis:

Effects of squalamine on motility and IPAN excitability in WT and PD model mice were assessed in paired experiments following Krebs control and subsequent squalamine exposure. Unpaired comparisons were performed for experiments comparing PD control and PD model strains. Percent difference was calculated by (treatment-control)/control. Data are presented as mean±SEM. Ex vivo statistical comparisons were performed using paired or unpaired, two-tailed t-tests or 1-way ANOVA using Graphpad Prism software (Version 7.0). In vivo studies were analysed using 1-way and 2-way ANOVA. Statistical significance was determined when p<0.05.

Ex Vivo Colonic Motility:

Intraluminal squalamine increased colonic motility across three mouse strains, ex vivo. To determine whether squalamine exhibits GI prokinetic activity its effects on the colons from three commonly used mouse strains Swiss Webster (8), C57BL/6 (5), and CD-1 (3) ex vivo were studied. Squalamine (10-30 μM), introduced intraluminally, increased colonic motility independently of mouse strain (FIG. 19A-C), including the C57BL/6 background for transgenic A53T PD models used in other parts of this study. The velocity of PCCs was significantly increased across all three strains following intraluminal squalamine application for 20 min (mean±SEM) (FIG. 19D). Colonic PCC sample velocity was increased by 45% from 1.14±0.10 mm/s to 1.66±0.10 mm/s in Swiss Webster mice (P<0.0001). In C57BL/6 mice, PCC velocity increased by 38% from 1.31±0.10 mm/s to 1.80±0.20 mm/s (P<0.05) after application of squalamine. PCC velocity increased by 81% from 0.96±0.1 mm/s to 1.74±0.1 mm/s (P<0.01) in CD-1 mice. Thus, squalamine has the capacity to stimulate an isolated segment of colon in such a manner that it increases the velocity of propulsive contractions while preserving the normal polarity (oral to anal) of peristalsis.

In contrast, squalamine had little effect on amplitude of colonic PCCs across the three strains (FIG. 19D). Squalamine decreased PCC amplitude in Swiss Webster mice by 3% from 0.62±0.05 cm to 0.61±0.05 cm (P=0.65). In C57BL/6 mice, squalamine increased PCC amplitude by 1% from 0.66±0.06 cm to 0.70±0.07 cm (P=0.27). Lastly, squalamine increased PCC amplitude by 12% from 0.64±0.19 cm to 0.71±0.19 cm (P=0.56) in CD-1 mice. Intraluminal squalamine also significantly increased colonic PCC frequency in the three strains (FIG. 19F). Squalamine increased PCC frequency by 35% in Swiss Webster mice from 0.009±0.001 Hz to 0.012±0.001 Hz (P<0.01). In C57BL/6 mice, PCC frequency increased by 51% from 0.007±0.001 Hz to 0.010±0.003 Hz (P=0.27) following squalamine treatment. Squalamine increased PCC frequency by 63% from 0.0099±0.0014 Hz to 0.0162±0.0026 Hz (P=0.06) in CD-1 mice. These studies demonstrate that intraluminal squalamine application increases the velocity and frequency of colonic propagating clusters (PCCs) in normal mice across several strains.

Squalamine ameliorated the reduced colonic motor activity in A53T mice, ex vivo. Homozygotic A53T human α-synuclein overexpressing mice and their wild-type (WT) littermate controls (7 months) were compared to assess the effect of α-synuclein aggregation on colonic motility using the same basic experimental procedure as in the previous section. In this engineered mouse model, human A53T expression is driven by a prion promoter resulting in the progressive accumulation of aggregates of A53T α-synuclein throughout the nervous system. When the homozygotes reach an age of 7-8 months they begin to develop progressive impairment of motor function so severe that they are eventually unable to support themselves to feed and succumb by about 16 months. In this experiment effect of intraluminal squalamine on the propulsive contraction velocity of the colon in its undistended state (baseline) and during pressure induced distension (mechanical threshold) was investigated (FIG. 20A-D).

The velocity of PCCs was reduced in colonic segments from A53T mice compared with WT controls (N=6-12 mice/group) at both the baseline state (1.2±0.2 mm/s compared to 1.7±0.3 mm/s) and upon colonic distension (1.6±0.3 mm/s compared to 3.0±0.7 mm/s) (P>0.05) (FIG. 20A), however this change was not found to be significant. Intraluminal squalamine (ENT-01, 30 μM) significantly increased PCC velocity from baseline to 2.8±0.4 mm/s in WT mice (P<0.05) and to 2.3±0.4 mm/s from baseline in A53T mice (P<0.05) (FIG. 20A). Upon colonic expansion, squalamine caused a small reduction in PCC velocity in WT (3.0±0.7 to 2.4±0.3 mm/s) and a small increase in PCC velocity in A53T mice (1.6±0.3 to 2.1±0.3 mm/s) (P>0.05). Thus, intraluminal squalamine increased the velocity of PCCs in the A53T mouse to a value that exceeded that of the WT colon evaluated in the baseline state and caused a small increase during colonic expansion. These observations suggest that colonic motility of the A53T mice is not irreversibly compromised and can be restored to normal under certain conditions by squalamine when evaluated ex vivo.

In Vivo Fecal Pellet Output:

Feeding of squalamine increased fecal pellet output, without substantially increasing water content, in vivo. To extend the ex vivo studies to the animal, both A53T and WT mice (N=10 mice/group/dose) were administered squalamine orally by gavage for 5 days (Day 1 to Day 5), at a range of doses from 0, 20, 40, 80 and 120 mg/kg. Fecal pellet output (FPO) within the first 15 minutes was measured following the gavage of vehicle control (on Day 0) or squalamine (on Day 5, at doses of 0, 20, 40, 80, and 120 mg/kg) (FIG. 20B). There was no significant difference in the FPO in the first 15 min in WT and A53T mice between Days 0 and 5, in the groups receiving only vehicle (P>0.05). Squalamine significantly increased FPO in the first 15 min in WT mice dosed with 40 and 120 mg/kg (P<0.01 and P<0.05, respectively) on Day 5 compared to WT mice on Day 0. Squalamine administration significantly increased FPO in the first 15 min in A53T mice dosed with 20, 40, and 80 mg/kg (P<0.005, P<0.0001, and P<0.01, respectively) on Day 5 compared to A53T mice on day 0. An increase in colonic motility should decrease the time of the stool within the colon and thereby increase the moisture content of the fecal pellets. Indeed, oral administration of squalamine significantly increased water content at 80 and 120 mg/kg in A53T mice (P=0.023 and 0.0004, respectively) and at 80 mg/kg in WT mice (P=0.025) (FIG. 20C-D). Thus, squalamine appears to increase colonic transit in vivo.

IPAN Excitability:

PD model mice have reduced IPAN excitability. To determine the mechanism by which squalamine stimulated intestinal motility, electrophysiological studies on single neurons within the intact myenteric plexus of PD mice and corresponding control animals were conducted using published methods. In this series of studies a mouse α-synuclein knock-out model that expressed four copies of human A53T α-synuclein driven by the endogenous α-synuclein promoter (FVB PD), with the control (FVB control) represented by a strain engineered to express two copies of the normal human α-synuclein protein, was used. The A53T strain exhibits a constipation phenotype that is more severe than that observed for the corresponding control strain.

Myenteric intrinsic primary afferent neurons (IPANs) have neurites that project to the epithelial layer where molecules present in the gut lumen can activate their chemosensitive endings to send impulses to the soma and thence to the myenteric plexus. IPAN activation and increased intrinsic excitability generate PCCs that move luminal contents in the oral to anal direction and, thus, an investigation was made into whether IPAN intrinsic excitability was reduced in FVB PD compared to FVB control mice and if squalamine administration to intestinal segments taken from FVB PD mice could facilitate IPAN excitability. For 14 AH cells from 14 FVB PD mice and 9 AH cells from 9 FVB control mice that were successfully injected with Neurobiotin at the end of the recording period, all had Dogiel type II morphology after histological processing that identified them as IPANs (FIGS. 22B and E).

Using whole-cell patch pipette recordings from IPANslthe threshold for action potential generation in response to intracellular injection of square depolarising current pulses (AP threshold), the number of action potentials generated in response to current injection of 2× threshold intensity (No. AP 2× threshold), the area under the curve (sAHP AUC) for the slow after-hyperpolarisation generated by 3 action potentials, and the resting membrane potential (RMP) were measured. IPANs from FVB PD animals were less excitable than IPANs recorded from FVB controls (FIG. 21A-H). The sample AP threshold (mean±SD (N)) was 46% smaller for FVB control (32.2±20.0 (16)) compared to FVB PD (59.2±46.1 (20)). The number of action potentials produced by a current 2× the threshold intensity was 145% larger, 3.9±5.1 (16) for FVB control versus 1.6±0.6 (19) for FVB PD. The area under of the curve for the sAHP was 42% smaller −49.5±63.7 (16) versus −85.5±78.2 (19). RMP was depolarized by 10% for the FVB control, −56±10 (16) versus −62±6 (20). Thus, as expected, the IPANs from the PD mouse strain exhibited a reduced excitability compared with those from the control animals.

Myenteric Primary Afferent Neuron Excitation:

Squalamine excites myenteric primary afferent neurons. The effect of squalamine on the excitability of an isolated intestinal segment from the FVB PD mouse was explored using divided hemi-dissection preparations so that neurons are exposed for only half the area of an opened small intestinal segment. In this experiment, the inquiry was whether squalamine influenced the activity of the IPAN through direct interaction or indirectly, by stimulating release of epithelial mediators that influenced IPAN behaviour. Addition of squalamine (30 μM) to Krebs buffer in either the epithelial or the myenteric plexus compartments of the divided hemi-dissection preparation increased IPAN excitability (FIG. 22A-F). Adding squalamine to the epithelium of the FVB PD mouse (N=15) decreased sample AP threshold by 44% from 63.7±50.4 to 35.7±22.3 pA and increased the number of APs produced by a current 2× the threshold intensity by 87% from 1.6±0.6 to 3.1±0.7. Addition of squalamine decreased the area under the curve of the sAHP by 77% from 86.8±88.2 to 20.3±25.3 mV·s, and depolarised RMP by 12% from −62±7 to −54±6 mV. Similarly, adding squalamine to the myenteric plexus of the FVB PD mouse (N=5) decreased sample AP threshold by 37% from 46.0±31.3 to 29.0±10.1 pA and increased the number of APs produced by a current 2× the threshold intensity by 214% from 1.4±0.5 to 4.4±2.8. Squalamine decreased the area under the curve of the sAHP by 87% from −71.9±60.1 to −9.6±15.1 mV·s, and depolarised RMP by 13% from −63±4 to −55±6 mV when added to the myenteric plexus of the FVB PD mouse.

These experiments demonstrate that squalamine can augment the reduced excitability of the IPANs in tissue taken from FVB PD mice. The experiments also demonstrate that squalamine can act directly on the IPAN, rather than indirectly through release of an epithelial mediator.

### Example 7—Normalization of Blood Pressure

The purpose of this example was to demonstrate the effectiveness of the use of aminosterol compositions to “normalize” blood pressure.

Subjects in Stage 2 of the clinical study described in Example 1 had their systolic blood pressure measured at various points during the clinical study. FIG. 30A graphically shows the results of measuring blood pressure of different patients both pre-medication and post medication. The results show that subjects with high blood pressure demonstrated lower blood pressure at post medication measurement, while subjects with low blood pressure demonstrated higher blood pressure at post medication measurement. Table 17 below provides the data upon which the FIG. 30A graph was generated.

TABLE 17

| | | Pre-medicating | Post med- v3 |
|---|---|---|---|
| | visit(s) | initial BP | final BP |
| series 1 | 1 + 2 | 121 | |
| series 2 | 1 + 2 | 137 | 152 ET |
| series 3 | 1 + 2 | 125.5 | 131 |
| series 4 | 1 + 2 | 122 | 120 |
| series 5 | 1 + 2 | 122.5 | 125 |
| series 6 | 1 + 2 | 121 | 132 |
| series 7 | 1 + 2 | 133 | 132 |
| series 8 | 1 + 2 | 131 | 114 |
| series 9 | 1 + 2 | 150 | 136 ET |
| series 10 | 1 + 2 | 143 | 137 |
| series 11 | 1 + 2 | 143 | 138 |
| series 12 | 1 + 2 | 140 | 140 ET |
| series 13 | 1 + 2 | 153 | 125 |
| series 14 | 1 + 2 | 132 | 142 |
| series 15 | 1 + 2 | 129.5 | 137 |
| series 16 | 1 + 2 | 124.5 | 119 |
| series 17 | 1 + 2 | 122.5 | 135 |

Similarly, FIG. 30B graphically shows a comparison between initial and final blood pressure for subjects participating in the clinical trial described in Example 1. The results show that subjects with high blood pressure demonstrated lower blood pressure at post medication measurement, while subjects with low blood pressure demonstrated higher blood pressure at post medication measurement. Table 18 below provides the data upon which the FIG. 8A graph was generated.

TABLE 18

| | | inital | final |
|---|---|---|---|
| | visits | BP | BP- v3 |
| series 1 | | 1 | |
| series 2 | 1 + 2 | 151 | 108 |
| series 3 | 1 + 2 | 109 | 111 |
| series 4 | 1 + 2 | 119.5 | 92 |
| series 5 | 1 + 2 | 134 | 132 |
| series 6 | 1 + 2 | 133.5 | 130 |
| series 7 | 1 + 2 | 152 | 142 |
| series 9 | 1 + 2 | 150 | 136 ET |
| series 10 | 1 + 2 | 143 | 137 |
| series 11 | 1 + 2 | 143 | 138 |
| series 12 | 1 + 2 | 140 | 140 ET |
| seriss 13 | 1 + 2 | 153 | 125 |
| series 14 | 1 + 2 | 132 | 142 ET |
| series 15 | 1 + 2 | 129.5 | 137 |
| series 16 | 1 + 2 | 124.5 | 119 |
| series 17 | 1 + 2 | 122.5 | 135 |
| series 18 | | 1 | |
| series 19 | 1 + 2 | 151 | 108 |
| series 20 | 1 + 2 | 109 | 111 |
| series 21 | 1 + 2 | 119.5 | 92 ET |
| series 22 | 1 + 2 | 134 | 132 |
| series 23 | 1 + 2 | 133.5 | 130 |
| series 24 | 1 + 2 | 152 | 142 ET |
| series 25 | | 147 | 138 |
| series 26 | | 114 | 130 |
| series 27 | | 113 | 121 |
| series 28 | | 110 | 110 |
| series 29 | | 121 | 113 |
| series 30 | | 128 | 122 |
| series 31 | | 132 | 147 |
| series 32 | | 149 | 144 |
| series 33 | | 166 | 154 |
| series 34 | | 116 | 115 |
| series 35 | | 138 | 110 |
| | mean | 133.4219 | 126.34375 |
| | SD | 15.13693 | 15.45479168 |
| | p = | 0.011638 | |

Moreover, FIG. 30C also graphically shows a comparison between initial BP pre medication and then 2 hours post dose. The results show that subjects with high blood pressure demonstrated lower blood pressure at post medication measurement, while subjects with low blood pressure demonstrated higher blood pressure at post medication measurement. Table 19 below provides the data upon which the FIG. 30A graph was generated.

TABLE 19

| Mean | Subject | | Initial | FD- V4 | V4 + 2 hrs |
|---|---|---|---|---|---|
| V1 + V2 | ID | subject | BP | pre dose | post dose |
| 125.5 | 3/2/2001 | subject 1 | 147 | 138 | 132 |
| 122 | 4/19/2002 | subject 2 | 114 | 130 | 126 |
| 122.5 | 5/9/2001 | subject 3 | 113 | 121 | 131 |
| 121 | 6/17/2001 | subject 4 | 110 | 110 | 115 |
| 133 | 7/9/2002 | subject 5 | 121 | 113 | 132 |
| 131 | 9/13/2001 | subject 6 | 128 | 122 | 113 |
| 143 | 12/14/2001 | subject 7 | 132 | 147 | 163 |
| 143 | 13-02-2003 | subject 8 | 149 | 144 | 138 |
| 153 | 15-10-2001 | subject 9 | 166 | 154 | |
| 132 | 16-08-2001 | subject 10 | 116 | 115 | 105 |
| 129.5 | 17-15-2001 | subject 11 | 138 | 110 | 111 |

Finally, FIG. 31 graphically shows normalization of systolic BP following a single dose of an aminosterol composition (ENT-01).

### Example 8—Constipation

This prophetic example describes an exemplary method of (i) treating constipation and/or (ii) treating and/or preventing a disorder in which constipation is a known symptom (e.g., a constipation associated disorder) in a subject.

Patients are selected based on the constipation criteria described in Example 1. Patients are grouped based on having a particular constipation associated disorder or having constipation with no underlying disorder. The groups are then subdivided into a control subgroup and a treatment subgroup. A “fixed dose” of an aminosterol or a salt or derivative thereof for each of the patients in the treatment subgroup is determined using the method described in Example 1 and in the application supra. Treatment and wash-out periods mirror Example 1. Patients are monitored for changes in the severity or occurrence of the symptoms. Patients with an underlying disorder are also monitored for changes in other symptoms associated with the disorder. Patients with no underlying disorder are monitored for the development of a constipation associated disorder.

Patients having more severe constipation, e.g., less than 1 spontaneous bowel movement per week, are started at a dose of 75 mg or more. Patients having less severe constipation, e.g., 1 or more SBM/week, are started at a lower dose of aminosterol, e.g., a starting dose of less than 75 mg, for example a dose of 25 mg/day. Thus, the starting aminosterol dose is dependent upon constipation severity. The full aminosterol dosing range is from about 1 to about 500 mg. Once a prokinetic dose has been identified for a patient, the subject is started at that same dose following drug cessation and reintroduction of drug dosing; e.g., there is no need to ramp up dosing once a prokinetic dose for a patient has been identified.

### Example 9—Hallucinations

This prophetic example describes an exemplary method of (i) treating hallucinations and/or (ii) treating and/or preventing a disorder in which hallucinations are a known symptom (a hallucination-associated disorder) in a subject.

Patients are selected based on having hallucinations. Patients are grouped based on having a particular hallucination-associated disorder or having hallucinations with no underlying disorder. The groups are then subdivided into a control subgroup and a treatment subgroup. A “fixed dose” of an aminosterol or a salt or derivative thereof for each of the patients in the treatment subgroup is determined using the method described in Example 1, using the improvement of hallucination symptoms as an endpoint. Treatment and wash-out periods mirror Example 1. Patients are monitored for changes in the severity or occurrence of the symptoms. Patients with an underlying disorder are also monitored for changes in other symptoms associated with the disorder. Patients with no underlying disorder are monitored for the development of a hallucination associated disorder.

### Example 10—REM Disturbed Sleep Disorder

This prophetic example describes an exemplary method of (i) treating REM disturbed sleep and/or (ii) treating and/or preventing a disorder in which REM disturbed sleep is a known symptom (a REM disturbed sleep associated disorder) in a subject having REM disturbed sleep.

Patients are selected based on having REM disturbed sleep. Patients are grouped based on having a particular REM disturbed sleep associated disorder or having REM disturbed sleep with no underlying disorder. The groups are then subdivided into a control subgroup and a treatment subgroup. A “fixed dose” of an aminosterol or a salt or derivative thereof for each of the patients in the treatment subgroup is determined using the method described in Example 1, using the improvement of REM disturbed sleep symptoms as an endpoint. Treatment and wash-out periods mirror Example 1. Patients are monitored for changes in the severity or occurrence of the symptoms. Patients with an underlying disorder are also monitored for changes in other symptoms associated with the disorder. Patients with no underlying disorder are monitored for the development of a REM disturbed sleep associated disorder.

### Example 11—Circadian Rhythm Dysfunction

This prophetic example describes an exemplary method of (i) treating circadian rhythm dysfunction and/or (ii) treating and/or preventing a disorder in which circadian rhythm dysfunction is a known symptom (a circadian rhythm dysfunction associated disorder) in a subject having circadian rhythm dysfunction.

Patients are selected based on having circadian rhythm dysfunction. Patients are grouped based on having a particular circadian rhythm dysfunction associated disorder or having circadian rhythm dysfunction with no underlying disorder. The groups are then subdivided into a control subgroup and a treatment subgroup. A “fixed dose” of an aminosterol or a salt or derivative thereof for each of the patients in the treatment subgroup is determined using the method described in Example 1, using either the improvement of circadian rhythm dysfunction symptoms as an endpoint. Treatment and wash-out periods mirror Example 1. Patients are monitored for changes in the severity or occurrence of the symptoms. Patients with an underlying disorder are also monitored for changes in other symptoms associated with the disorder. Patients with no underlying disorder are monitored for the development of a circadian rhythm dysfunction associated disorder.

### Example 12—Alzheimer's Disease

This prophetic example describes an exemplary method of treating and/or preventing Alzheimer's disease in a subject in need thereof.

Patients are selected based on being diagnosed with Alzheimer's disease, i.e., having Alzheimer's, or exhibiting known risk factors of Alzheimer's disease, i.e., at risk for developing Alzheimer's. Patients are grouped based on having Alzheimer's or at risk for developing Alzheimer's. The groups are then subdivided into a control subgroup and a treatment subgroup. A “fixed dose” of an aminosterol or a salt or derivative thereof for each of the patients in the treatment subgroup is determined using the method described in Example 1, using either the improvement of constipation or another symptom of Alzheimer's disease as an endpoint. Treatment and wash-out periods mirror Example 1. Patients are monitored for changes in the severity or occurrence of the symptoms. Patients having Alzheimer's are monitored for changes in other symptoms associated with the disorder. Patients at risk for developing Alzheimer's are monitored for the development of Alzheimer's.

### Example 13—Multiple System Atrophy

This prophetic example describes an exemplary method of treating and/or preventing multiple system atrophy (MSA) in a subject in need thereof.

Patients are selected based on being diagnosed with MSA, i.e., having MSA, or exhibiting known risk factors for MSA, i.e., at risk for developing MSA. Patients are grouped based on having MSA or at risk for developing MSA. The groups are then subdivided into a control subgroup and a treatment subgroup. A “fixed dose” of an aminosterol or a salt or derivative thereof for each of the patients in the treatment subgroup is determined using the method described in Example 1, using either the improvement of constipation or another symptom of MSA as an endpoint. Treatment and wash-out periods mirror Example 1. Patients are monitored for changes in the severity or occurrence of the symptoms. Patients having MSA are monitored for changes in other symptoms associated with the disorder. Patients at risk for developing MSA are monitored for the development of MSA.

### Example 14—Cognitive Impairment

This prophetic example describes an exemplary method of (i) treating cognitive impairment and/or (ii) treating and/or preventing a disorder in which cognitive impairment is a known symptom (a cognitive impairment related disorder) in a subject having cognitive impairment.

Patients are selected based on having cognitive impairment. Patients are grouped based on having a particular cognitive impairment associated disorder or having cognitive impairement with no underlying disorder. The groups are then subdivided into a control subgroup and a treatment subgroup. A “fixed dose” of an aminosterol or a salt or derivative thereof for each of the patients in the treatment subgroup is determined using the method described in Example 1, using either the improvement of constipation or cognitive impairment symptoms as an endpoint. Treatment and wash-out periods mirror Example 1. Patients are monitored for changes in the severity or occurrence of the symptoms. Patients with an underlying disorder are also monitored for changes in other symptoms associated with the disorder. Patients with no underlying disorder are monitored for the development of a cognitive impairment associated disorder.

### Example 15—Schizophrenia

This prophetic example describes an exemplary method of treating and/or preventing schizophrenia in a subject in need thereof.

Patients are selected based on being diagnosed with schizophrenia, i.e., having schizophrenia, or exhibiting known risk factors for schizophrenia, i.e., at risk for developing schizophrenia. Patients are grouped based on having schizophrenia or at risk for developing schizophrenia. The groups are then subdivided into a control subgroup and a treatment subgroup. A “fixed dose” of an aminosterol or a salt or derivative thereof for each of the patients in the treatment subgroup is determined using the method described in Example 1, using either the improvement of constipation or another symptom of schizophrenia as an endpoint.

Treatment and wash-out periods mirror Example 1. Patients are monitored for changes in the severity or occurrence of the symptoms. Patients having schizophrenia are monitored for changes in other symptoms associated with the disorder. Patients at risk for developing schizophrenia are monitored for the development of schizophrenia.

### Example 16—Autism

This prophetic example describes an exemplary method of treating and/or preventing autism in a subject in need thereof.

Patients are selected based on being diagnosed with autism. Patients are then divided into a control group and a treatment group. A “fixed dose” of an aminosterol or a salt or derivative thereof for each of the patients in the treatment group is determined using the method described in Example 1, using either the improvement of constipation or another symptom of autism as an endpoint. Treatment and wash-out periods mirror Example 1. Patients are monitored for changes in the severity or occurrence of the symptoms. Patients having autism are monitored for changes in other symptoms associated with the disorder.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

### REFERENCES

Aarsland et al., “Neuropsychiatric symptoms in patients with Parkinson's disease and dementia: frequency, profile and associated care giver stress,”J. Neurol. Neurosurg. Psychiatry, 78:36-42 (2007).
Albert et al., “The Diagnosis of Mild Cognitive Impairment Due to Alzheimer's Disease: Recommendations from the National Institute on Aging—Alzheimer's Association Workgroups on Diagnostic Guidelines for Alzheimer's Disease,” Alzheimer's & Dementia, 7(3):270-279 (2011).
Andresen et al., “Effect of 5 days linaclotide on transit and bowel function in females with constipation-predominant irritable bowel syndrome,” Gastroenterology, 133:761-8 (2007).
Antonio-Rubio et al., “Abnormal thermography in Parkinson's disease,” Parkinsonism Relat. Disord., 21:852-7 (2015).
Auyeung et al., “Ten year survival and outcomes in a prospective cohort of new onset Chinese Parkinson's disease patients,” J. Neurol. Neurosurg. Psychiatry, 83:607-11 (2012).
Berg et al., “MDS Research Criteria for Prodromal Parkinson's Disease,” Mov. Disord., 30:1600-1611 (2015).
Bhargava et al., “A phase I and pharmacokinetic study of squalamine, a novel antiangiogenic agent, in patients with advanced cancers,” Clin. Cancer Res., 7:3912-9 (2001).
Braak et al., “Idiopathic Parkinson's disease: possible routes by which vulnerable neuronal types may be subject to neuroinvasion by an unknown pathogen,” J. Neural. Transm. (Vienna), 110:517-36 (2003).
Braak et al., “Staging of brain pathology related to sporadic Parkinson's disease,” Neurobiol. Aging, 24:197-211 (2003).
Braak et al., “Gastric alpha-synuclein immunoreactive inclusions in Meissner' s and Auerbach's plexuses in cases staged for Parkinson's disease-related brain pathology,” Neuroscience Letters, 396:67-72 (2006).
Breen et al., “Sleep and circadian rhythm regulation in early Parkinson disease,” JAMA Neurol., 71:589-95 (2014).
Breen, D. P. & Lang, A. E., “Tracking the Course of Prodromal Parkinson's Disease,” Brain, 140:259-262 (2017).
Bronner et al., “Management of sexual dysfunction in Parkinson's disease.” Ther Adv Neurol Disord. 2011 November; 4(6): 375-383.
Brundin et al., “Therapeutic approaches to target alpha-synuclein pathology,” Exp. Neurology, 298(B):225-235 (2017).
Chang et al., “A Meta-Analysis of Genome-Wide Association Studies Identifies 17 New Parkinson's Disease Risk Loci,” Nat. Genet., 49:1511-1516 (2017).
Cordell et al., “Alzheimer's Association Recommendations for Operationalizing the Detection of Cognitive Impairment During the Medicare Annual Wellness Visit in a Primary Care Setting,” Alzheimer's & Dementia, 9(2):141-150 (2013).
Darweesh et al., “Trajectories of Prediagnostic Functioning in Parkinson's Disease,” Brain, 140:429-441 (2017).
Diederich et al., “Hallucinations in Parkinson disease,” Nat. Rev. Neurol., 5:331-42 (2006).
Dean et al., “Physiology of Penile Erection and Pathophysiology of Erectile Dysfunction” Urol Clin North Am. 2005 November; 32(4): 379-v.
Fahn S ER, Members of the UPDRS Development Committee. UNIFIED PARKINSON'S DISEASE RATING SCALE. Florham Park, N.J.: Macmillan Health Care Information (1987).
Fereshtehnejad et al. 2014
Frank et al., “Psychometric validation of a constipation symptom assessment questionnaire,” Scand. J. Gastroenterol., 34:870-7 (1999).
Friedman J H, Akbar U., “Psychosis in Parkinson's disease: unexplained observations in a seemingly simple model,” Expert Rev. of Neurotherapeutics, 16:595-6 (2016).
Frisina et al., “The neuropathological basis for depression in Parkinson's disease” Parkinsonism Relat Disord. 2009 February; 15(2): 144-148.
Giuliano et al., “Dopamine and male sexual function,” J. Eur Urol., 40(6):601-8. (December 2001).
Gjerstad et al., “Excessive daytime sleepiness in Parkinson disease: is it the drugs or the disease?” Neurology, 67:853-8 (2006).
Goetz C G, Stebbins G T., “Risk factors for nursing home placement in advanced Parkinson's disease,” Neurology, 43:2227-9 (1993).
Hao et al., “A Phase I and pharmacokinetic study of squalamine, an aminosterol angiogenesis inhibitor,” Clin. Cancer Res., 9:2465-71 (2003).
Heaton et al., “Defecation frequency and timing, and stool form in the general population: a prospective study,” Gut, 33:818-24 (1992).
Holmqvist et al., “Direct evidence of Parkinson pathology spread from the gastrointestinal tract to the brain in rats,” Acta Neuropathol., 128:805-20 (2014).
Holmqvist et al., “Direct evidence of Parkinson pathology spread from the gastrointestinal tract to the brain in rats,” Acta Neuropathol., 128:805-20 (2014).
Hughes et al., “Accuracy of clinical diagnosis of idiopathic Parkinson's disease: a clinico-pathological study of 100 cases,” J. Neurol. Neurosurg. Psychiatry, 55:181-4 (1992).
Hughes et al., “Associations of Probable REM Sleep Behavior Disorder, Constipation, and Hyposmia with PD” (2017), in Movement Disorder Society: Proceedings of the International Congress of Parkinson's Disease and Movement Disorders; Marsili et al., 2018. Diagnostic Criteria for Parkinson's Disease: From James Parkinson to the Concept of Prodromal Disease. Front. Neurol., Online 23 Mar. 2018.
Fleming et al., “Impaired baroreflex function in mice overexpressing alpha-synuclein,” Front. Neurol., 4:103 (2013).
Fukui et al., “Protective effect of telmisartan against progressive oxidative brain damage and synuclein phosphorylation in stroke-resistant spontaneously hypertensive rats,” J. Stroke Cerebrovasc. Dis., 23(6):1545-53 (2014).
Frisina et al., “The neuropathological basis for depression in Parkinson's disease” Parkinsonism Relat Disord. 2009 February; 15(2): 144-148.
Griffioen et al., “Dietary Energy Intake Modifies brainstem Autonomic Dysfunction Caused by Mutant α-Synuclein,” Neurobiol Aging., 34(3):928-935 (2013).
Hall et al., Guyton and Hall Textbook of Medical Physiology. 12. edition. Saunders/Elsevier, 2010. ISBN 1416045740.
Igarashi et al., “Systemic Sclerosis Associated With Multiple Sclerosis,” Arch Dermatol., 125(8):1145 (1989).
Joers et al., “Modeling and imaging cardiac sympathetic neurodegeneration in Parkinson's disease” Am J Nucl Med Mol Imaging 2014; 4(2): 125-159.
Jack et al., “Introduction to the Recommendations from the National Institute on Aging—Alzheimer's Association Workgroups on Diagnostic Guidelines for Alzheimer's Disease,” Alzheimer's & Dementia, 7(3):257-262 (2011).
Jennings et al., “Hyposmic and Dopamine Transporter-Deficit Prodromal Cohort,” JAMA Neurol., 74:933-940 (2017).
Jorm, A. F., “The Informant Questionnaire on Cognitive Decline in the Elderly (IQCODE): A Review,” International Psychogeriatrics, 16:1-19 (2004).
Kumar “Epidemiology of hypertension” Clinical Queries: Nephrology Volume 2, Issue 2, April-June 2013, Pages 56-61
Kirkevold, O. & Selbaek, G., “The Agreement Between the MMSE and IQCODE Tests in a Community-Based Sample of Subjects Aged 70 Years or Older Receiving In-Home Nursing: An Explorative Study,” Dement Geriatr. Cogn. Dis. Extra, 5(1):32-41 (2015).
Lewis S J, Heaton K W., “Stool form scale as a useful guide to intestinal transit time,” Scand. J. Gastroenterol., 32:920-4 (1997).
Lin et al., “Risk of Parkinson's disease following severe constipation: a nationwide population-based cohort study,” Parkinsonism Relat. Disord., 20:1371-5 (2014).
Lingala et al. “Parkinson's disease and comorbidity: A US national perspective” International Parkinson and Movement Disorder Society. 21^{st} International Congress. 2017.
Lineback, “High Blood Pressure Predicts Motor Decline in Parkinson's Disease,” Neurology Reviews, 24(6):41 (2016)
Madrid-Navarro et al., “Multidimensional Circadian Monitoring by Wearable Biosensors in Parkinson's Disease,” Front. Neurol., 9:157 (2018).
Mahlknecht et al., “Prodromal Parkinson's Disease as Defined per MDS Research Criteria in the General Elderly Community,” Mov. Disord., 31:1405-1408 (2016).
Marquis et al., “Development and validation of the Patient Assessment of Constipation Quality of Life questionnaire,” Scand. J. Gastroenterol., 40:540-51 (2005).
Marsili et al., “Diagnostic Criteria for Parkinson's Disease: From James Parkinson to the Concept of Prodromal Disease,” Front. Neurol., Online 23 Mar. 2018.
Mearin et al., “Bowel Disorders,” Gastroenterology, 150(6):1393-1407 (2016).
McKhann et al., “The Diagnosis of Dementia Due to Alzheimer's Disease: Recommendations from the National Institute on Aging—Alzheimer' s Association Workgroups on Diagnostic Guidelines for Alzheimer's Disease,” Alzheimer's & Dementia, 7(3):263-269 (2011).
S. Morairty, “Detecting Neurodegenerative Diseases Before Damage Is Done,” SRI International (Jul. 26, 2013) (https://www.sri.com/blog/detecting-neurodegenerative-diseases).
Ondo et al., “Daytime sleepiness and other sleep disorders in Parkinson's disease,” Neurology, 57:1392-6 (2001).
Ondo et al., “Placebo-controlled trial of lubiprostone for constipation associated with Parkinson disease,” Neurology, 78:1650-4 (2012).
Ortiz-Tudela et al., “Ambulatory circadian monitoring (ACM) based on thermometry, motor activity and body position (TAP): a comparison with polysomnography,” Physiol. Behav., 126:30-8 (2014).
Pagano, G., “Imaging in Parkinson's Disease,” Clin. Med., 16:371-375 (2016).
Palma et al., “Treatment of autonomic dysfunction in Parkinson disease and other synucleinopathies”. Mov. Disord. (Review), 33(3):372-90 (March 2018).
Palsetia et al., “The Clock Drawing Test versus Mini-mental Status Examination as a Screening Tool for Dementia: A Clinical Comparison,” Indian J. Psychol. Med., 40:1-10 (2018).
Papapetropoulos et al., “A questionnaire-based (UM-PDHQ) study of hallucinations in Parkinson's disease,” BMC Neurol., 8:21 (2008).
Papatsoris et al., 2006
Perni et al., “A natural product inhibits the initiation of alpha-synuclein aggregation and suppresses its toxicity,” PNAS, USA, 114:E1009-E17 (2017).
Phillips et al., “Alpha-synuclein-immunopositive myenteric neurons and vagal preganglionic terminals: autonomic pathway implicated in Parkinson's disease?” Neuroscience, 153:733-50 (2008).
Phillips et al., “Alpha-synuclein-immunopositive myenteric neurons and vagal preganglionic terminals: autonomic pathway implicated in Parkinson's disease?” Neuroscience, 153:733-50 (2008).
Plassman et al., “Prevalance of Cognitive Impairment Without Dementia in the United States,” Ann. Intern. Med., 148(6):427-434 (2009).
Postuma et al., “The New Definition and Diagnostic Criteria of Parkinson's Disease,” Lancet Neurol., 15:546-548 (2016).
Postuma et al., “The New Definition and Diagnostic Criteria of Parkinson's Disease,” Lancet Neurol. 15:546-548 (2016).
Rocca et al., “The Role of T1-Weighted Derived Measures of Neurodegeneration for Assessing Disability Progression in Multiple Sclerosis,” Front Neurol., 8:433 (Sep. 4, 2017).
Sato et al., “Telmisartan reduces progressive oxidative stress and phosphorylated α-synuclein accumulation in stroke-resistant spontaneously hypertensive rats after transient middle cerebral artery occlusion,” J Stroke Cerebrovasc. Dis., 23(6): 1554-63 (2014).
Sarabia et al., “Circadian rhythm of wrist temperature in normal-living subjects A candidate of new index of the circadian system,” Physiol. Behav., 95:570-80 (2008).
Scorza et al., “Cardiac abnormalities in Parkinson's disease and Parkinsonism” J Clin Neurosci. 2018 July; 53:1-5.
Shehata et al., “Neuronal stimulation induces autophagy in hippocampal neurons that is involved in AMPA receptor degradation after chemical long-term depression,” J Neurosci., 32:10413-22 (2012).
Shen et al., “Role of the Autonomic Nervous System in Modulating Cardiac Arrhythmias,” Circulation Research, 114(6):1004-1021 (2014).
Singleton et al., “Association between cardiac denervation and parkinsonism caused by α-synuclein gene triplication” Brain, Volume 127, Issue 4, 1 Apr. 2004, Pages 768-772.
Jon Stoessl, “Neuroimaging in the early diagnosis of neurodegenerative disease,” Transl. Neurodegener., 1: 5 (2012).
Steer et al., “Use of the Beck Depression Inventory-II with depressed geriatric inpatients,” Behav. Res. Ther., 38:311-8 (2000).
Stiasny-Kolster et al., “The REM sleep behavior disorder screening questionnaire—a new diagnostic instrument,” Movement disorders: Official J. of the Movement Dis. Soc., 22:2386-93 (2007).
Stolzenberg et al., “A Role for Neuronal Alpha-Synuclein in Gastrointestinal Immunity,” J. Innate Immun., 9:456-63 (2017).
Svensson et al., “Does vagotomy reduce the risk of Parkinson's disease: The authors reply” Ann. Neurol., 78:1012-3 (2015).
Tang et al., “Loss of mTOR-Dependent Macroautophagy Causes Autistic-like Synaptic Pruning Deficits,” Neuron, 83(5):1131-1143 (2014).
Videnovic A, Golombek D., “Circadian Dysregulation in Parkinson's Disease,” Neurobiol. Sleep Circadian Rhythms, 2:53-8 (2017).
West et al., “Squalamine increases vagal afferent firing frequency in aging mice,” J. of the Canadian Association of Gastroenterology, 1 (2018).
Wimo et al., “The worldwide economic impact of dementia 2010,” Alzheimer's Dement., 9: 1-11 (2013).
Zahodne et al., “Mood and motor trajectories in Parkinson's disease: multivariate latent growth curve modeling,” Neuropsychology, 26:71-80 (2012).
Zinsmeister et al., “Pharmacodynamic and clinical endpoints for functional colonic disorders: statistical considerations,” Dig. Dis. Sci., 58:509-18 (2013).
Learning About Parkinson's Disease, NIH Nat'l Human Genome Research Inst., https://www.genome.gov/10001217/learning-about-parkinsons-disease/ (last updated Mar. 14, 2014); Parkinson Disease, NIH U.S. Nat'l Library of Med.: Genetics Home Reference, https://ghr.nlm.nih.gov/condition/parkinson-disease#genes (last updated Jul. 3, 2018).

1. A method of treating, preventing, and/or slowing the onset or progression in a subject in need of a condition selected from the group consisting of Parkinson's disease (PD) and/or a related symptom, autism spectrum disorder (ASD) and/or a related symptom, Alzheimer's disease (AD) and/or a related symptom, depression and/or a related symptom, or constipation and/or a related symptom,
wherein the method comprises administering to the subject a therapeutically effective amount of at least one aminosterol, or a salt or derivative thereof, provided that the method of administering does not comprise oral administration.

2. A method of treating, preventing, and/or slowing the onset or progression in a subject in need of a condition selected from the group consisting of schizophrenia and/or a related symptom, erectile dysfunction and/or a related symptom, high blood pressure (HBP) and/or a related condition, low blood pressure (LBP) and/or a related condition, multiple system atrophy and/or a related symptom, Cardiac Conduction Defects and/or a related symptom
wherein the method comprises administering to the subject a therapeutically effective amount of at least one aminosterol, or a salt or derivative thereof.

3. The method of claim 2, wherein the method of administration comprises oral, nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof.

4. The method of claim 1, wherein the therapeutically effective amount of at least one aminosterol, or a salt or derivative thereof:
(a) comprises about 0.1 to about 20 mg/kg body weight of the subject; and/or
(b) comprises about 0.1 to about 15 mg/kg body weight of the subject; and/or
(c) comprises about 0.1 to about 10 mg/kg body weight of the subject; and/or
(d) comprises about 0.1 to about 5 mg/kg body weight of the subject; and/or
(e) comprises about 0.1 to about 2.5 mg/kg body weight of the subject; and/or
(f) comprises about 0.001 to about 500 mg/day; and/or
(g) comprises about 0.001 to about 250 mg/day; and/or
(h) comprises about 0.001 to about 125 mg/day; and/or
(i) comprises about 0.001 to about 50 mg/day; and/or
(j) comprises about 0.001 to about 25 mg/day; and/or
(k) comprises about 0.001 to about 10 mg/day; and/or
(l) comprises about 0.001 to about 6 mg/day administered intranasal; and/or
(m) comprises about 0.001 to about 4 mg/day administered intranasal; and/or
(n) comprises about 0.001 to about 2 mg/day administered intranasal; and/or
(o) comprises about 0.001 to about 1 mg/day administered intranasal; and/or
(p) comprises about 1 to about 300 mg/day administered orally; and/or
(q) comprises about 25 to about 300 mg/day administered orally.

5. A method of treating a subject in need, wherein the subject has a condition amenable to treatment and/or prevention and/or amelioration with an aminosterol, comprising determining a dose of an aminosterol or a salt or derivative thereof for the subject, wherein the aminosterol dose is determined based on the effectiveness of the aminosterol dose in improving or resolving a symptom being evaluated, wherein the symptom is related to the condition, followed by administering the aminosterol dose to the subject for a period of time, wherein the method comprises:
(a) identifying a symptom to be evaluated;
(b) identifying a starting aminosterol dose for the subject;
(c) administering an escalating dose of the aminosterol to the subject over a period of time until an effective dose for the symptom being evaluated is identified, wherein the effective dose is the dose where improvement or resolution of the symptom is observed, and fixing the aminosterol dose at that level for that particular symptom in that particular subject; and
(d) optionally wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

6. The method of claim 5, wherein the aminosterol or a salt or derivative thereof is administered orally, intranasally, or a combination thereof.

7. The method of claim 5, wherein:
(a) the aminosterol or a salt or derivative thereof is administered orally and the starting dose of the aminosterol or a salt or derivative thereof ranges from about 1 mg up to about 175 mg/day; and/or
(b) the aminosterol or a salt or derivative thereof is administered orally and the starting oral aminosterol dose is about 25 mg/day; and/or
(c) the aminosterol or a salt or derivative thereof is administered orally and the dose of the aminosterol or a salt or derivative thereof for the subject following escalation is fixed at a range of from about 1 mg up to about 500 mg/day; and/or
(d) the aminosterol or a salt or derivative thereof is administered orally and the dose of the aminosterol or a salt or derivative thereof for the subject following escalation is fixed at a dose of about 1, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, or about 500 mg/day; and/or
(e) the aminosterol or a salt or derivative thereof is administered orally and the starting oral aminosterol dose is about 10, about 15, about 20, about 25, about 30, about 35, about 40, about 45, about 60, about 65, about 70, or about 75 mg/day; and/or
(f) the aminosterol or a salt or derivative thereof is administered orally and the dose of the aminosterol or a salt or derivative thereof is escalated in about 25 mg increments; and/or
(g) the aminosterol or a salt or derivative thereof is formulated for oral administration in a composition which is a liquid, capsule, or tablet designed to disintegrate in either the stomach, upper small intestine, or more distal portions of the intestine; and/or
(h) the aminosterol or a salt or derivative thereof is administered intranasally and the starting dose of the aminosterol or a salt or derivative thereof ranges from about 0.001 mg to about 3 mg/day; and/or
(i) the aminosterol or a salt or derivative thereof is administered intranasally and the dose of the aminosterol or a salt or derivative thereof for the subject following escalation is fixed at a range of from about 0.001 mg up to about 6 mg/day; and/or
(j) the aminosterol or a salt or derivative thereof is administered intranasally and the dose of the aminosterol or a salt or derivative thereof for the subject following escalation is a dose which is subtherapeutic when administered orally or by injection; and/or
(k) the aminosterol or a salt or derivative thereof is administered intranasally and the dose of the aminosterol or a salt or derivative thereof is escalated in increments of about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg; and/or
(l) the aminosterol or a salt or derivative thereof is formulated for intranasal administration in a composition which is a dry powder nasal spray or liquid nasal spray; and/or
(m) the dose of the aminosterol or a salt or derivative thereof is escalated every about 3 to about 5 days; and/or
(n) the dose of the aminosterol or a salt or derivative thereof is escalated every about 1 to about 14 days; and/or
(o) the dose of the aminosterol or a salt or derivative thereof is escalated every about 1, about 2, about 3, about 4, about 5, about 6, about 7, about 8, about 9, about 10, about 11, about 12, about 13, or about 14 days; and/or
(p) the dose of the aminosterol or a salt or derivative thereof is escalated about 1×/week, about 2×/week, about every other week, or about 1×/month; and/or
(q) the fixed dose of the aminosterol or a salt or derivative thereof is administered once per day, every other day, once per week, twice per week, three times per week, four times per week, five times per week, six times per week, every other week, or every few days; and/or
(r) the fixed dose of the aminosterol or a salt or derivative thereof is administered for a first defined period of time of administration, followed by a cessation of administration for a second defined period of time, followed by resuming administration upon recurrence of ASD or a symptom of ASD; and/or
(s) the fixed dose of the aminosterol or a salt or derivative thereof is incrementally reduced after the fixed dose of aminosterol or a salt or derivative thereof has been administered to the subject for a defined period of time; and/or
(t) the fixed dose of the aminosterol or a salt or derivative thereof is varied plus or minus a defined amount to enable a modest reduction or increase in the fixed dose; and/or
(u) the fixed dose of the aminosterol or a salt or derivative thereof is varied plus or minus a defined amount to enable a modest reduction or increase in the fixed dose, and the fixed aminosterol dose is increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%; and/or
(v) the fixed dose of the aminosterol or a salt or derivative thereof is administered once per day, every other day, once per week, twice per week, three times per week, four times per week, five times per week, six times per week, every other week, or every few days;

8. The method of claim 5, wherein:
(a) the starting dose of the aminosterol or a salt or derivative thereof is higher if the condition or related symptom being evaluated is severe; and/or
(b) progression or onset of the condition is slowed, halted, or reversed over a defined period of time following administration of the fixed escalated dose of the aminosterol or a salt or derivative thereof, as measured by a medically-recognized technique; and/or
(c) the progression or onset of the condition, and/or a related symptom, is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; and/or
(d) the condition is positively impacted by the fixed escalated dose of the aminosterol or a salt or derivative thereof, as measured by a medically-recognized technique; and/or
(e) the fixed escalated dose of the aminosterol or a salt or derivative thereof reverses dysfunction caused by the condition and treats, prevents, improves, and/or resolves the condition-related symptom being evaluated; and/or
(g) the improvement or resolution of the condition-related symptom is measured using a clinically recognized scale or tool; and/or
(i) the improvement in the condition-related symptom is at least about 3%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale or tool.

9. The method of claim 5, wherein the condition is selected from the group consisting of a neurodegenerative disease, Parkinson's disease, Alzheimer's disease, schizophrenia, autism spectrum disorder, depression, erectile dysfunction, cardiac conduction defects, high blood pressure, low blood pressure, cognitive impairment, multiple system atrophy, and constipation.

10. The method of claim 5, wherein the condition is neurodegeneration, and the subject is at risk for developing, or is suffering from, neurodegeneration, and wherein:
(a) the method results in treating, preventing, and/or delaying the progression and/or onset of neurodegeneration in the subject; and/or
(b) the neurodegeneration is age-related; and/or
(c) the neurodegeneration is correlated with age-related dementia; and/or
(d) the neurodegeneration is correlated with a neurodisease; and/or
(e) the neurodegeneration is correlated with one or more conditions or diseases selected from the group consisting of Alzheimer's disease, Parkinson's disease, Lewy Body dementia, fronto temperal dementia, supranuclear palsy, multi-system atrophy, Parkinsonism, amyotrophic lateral sclerosis (ALS), Huntington's Disease, schizophrenia, Friedreich's ataxia, Multiple sclerosis (MS), spinal muscular atrophy, progressive nuclear palsy, degenerative processes associated with aging, dementia of aging, Guadeloupian Parkinsonism, spinocerebellar ataxia, and vascular dementia; and/or
(f) progression or onset of the neurodegeneration is slowed, halted, or reversed over a defined time period following administration of the fixed escalated dose of the aminosterol or a salt or derivative thereof, as measured by a medically-recognized technique; and/or
(g) the neurodegeneration is positively impacted by the fixed escalated dose of the aminosterol or a salt or derivative thereof, as measured by a medically-recognized technique; and/or
(h) the positive impact and/or progression of neurodegeneration is measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis; and/or
(i) the progression or onset of neurodegeneration is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

11. The method of claim 5, wherein:
(a) the subject is at risk of developing, or suffers from, a condition which is a sleep disorder or sleep disturbance, and wherein administration of the fixed escalated aminosterol dose decreases the occurrence of at least one symptom of the sleep disorder or disturbance; and/or
(b) the subject suffers from, is or at risk of developing, a condition which is depression, wherein:
(i) the method results in improvement in a subject's depression, as measured by one or more clinically-recognized depression rating scale; and/or
(ii) the improvement is in one or more depression characteristics selected from the group consisting of mood, behavior, bodily functions such as eating, sleeping, energy, and sexual activity, and/or episodes of sadness or apathy; and/or
(iii) the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%; and/or
(c) the subject suffers from, is or at risk of developing, a condition which is autism spectrum disorder, wherein:
(i) the method results in improvement in one or more of the subject's autism characteristics or behaviors, as measured by a clinically-recognized rating scale; and/or in one or more autism characteristics or behaviors selected from the group consisting of social skills, repetitive behaviors, speech, nonverbal communication, sensory sensitivity, behavior, social interaction, and communication skills, as measured using a clinically-recognized scale;
(ii) the improvement a subject experiences following treatment in one or more autism characteristics or behaviors is about 5, about 10, about 15, about 20, about 25, about 30, about 35, about 40, about 45, about 50, about 55, about 60, about 65, about 70, about 75, about 80, about 85, about 90, about 95 or about 100%; and/or
(d) the subject suffers from, is or at risk of developing, a condition which is schizophrenia, and wherein:
(i) the method results in improvement in one or more schizophrenia characteristics or behaviors, as measured using a clinically recognized rating scale; and/or
(ii) the schizophrenia characteristics or behaviors are selected from the group consisting of unclear or confusing thinking, reduced social engagement, reduced emotional expression, abnormal social behavior, failure to understand reality, lack of motivation, and hearing voices that others do not hear, as measured using a clinically-recognized scale; and/or
(iii) the improvement a subject experiences in one or more schizophrenia characteristics or behaviors following treatment is about 5, about 10, about 15, about 20, about 25, about 30, about 35, about 40, about 45, about 50, about 55, about 60, about 65, about 70, about 75, about 80, about 85, about 90, about 95 or about 100%; and/or
(e) the subject suffers from, is or at risk of developing, a condition which is an inflammatory disease or condition caused by excessive expression or concentration of alpha synuclein in the subject, wherein:
(i) the method results in a decrease in intensity of inflammation, blood levels of inflammatory markers, inflammatory markers in tissue, number of inflammatory cells in tissue, or any combination thereof, as compared to a control or as compared to the qualitative or quantitative amount from the same patient or subject prior to treatment; and/or
(ii) the method results in a decrease in concentration of alpha synuclein in the subject; and/or
(iii) the method results in a decrease in concentration of alpha synuclein in the subject and the decrease in alpha-synuclein concentration in is measured qualitatively, quantitatively, or semi-quantitatively by one or more methods selected from the group consisting of:
(1) first determining the concentration of alpha-synuclein in a tissue sample from the subject prior to treatment, followed by: (i) after treatment determining the alpha-synuclein concentration in the same tissue type from the same subject; or (ii) after treatment comparing the alpha-synuclein concentration in the same tissue type to a control;
(2) measuring the intensity of inflammation over time;
(3) measuring the amount of inflammatory markers over time;
(4) measuring the amount of inflammatory markers in blood, plasma, or tissue over time, either qualitatively or quantitatively;
(5) measuring the amount of one or more inflammatory marker cytokines in blood, plasma, or tissue over time, either qualitatively or quantitatively;
(6) measuring the amount of one or more plasma markers of inflammation such as TNF, IL-8, or CRP in blood, plasma, or tissue over time, either qualitatively or quantitatively; and
(7) measuring the amount of inflammatory cells in blood, plasma, or tissue over time, either qualitatively or quantitatively; and/or
(iv) the decrease in alpha-synuclein concentration is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

12. The method of claim 5, wherein the condition is Parkinson's disease and the symptom to be evaluated is selected from the group consisting of:
(a) at least one non-motor aspect of experiences of daily living as defined by Part I of the Unified Parkinson's Disease Rating Scale selected from the group consisting of cognitive impairment, hallucinations and psychosis, depressed mood, anxious mood, apathy, features of dopamine dysregulation syndrome, sleep problems, daytime sleepiness, pain, urinary problems, constipation problems, lightheadedness on standing, and fatigue;
(b) at least one motor aspect of experiences of daily living as defined by Part II of the Unified Parkinson's Disease Rating Scale selected from the group consisting of speech, saliva and drooling, chewing and swallowing, eating tasks, dressing, hygiene, handwriting, turning in bed, tremors, getting out of a bed, a car, or a deep chair, walking and balance, and freezing;
(c) at least one motor symptom identified in Part III of the Unified Parkinson's Disease Rating Scale selected from the group consisting of speech, facial expression, rigidity, finger tapping, hand movements, pronation-supination movements of hands, toe tapping, leg agility, arising from chair, gait, freezing of gait, postural stability, posture, body bradykinesia, postural tremor of the hands, kinetic tremor of the hands, rest tremor amplitude, and constancy of rest tremor; and/or
(d) at least one motor complication identified in Part IV of the Unified Parkinson's Disease Rating Scale selected from the group consisting of time spent with dyskinesias, functional impact of dyskinesias, time spent in the off state, functional impact of fluctuations, complexity of motor fluctuations, and painful off-state dystonia; and/or
(e) constipation;
(f) depression;
(g) cognitive impairment;
(h) sleep problems or sleep disturbances;
(i) circadian rhythm dysfunction;
(j) hallucinations;
(k) fatigue;
(l) REM disturbed sleep;
(m) REM behavior disorder;
(n) erectile dysfunction;
(o) apnea;
(p) postural hypotension;
(q) correction of blood pressure or orthostatic hypotension;
(r) nocturnal hypertension;
(s) regulation of temperature;
(t) improvement in breathing or apnea;
(u) correction of cardiac conduction defect;
(v) amelioration of pain;
(w) restoration of bladder sensation and urination;
(x) urinary incontinence; and/or
(z) control of nocturia.

13. The method of claim 12, wherein the symptom to be evaluated is constipation, and the fixed escalated aminosterol dose for constipation is defined as the aminosterol dose that results in a complete spontaneous bowel movement (CSBM) within 24 hours of dosing on at least 2 of 3 days at a given dose.

14. The method of claim 5, comprising a first aminosterol which is aminosterol 1436 or a salt or derivative thereof administered intranasally and a second aminosterol which is squalamine or a salt or derivative thereof administered orally.

15. The method of claim 5, wherein:
(a) the method is applied to a patient population susceptible to excessive expression of alpha-synuclein, resulting in an excessive or high concentration of alpha-synuclein; and/or
(b) each aminosterol dose is taken on an empty stomach, optionally within two hours of the subject waking; and/or
(c) no food is taken or consumed after about 60 to about 90 minutes of taking the aminosterol dose; and/or
(d) the aminosterol or a salt or derivative thereof is a pharmaceutically acceptable grade of at least one aminosterol or a pharmaceutically acceptable salt or derivative thereof; and/or
(e) the aminosterol composition further comprises one or more of the following: an aqueous carrier; a buffer; a sugar; and/or a polyol compound; and/or
(f) the subject is a human; and/or
(g) the subject is at risk, or is a member of a patient population at risk, of developing the condition.

16. The method of claim 1, wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

17. The method of claim 5, wherein the aminosterol or a salt or derivative thereof is selected from the group consisting of:
(a) isolated from the liver of Squalus acanthias; and/or
(b) a squalamine isomer; and/or
(c) squalamine or a pharmaceutically acceptable salt thereof; and/or
(d) a phosphate salt of squalamine; and/or
(e) aminosterol 1436 or a pharmaceutically acceptable salt thereof; and/or
(f) an isomer of aminosterol 1436; and/or
(g) a phosphate salt of aminosterol 1436; and/or
(h) comprises a sterol nucleus and a polyamine attached at any position on the sterol, such that the molecule exhibits a net charge of at least +1; and/or
(i) comprises a bile acid nucleus and a polyamine, attached at any position on the bile acid, such that the molecule exhibits a net charge of at least +1; and/or
(h) a derivative modified to include one or more of the following:
(i) substitutions of the sulfate by a sulfonate, phosphate, carboxylate, or other anionic moiety chosen to circumvent metabolic removal of the sulfate moiety and oxidation of the cholesterol side chain;
(ii) replacement of a hydroxyl group by a non-metabolizable polar substituent, such as a fluorine atom, to prevent its metabolic oxidation or conjugation; and
(iii) substitution of one or more ring hydrogen atoms to prevent oxidative or reductive metabolism of the steroid ring system; and/or
(k) a derivative of squalamine modified through medical chemistry to improve bio-distribution, ease of administration, metabolic stability, or any combination thereof; and/or
(l) a synthetic aminosterol; and/or
(m) is selected from the group consisting of:

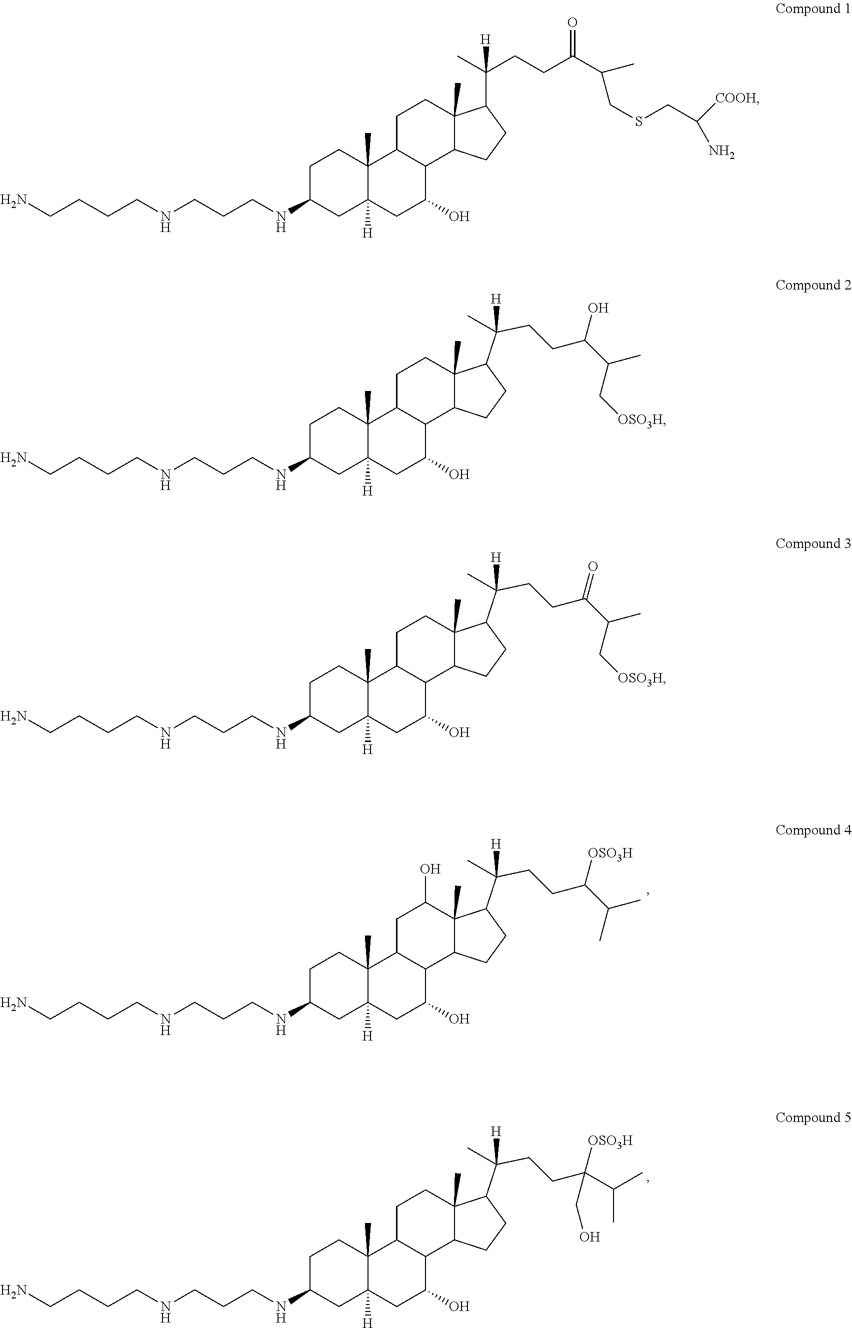

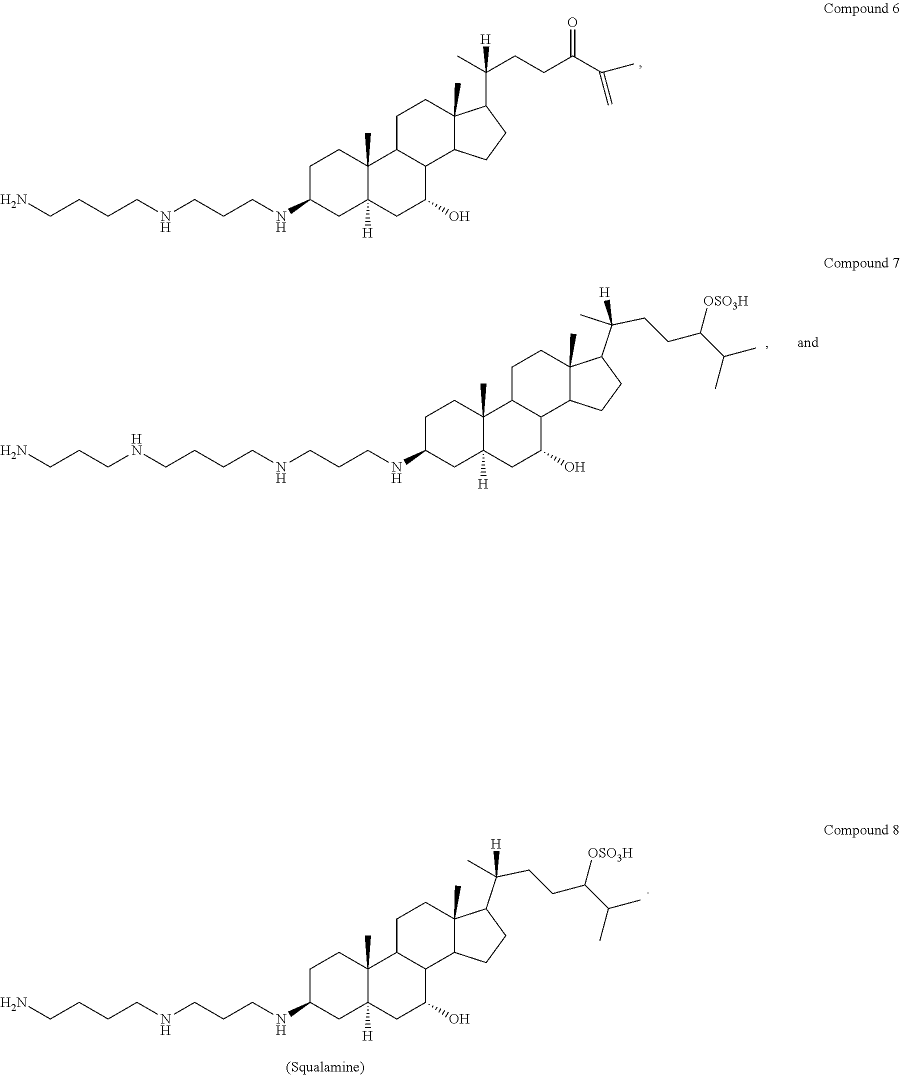

18. The method of claim 5, wherein the aminosterol is squalamine.

19. The method of claim 5, wherein the aminosterol is a phosphate salt of squalamine.

20. The method of claim 5, wherein the aminosterol is aminosterol 1436.
